SEQUENCE LISTING (772 sequences)

SEQ ID NO: 1 - protein, 94 aa, Homo Sapien
VSDVPRDLEVVAATPTSLLISWDAPAVTVRYYRITYGETGGNSPVQEFTVPGSKSTATISGLKPGVDYTITVYAVTGRGDSPASSKPISINYRT

SEQ ID NO: 2 - protein, 172 aa, Artificial Sequence
  GP41 external domain
AVGIGAMFLGFLGAAGSTMGAASVTLTVQARQLLSGIVQQQNNLLRAIEAQQHLLQLTVWGIKQLQARVLAVERYLKDQQLLGIWGCSGKLICTTAVPWNASWSNKSLDEIWNNMTWMEWEREIDNYTGLIYTLIEESQNQQEKNEQELLELDKWASLWNWFDITNWLWYIK

SEQ ID NO: 3 - protein, 96 aa, Artificial Sequence
  Adnectin
GVSDVPRDLEVVAATPTSLLISWDAPAVTVHSYHIQYWPLGSYQRYQVFSVPGSKSTATISGLKPGVEYQIRVYAETGRGESDQSLGWIQIGYRTE

SEQ ID NO: 4 - protein, 96 aa, Artificial Sequence
  Adnectin
GVSDVPRDLEVVAATPTSLLISWDAPAVTVHSYHIQYWPLGSYQRYQVFSVPGSKSTATISGLKPGVEYQIRVYAETGRGESDQSLGWIQIGYRTP

SEQ ID NO: 5 - protein, 96 aa, Artificial Sequence
  Adnectin
GVSDVPRDLEVVAATPTSLLISWDAPAVTVHSYHIQYWPLGSYQRYQVFSVPGSKSTATISGLKPGVEYQIRVYAETGGADSDQSFGWIQIGYRTP

SEQ ID NO: 6 - protein, 215 aa, Artificial Sequence
  bnAb 10E8v4 Light Chain
ASELTQDPAVSVALKQTVTITCRGDSLRSHYASWYQKKPGQAPVLLFYGKNNRPSGIPDRFSGSASGNRASLTITGAQAEDEADYYCSSRDKSGSRLSVFGGGTKLTVLSQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 7 - protein, 459 aa, Artificial Sequence
  bnAb 10E8v4 Heavy Chain
EVRLVESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWSGYPPGEEYFQDWGQGTLVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 8 - protein, 461 aa, Artificial Sequence
  bnAb 10E8v4 Heavy Chain
EVRLVESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWSGYPPGEEYFQDWGQGTLVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 580 aa, Artificial Sequence
  bnAb-Adnectin fusion polypeptide
EVRLVESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWSGYPPGEEYFQDWGQGTLVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGVSDVPRDLEVVAATPTSLLISWDAPAVTVHSYHIQYWPLGSYQRYQVFSVPGSKSTATISGLKPGVEYQIRVYAETGGADSDQSFGWIQIGYRTP

SEQ ID NO: 10 - protein, 576 aa, Artificial Sequence
  bnAb-Adnectin fusion polypeptide
EVRLVESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWSGYPPGEEYFQDWGQGTLVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPESPEPETPEDESPEPETPEDEGVSDVPRDLEVVAATPTSLLISWDAPAVTVHSYHIQYWPLGSYQRYQVFSVPGSKSTATISGLKPGVEYQIRVYAETGGADSDQSFGWIQIGYRTP

SEQ ID NO: 11 - protein, 336 aa, Artificial Sequence
  bnAb-Adnectin fusion polypeptide
ASELTQDPAVSVALKQTVTITCRGDSLRSHYASWYQKKPGQAPVLLFYGKNNRPSGIPDRFSGSASGNRASLTITGAQAEDEADYYCSSRDKSGSRLSVFGGGTKLTVLSQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSGGGGSGGGGSGGGGSGGGGSGVSDVPRDLEVVAATPTSLLISWDAPAVTVHSYHIQYWPLGSYQRYQVFSVPGSKSTATISGLKPGVEYQIRVYAETGGADSDQSFGWIQIGYRTP

SEQ ID NO: 12 - protein, 332 aa, Artificial Sequence
  bnAb-Adnectin fusion polypeptide
ASELTQDPAVSVALKQTVTITCRGDSLRSHYASWYQKKPGQAPVLLFYGKNNRPSGIPDRFSGSASGNRASLTITGAQAEDEADYYCSSRDKSGSRLSVFGGGTKLTVLSQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSESPEPETPEDESPEPETPEDEGVSDVPRDLEVVAATPTSLLISWDAPAVTVHSYHIQYWPLGSYQRYQVFSVPGSKSTATISGLKPGVEYQIRVYAETGGADSDQSFGWIQIGYRTP

SEQ ID NO: 13 - protein, 20 aa, Artificial Sequence
  Adnectin
HSYHIQYWPLGSYQRYQVFS

SEQ ID NO: 14 - protein, 25 aa, Artificial Sequence
  Adnectin
EYQIRVYAETGGADSDQSMGWIQIG

SEQ ID NO: 15 - protein, 20 aa, Artificial Sequence
  Adnectin
LSYHIQYWPLGLYQAYQVFS

SEQ ID NO: 16 - protein, 25 aa, Artificial Sequence
  Adnectin
EYQIRVYAETGRGESPASFGWIQIG

SEQ ID NO: 17 - protein, 20 aa, Artificial Sequence
  Adnectin
HAYHIQYWPLGFYQGYQVFS

SEQ ID NO: 18 - protein, 25 aa, Artificial Sequence
  Adnectin
EYQIRVYAETGLGDAHQSLGWIQIG

SEQ ID NO: 19 - protein, 20 aa, Artificial Sequence
  Adnectin
LAYHIQYWPLGWYQRYQIFS

SEQ ID NO: 20 - protein, 20 aa, Artificial Sequence
  Adnectin
LAYHIQYWPLGWYQRYQVFS

SEQ ID NO: 21 - protein, 20 aa, Artificial Sequence
  Adnectin
HFYHIQYWPLGLYHLYQVFS

SEQ ID NO: 22 - protein, 20 aa, Artificial Sequence
  Adnectin
YSYHIQYWPLGWYHRYQVFS

SEQ ID NO: 23 - protein, 25 aa, Artificial Sequence
  Adnectin
EYQIRVYAETGADDPVQALGWIQIG

SEQ ID NO: 24 - protein, 20 aa, Artificial Sequence
  Adnectin
RCYHIQYWPLGLYPLYQVFS

SEQ ID NO: 25 - protein, 25 aa, Artificial Sequence
  Adnectin
EYQIRVYAETGDESSVQPFGWIQIG

SEQ ID NO: 26 - protein, 20 aa, Artificial Sequence
  Adnectin
YSYHIQYWPLGWYQRYQVFS

SEQ ID NO: 27 - protein, 25 aa, Artificial Sequence
  Adnectin
EYQIRVYAETDGGRSQQSFGWIQIG

SEQ ID NO: 28 - protein, 20 aa, Artificial Sequence
  Adnectin
SSYHIQYWPLGAYQRYQVFS

SEQ ID NO: 29 - protein, 20 aa, Artificial Sequence
  Adnectin
HAYHIQYWPLGLYQRYQVFS

SEQ ID NO: 30 - protein, 20 aa, Artificial Sequence
  Adnectin
HAYYIQYWPLGSYQFYQVFA

SEQ ID NO: 31 - protein, 20 aa, Artificial Sequence
  Adnectin
HSYHIQYWPLGSYLRYQVFS

SEQ ID NO: 32 - protein, 20 aa, Artificial Sequence
  Adnectin
LSYHIQYWPLGFYQRYQVFS

SEQ ID NO: 33 - protein, 20 aa, Artificial Sequence
  Adnectin
SAYHIQYWPLGWYHRYQIFS

SEQ ID NO: 34 - protein, 20 aa, Artificial Sequence
  Adnectin
YSYHIQYWPLGAYSRHQLFS

SEQ ID NO: 35 - protein, 25 aa, Artificial Sequence
  Adnectin
EYQIRVYAETGGDGSEMYFGWIQIG

SEQ ID NO: 36 - protein, 20 aa, Artificial Sequence
  Adnectin
LAYHIQYWPLGWYHLYQVFS

SEQ ID NO: 37 - protein, 20 aa, Artificial Sequence
  Adnectin
LAYHIQYWPLGWYQLYKVFS

SEQ ID NO: 38 - protein, 25 aa, Artificial Sequence
  Adnectin
DYQIRVYAETSGESSEQYLGWIQIG

SEQ ID NO: 39 - protein, 20 aa, Artificial Sequence
  Adnectin
HSYHIQYWPLGWYQLYQVFS

SEQ ID NO: 40 - protein, 25 aa, Artificial Sequence
  Adnectin
EYQIRVYAETEVDSGQHSFGWIQIG

SEQ ID NO: 41 - protein, 20 aa, Artificial Sequence
  Adnectin
LAYHIQYWPLGWYQRYQIFS

SEQ ID NO: 42 - protein, 25 aa, Artificial Sequence
  Adnectin
EYQIRVYAETGESGAQQSFGWIQIG

SEQ ID NO: 43 - protein, 20 aa, Artificial Sequence
  Adnectin
QSYHIQYWPLGAYQLYQLFS

SEQ ID NO: 44 - protein, 20 aa, Artificial Sequence
  Adnectin
HAYHIQYWPLGFYQGYQVFS

SEQ ID NO: 45 - protein, 25 aa, Artificial Sequence
  Adnectin
EYQIRVYADTGRGYQLSYSWIQIGY

SEQ ID NO: 46 - protein, 20 aa, Artificial Sequence
  Adnectin
FRYHIQYWPLGGYERYQVFT

SEQ ID NO: 47 - protein, 20 aa, Artificial Sequence
  Adnectin
HSYHIQYWPLGSYHLYQLFS

SEQ ID NO: 48 - protein, 20 aa, Artificial Sequence
  Adnectin
HSYHIQYWPLGWYQLYQVFT

SEQ ID NO: 49 - protein, 25 aa, Artificial Sequence
  Adnectin
EYQIRVYAETGGFGSPPNFGWIQIG

SEQ ID NO: 50 - protein, 20 aa, Artificial Sequence
  Adnectin
QFYHIQYWPLGSYQRYQVFS

SEQ ID NO: 51 - protein, 20 aa, Artificial Sequence
  Adnectin
NSYHIQYWPLGWYHRYQVFS

SEQ ID NO: 52 - protein, 20 aa, Artificial Sequence
  Adnectin
HSYHIQYWPLGRYQLYQVFS

SEQ ID NO: 53 - protein, 20 aa, Artificial Sequence
  Adnectin
LAYHIQYWPLGWYHLYQIFS

SEQ ID NO: 54 - protein, 25 aa, Artificial Sequence
  Adnectin
EYQIRVYAETGGVGWHHSFGWIQIG

SEQ ID NO: 55 - protein, 20 aa, Artificial Sequence
  Adnectin
HVYHIQYWPLGWYPRYQVFS

SEQ ID NO: 56 - protein, 20 aa, Artificial Sequence
  Adnectin
HSYHIPYWELAWYQRYQVFS

SEQ ID NO: 57 - protein, 20 aa, Artificial Sequence
  Adnectin
ESYHIQYWPLGLYHRYQVFS

SEQ ID NO: 58 - protein, 20 aa, Artificial Sequence
  Adnectin
LAYHIQYWPLGWYQAYQVFS

SEQ ID NO: 59 - protein, 20 aa, Artificial Sequence
  Adnectin
YLYHIQYWPLGWYHRYQVFT

SEQ ID NO: 60 - protein, 20 aa, Artificial Sequence
  Adnectin
RFYHIQYWPLGWYHCYQVFV

SEQ ID NO: 61 - protein, 25 aa, Artificial Sequence
  Adnectin
EYQIRVYAQTGDGSSQEYFGWIQIG

SEQ ID NO: 62 - protein, 20 aa, Artificial Sequence
  Adnectin
HSYHIQYWPLGWYYRYQVFS

SEQ ID NO: 63 - protein, 25 aa, Artificial Sequence
  Adnectin
EYQIRVYAETGGSGSQQYWGWIQIG

SEQ ID NO: 64 - protein, 20 aa, Artificial Sequence
  Adnectin
HAYHIQYWPLGFYQGYQVFS

SEQ ID NO: 65 - protein, 20 aa, Artificial Sequence
  Adnectin
HSYHIQYWPLGLYVLYQVFS

SEQ ID NO: 66 - protein, 25 aa, Artificial Sequence
  Adnectin
EYQIRVYAETGAGGSEHSFGWIQIG

SEQ ID NO: 67 - protein, 20 aa, Artificial Sequence
  Adnectin
LSYHIQYWPLGRYERYQVFS

SEQ ID NO: 68 - protein, 25 aa, Artificial Sequence
  Adnectin
EYQIRVYAETVGGESLDSFSWIQIG

SEQ ID NO: 69 - protein, 20 aa, Artificial Sequence
  Adnectin
LSYHIQYWPLGWYQLYQVFY

SEQ ID NO: 70 - protein, 25 aa, Artificial Sequence
  Adnectin
EYQIRVYAETRVGGSVASFGWIQIG

SEQ ID NO: 71 - protein, 20 aa, Artificial Sequence
  Adnectin
LAYHIQYWPLGRYQLYQVFS

SEQ ID NO: 72 - protein, 111 aa, Artificial Sequence
  Adnectin
MASTSGVSDVPRDLEVVAATPTSLLISWDAPAVTVHSYHIQYWPLGWYQRYQVFSVPGSKSTATISGLKPEVEYQIRVYAETGGGGSQQSFGWIQIGYRTEGSGSHHHHHH

SEQ ID NO: 73 - protein, 109 aa, Artificial Sequence
  Adnectin
MGVSDVPRDLEVVAATPTSLLISWDAPAVTVQSYHIQYWPLGSYQRYQVFSVPGSKSTATISGLKPGVEYQIRVYAETGGADSDQSMGWIQIGYRTEGDKPSQHHHHHH

SEQ ID NO: 74 - protein, 109 aa, Artificial Sequence
  Adnectin
MGVSDVPRDLEVVAATPTSLLISWDAPAVTVHSYHIQYWPLGWYQRYQVFSVPGSKSTATISGLKPGVEYQIRVYAETRSGLADESFGWIQIGYRTEGDKPSQHHHHHH

SEQ ID NO: 75 - protein, 109 aa, Artificial Sequence
  Adnectin
MGVSDVPRDLEVVAATPTSLLISWDAPAVTVHSYHIQYWPLGSYQRYQVFSVPGSKSTATISGLKPGVEYQIRVYAETGGADSDQSMGWIQIGYRTEGDKPSQHHHHHH

SEQ ID NO: 76 - protein, 107 aa, Artificial Sequence
  Adnectin
MGHHHHHHGGVSDVPRDLEVVAATPTSLLISWDAPAVTVHSYHIQYWPLGSYQRYQVFSVPGSKSTATISGLKPGVEYQIRVYAETGGADSDQSFGWIQIGYRTPES

SEQ ID NO: 77 - protein, 115 aa, Artificial Sequence
  Adnectin
MASTSGSSSYLMPSDLEVVAATPTSLYIHWYPIASTIINFRITYGETGGNSPVQEFTVPGSQVHATISGLKPGVDYTITVYAVHYEHKYSELWMGHPISINYRTEGSGSHHHHHH

SEQ ID NO: 78 - protein, 115 aa, Artificial Sequence
  Adnectin
MASTSGSASYLIPSDLEVVAATPTSLSIYWYPVASTIINFRITYVETGGNSPVQEFTVPGSKSTATISGLKPGVDYTITVYAVHYEQKYSEYWIGHPISINYRTEGSGSHHHHHH

SEQ ID NO: 79 - protein, 115 aa, Artificial Sequence
  Adnectin
MASTSGSSPYLMPYDLEVVAATPTSLFIRWYGSASSIVKFRITYGETGGNSPVQEFTVGGTQLHATISGLKPGVDYTITVYAVHFEHKYSELWIGHPISINYRTEGSGSHHHHHH

SEQ ID NO: 80 - protein, 115 aa, Artificial Sequence
  Adnectin
MASTSGYTSYPIPYDLEVVAATPTSLYIHWYWIAATIISFRITYGETGGNSPVQEFTVPAGQDHATISGLKPGVDYTITVYAVHYEEEYSEFWTGHPISINYRTEGSGSHHHHHH

SEQ ID NO: 81 - protein, 116 aa, Artificial Sequence
  Adnectin
MASTSGTHWFYSIPHDLEVVAATPTSLTIAWEPPHHTAMGYRITYGETGGNSPVQEFTVPGGYTTAYISGLKPGVDYTITVYAAYYEREYSEHWISHPISINYRTEGSGSHHHHHH

SEQ ID NO: 82 - protein, 118 aa, Artificial Sequence
  Adnectin
MASTSGEFYHTKYPYDLEVVAATPTSLEISWRSPTRDWQWFRITYGETGGNSPVQEFTVAGPYRNAIISGLKPGVDYTITVYADVYMPSEGGLVVDTYHPISINYRTEGSGSHHHHHH

SEQ ID NO: 83 - protein, 116 aa, Artificial Sequence
  Adnectin
MASTSGQAYPEYYFVDLEVVAATPTSLLISWSKPYYNAYSYRITYGETGGNSPVQEFTVLGHDTRAVISGLKPGVDYTITVYAMFIEYIDQEIWHAHPISINYRTEGSGSHHHHHH

SEQ ID NO: 84 - protein, 109 aa, Artificial Sequence
  Adnectin
MGVSDVPRDLEVVAATPTSLLISWDEHTDIYRYYRITYGETGGNSPVQEFTVPAMEHTATISGLKPGVDYTITVYAVTHVYPIMIHQYPISINYRTEIDKPSQHHHHHH

SEQ ID NO: 85 - protein, 111 aa, Artificial Sequence
  Adnectin
MASTSGVSDVPRDLEVVAATPTSLLISWDAPAVTVLEYQIDYHPAAVWHALQRFTVPGSKSTATISGLKPGVHYKISVTATTHADNESIMWHPISIYYRTEGSGSHHHHHH

SEQ ID NO: 86 - protein, 109 aa, Artificial Sequence
  Adnectin
MGVSDVPRDLEVVAATPTSLLISWDYPTVTPRYYRITYGETGGNSPVQEFTVPEYIGTATISGLKPGVDYTITVYAVTNDTTIYSISRPISINYRTEIDKPSQHHHHHH

SEQ ID NO: 87 - protein, 107 aa, Artificial Sequence
  Adnectin
MGHHHHHHGGVSDVPRDLEVVAATPTSLLISWDAPAVTVHSYHIQYWPLGSYQRYQVFSVPGSKSTATISGLKPGVEYQIRVYAETGRGESDQSLGWIQIGYRTEES

SEQ ID NO: 88 - protein, 107 aa, Artificial Sequence
  Adnectin
MGHHHHHHGGVSDVPRDLEVVAATPTSLLISWDAPAVTVHSYHIQYWPLGSYQRYQVFSVPGSKSTATISGLKPGVEYQIRVYAETGRGESDQSFGWIQIGYRTEES

SEQ ID NO: 89 - protein, 107 aa, Artificial Sequence
  Adnectin
MGHHHHHHGGVSDVPRDLEVVAATPTSLLISWDAPAVTVHSYHIQYWPLGSYQRYQVFSVPGSKSTATISGLKPGVEYQIRVYAETGRGESDQSLGWIQIGYRTPES

SEQ ID NO: 90 - protein, 107 aa, Artificial Sequence
  Adnectin
MGHHHHHHGGVSDVPRDLEVVAATPTSLLISWDAPAVTVHSYHIQYWPLGSYQRYQVFSVPGSKSTATISGLKPGVEYQIRVYAETGRGESDQSFGWIQIGYRTPES

SEQ ID NO: 91 - protein, 107 aa, Artificial Sequence
  Adnectin
MGHHHHHHGGVSDVPRDLEVVAATPTSLLISWDAPAVTVHAYHIQYWPLGFYQGYQVFSVPGSKSTATISGLKPGVEYQIRVYAETGLGDAHQSLGWIQIGYRTPES

SEQ ID NO: 92 - protein, 105 aa, Artificial Sequence
  Adnectin
MGVSDVPRDLEVVAATPTSLLISWEYKVNNYRYYRITYGETGGNSPVQEFTVPSVLSTATISGLKPGVDYTITVYAVTYGVHSSPISINYRTEIDKPSQHHHHHH

SEQ ID NO: 93 - protein, 111 aa, Artificial Sequence
  Adnectin
MASTSGVSDVPRDLEVVAATPTSLLISWDAPAVTVEQYYIAYYVEGEPSSYQYFRVPGSKSTATISGLKPGVLYHIYVNAVTGSGLRPEFSLPIRIKYRTEGSGSHHHHHH

SEQ ID NO: 94 - protein, 104 aa, Artificial Sequence
  Adnectin
MGHHHHHHGGSVSDVPRDLEVVAATPTSLLISWEYKVHPYRYYRITYGETGGNSPVQEFTVPSVLSTATISGLKPGVDYTITVYAVTYGDDSAPISINYRTEID

SEQ ID NO: 95 - protein, 105 aa, Artificial Sequence
  Adnectin
MGVSDVPRDLEVVAATPTSLLISWKYKVHPYRYYRITYGETGGNSPVQEFTVPSVLSTATISGLKPGVDYTITVYAVTYGVNSLPISINYRTEIDKPSQHHHHHH

SEQ ID NO: 96 - protein, 107 aa, Artificial Sequence
  Adnectin
MGVSDVPRDLEVVAATPTSLLISWDAPAVTVGWYHIGYNVEGEPASYQYFRVPGSKSTATISGLKPGVEYMIFVNAVTGSGAREEFSLPISINYRTEGSGSHHHHHH

SEQ ID NO: 97 - protein, 103 aa, Artificial Sequence
  Adnectin
MGHHHHHHGGVSDVPRDLEVVAATPTSLLISWEYNVNPYRYYRITYGETGGNSPVQEFTVPSVLSSAQISGLKPGVDYTITVYAVTRGVDSAPISINYRTPGG

SEQ ID NO: 98 - protein, 103 aa, Artificial Sequence
  Adnectin
MGHHHHHHGGVSDVPRDLEVVAATPTSLLISWEYNVNPYRYYRITYGETGGNSPVQEFTVPSVLSTAEISGLKPGVDYTITVYAVTYGVDSDPISINYRTPGG

SEQ ID NO: 99 - protein, 103 aa, Artificial Sequence
  Adnectin
MGHHHHHHGGVSDVPRDLEVVAATPTSLLISWQYKVHPYRYYRITYGETGGNSPVQEFTVPSVLSTAEISGLKPGVDYTITVYAVTRGVDSAPISINYRTPGG

SEQ ID NO: 100 - protein, 105 aa, Artificial Sequence
  Adnectin
MGVSDVPRDLEVVAATPTSLLISWEYKVHPYRYYRITYGETGGNSPVQEFTVPSVLSTATISGLKPGVDYTITVYAVTRGVDSAPISINYRTEIDKPSQHHHHHH

SEQ ID NO: 101 - protein, 105 aa, Artificial Sequence
  Adnectin
MGVSDVPRDLEVVAATPTSLLISWEYKVHPYRYYRITYGETGGNSPVQEFTVPSVLSTAEISGLKPGVDYTITVYAVTRGVDSAPISINYRTPIDKPSQHHHHHH

SEQ ID NO: 102 - protein, 103 aa, Artificial Sequence
  Adnectin
MGHHHHHHGGVSDVPRDLEVVAATPTSLLISWEYKVHPYRYYRITYGETGGNSPVQEFTVPSVLSTATISGLKPGVDYTITVYAVTYGVQSDPISINYRTPGG

SEQ ID NO: 103 - protein, 105 aa, Artificial Sequence
  Adnectin
MGVSDVPRDLEVVAATPTSLLISWEYKVHPYRYYRITYGETGGNSPVQEFTVPSVLSTATISGLKPGVDYTITVYAVTYGIQSPPISINYRTEIDKPSQHHHHHH

SEQ ID NO: 104 - protein, 103 aa, Artificial Sequence
  Adnectin
MGHHHHHHGGVSDVPRDLEVVAATPTSLLISWQYKVHPYRYYRITYGETGGNSPVQEFTVPSVLSSAQISGLKPGVDYTITVYAVTYGIESSPISINYRTPGG

SEQ ID NO: 105 - protein, 103 aa, Artificial Sequence
  Adnectin
MGHHHHHHGGVSDVPRDLEVVAATPTSLLISWEYKVHPYRYYRITYGETGGNSPVQEFTVPSVLSSAEISGLKPGVDYTITVYAVTYGIDSSPISINYRTPGG

SEQ ID NO: 106 - protein, 105 aa, Artificial Sequence
  Adnectin
MGVSDVPRDLEVVAATPTSLLISWEYKVHPYRYYRITYGETGGNSPVQEFTVPSVLSSAEISGLKPGVDYTITVYAVTYGIDSPPISINYRTEIDKPSQHHHHHH

SEQ ID NO: 107 - protein, 104 aa, Artificial Sequence
  Adnectin
MGHHHHHHGGSVSDVPRDLEVVAATPTSLLISWEYNVHYDRYYRITYGETGGNSPVQEFTVPSVLSTATISGLKPGVDYTITVYAVTDGVHSSPISINYRTEID

SEQ ID NO: 108 - protein, 104 aa, Artificial Sequence
  Adnectin
MGHHHHHHGGSVSDVPRDLEVVAATPTSLLISWEYKVHPYRYYRITYGETGGNSPVQEFTVPSVLSTATISGLKPGVDYTITVYAVTYGDLSAPISINYRTEID

SEQ ID NO: 109 - protein, 108 aa, Artificial Sequence
  Adnectin
MGHHHHHHGGSVSDVPRDLEVVAATPTSLLISWDAPAVTVEEYYIGYYVEFEPSSYQWFTVPGSKSTATISGLKPGVEYSIYVNAVTGMGMQPEMSLPISINYRTEGS

SEQ ID NO: 110 - protein, 104 aa, Artificial Sequence
  Adnectin
MGHHHHHHGGSVSDVPRDLEVVAATPTSLLISWEYKVHYDRYYRITYGETGGNSPVQEFTVPSVLSTATISGLKPGVDYTITVYAVTYGVDSDPISINYRTEID

SEQ ID NO: 111 - protein, 108 aa, Artificial Sequence
  Adnectin
MGHHHHHHGGSVSDVPRDLEVVAATPTSLLISWDAPAVTVEQYYIAYYDEKEPSSYQYFRVPGSKSTATISGLKPGVEYAIFVNAVTRSGVLPEFSLPISINYRTEGS

SEQ ID NO: 112 - protein, 104 aa, Artificial Sequence
  Adnectin
MGHHHHHHGGSVSDVPRDLEVVAATPTSLLISWKYNVNAYRYYRITYGETGGNSPVQEFTVPSVLSTATISGLKPGVDYTITVYAVTYGVHSSPISINYRTEID

SEQ ID NO: 113 - protein, 108 aa, Artificial Sequence
  Adnectin
MGHHHHHHGGSVSDVPRDLEVVAATPTSLLISWDAPAVTVEQYYIGYYVEAEPSSYQYFFVPGSKSTATISGLKPGVDYAIFVNAVTASGRGPEYSLPISINYRTEGS

SEQ ID NO: 114 - protein, 109 aa, Artificial Sequence
  Adnectin
MGVSDVPRDLEVVAATPTSLLISWHDGIGEERYYRITYGETGGNSPVQEFTVPMDDITATISGLKPGVDYTITVYAVTVGDVISVLHEPISINYRTEIDKPSQHHHHHH

SEQ ID NO: 115 - protein, 108 aa, Artificial Sequence
  Adnectin
MGVSDVPRDLEVVAATPTSLLISWHYPFEGYVTYYRITYGETGGNSHVQEFTVPVGYTTATISGLKPGVDYTITVYAVTSSKGYVYFPISINYRTEIDKPSQHHHHHH

SEQ ID NO: 116 - protein, 109 aa, Artificial Sequence
  Adnectin
MGVSDVPRDLEVVAATPTSLLISWEDPEAAVRYYRITYGETGGNSPVQEFTVPINDLHSYLSGLKPGVDYTITVYAVTEATVMYVLDEPISINYRTEIDKPSQHHHHHH

SEQ ID NO: 117 - protein, 109 aa, Artificial Sequence
  Adnectin
MGVSDVPRDLEVVAATPTSLLISWDLLEDMSRYYRITYGETGGNSPVQEFTVPTDAYTATISGLKPGVDYTITVYAVTQDSHVIELSYPISINYRTEIDKPSQHHHHHH

SEQ ID NO: 118 - protein, 105 aa, Artificial Sequence
  Adnectin
MGVSDVPRDLEVVAATPTSLLISWQYKVHPYRYYRITYGETGGNSPVQEFTVPSVLSTATISGLKPGVDYTITVYAVTYGIQSPPISINYRTEIDKPSQHHHHHH

SEQ ID NO: 119 - protein, 105 aa, Artificial Sequence
  Adnectin
MGVSDVPRDLEVVAATPTSLLISWRYRVHPYRYYRITYGETGGNSPVQEFTVPSVLSTATISGLKPGVDYTITVYAVTDGVQSSPISINYRTEIDKPSQHHHHHH

SEQ ID NO: 120 - protein, 105 aa, Artificial Sequence
  Adnectin
MGVSDVPRDLEVVAATPTSLLISWEYNVNPYRYYRITYGETGGNSPVQEFTVPSVLSTATISGLKPGVDYTITVYAVTYGIESSPISINYRTEIDKPSQHHHHHH

SEQ ID NO: 121 - protein, 105 aa, Artificial Sequence
  Adnectin
MGVSDVPRDLEVVAATPTSLLISWQYKVHPYRYYRITYGETGGNSPVQEFTVPSVLSTATISGLKPGVDYTITVYAVTYGVESSPISINYRTEIDKPSQHHHHHH

SEQ ID NO: 122 - protein, 105 aa, Artificial Sequence
  Adnectin
MGVSDVPRDLEVVAATPTSLLISWEYKVHPYRYYRITYGETGGNSPVQEFTVPSVLSTATISGLKPGVDYTITVYAVTYGVNSLPISINYRTEIDKPSQHHHHHH

SEQ ID NO: 123 - protein, 105 aa, Artificial Sequence
  Adnectin
MGVSDVPRDLEVVAATPTSLLISWEYKVHPYRYYRITYGETGGNSPVQEFTVPSVLSTATISGLKPGVDYTITVYAVTYGIDSPPISINYRTEIDKPSQHHHHHH

SEQ ID NO: 124 - protein, 105 aa, Artificial Sequence
  Adnectin
MGVSDVPRDLEVVAATPTSLLISWEYKVHPYRYYRITYGETGGNSPVQEFTVPSVLSTATISGLKPGVDYTITVYAVTYGVDSDPISINYRTEIDKPSQHHHHHH

SEQ ID NO: 125 - protein, 105 aa, Artificial Sequence
  Adnectin
MGVSDVPRDLEVVAATPTSLLISWEYNVNPYRYYRITYGETGGNSPVQEFTVPSVLSTATISGLKPGVDYTITVYAVTYGIDSPPISINYRTEIDKPSQHHHHHH

SEQ ID NO: 126 - protein, 104 aa, Artificial Sequence
  Adnectin
MGHHHHHHGGSVSDVPRDLEVVAATPTSLLISWEYKVHPYRYYRITYGETGGNSPVQEFTVPSVLSTATISGLKPGVDYTITVYAVTYGKDSAPISINYRTEID

SEQ ID NO: 127 - protein, 105 aa, Artificial Sequence
  Adnectin
MGVSDVPRDLEVVAATPTSLLISWEYNVNPYRYYRITYGETGGNSPVQEFTVPSVLSTATISGLKPGVDYTITVYAVTYGVESSPISINYRTEIDKPSQHHHHHH

SEQ ID NO: 128 - protein, 105 aa, Artificial Sequence
  Adnectin
MGVSDVPRDLEVVAATPTSLLISWEYKVHPYRYYRITYGETGGNSPVQEFTVPSVLSTATISGLKPGVDYTITVYAVTYGIDSSPISINYRTEIDKPSQHHHHHH

SEQ ID NO: 129 - protein, 105 aa, Artificial Sequence
  Adnectin
MGVSDVPRDLEVVAATPTSLLISWEYNVNPYRYYRITYGETGGNSPVQEFTVPSVLSTATISGLKPGVDYTITVYAVTYGVQSDPISINYRTEIDKPSQHHHHHH

SEQ ID NO: 130 - protein, 104 aa, Artificial Sequence
  Adnectin
MGHHHHHHGGSVSDVPRDLEVVAATPTSLLISWEYKVHPYRYYRITYGETGGNSPVQEFTVPSVLSTAQISGLKPGVDYTITVYAVTYGDDSAPISINYRTEID

SEQ ID NO: 131 - protein, 105 aa, Artificial Sequence
  Adnectin
MGVSDVPRDLEVVAATPTSLLISWEYNVNPYRYYRITYGETGGNSPVQEFTVPSVLSTATISGLKPGVDYTITVYAVTYGVNSLPISINYRTEIDKPSQHHHHHH

SEQ ID NO: 132 - protein, 105 aa, Artificial Sequence
  Adnectin
MGVSDVPRDLEVVAATPTSLLISWQYKVHPYRYYRITYGETGGNSPVQEFTVPSVLSTATISGLKPGVDYTITVYAVTHGVHSAPISINYRTEIDKPSQHHHHHH

SEQ ID NO: 133 - protein, 105 aa, Artificial Sequence
  Adnectin
MGVSDVPRDLEVVAATPTSLLISWEYKVNPWRYYRITYGETGGNSPVQEFTVPSVLSTATISGLKPGVDYTITVYAVTYGIESSPISINYRTEIDKPSQHHHHHH

SEQ ID NO: 134 - protein, 105 aa, Artificial Sequence
  Adnectin
MGVSDVPRDLEVVAATPTSLLISWQYKVHPYRYYRITYGETGGNSPVQEFTVPSVLSTATISGLKPGVDYTITVYAVTYGVNSIPISINYRTEIDKPSQHHHHHH

SEQ ID NO: 135 - protein, 105 aa, Artificial Sequence
  Adnectin
MGVSDVPRDLEVVAATPTSLLISWEYKVHYDRYYRITYGETGGNSPVQEFTVPSVLSTATISGLKPGVDYTITVYAVTYGIQSPPISINYRTEIDKPSQHHHHHH

SEQ ID NO: 136 - protein, 104 aa, Artificial Sequence
  Adnectin
MGHHHHHHGGSVSDVPRDLEVVAATPTSLLISWEYKVHPYRYYRITYGETGGNSPVQEFTVPSVLSTATISGLKPGVDYTITVYAVTYGDDSAPISIPYRTPGG

SEQ ID NO: 137 - protein, 105 aa, Artificial Sequence
  Adnectin
MGVSDVPRDLEVVAATPTSLLISWEYKVDPYRYYRITYGETGGNSPVQEFTVPSVLSTATISGLKPGVDYTITVYAVTYGVESSPISINYRTEIDKPSQHHHHHH

SEQ ID NO: 138 - protein, 104 aa, Artificial Sequence
  Adnectin
MGHHHHHHGGSVSDVPRDLEVVAATPTSLLISWEYKVHPYRYYRITYGETGGNSPVQEFTVPSVLSSAEISGLKPGVDYTITVYAVTYGDDSAPISINYRTEID

SEQ ID NO: 139 - protein, 104 aa, Artificial Sequence
  Adnectin
MGHHHHHHGGSVSDVPRDLEVVAATPTSLLISWEYKVHPYRYYRITYGETGGNSPVQEFTVPSVLSSAQISGLKPGVDYTITVYAVTYGDDSAPISINYRTEID

SEQ ID NO: 140 - protein, 104 aa, Artificial Sequence
  Adnectin
MGHHHHHHGGSVSDVPRYLEVVAATPTSLLISWQYKVHPYRWYRITYGETGGNSPVQEFTVPSVLSTATISGLKPGVDYTITVYAVTYGVNSIPISINYRTEID

SEQ ID NO: 141 - protein, 104 aa, Artificial Sequence
  Adnectin
MGHHHHHHGGSVSDVPRDLEVVAATPTSLLISWKYQVHAYRYYRITYGETGGNSPVQEFTVPSVLSTATISGLKPGVDYTITVYAVTYGVYSAPISINYRTEID

SEQ ID NO: 142 - protein, 104 aa, Artificial Sequence
  Adnectin
MGHHHHHHGGSVSDVPRDLEVVAATPTSLLISWEYKVNPYRYYRITYGETGGNSPVQEFTVPSVLSTATISGLKPGVDYTITVYAVTYGVHSSPISINYRTEID

SEQ ID NO: 143 - protein, 104 aa, Artificial Sequence
  Adnectin
MGHHHHHHGGSVSDVPRDLEVVAATPTSLLISWKYNLHPYRYYRITYGETGGNSPVQEFTVPSVLSTATISGLKPGVDYTITVYAVTYGIISEPISINYRTEID

SEQ ID NO: 144 - protein, 104 aa, Artificial Sequence
  Adnectin
MGHHHHHHGGSVSDVPRDLEVVAATPTSLLISWEYRVNPYRYYRITYGETGGNSPVQEFTVPSVLSTATISGLKPGVDYTITVYAVTYGVQSPPISINYRTEID

SEQ ID NO: 145 - protein, 104 aa, Artificial Sequence
  Adnectin
MGHHHHHHGGSVSDVPRDLEVVAATPTSLLISWEYKVNAYRYYRITYGETGGNSPVQEFTVPSVLSTATISGLKPGVDYTITVYAVTYGVLSPPISINYRTEID

SEQ ID NO: 146 - protein, 104 aa, Artificial Sequence
  Adnectin
MGHHHHHHGGSVSDVPRDLEVVAATPTSLLISWEYNVNPYRYYRITYGETGGNSPVQEFTVPSVLSTATISGLKPGVDYTITVYAVTRGVDSAPISINYRTEID

SEQ ID NO: 147 - protein, 104 aa, Artificial Sequence
  Adnectin
MGHHHHHHGGSVSDVPRDLEVVAATPTSLLISWEYKVSPYRYYRITYGETGGNSPVQEFTVPSVLSTATISGLKPGVDYTITVYAVTFGIRSSPISINYRTEID

SEQ ID NO: 148 - protein, 104 aa, Artificial Sequence
  Adnectin
MGHHHHHHGGSVSDVPRDLEVVAATPTSLLISWKYQVHAYRYYRITYGETGGNSPVQEFTVPSVLSTATISGLKPGVDYTITVYAVTYGIISEPISINYRTEID

SEQ ID NO: 149 - protein, 104 aa, Artificial Sequence
  Adnectin
MGHHHHHHGGSVSDVPRDLEVVAATPTSLLISWEYKVDPYRYYRITYGETGGNSPVQEFTVPSVLSTATISGLKPGVDYTITVYAVTYGIDSSPISINYRTEID

SEQ ID NO: 150 - protein, 104 aa, Artificial Sequence
  Adnectin
MGHHHHHHGGAVSDVPRDLEVVAATPTSLLISWEYKVNPWRYYRITYGETGGNSPVQEFTVPSVLSTATISGLKPGVDYTITVYAVTYGVHSSPISINYRTEID

SEQ ID NO: 151 - protein, 104 aa, Artificial Sequence
  Adnectin
MGHHHHHHGGSVSDVPRDLEVVAATPTSLLISWEYNVNAYRYYRITYGETGGNSPVQEFTVPSVLSTATISGLKPGVDYTITVYAVTYGIISEPISINYRTEID

SEQ ID NO: 152 - protein, 104 aa, Artificial Sequence
  Adnectin
MGHHHHHHGGSVSDVPRDLEVVAATPTSLLISWEYKVHYDRYYRITYGETGGNSPVQEFTVPSVLSTATISGLKPGVDYTITVYAVTYGVQSTPISINYRTEID

SEQ ID NO: 153 - protein, 104 aa, Artificial Sequence
  Adnectin
MGHHHHHHGGSVSDVPRDLEVVAATPTSLLISWEYKVHHDRYYRITYGETGGNSPVQEFTVPSVLSTATISGLKPGVDYTITVYAVTYGIESSPISINYRTEID

SEQ ID NO: 154 - protein, 104 aa, Artificial Sequence; identical to SEQ ID NO: 136
  Adnectin

SEQ ID NO: 155 - protein, 104 aa, Artificial Sequence
  Adnectin
  misc_feature (90)..(90): Xaa can be any naturally occurring amino acid
  misc_feature (93)..(93): Xaa can be any naturally occurring amino acid
MGHHHHHHGGSVSDVPRDLEVVAATPTSLLISWEYKVHPYRYYRITYGETGGNSPVQEFTVPSVLSTATISGLKPGVDYTITVYAVTYGXDSXPISINYRTEID

SEQ ID NO: 156 - protein, 104 aa, Artificial Sequence
  Adnectin
MGHHHHHHGGSVSDVPRDLEVVAATPTSLLISWEYKVHPYRYYRITYGETGGNSPVQEFTVPSVLSTATISGLKPGVDYTITVYAVTYGDDSRPISINYRTEID

SEQ ID NO: 157 - protein, 104 aa, Artificial Sequence; identical to SEQ ID NO: 126
  Adnectin

SEQ ID NO: 158 - protein, 104 aa, Artificial Sequence
  Adnectin
MGHHHHHHGGSVSDVPRDLEVVAATPTSLLISWEYKVHPYRYYRITYGETGGNSPVQEFTVPSVLSTATISGLKPGVDYTITVYAVTYGDHSAPISINYRTEID

SEQ ID NO: 159 - protein, 104 aa, Artificial Sequence
  Adnectin
MGHHHHHHGGSVSDVPRDLEVVAATPTSLLISWEYKVHPYRYYRITYGETGGNSPVQEFTVPSVLSTATISGLKPGVDYTITVYAVTYGDQSAPISINYRTEID

SEQ ID NO: 160 - protein, 104 aa, Artificial Sequence
  Adnectin
MGHHHHHHGGSVSDVPRDLEVVAATPTSLLISWEYKVHPYRYYRITYGETGGNSPVQEFTVPSVLSTATISGLKPGVDYTITVYAVTYGIDSAPISINYRTEID

SEQ ID NO: 161 - protein, 104 aa, Artificial Sequence
  Adnectin
MGHHHHHHGGSVSDVPRDLEVVAATPTSLLISWEYKVHPYRYYRITYGETGGNSPVQEFTVPSVLSTATISGLKPGVDYTITVYAVTYGDVSAPISINYRTEID

SEQ ID NO: 162 - protein, 104 aa, Artificial Sequence
  Adnectin
  misc_feature (90)..(90): Xaa can be any naturally occurring amino acid
MGHHHHHHGGSVSDVPRDLEVVAATPTSLLISWEYKVHPYRYYRITYGETGGNSPVQEFTVPSVLSTATISGLKPGVDYTITVYAVTYGXDSAPISINYRTEID

SEQ ID NO: 163 - protein, 104 aa, Artificial Sequence
  Adnectin
MGHHHHHHGGSVSDVPRDLEVVAATPTSLLISWEYKVHPYRYYRITYGETGGNSPVQEFTVPSVLSTATISGLKPGVDYTITVYAVTYGEDSAPISINYRTEID

SEQ ID NO: 164 - protein, 104 aa, Artificial Sequence
  Adnectin
MGHHHHHHGGSVSDVPRDLEVVAATPTSLLISWEYKVHPYRYYRITYGETGGNSPVQEFKVPSVLSTATISGLKPGVDYTITVYAVTYGDDSAPISINYRTEID

SEQ ID NO: 165 - protein, 104 aa, Artificial Sequence; identical to SEQ ID NO: 108
  Adnectin

SEQ ID NO: 166 - protein, 104 aa, Artificial Sequence; identical to SEQ ID NO: 126
  Adnectin

SEQ ID NO: 167 - protein, 104 aa, Artificial Sequence
  Adnectin
MGHHHHHHGGSVSDVPRDLEVVAATPTSLLISWEYKVHPYRYYRITYGETGGNSPVQEFTVPSVLSTATISGLKPGVDYTITVYAVTYGADSAPISINYRTEID

SEQ ID NO: 168 - protein, 104 aa, Artificial Sequence
  Adnectin
MGHHHHHHGGSVSDVPRDLEVVAATPTSLLISWEYKVHPYRYYRITYGETGGNSPVQEFTVPSVLSTATISGLKPGVDYTITVYAVTYGTDSAPISINYRTEID

SEQ ID NO: 169 - protein, 104 aa, Artificial Sequence; identical to SEQ ID NO: 168
  Adnectin

SEQ ID NO: 170 - protein, 104 aa, Artificial Sequence; identical to SEQ ID NO: 126
  Adnectin

SEQ ID NO: 171 - protein, 104 aa, Artificial Sequence
  Adnectin
MGHHHHHHGGSVSDVPRDLEVVAATPTSLLISWEYKVHPYRYYRITYGETGGNSPVQEFTVPSVLSTATISGLKPGVDYTITVYAVTYGDPSAPISINYRTEID

SEQ ID NO: 172 - protein, 104 aa, Artificial Sequence
  Adnectin
MGHHHHHHGGSVSDVPRDLEVVAATPTSLLISWEYKVHPYRYYRITYGETGGNSPVQEFTVPSVLSTATISGLKPGVDYTITVYAVTYGDDSQPISINYRTEID

SEQ ID NO: 173 - protein, 104 aa, Artificial Sequence; identical to SEQ ID NO: 161
  Adnectin

SEQ ID NO: 174 - protein, 104 aa, Artificial Sequence
  Adnectin
MGHHHHHHGGSVSDVPRDLEVVAATPTSLLISWEYKVHPYRYYRITYGETGGNSPVQEFTVPSVLSTATISGLKPGVDYTITVYAVTYGDDSGPISINYRTEID

SEQ ID NO: 175 - protein, 104 aa, Artificial Sequence
  Adnectin
MGHHHHHHGGSVSDVPRDLEVVAATPTSLLISWEYKVHPYRYYRITYGETGGNSPVQEFTVPSVLSTATISGLKPGVDYTITVYAVTYGDDSAPISIQYRTEID

SEQ ID NO: 176 - protein, 104 aa, Artificial Sequence; identical to SEQ ID NO: 172
  Adnectin

SEQ ID NO: 177 - protein, 104 aa, Artificial Sequence
  Adnectin
MGHHHHHHGGSVSDVPRDLEVVAATPTSLLISWEYKVHPYRYYRITYTETGGNSPVQEFTVPSVLSTATISGLKPGVDYTITVYAVTYGDDSAPISINYRTEID

SEQ ID NO: 178 - protein, 104 aa, Artificial Sequence; identical to SEQ ID NO: 159
  Adnectin

SEQ ID NO: 179 - protein, 104 aa, Artificial Sequence
  Adnectin
MGHHHHHHGGSVSDVPRDLEVVAATPTSLLISWEYKVHPYRYYRITYGETGGNSPVQEFTVPSVLSTATISGLKPGVDYTITVYAVTYGDDSKPISINYRTEID

SEQ ID NO: 180 - protein, 104 aa, Artificial Sequence; identical to SEQ ID NO: 174
  Adnectin

SEQ ID NO: 181 - protein, 104 aa, Artificial Sequence
  Adnectin
MGHHHHHHGGSVSDVPRDLEVVAATPTSLLISWEYKVHPYRYYRITYRETGGNSPVQEFTVPSVLSTATISGLKPGVDYTITVYAVTYGDDSAPISINYRTEID

SEQ ID NO: 182 - protein, 104 aa, Artificial Sequence
  Adnectin
MGHHHHHHGGSVSDVPRDLEVVAATPTSLLISWEYDVHPYRYYRITYGETGGNSPVQEFTVPSVLSTATISGLKPGVDYTITVYAVTYGDDSAPISINYRTEID

SEQ ID NO: 183 - protein, 104 aa, Artificial Sequence; identical to SEQ ID NO: 179
  Adnectin

SEQ ID NO: 184 - protein, 104 aa, Artificial Sequence
  Adnectin
MGHHHHHHGGSVSDVPRDLEVVAATPTSLLISWEYKVHPYRYYRITYWETGGNSPVQEFTVPSVLSTATISGLKPGVDYTITVYAVTYGDDSAPISINYRTEID

SEQ ID NO: 185 - protein, 104 aa, Artificial Sequence
  Adnectin
MGHHHHHHGGSVSDVPRDLEVVAATPTSLLISWEYKVHPYRYYRITYGETGGNSPVQEFTVPSVLSTATISGLKPGVDYTITVYAVTYGDDSTPISINYRTEID

SEQ ID NO: 186 - protein, 104 aa, Artificial Sequence; identical to SEQ ID NO: 185
  Adnectin

SEQ ID NO: 187 - protein, 104 aa, Artificial Sequence
  Adnectin
MGHHHHHHGGSVSDVPRDLEVVAATPTSLLISWEYKVHPYRYYRITYGETGGNSPVQEFTVPSVLSTATISGLKPGVDYTITVYAVTYGDDSSPISINYRTEID

SEQ ID NO: 188 - protein, 104 aa, Artificial Sequence
  Adnectin
MGHHHHHHGGSVSDVPRDLEVVAATPTSLLISWEYKVHPYRYYRITYGETGGNSPVQEFTVPSVLSTATISGLKPGVDYTITVYAVTRGDDSAPISINYRTEID

SEQ ID NO: 189 - protein, 104 aa, Artificial Sequence
  Adnectin
MGHHHHHHGGSVSDVPRDLEVVAATPTSLLISWEYKVHPYRYYRITYGETGGNSPVQEFTVPSVLSTATISGLKPGVDYTITVYAVTHGDDSAPISINYRTEID

SEQ ID NO: 190 - protein, 104 aa, Artificial Sequence
  Adnectin
MGHHHHHHGGSVSDVPRDLEVVAATPTSLLISWEYKVHPYRYYRITYGETGGNSPVQEFTVPSVLSTATISGLKPGVDYTITVYAVTYGDDSWPISINYRTEID

SEQ ID NO: 191 - protein, 104 aa, Artificial Sequence; identical to SEQ ID NO: 189
  Adnectin

SEQ ID NO: 192 - protein, 104 aa, Artificial Sequence
  Adnectin
MGHHHHHHGGSVSDVPRDLEVVAATPTSLLISWKYDVHPYRYYRITYGETGGNSPVQEFTVPSVLSTATISGLKPGVDYTITVYAVTYGDDSAPISINYRTEID

SEQ ID NO: 193 - protein, 104 aa, Artificial Sequence
  Adnectin
MGHHHHHHGGSVSDVPRDLEVVAATPTSLLISWEYKVHPYRYYRITYGETGGNSPVQEFTVPSVLSTATISDLKPGVDYTITVYAVTYGDDSHPISINYRTEID

SEQ ID NO: 194 - protein, 104 aa, Artificial Sequence; identical to SEQ ID NO: 188
  Adnectin

SEQ ID NO: 195 - protein, 104 aa, Artificial Sequence
  Adnectin
MGHHHHHHGGSVSDVPRDLEVVAATPTSLLISWEYKVHPYRYYRITYGETGGNSPVQEFTVPSVLSTATIRGLKPGVDYTITVYAVTYGDDSAPISINYRTEID

SEQ ID NO: 196 - protein, 104 aa, Artificial Sequence
  Adnectin
MGHHHHHHGGSVSDVPRDLEVVAATPTSLLISWKYKVHPYRYYRITYGETGGNSPVQEFTVPSVLSTATISGLKPGVDYTITVYAVTYGDDSAPISINYRTEID

SEQ ID NO: 197 - protein, 104 aa, Artificial Sequence
  Adnectin
MGHHHHHHCGSVSDVPRDLEVVAATPTSLLISWEYKVAHNRYYRITYGETGGNSPVQEFTVPSVLSTATISGLKPGVDYTITVYAVTYGDDSAPISINYRTEID

SEQ ID NO: 198 - protein, 104 aa, Artificial Sequence
  Adnectin
MGHHHHHHGGSVSDVPRDLEVVAATPTSLLISWEYKVYGYRYYRITYGETGGNSPVQEFTVPSVLSTATISGLKPGVDYTITVYAVTYGDDSAPISINYRTEID

SEQ ID NO: 199 - protein, 104 aa, Artificial Sequence
  Adnectin
MGHHHHHHGGSVSDVPRDLEVVAATPTSLLISWEYKVDHQRYYRITYGETGGNSPVQEFTVPSVLSTATISGLKPGVDYTITVYAVTYGDDSAPISINYRTEID

SEQ ID NO: 200 - protein, 104 aa, Artificial Sequence
  Adnectin
MGHHHHHHGGSVSDVPRDLEVVAATPTSLLISWEYKVDYRRYYRITYGETGGNSPVQEFTVPSVLSTATISGLKPGVDYTITVYAVTYGDDSAPISINYRTEID

SEQ ID NO: 201 - protein, 104 aa, Artificial Sequence
  Adnectin
MGHHHHHHGGSVSDVPRDLEVVAATPTSLLISWEYKVAYDRYYRITYGETGGNSPVQEFTVPSVLSTATISGLKPGVDYTITVYAVTYGDDSAPISINYRTEID

SEQ ID NO: 202 - protein, 104 aa, Artificial Sequence
  Adnectin
MGHHHHHHGGSVSDVPRDLEVVAATPTSLLISWEYKVTSYRYYRITYGETGGNSPVQEFTVPSVLSTATISGLKPGVDYTITVYAVTYGDDSAPISINYRTEID

SEQ ID NO: 203 - protein, 103 aa, Artificial Sequence
  Adnectin
MGHHHHHHGGSVSDVPRDLEVVAATPTSLLISWEYKVHPYRYYRIIHETGGNSPVQEFTVPSVLSTATISGLKPGVDYTITVYAVTYGDDSAPISINYRTEID

SEQ ID NO: 204 - protein, 105 aa, Artificial Sequence
  Adnectin
MGHHHHHHGGSVSDVPRDLEVVAATPTSLLISWEYKVHPYRYYRITYGETGGNSPVQEFTVPSVLSTATISGLKPGVDYTITVYAVTYGDDSAPISINYRKEAEL

SEQ ID NO: 205 - protein, 104 aa, Artificial Sequence
  Adnectin
MGHHHHHHGGSVSDVPRDLEVVAATPTSLLISWEYKVNHQRYYRITYGETGGNSPVQEFTVPSVLSTATISGLKPGVDYTITVYAVTYGDDSAPISINYRTEID

SEQ ID NO: 206 - protein, 104 aa, Artificial Sequence
  Adnectin
MGHHHHHHGGSVSDVPRDLEVVAATPTSLLISWEYKVDHRRYYRITYGETGGNSPVQEFTVPSVLSTATISGLKPGVDYTITVYAVTYGDDSAPISINYRTEID

SEQ ID NO: 207 - protein, 104 aa, Artificial Sequence
  Adnectin
MGVSDVPRDLEVVAATPTSLLISWEYKVHPYRYYRITYGETGGNSPVQEFTVPSVLSTATISGLKPGVDYTITVYAVTYGVDSDPISINYRTDDKPSQHHHHHH

SEQ ID NO: 208 - protein, 105 aa, Artificial Sequence
  Adnectin
MGVSDVPRDLEVVAATPTSLLISWEYKVHPYRYYRITYGETGGNSPVQEFTVPSVLSTATISGLKPGVDYTITVYAVTYGVNSIPISINYRTEIDKPSQHHHHHH

SEQ ID NO: 209 - protein, 105 aa, Artificial Sequence
  Adnectin
MGVSDVPRDLEVVAATPTSLLISWEYKVNAYRYYRITYGETGGNSPVQEFTVPSVLSTATISGLKPGVDYTITVYAVTYGIESSPISINYRTEIDKPSQHHHHHH

SEQ ID NO: 210 - protein, 105 aa, Artificial Sequence
  Adnectin
MGVSDVPRDLEVVAATPTSLLISWEYKVHPYRYYRITYGETGGNSPVQEFTVPSVLSTATISGLKPGVDYTITVYAVTDGVQSSPISINYRTEIDKPSQHHHHHH

SEQ ID NO: 211 - protein, 105 aa, Artificial Sequence
  Adnectin
MGVSDVPRDLEVVAATPTSLLISWEYKVNAYRYYRITYGETGGNSPVQEFTVPSVLSTATISGLKPGVDYTITVYAVTYGIDSSPISINYRTEIDKPSQHHHHHH

SEQ ID NO: 212 - protein, 105 aa, Artificial Sequence
  Adnectin
MGVSDVPRDLEVVAATPTSLLISWEYKVHYDRYYRITYGETGGNSPVQEFTVPSVLSTATISGLKPGVDYTITVYAVTNGVLSSPISINYRTEIDKPSQHHHHHH

SEQ ID NO: 213 - protein, 105 aa, Artificial Sequence
  Adnectin
MGVSDVPRDLEVVAATPTSLLISWEYKVNPWRYYRITYGETGGNSPVQEFTVPSVLSTATISGLKPGVDYTITVYAVTRGVDSAPISINYRTEIDKPSQHHHHHH

SEQ ID NO: 214 - protein, 105 aa, Artificial Sequence
  Adnectin
  misc_feature (31)..(31): Xaa can be any naturally occurring amino acid
  misc_feature (84)..(84): Xaa can be any naturally occurring amino acid
MGVSDVPRDLEVVAATPTSLLISWEYKVHPXRYYRITYGETGGNSPVQEFTVPSVLSTATISGLKPGVDYTITVYAVTYGVBSXPISINYRTEIDKPSQHHHHHH

SEQ ID NO: 215 - protein, 105 aa, Artificial Sequence
  Adnectin
MGVSDVPRDLEVVAATPTSLLISWEYKVNPWRYYRITYGETGGNSPVQEFTVPSVLSTATISGLKPGVDYTITVYAVTYGIQSPPISINYRTEIDKPSQHHHHHH

SEQ ID NO: 216 - protein, 105 aa, Artificial Sequence
  Adnectin
MGVSDVPRDLEVVAATPTSLLISWQYKVHPYRWYRITYGETGGNSPVQEFTVPSVLSTATISGLKPGVDYTITVYAVTYGVNSIPISINYRTEIDKPSQHHHHHH

SEQ ID NO: 217 - protein, 105 aa, Artificial Sequence
  Adnectin
MGVSDVPRDLEVVAATPTSLLISWEYKVSPYRYYRITYGETGGNSPVQEFTVPSVLSTATISGLKPGVDYTITVYAVTYGVNSIPISINYRTEIDKPSQHHHHHH

SEQ ID NO: 218 - protein, 105 aa, Artificial Sequence
  Adnectin
MGVSDVPRDLEVVAATPTSLLISWEYKVNPWRYYRITYGETGGNSPVQEFTVPSVLSTATISGLKPGVDYTITVYAVTYGVNSLPISINYRTEIDKPSQHHHHHH

SEQ ID NO: 219 - protein, 105 aa, Artificial Sequence
  Adnectin
MGVSDVPRDLEVVAATPTSLLISWEYNVHYDRYYRITYGETGGNSPVQEFTVPSVLSTATISGLKPGVDYTITVYAVTYGVDSDPISINYRTEIDKPSQHHHHHH

SEQ ID NO: 220 - protein, 105 aa, Artificial Sequence
  Adnectin
MGVSDVPRDLEVVAATPTSLLISWEYKVNPWRYYRITYGETGGNSPVQEFTVPSVLSTATISGLKPGVDYTITVYAVTYGVDSDPISINYRTEIDKPSQHHHHHH

SEQ ID NO: 221 - protein, 105 aa, Artificial Sequence
  Adnectin
MGVSDVPRDLEVVAATPTSLLISWEYKVDPYRYYRITYGETGGNSPVQEFTVPSVLSTATISGLKPGVDYTITVYAVTYGVDSDPISINYRTEIDKPSQHHHHHH

SEQ ID NO: 222 - protein, 104 aa, Artificial Sequence
  Adnectin
MGHHHHHHGGSVSDVPRDLEVVAATPTSLLISWKYQVHAYRYYRITYGETGGNSPVQEFTVPSVLSTATINGLKPGVDYTITVYAVTYGIISEPISINYRTEID

SEQ ID NO: 223 - protein, 104 aa, Artificial Sequence
  Adnectin
MGHHHHHHGGSVSDVPRDLEVVAATPTSLLISWEYKVNYNRYYRITYGETGGNSPVQEFTVPSVLSTATISGLKPGVDYTITVYAVTEGVQSAPISINYRTEID

SEQ ID NO: 224 - protein, 104 aa, Artificial Sequence
  Adnectin
MGHHHHHHGGSVSDVPRDLEVVAATPTSLLISWEYKVHPYRYYRITYGETGGNSPVQEFTVPSVLSTATISGLKPGVDYTITVYAVTYGDDSAPISINYRTPGG

SEQ ID NO: 225 - protein, 104 aa, Artificial Sequence
  Adnectin
MGHHHHHHGGSVSDVPRDLEVVAATPTSLLISWEYKVHPYRYYRITYGETGGNTPVQEFRVPSVLSTATISGLKPGVDYTITVYAVTYADASAPISINYRTEID

SEQ ID NO: 226 - protein, 104 aa, Artificial Sequence
  Adnectin
MGHHHHHHGGSVSDVPRDLEVVAATPTSLLISWEYKVHPYRYYRITYGETGGNSPVQEFTVPSVLSSATLNGLKPGVDYTITVYAVTYGDDSAPISINYRTEID

SEQ ID NO: 227 - protein, 104 aa, Artificial Sequence
  Adnectin
MGHHHHHHGGSVSDVPRDLEVVAATPTSLLISWEYKVHPYRYYRITYGETGGNSPVQEFTVPSVLSSAKISGLKPGVDYTITVYAVTYGDDSAPISINYRTEID

SEQ ID NO: 228 - protein, 104 aa, Artificial Sequence
  Adnectin
MGHHHHHHGGSVSDVPRDLEVVAATPTSLLISWEYKVHPYRYYRITYGETGGNSPVQEFNVPSVLSTATISGLKPGVDYTITVYAVTYGDDSAPISINYRTEID

SEQ ID NO: 229 - protein, 104 aa, Artificial Sequence
  Adnectin
MGHHHHHHGGSVSDVPRDLEVVAATPTSLLISWEYKVAHRRYYRITYGETGGNSPVQEFTVPSVLSTATISGLKPGVDYTITVYAVTYGDDSAPISINYRTEID

SEQ ID NO: 230 - protein, 104 aa, Artificial Sequence
  Adnectin
MGHHHHHHGGSVSDVPRDLEVVAATPTSLLISWEYKVDAYRYYRITYGETGGNSPVQEFTVPSVLSTATISGLKPGVDYTITVYAVTYGDDSAPISINYRTEID

SEQ ID NO: 231 - protein, 104 aa, Artificial Sequence
  Adnectin
MGHHHHHHGGSVSDVPRDLEVVAATPTSLLISWEYKVDSYRYYRITYGETGGNSPVQEFTVPSVLSTATISGLKPGVDYTITVYAVTYGDDSAPISINYRTEID

SEQ ID NO: 232 - protein, 104 aa, Artificial Sequence; identical to SEQ ID NO: 94
  Adnectin

SEQ ID NO: 233 - protein, 104 aa, Artificial Sequence
  Adnectin
MGHHHHHHGGSVSDVPRDLEVVAATPTSLLISWEYKVHPYRYYRITYGETGGNSPVQEFTVPSVLSTATISGLKPGVDYTITVYAVTYGDASAPISINYRTEID

SEQ ID NO: 234 - protein, 104 aa, Artificial Sequence
  Adnectin
MGHHHHHHGGSVSDVPRDLEVVAATPTSLLISWEYKVHPYRYYRITYGETGGNSPVQEFTVPSVLSTATISGLKPGVDYTITVYAVTNGDDSAPISINYRTEID

SEQ ID NO: 235 - protein, 104 aa, Artificial Sequence
  Adnectin
MGHHHHHHGGSVSDVPRDLEVVAATPTSLLISWEYKVHPYRYYRITYGETGGNSPVQEFTVPSVLSSATIRGLKPGVDYTITVYAVTYGDDSAPISINYRTEID

SEQ ID NO: 236 - protein, 104 aa, Artificial Sequence
  Adnectin
MGHHHHHHGGSVSDVPRDLEVVAATPTSLLISWEYKVHPYRYYRITYGETGGNSPVQEFTVPSVLSTAKISGLKPGVDYTITVYAVTYGDDSAPISINYRTEID

SEQ ID NO: 237 - protein, 104 aa, Artificial Sequence
  Adnectin
MGHHHHHHGGSVSDVPRDLEVVAATPTSLLISWEYKVHPYRYYRITYGETGGNSPVQEFRVPSVLSTATISGLKPGVDYTITVYAVTYGDDSAPISINYRTEID

SEQ ID NO: 238 - protein, 104 aa, Artificial Sequence
  Adnectin
MGHHHHHHGGSVSDVPRDLEVVAATPTSLLISWEYKVAPWRYYRITYGETGGNSPVQEFTVPSVLSTATISGLKPGVDYTITVYAVTYGDDSAPISINYRTEID

SEQ ID NO: 239 - protein, 104 aa, Artificial Sequence
  Adnectin
MGHHHHHHGGSVSDVPRDLEVVAATPTSLLISWEYKVDGWRYYRITYGETGGNSPVQEFTVPSVLSTATISGLKPGVDYTITVYAVTYGDDSAPISINYRTEID

SEQ ID NO: 240 - protein, 104 aa, Artificial Sequence
  Adnectin
MGHHHHHHGGSVSDVPRDLEVVAATPTSLLISWEYKVDTWRYYRITYGETGGNSPVQEFTVPSVLSTATISGLKPGVDYTITVYAVTYGDDSAPISINYRTEID

SEQ ID NO: 241 - protein, 104 aa, Artificial Sequence
  Adnectin
  misc_feature (28)..(28): Xaa can be any naturally occurring amino acid
MGHHHHHHGGSVSDVPRDLEVVAATPTXLLISWEYKVHPYRYYRITYGETGGNSPVQEFTVPSVLSTATISGLKPGVDYTITVYAVTYGDDSAPISINYRTEID

SEQ ID NO: 242 - protein, 104 aa, Artificial Sequence; identical to SEQ ID NO: 167
  Adnectin

SEQ ID NO: 243 - protein, 104 aa, Artificial Sequence; identical to SEQ ID NO: 188
  Adnectin

SEQ ID NO: 244 - protein, 104 aa, Artificial Sequence
  Adnectin
MGHHHHHHGGSVSDVPRDLEVVAATPTSLLISWEYKVHPYRYYRITYGETGGNSPVQDFTVPSVLSTATIRGLKPGVDYTITVYAVTYGDDSAPISINYRTEID

SEQ ID NO: 245 - protein, 104 aa, Artificial Sequence
  Adnectin
MGHHHHHHGGSVSDVPRDLEVVAATPTSLLISWEYKVHPYRYYRITYGETGGNSPVQEFTVPSVLSTANISGLKPGVDYTITVYAVTYGDDSAPISINYRTEID

SEQ ID NO: 246 - protein, 104 aa, Artificial Sequence
  Adnectin
MGHHHHHHGGSVSDVPRDLEVVAATPTSLLISWEYKVHPYRYYRITYGETGGNSPVQAFTVPSVLSTATISGLKPGVDYTITVYAVTYGDDSAPISINYRTEID

SEQ ID NO: 247 - protein, 104 aa, Artificial Sequence
  Adnectin
MGHHHHHHGGSVSDVPRDLEVVAATPTSLLISWEYKVAPYRYYRITYGETGGNSPVQEFTVPSVLSTATISGLKPGVDYTITVYAVTYGDDSAPISINYRTEID

SEQ ID NO: 248 - protein, 104 aa, Artificial Sequence
  Adnectin
MGHHHHHHGGSVSDVPRDLEVVAATPTSLLISWEYKVDGYRYYRITYGETGGNSPVQEFTVPSVLSTATISGLKPGVDYTITVYAVTYGDDSAPISINYRTEID

SEQ ID NO: 249 - protein, 104 aa, Artificial Sequence
  Adnectin
MGHHHHHHGGSVSDVPRDLEVVAATPTSLLISWEYKVDTYRYYRITYGETGGNSPVQEFTVPSVLSTATISGLKPGVDYTITVYAVTYGDDSAPISINYRTEID

SEQ ID NO: 250 - protein, 104 aa, Artificial Sequence
  Adnectin
MGHHHHHHGGSVSDVPRDLEVVAATPTSLLISWEYEVHPYRYYRITYGETGGNSPVQEFTVPSVLSTATISGLKPGVDYTITVYAVTYGDDSAPISINYRTEID

SEQ ID NO: 251 - protein, 104 aa, Artificial Sequence
  Adnectin
MGHHHHHHGGSVSDVPRDLEVVAATPTSLLISWEYRVHPYRYYRITYGETGGNSPVQEFTVPSVLSTATISGLKPGVDYTITVYAVTYGDDSAPISINYRTEID

SEQ ID NO: 252 - protein, 104 aa, Artificial Sequence
  Adnectin
MGHHHHHHGGSVSDVPRDLEVVAATPTSLLISWEYKVHPYRYYRITYGETGGNSPVQSFTVPSVLSTATISGLKPGVDYTITVYAVTYGADSAPISINYRTEID

SEQ ID NO: 253 - protein, 104 aa, Artificial Sequence; identical to SEQ ID NO: 195
  Adnectin

SEQ ID NO: 254 - protein, 104 aa, Artificial Sequence
  Adnectin
MGHHHHHHGGSVSDVPRDLEVVAATPTSLLISWEYKVHPYRYYRITYGETGGNSPVQDFTVPSVLSTATISGLKPGVDYTITVYAVTYGDDSAPISINYRTEID

SEQ ID NO: 255 - protein, 104 aa, Artificial Sequence
  Adnectin
MGHHHHHHGGSVSDVPRDLEVVAATPTSLLISWEYKVARWRYYRITYGETGGNSPVQEFTVPSVLSTATISGLKPGVDYTITVYAVTYGDDSAPISINYRTEID

SEQ ID NO: 256 - protein, 104 aa, Artificial Sequence; identical to SEQ ID NO: 199
  Adnectin

SEQ ID NO: 257 - protein, 104 aa, Artificial Sequence
  Adnectin
MGHHHHHHGGSVSDVPRDLEVVAATPTSLLISWEYKVDYGRYYRITYGETGGNSPVQEFTVPSVLSTATISGLKPGVDYTITVYAVTYGDDSAPISINYRTEID

SEQ ID NO: 258 - protein, 104 aa, Artificial Sequence
  Adnectin
MGHHHHHHGGSVSDVPRDLEVVAATPTSLLISWKYGVHPYRYYRITYGETGGNSPVQEFTVPSVLSTATISGLKPGVDYTITVYAVTYGDDSAPISINYRTEID

SEQ ID NO: 259 - protein, 104 aa, Artificial Sequence
  Adnectin
MGHHHHHHGGSVSDVPRDLEVVAATPTSLLISWEYSVHPYRYYRITYGETGGNSPVQEFTVPSVLSTATISGLKPGVDYTITVYAVTYGDDSAPISINYRTEID

SEQ ID NO: 260 - protein, 104 aa, Artificial Sequence
  Adnectin
MGHHHHHHGGSVSDVPRDLEVVAATPTSLLISWEYKVHPYRYYRITYGETGGNSPVQEFTVPSVLSTATISGLKPGVDYTITVYAVTDGDDSAPISINYRTEID

SEQ ID NO: 261 - protein, 104 aa, Artificial Sequence
  Adnectin
MGHHHHHHGGSVSDVPRDLEVVAATPTSLLISWEYKVHPYRYYRITYGETGGNSPVQEFTVPSVLSTATISGLKPGVDYTITVYAVTSGDDSAPISINYRTEID

SEQ ID NO: 262 - protein, 104 aa, Artificial Sequence
  Adnectin
MGHHHHHHGGSVSDVPRDLEVVAATPTSLLISWEYKVHPYRYYRITYGETGGNSPVQEFTVPSVLSSATLRGLKPGVDYTITVYAVTYGDDSAPISINYRTEID

SEQ ID NO: 263 - protein, 104 aa, Artificial Sequence
  Adnectin
MGHHHHHHGGSVSDVPRDLEVVAATPTSLLISWEYKVAAWRYYRITYGETGGNSPVQEFTVPSVLSTATISGLKPGVDYTITVYAVTYGDDSAPISINYRTEID

SEQ ID NO: 264 - protein, 104 aa, Artificial Sequence
  Adnectin
MGHHHHHHGGSVSDVPRDLEVVAATPTSLLISWEYKVATWRYYRITYGETGGNSPVQEFTVPSVLSTATISGLKPGVDYTITVYAVTYGDDSAPISINYRTEID

SEQ ID NO: 265 - protein, 104 aa, Artificial Sequence
  Adnectin
MGHHHHHHGGSVSDVPRDLEVVAATTTSLLISWEYKVDYHRYYRITYGETGGNSPVQEFTVPSVLSTATISGLKPGVDYTITVYAVTYGDDSAPISINYRTEID

SEQ ID NO: 266 - protein, 104 aa, Artificial Sequence
  Adnectin
MGHHHHHHGGSVSDVPRDLEVVAATPTSLLISWEYGVHPYRYYRITYGETGGNSPVQEFTVPSVLSTATISGLKPGVDYTITVYAVTYGDDSAPISINYRTEID

SEQ ID NO: 267 - protein, 104 aa, Artificial Sequence
  Adnectin
  misc_feature (36)..(36): Xaa can be any naturally occurring amino acid
MGHHHHHHGGSVSDVPRDLEVVAATPTSLLISWKYXVHPYRYYRITYGETGGNSPVQEFTVPSVLSTATISGLKPGVDYTITVYAVTYGDDSAPISINYRTEID

SEQ ID NO: 268 - protein, 104 aa, Artificial Sequence
  Adnectin
MGHHHHHHGGSVSDVPRDLEVVAATPTSLLISWEYKVHPYRYYRITYGETGGNSPVQEFTVPSVLSTATISGLKPGVDYTITVYAVTGGDDSAPISINYRTEID

SEQ ID NO: 269 - protein, 104 aa, Artificial Sequence
  Adnectin
MGHHHHHHGGSVSDVPRDLEVVAATPTSLLISWEYKVHPYRYYRITYGETGGNSPVQEFTVPSVLSTATIHGLKPGVDYTITVYAVTYGDDSAPISINYRTEID

SEQ ID NO: 270 - protein, 104 aa, Artificial Sequence
  Adnectin
MGHHHHHHGGSVSDVPRDLEVVAATPTSLLISWEYKVHPYRYYRITYGETGGNSPVQEFTVPSVLSTATLRGLKPGVDYTITVYAVTYGDDSAPISINYRTEID

SEQ ID NO: 271 - protein, 104 aa, Artificial Sequence
  Adnectin
MGHHHHHHGGSVSDVPRDLEVVAATPTSLLISWEYKVHPYRYYRITYGETGGNSPVQEFTVPSVLSSATISGLKPGVDYTITVYAVTYGDDSAPISINYRTEID

SEQ ID NO: 272 - protein, 104 aa, Artificial Sequence
  Adnectin
MGHHHHHHGGSVSDVPRDLEVVAATPTSLLISWEYKVAAYRYYRITYGETGGNSPVQEFTVPSVLSTATISGLKPGVDYTITVYAVTYGDDSAPISINYRTEID

SEQ ID NO: 273 - protein, 104 aa, Artificial Sequence
  Adnectin
MGHHHHHHGGSVSDVPRDLEVVAATPTSLLISWEYKVATYRYYRITYGETGGNSPVQEFTVPSVLSTATISGLKPGVDYTITVYAVTYGDDSAPISINYRTEID

SEQ ID NO: 274 - protein, 104 aa, Artificial Sequence
  Adnectin
MGHHHHHHGGSVSDVPRDLEVVAATPTSLLISWEYKVDPWRYYRITYGETGGNSPVQEFTVPSVLSTATISGLKPGVDYTITVYAVTYGDDSAPISINYRTEID

SEQ ID NO: 275 - protein, 104 aa, Artificial Sequence
  Adnectin
MGHHHHHHGGSVSDVPRDLEVVAATPTSLLISWEYKVDYQRYYRITYGETGGNSPVQEFTVPSVLSTATISGLKPGVDYTITVYAVTYGDDSAPISINYRTEID

SEQ ID NO: 276 - protein, 104 aa, Artificial Sequence; identical to SEQ ID NO: 196
  Adnectin

SEQ ID NO: 277 - protein, 104 aa, Artificial Sequence
  Adnectin
  misc_feature (36)..(36): Xaa can be any naturally occurring amino acid
MGHHHHHHGGSVSDVPRDLEVVAATPTSLLISWEYXVHPYRYYRITYGETGGNSPVQEFTVPSVLSTATISGLKPGVDYTITVYAVTYGDDSAPISINYRTEID

SEQ ID NO: 278 - protein, 105 aa, Artificial Sequence
  Adnectin
MGHHHHHHGGSVSDVPRDLEVVAATPTSLLISWEYKVHPYRYYRITYGETGGNSPVQEFTVPSVLSTATISGLKPGVDYTITVYAVTHGDDFAPISINYRTEMLI

SEQ ID NO: 279 - protein, 104 aa, Artificial Sequence
  Adnectin
MGHHHHHHGGSVSDVPRDLEVVAATPTSLLISWEYKVHPYRYYRITYGETGGNSPVQEFTVPSVLSSATINGLKPGVDYTITVYAVTYGDDSAPISINYRTEID

SEQ ID NO: 280 - protein, 104 aa, Artificial Sequence; identical to SEQ ID NO: 164
  Adnectin

SEQ ID NO: 281 - protein, 104 aa, Artificial Sequence
  Adnectin
MGHHHHHHGGSVSDVPRDLEVVAATPTSLLISWEYKVAHDRYYRITYGETGGNSPVQEFTVPSVLSTATISGLKPGVDYTITVYAVTYGDDSAPISINYRTEID

SEQ ID NO: 282 - protein, 104 aa, Artificial Sequence
  Adnectin
MGHHHHHHGGSVSDVPRDLEVVAATPTSLLISWEYKVDPYRYYRITYGETGGNSPVQEFTVPSVLSTATISGLKPGVDYTITVYAVTYGDDSAPISINYRTEID

SEQ ID NO: 283 - protein, 104 aa, Artificial Sequence; identical to SEQ ID NO: 200
  Adnectin

SEQ ID NO: 284 - protein, 104 aa, Artificial Sequence
  Adnectin
MGHHHHHHGGSVSDVPRDLEVVAATPTGLLISWEYKVHPYRYYRITYGETGGNSPVQEFTVPSVLSTATISGLKPGVDYTITVYAVTYGDDSAPISINYRTEID

SEQ ID NO: 285 - protein, 104 aa, Artificial Sequence
  Adnectin
MGHHHHHHGGSVSDVPRDLEVVAATPTSLLISWEYKVNAWRYYRITYGETGGNSPVQEFTVPSVLSTATISGLKPGVDYTITVYAVTYGDDSAPISINYRTEID

SEQ ID NO: 286 - protein, 104 aa, Artificial Sequence; identical to SEQ ID NO: 189
  Adnectin

SEQ ID NO: 287 - protein, 104 aa, Artificial Sequence
  Adnectin
MGHHHHHHGGSVSDVPRDLEVVAATPTSLLISWEYKVHPYRYYRITYGETGGNSPVQEFTVPSVLSTATINGLKPGVDYTITVYAVTYGDDSAPISINYRTEID

SEQ ID NO: 288 - protein, 104 aa, Artificial Sequence
  Adnectin
MGHHHHHHGGSVSDVPRDLEVVAATPTSLLISWEYKVHPYRYYRITYGETGGNSPVQEFTVPSVLSTAEISGLKPGVDYTITVYAVTYGDDSAPISINYRTEID

SEQ ID NO: 289 - protein, 104 aa, Artificial Sequence
  Adnectin
MGHHHHHHGGSVSDVPRDLEVVAATPTSLLISWEYKVHPYRYYRITYGETGGNSPVQEFLVPSVLSTATISGLKPGVDYTITVYAVTYGDDSAPISINYRTEID

SEQ ID NO: 290 - protein, 104 aa, Artificial Sequence
  Adnectin
MGHHHHHHGGSVSDVPRDLEVVAATPTSLLISWEYKVAHGRYYRITYGETGGNSPVQEFTVPSVLSTATISGLKPGVDYTITVYAVTYGDDSAPISINYRTEID

SEQ ID NO: 291 - protein, 104 aa, Artificial Sequence
  Adnectin
MGHHHHHHGGSVSDVPRDLEVVAATPTSLLISWEYKVAYKRYYRITYGETGGNSPVQEFTVPSVLSTATISGLKPGVDYTITVYAVTYGDDSAPISINYRTEID

SEQ ID NO: 292 - protein, 104 aa, Artificial Sequence
  Adnectin
MGHHHHHHGGSVSDVPRDLEVVAATPTSLLISWEYKVDRWRYYRITYGETGGNSPVQEFTVPSVLSTATISGLKPGVDYTITVYAVTYGDDSAPISINYRTEID

SEQ ID NO: 293 - protein, 104 aa, Artificial Sequence
  Adnectin
MGHHHHHHGGSVSDVPRDLEVVAATPTSLLISWEYAVHPYRYYRITYGETGGNSPVQEFTVPSVLSTATISGLKPGVDYTITVYAVTYGDDSAPISINYRTEID

SEQ ID NO: 294 - protein, 104 aa, Artificial Sequence
  Adnectin
MGHHHHHHGGSVSDVPRDLEVVAATPTSLLISWEYKVNAYRYYRITYGETGGNSPVQEFTVPSVLSTATISGLKPGVDYTITVYAVTYGDDSAPISINYRTEID

SEQ ID NO: 295 - protein, 104 aa, Artificial Sequence
  Adnectin
MGHHHHHHGGSVSDVPRDLEVVAATPTSLLISWEYKVNGYRYYRITYGETGGNSPVQEFTVPSVLSTATISGLKPGVDYTITVYAVTYGDDSAPISINYRTEID

SEQ ID NO: 296 - protein, 104 aa, Artificial Sequence
  Adnectin
MGHHHHHHGGSVSDVPRDLEVVAATPTSLLISWEYKVSHGRYYRITYGETGGNSPVQEFTVPSVLSTATISGLKPGVDYTITVYAVTYGDDSAPISINYRTEID

SEQ ID NO: 297 - protein, 104 aa, Artificial Sequence
  Adnectin
MGHHHHHHGGSVSDVPRDLEVVAATPTSLLISWEYKVTTWRYYRITYGETGGNSPVQEFTVPSVLSTATISGLKPGVDYTITVYAVTYGDDSAPISINYRTEID

SEQ ID NO: 298 - protein, 104 aa, Artificial Sequence
  Adnectin
MGHHHHHHGGSVSDVPRDLEVVAATPTSLLISWEYKVHPYRYYRITYGETGGNSPVQEFTVPSVLSSAZLSGLKPGVDYTITVYAVTYGDDSAPISINYRTEID

SEQ ID NO: 299 - protein, 104 aa, Artificial Sequence; identical to SEQ ID NO: 168
  Adnectin

SEQ ID NO: 300 - protein, 104 aa, Artificial Sequence; identical to SEQ ID NO: 159
  Adnectin

SEQ ID NO: 301 - protein, 104 aa, Artificial Sequence
  Adnectin
MGHHHHHHGGSVSDVPRDLEVVAATPTSLLISWEYKVHPYRYYRITYGETGGNSPVQEFTVPSVLSTATISGLKPGVDYTITVYAVTYGDDSNPISINYRTEID

SEQ ID NO: 302 - protein, 104 aa, Artificial Sequence
  Adnectin
MGHHHHHHGGSVSDVPRDLEVVAATPTSLLISWEYKVNHDRYYRITYGETGGNSPVQEFTVPSVLSTATISGLKPGVDYTITVYAVTYGDDSAPISINYRTEID

SEQ ID NO: 303 - protein, 104 aa, Artificial Sequence
  Adnectin
MGHHHHHHGGSVSDVPRDLEVVAATPTSLLISWEYKVSHRRYYRITYGETGGNSPVQEFTVPSVLSTATISGLKPGVDYTITVYAVTYGDDSAPISINYRTEID

SEQ ID NO: 304 - protein, 104 aa, Artificial Sequence
  Adnectin
MGHHHHHHGGSVSDVPRDLEVVAATPTSLLISWEYKVTYERYYRITYGETGGNSPVQEFTVPSVLSTATISGLKPGVDYTITVYAVTYGDDSAPISINYRTEID

SEQ ID NO: 305 - protein, 104 aa, Artificial Sequence
  Adnectin
  misc_feature (70)..(71): Xaa can be any naturally occurring amino acid
MGHHHHHHGGSVSDVPRDLEVVAATPTSLLISWEYKVHPYRYYRITYGETGGNSPVQEFTVPSVLSSATXXGLKPGVDYTITVYAVTYGDDSAPISINYRTEID

SEQ ID NO: 306 - protein, 104 aa, Artificial Sequence; identical to SEQ ID NO: 161
  Adnectin

SEQ ID NO: 307 - protein, 104 aa, Artificial Sequence
  Adnectin
MGHHHHHHGGSVSDVPRDLEVVAATPTSLLISWEYKVHPYRYYRITYGETGGNSPVQEFTVPSVLSTATISGLKPGVDYTITVYAVTYGDDSPPISINYRTEID

SEQ ID NO: 308 - protein, 104 aa, Artificial Sequence
  Adnectin
  misc_feature (93)..(93): Xaa can be any naturally occurring amino acid
MGHHHHHHGGSVSDVPRDLEVVAATPTSLLISWEYKVHPYRYYRITYGETGGNSPVQEFTVPSVLSTATISGLKPGVDYTITVYAVTYGDDSXPISINYRTEID

SEQ ID NO: 309 - protein, 104 aa, Artificial Sequence
  Adnectin
MGHHHHHHGGSVSDVPRDLEVVAATPTSLLISWEYKVNRWRYYRITYGETGGNSPVQEFTVPSVLSTATISGLKPGVDYTITVYAVTYGDDSAPISINYRTEID

SEQ ID NO: 310 - protein, 104 aa, Artificial Sequence
  Adnectin
MGHHHHHHGGSVSDVPRDLEVVAATPTSLLISWEYKVSPWRYYRITYGETGGNSPVQEFTVPSVLSTATISGLKPGVDYTITVYAVTYGDDSAPISINYRTEID

SEQ ID NO: 311 - protein, 104 aa, Artificial Sequence
  Adnectin
MGHHHHHHGGSVSDVPRDLEVVAATPTSLLISWEYKVTYRRYYRITYGETGGNSPVQEFTVPSVLSTATISGLKPGVDYTITVYAVTYGDDSAPISINYRTEID

SEQ ID NO: 312 - protein, 104 aa, Artificial Sequence
  Adnectin
MGHHHHHHGGSVSDVPRDLEVVAATPTSLLISWEYKVHPYRYYRITYGETGGNSPVQEFTVPSVLSTATISGLKPGVDYTITVYAVTYGDESAPISINYRTEID

SEQ ID NO: 313 - protein, 104 aa, Artificial Sequence
  Adnectin
MGHHHHHHGGSVSDVPRDLEVVAATPTSLLISWEYKVHPYRYYRITYGETGGNSPVQEFTVPSVLSTATISGLKPGVDYTITVYAVTYGDDSDPISINYRTEID

SEQ ID NO: 314 - protein, 104 aa, Artificial Sequence
  Adnectin
MGHHHHHHGGSVSDVPRDLEVVAATPTSLLISWEYKVHPYRYYRITYGETGGNSPVQEFTVPSVLSTATISGLKPGVDYTITVYAVTYGDDSYPISINYRTEID

SEQ ID NO: 315 - protein, 104 aa, Artificial Sequence
  Adnectin
MGHHHHHHGGSVSDVPRDLEVVAATPTSLLISWEYKVNRYRYYRITYGETGGNSPVQEFTVPSVLSTATISGLKPGVDYTITVYAVTYGDDSAPISINYRTEID

SEQ ID NO: 316 - protein, 104 aa, Artificial Sequence
  Adnectin
MGHHHHHHGGSVSDVPRDLEVVAATPTSLLISWEYKVSRWRYYRITYGETGGNSPVQEFTVPSVLSTATISGLKPGVDYTITVYAVTYGDDSAPISINYRTEID

SEQ ID NO: 317 - protein, 104 aa, Artificial Sequence
  Adnectin
  misc_feature (38)..(39): Xaa can be any naturally occurring amino acid
MGHHHHHHGGSVSDVPRDLEVVAATPTSLLISWEYKVXXYRYYRITYGETGGNSPVQEFTVPSVLSTATISGLKPGVDYTITVYAVTYGDDSAPISINYRTEID

SEQ ID NO: 318 - protein, 104 aa, Artificial Sequence
  Adnectin
MGHHHHHHGGSVSDVPRDLEVVAATPTSLLISWEYKVHPYRYYRITYGETGGNSPVQSFTVPSVLSTATISGLKPGVDYTITVYAVTYGDDSAPISINYRTEID

SEQ ID NO: 319 - protein, 104 aa, Artificial Sequence; identical to SEQ ID NO: 160
  Adnectin

SEQ ID NO: 320 - protein, 104 aa, Artificial Sequence; identical to SEQ ID NO: 158
  Adnectin

SEQ ID NO: 321 - protein, 104 aa, Artificial Sequence
  Adnectin
MGHHHHHHGGSVSDVPRDLEVVAATPTSLLISWEYKVHPYRYYRITYGETGGNSPVQEFTVPSVLSTATISGLKPGVDYTITVYAVTYGDDSEPISINYRTEID

SEQ ID NO: 322 - protein, 104 aa, Artificial Sequence; identical to SEQ ID NO: 156
  Adnectin

SEQ ID NO: 323 - protein, 104 aa, Artificial Sequence; identical to SEQ ID NO: 175
  Adnectin

SEQ ID NO: 324 - protein, 104 aa, Artificial Sequence
  Adnectin
MGHHHHHHGGSVSDVPRDLEVVAATPTSLLISWEYKVNYRRYYRITYGETGGNSPVQEFTVPSVLSTATISGLKPGVDYTITVYAVTYGDDSAPISINYRTEID

SEQ ID NO: 325 - protein, 104 aa, Artificial Sequence
  Adnectin
MGHHHHHHGGSVSDVPRDLEVVAATPTSLLISWEYKVSTWRYYRITYGETGGNSPVQEFTVPSVLSTATISGLKPGVDYTITVYAVTYGDDSAPISINYRTEID

SEQ ID NO: 326 - protein, 104 aa, Artificial Sequence
  Adnectin
MGHHHHHHGGSVSDVPRDLEVVAATPTSLLISWEYKVYAYRYYRITYGETGGNSPVQEFTVPSVLSTATISGLKPGVDYTITVYAVTYGDDSAPISINYRTEID

SEQ ID NO: 327 - protein, 104 aa, Artificial Sequence
  Adnectin
MGHHHHHHGGSVSDVPRDLEVVAATPTSLLISWEYKVHPYRYYRITYGETGGNSPVQTFTVPSVLSTATISGLKPGVDYTITVYAVTYGDDSAPISINYRTEID

SEQ ID NO: 328 - protein, 104 aa, Artificial Sequence
  Adnectin
MGHHHHHHGGSVSDVPRDLEVVAATPTSLLISWEYKVHPYRYYRITYGETGGNSPVQAFTVPSVLSTATISGLKPGVDYTITVYAVTYGKDSAPISINYRTEID

SEQ ID NO: 329 - protein, 104 aa, Artificial Sequence; identical to SEQ ID NO: 108
  Adnectin

SEQ ID NO: 330 - protein, 104 aa, Artificial Sequence; identical to SEQ ID NO: 174
  Adnectin

SEQ ID NO: 331 - protein, 104 aa, Artificial Sequence
  Adnectin
MGHHHHHHGGSVSDVPRDLEVVAATPTSLLISWEYKVSAWRYYRITYGETGGNSPVQEFTVPSVLSTATISGLKPGVDYTITVYAVTYGDDSAPISINYRTEID

SEQ ID NO: 332 - protein, 104 aa, Artificial Sequence
  Adnectin
MGHHHHHHGGSVSDVPRDLEVVAATPTSLLISWEYKVSTYRYYRITYGETGGNSPVQEFTVPSVLSTATISGLKPGVDYTITVYAVTYGDDSAPISINYRTEID

SEQ ID NO: 333 - protein, 104 aa, Artificial Sequence
  Adnectin
MGHHHHHHGGSVSDVPRDLEVVAATPTSLLISWEYKVHPYRYYRITYGETGGNSPVQEFTVPSVLSTAELSGLKPGVDYTITVYAVTYGDDSAPISINYRTEID

SEQ ID NO: 334 - protein, 104 aa, Artificial Sequence; identical to SEQ ID NO: 108
  Adnectin

SEQ ID NO: 335 - protein, 104 aa, Artificial Sequence; identical to SEQ ID NO: 185
  Adnectin

SEQ ID NO: 336 - protein, 104 aa, Artificial Sequence
  Adnectin
MGHHHHHHGGSVSDVPRDLEVVAATPTSLLISWEYKVSAYRYYRITYGETGGNSPVQEFTVPSVLSTATISGLKPGVDYTITVYAVTYGDDSAPISINYRTEID

SEQ ID NO: 337 - protein, 104 aa, Artificial Sequence
  Adnectin
MGHHHHHHGGSVSDVPRDLEVVAATPTSLLISWEYKVTGYRYYRITYGETGGNSPVQEFTVPSVLSTATISGLKPGVDYTITVYAVTYGDDSAPISINYRTEID

SEQ ID NO: 338 - protein, 104 aa, Artificial Sequence
  Adnectin
MGHHHHHHGGSVSDVPRDLEVVAATPTSLLISWEYKVYHGRYYRITYGETGGNSPVQEFTVPSVLSTATISGLKPGVDYTITVYAVTYGDDSAPISINYRTEID

SEQ ID NO: 339 - protein, 104 aa, Artificial Sequence
  Adnectin
MGHHHHHHGGSVSDVPRDLEVVAATPTSLLISWEYKVHPYRYYRITYGETGGNSPVQEFTVPSVLSTAKLSGLKPGVDYTITVYAVTYGDDSAPISINYRTEID

SEQ ID NO: 340 - protein, 104 aa, Artificial Sequence
  Adnectin
MGHHHHHHGGSVSDVPRDLEVVAATPTSLLISWEYKVHPYRYYRITYGETGGNSPVQEFTVPSVLSTATISGLKPGVDYTITVYAVTYGNDSAPISINYRTEID

SEQ ID NO: 341 - protein, 104 aa, Artificial Sequence; identical to SEQ ID NO: 171
  Adnectin

SEQ ID NO: 342 - protein, 104 aa, Artificial Sequence
  Adnectin
MGHHHHHHGGSVSDVPRDLEVVAATPTSLLISWEYKVSGYRYYRITYGETGGNSPVQEFTVPSVLSTATISGLKPGVDYTITVYAVTYGDDSAPISINYRTEID

SEQ ID NO: 343 - protein, 104 aa, Artificial Sequence
  Adnectin
MGHHHHHHGGSVSDVPRDLEVVAATPTSLLISWEYKVYHNRYYRITYGETGGNSPVQEFTVPSVLSTATISGLKPGVDYTITVYAVTYGDDSAPISINYRTEID

SEQ ID NO: 344 - protein, 104 aa, Artificial Sequence
  Adnectin
MGHHHHHHGGSVSDVPRDLEVVAATPTSLLISWEYKVHPYRYYRITYGETGGNSPVQEFTVPSVLSSAQLSGLKPGVDYTITVYAVTYGDDSAPISINYRTEID

SEQ ID NO: 345 - protein, 104 aa, Artificial Sequence; identical to SEQ ID NO: 168
  Adnectin

SEQ ID NO: 346 - protein, 104 aa, Artificial Sequence; identical to SEQ ID NO: 171
  Adnectin

SEQ ID NO: 347 - protein, 104 aa, Artificial Sequence; identical to SEQ ID NO: 179
  Adnectin

SEQ ID NO: 348 - protein, 104 aa, Artificial Sequence
  Adnectin
MGHHHHHHGGSVSDVPRDLEVVAATPTSLLISWEYKVHPYRYYRITYGETGGNSPVQEFTVPSVLSTATISGLKPGVDYTITVYAVTYGIDSTPISINYRTEID

SEQ ID NO: 349 - protein, 33 aa, Artificial Sequence
  HIV Fusion Peptide Inhibitor
SEYEARIEALIRAAQEQQEKNEAALRELYKWAL

SEQ ID NO: 350 - protein, 33 aa, Artificial Sequence
  HIV Fusion Peptide Inhibitor
SEYEARIEALIRAAQEQQEKNEAALRELWKWAS

SEQ ID NO: 351 - protein, 35 aa, Artificial Sequence
  HIV Fusion Peptide Inhibitor
TIAEYAARIEALIRAAQEQQEKNEAALRELYKWAS

SEQ ID NO: 352 - protein, 36 aa, Artificial Sequence
  HIV Fusion Peptide Inhibitor
ARIEEYAARIEALIRAAQEQQEKNEAALRELYKWAS

SEQ ID NO: 353 - protein, 33 aa, Artificial Sequence
  HIV Fusion Peptide Inhibitor
SEYEARIEALIRAAQEQQEKNEAALRELYKWAS

SEQ ID NO: 354 - protein, 36 aa, Artificial Sequence
  HIV Fusion Peptide Inhibitor
TEYEARIEALIRAAQEQQEKNEAALRELKEWASIWN

SEQ ID NO: 355 - protein, 41 aa, Artificial Sequence
  HIV Fusion Peptide Inhibitor
SEYEARIEALIRAAQEQQEKNEAALRELDKWTGVWGNYEKV

SEQ ID NO: 356 - protein, 29 aa, Artificial Sequence
  HIV Fusion Peptide Inhibitor
SRIEALIRAAQEQQEKNEAALRELFKWAS

SEQ ID NO: 357 - protein, 29 aa, Artificial Sequence
  HIV Fusion Peptide Inhibitor
SRIEALIRAAQEQQQKNEAALRELDKWAS

SEQ ID NO: 358 - protein, 29 aa, Artificial Sequence
  HIV Fusion Peptide Inhibitor
SRIEALIRAAQEQQEKNEAALRELYKWAS

SEQ ID NO: 359 - protein, 29 aa, Artificial Sequence
  HIV Fusion Peptide Inhibitor
SRIEALIRAAQEQQEKNEAALRELLKWAS

SEQ ID NO: 360 - protein, 29 aa, Artificial Sequence
  HIV Fusion Peptide Inhibitor
SRIEALIRAAQEQQEKNEAALRELQKWAS

SEQ ID NO: 361 - protein, 29 aa, Artificial Sequence
  HIV Fusion Peptide Inhibitor
SRIEALIRAAQEQQEKNEAALRELDKWAS

SEQ ID NO: 362 - protein, 35 aa, Artificial Sequence
  HIV Fusion Peptide Inhibitor
AIAEYAARIEALIRAAQEQQEKNEAALRELDKWAS

SEQ ID NO: 363 - protein, 30 aa, Artificial Sequence
  HIV Fusion Peptide Inhibitor
TEYEARIEALIRAAQEQQEKNEAALRELDK

SEQ ID NO: 364 - protein, 29 aa, Artificial Sequence
  HIV Fusion Peptide Inhibitor
SRIEALIRAAQEQQEKNEAALRELYKWTS

SEQ ID NO: 365 - protein, 32 aa, Artificial Sequence
  HIV Fusion Peptide Inhibitor
SRIEALIRAAQEQQEKNEAALRELYKWASLWI

SEQ ID NO: 366 - protein, 32 aa, Artificial Sequence
  HIV Fusion Peptide Inhibitor
SRIEALIRAAQEQQEKNEAALRELYKWASRWN

SEQ ID NO: 367 - protein, 32 aa, Artificial Sequence
  HIV Fusion Peptide Inhibitor
SRIEALIRAAQEQQEKNEAALRELYKWASSWN

SEQ ID NO: 368 - protein, 29 aa, Artificial Sequence
  HIV Fusion Peptide Inhibitor
SRIEALIRAAQEQQEKNEAALRELYKWGS

SEQ ID NO: 369 - protein, 26 aa, Artificial Sequence
  HIV Fusion Peptide Inhibitor
SRIEALIRAAQEQQEKNEAALRELDK

SEQ ID NO: 370 - protein, 14 aa, Artificial Sequence
  linker peptide
GSGSGSGSGSGSGS

SEQ ID NO: 371 - protein, 13 aa, Artificial Sequence
  linker peptide
GGSGSGSGSGSGS

SEQ ID NO: 372 - protein, 14 aa, Artificial Sequence
  linker peptide
GGSGSGSGSGSGSG

SEQ ID NO: 373 - protein, 15 aa, Artificial Sequence
  linker peptide
GSEGSEGSEGSEGSE

SEQ ID NO: 374 - protein, 8 aa, Artificial Sequence
  linker peptide
GGSEGGSE

SEQ ID NO: 375 - protein, 8 aa, Artificial Sequence
  linker peptide
GSGSGSGS

SEQ ID NO: 376 - protein, 35 aa, Artificial Sequence
  linker peptide
GGGGSGGGGSGGGGSGGGGSGGGGSGGGGSGGGGS

SEQ ID NO: 377 - protein, 25 aa, Artificial Sequence
  linker peptide
GGGGSGGGGSGGGGSGGGGSGGGGS

SEQ ID NO: 378 - protein, 20 aa, Artificial Sequence
  linker peptide
GGGGSGGGGSGGGGSGGGGS

SEQ ID NO: 379 - protein, 15 aa, Artificial Sequence
  linker peptide
GGGGSGGGGSGGGGS

SEQ ID NO: 380 - protein, 7 aa, Artificial Sequence
  linker peptide
GPGPGPG

SEQ ID NO: 381 - protein, 11 aa, Artificial Sequence
  linker peptide
GPGPGPGPGPG

SEQ ID NO: 382 - protein, 6 aa, Artificial Sequence
  linker peptide
PAPAPA

SEQ ID NO: 383 - protein, 12 aa, Artificial Sequence
  linker peptide
PAPAPAPAPAPA

SEQ ID NO: 384 - protein, 18 aa, Artificial Sequence
  linker peptide
PAPAPAPAPAPAPAPAPA

SEQ ID NO: 385 - protein, 11 aa, Artificial Sequence
  linker peptide
ESPEPETPEDE

SEQ ID NO: 386 - protein, 21 aa, Artificial Sequence
  linker peptide
ESPEPETPEDESPEPETPEDE

SEQ ID NO: 387 - protein, 5 aa, Artificial Sequence
  linker peptide
GGGGS

SEQ ID NO: 388 - protein, 6 aa, Artificial Sequence
  linker peptide
TVAAPS

SEQ ID NO: 389 - protein, 7 aa, Artificial Sequence
  linker peptide
ASTKGPT

SEQ ID NO: 390 - protein, 7 aa, Artificial Sequence
  linker peptide
ASTKGPS

SEQ ID NO: 391 - protein, 2 aa, Artificial Sequence
  linker peptide
GS

SEQ ID NO: 392 - protein, 8 aa, Artificial Sequence
  linker peptide
TVAAPSGS

SEQ ID NO: 393 - protein, 8 aa, Artificial Sequence
  linker peptide
GSTVAAPS

SEQ ID NO: 394 - protein, 10 aa, Artificial Sequence
  linker peptide
GSTVAAPSGS

SEQ ID NO: 395 - protein, 18 aa, Artificial Sequence
  linker peptide
GSTVAAPSGSTVAAPSGS

SEQ ID NO: 396 - protein, 26 aa, Artificial Sequence
  linker peptide
GSTVAAPSGSTVAAPSGSTVAAPSGS

SEQ ID NO: 397 - protein, 34 aa, Artificial Sequence
  linker peptide
GSTVAAPSGSTVAAPSGSTVAAPSGSTVAAPSGS

SEQ ID NO: 398 - protein, 42 aa, Artificial Sequence
  linker peptide
GSTVAAPSGSTVAAPSGSTVAAPSGSTVAAPSGSTVAAPSGS

SEQ ID NO: 399 - protein, 50 aa, Artificial Sequence
  linker peptide
GSTVAAPSGSTVAAPSGSTVAAPSGSTVAAPSGSTVAAPSGSTVAAPSGS

SEQ ID NO: 400 - protein, 14 aa, Artificial Sequence
  linker peptide
TVAAPSTVAAPSGS

SEQ ID NO: 401 - protein, 20 aa, Artificial Sequence
  linker peptide
TVAAPSTVAAPSTVAAPSGS

SEQ ID NO: 402 - protein, 17 aa, Artificial Sequence
  linker peptide
ELQLEESAAEAQEGELE

SEQ ID NO: 403 - protein, 15 aa, Artificial Sequence
  linker peptide
EAAAKEAAAKEAAAK

SEQ ID NO: 404 - protein, 14 aa, Artificial Sequence
  linker peptide
EEEKRAAEERQRIK

SEQ ID NO: 405 - protein, 4 aa, Artificial Sequence
  linker peptide
GSGS

SEQ ID NO: 406 - protein, 31 aa, Artificial Sequence
  linker peptide
ESPEPETPEDESPEPETPEDESPEPETPEDE

SEQ ID NO: 407 - protein, 25 aa, Artificial Sequence
  linker peptide
GGGGSGGGGSGGGGSGGGGSGGGGS

SEQ ID NO: 408 - protein, 3 aa, Artificial Sequence
  Linker peptide
PAS

SEQ ID NO: 409 - protein, 6 aa, Artificial Sequence
  Linker peptide
PASPAS

SEQ ID NO: 410 - protein, 9 aa, Artificial Sequence
  Linker peptide
PASPASPAS

SEQ ID NO: 411 - protein, 12 aa, Artificial Sequence
  Linker peptide
PASPASPASPAS

SEQ ID NO: 412 - protein, 15 aa, Artificial Sequence
  Linker peptide
PASPASPASPASPAS

SEQ ID NO: 413 - protein, 18 aa, Artificial Sequence
  Linker peptide
PASPASPASPASPASPAS

SEQ ID NO: 414 - protein, 21 aa, Artificial Sequence
  Linker peptide
PASPASPASPASPASPASPAS

SEQ ID NO: 415 - protein, 24 aa, Artificial Sequence
  Linker peptide
PASPASPASPASPASPASPASPAS

SEQ ID NO: 416 - protein, 27 aa, Artificial Sequence
  Linker peptide
PASPASPASPASPASPASPASPASPAS

SEQ ID NO: 417 - protein, 30 aa, Artificial Sequence
  Linker peptide
PASPASPASPASPASPASPASPASPASPAS

SEQ ID NO: 418 - protein, 5 aa, Artificial Sequence
  Linker peptide
PASGS

SEQ ID NO: 419 - protein, 8 aa, Artificial Sequence
  Linker peptide
PASPASGS

SEQ ID NO: 420 - protein, 11 aa, Artificial Sequence
  Linker peptide
PASPASPASGS

SEQ ID NO: 421 - protein, 14 aa, Artificial Sequence
  Linker peptide
PASPASPASPASGS

SEQ ID NO: 422 - protein, 17 aa, Artificial Sequence
  Linker peptide
PASPASPASPASPASGS

SEQ ID NO: 423 - protein, 20 aa, Artificial Sequence
  Linker peptide
PASPASPASPASPASPASGS

SEQ ID NO: 424 - protein, 23 aa, Artificial Sequence
  Linker peptide
PASPASPASPASPASPASPASGS

SEQ ID NO: 425 - protein, 26 aa, Artificial Sequence
  Linker peptide
PASPASPASPASPASPASPASPASGS

SEQ ID NO: 426 - protein, 29 aa, Artificial Sequence
  Linker peptide
PASPASPASPASPASPASPASPASPASGS

SEQ ID NO: 427 - protein, 32 aa, Artificial Sequence
  Linker peptide
PASPASPASPASPASPASPASPASPASPASGS

SEQ ID NO: 428 - protein, 7 aa, Artificial Sequence
  Linker peptide
PASGSGS

SEQ ID NO: 429 - protein, 10 aa, Artificial Sequence
  Linker peptide
PASPASGSGS

SEQ ID NO: 430 - protein, 13 aa, Artificial Sequence
  Linker peptide
PASPASPASGSGS

SEQ ID NO: 431 - protein, 16 aa, Artificial Sequence
  Linker peptide
PASPASPASPASGSGS

SEQ ID NO: 432 - protein, 19 aa, Artificial Sequence
  Linker peptide
PASPASPASPASPASGSGS

SEQ ID NO: 433 - protein, 22 aa, Artificial Sequence
  Linker peptide
PASPASPASPASPASPASGSGS

SEQ ID NO: 434 - protein, 25 aa, Artificial Sequence
  Linker peptide
PASPASPASPASPASPASPASGSGS

SEQ ID NO: 435 - protein, 28 aa, Artificial Sequence
  Linker peptide
PASPASPASPASPASPASPASPASGSGS

SEQ ID NO: 436 - protein, 31 aa, Artificial Sequence
  Linker peptide
PASPASPASPASPASPASPASPASPASGSGS

SEQ ID NO: 437 - protein, 34 aa, Artificial Sequence
  Linker peptide
PASPASPASPASPASPASPASPASPASPASGSGS

SEQ ID NO: 438 - protein, 9 aa, Artificial Sequence
  Linker peptide
PASGSGSGS

SEQ ID NO: 439 - protein, 12 aa, Artificial Sequence
  Linker peptide
PASPASGSGSGS

SEQ ID NO: 440 - protein, 15 aa, Artificial Sequence
  Linker peptide
PASPASPASGSGSGS

SEQ ID NO: 441 - protein, 18 aa, Artificial Sequence
  Linker peptide
PASPASPASPASGSGSGS

SEQ ID NO: 442 - protein, 21 aa, Artificial Sequence
  Linker peptide
PASPASPASPASPASGSGSGS

SEQ ID NO: 443 - protein, 24 aa, Artificial Sequence
  Linker peptide
PASPASPASPASPASPASGSGSGS

SEQ ID NO: 444 - protein, 27 aa, Artificial Sequence
  Linker peptide
PASPASPASPASPASPASPASGSGSGS

SEQ ID NO: 445 - protein, 30 aa, Artificial Sequence
  Linker peptide
PASPASPASPASPASPASPASPASGSGSGS

SEQ ID NO: 446 - protein, 33 aa, Artificial Sequence
  Linker peptide
PASPASPASPASPASPASPASPASPASGSGSGS

SEQ ID NO: 447 - protein, 36 aa, Artificial Sequence
  Linker peptide
PASPASPASPASPASPASPASPASPASPASGSGSGS

SEQ ID NO: 448 - protein, 11 aa, Artificial Sequence
  Linker peptide
PASGSGSGSGS

SEQ ID NO: 449 - protein, 14 aa, Artificial Sequence
  Linker peptide
PASPASGSGSGSGS

SEQ ID NO: 450 - protein, 17 aa, Artificial Sequence
  Linker peptide
PASPASPASGSGSGSGS

SEQ ID NO: 451 - protein, 20 aa, Artificial Sequence
  Linker peptide
PASPASPASPASGSGSGSGS

SEQ ID NO: 452 - protein, 23 aa, Artificial Sequence
  Linker peptide
PASPASPASPASPASGSGSGSGS

SEQ ID NO: 453 - protein, 26 aa, Artificial Sequence
  Linker peptide
PASPASPASPASPASPASGSGSGSGS

SEQ ID NO: 454 - protein, 29 aa, Artificial Sequence
  Linker peptide
PASPASPASPASPASPASPASGSGSGSGS

SEQ ID NO: 455 - protein, 32 aa, Artificial Sequence
  Linker peptide
PASPASPASPASPASPASPASPASGSGSGSGS

SEQ ID NO: 456 - protein, 35 aa, Artificial Sequence
  Linker peptide
PASPASPASPASPASPASPASPASPASGSGSGSGS

SEQ ID NO: 457 - protein, 38 aa, Artificial Sequence
  Linker peptide
PASPASPASPASPASPASPASPASPASPASGSGSGSGS

SEQ ID NO: 458 - protein, 5 aa, Artificial Sequence
  Linker peptide
GGGGS

SEQ ID NO: 459 - protein, 10 aa, Artificial Sequence
  Linker peptide
GGGGSGGGGS

SEQ ID NO: 460 - protein, 15 aa, Artificial Sequence
  Linker peptide
GGGGSGGGGSGGGGS

SEQ ID NO: 461 - protein, 20 aa, Artificial Sequence
  Linker peptide
GGGGSGGGGSGGGGSGGGGS

SEQ ID NO: 462 - protein, 25 aa, Artificial Sequence
  Linker peptide
GGGGSGGGGSGGGGSGGGGSGGGGS

SEQ ID NO: 463 - protein, 30 aa, Artificial Sequence
  Linker peptide
GGGGSGGGGSGGGGSGGGGSGGGGSGGGGS

SEQ ID NO: 464 - protein, 35 aa, Artificial Sequence; identical to SEQ ID NO: 376
  Linker peptide

SEQ ID NO: 465 - protein, 40 aa, Artificial Sequence
  Linker peptide
GGGGSGGGGSGGGGSGGGGSGGGGSGGGGSGGGGSGGGGS

SEQ ID NO: 466 - protein, 45 aa, Artificial Sequence
  Linker peptide
GGGGSGGGGSGGGGSGGGGSGGGGSGGGGSGGGGSGGGGSGGGGS

SEQ ID NO: 467 - protein, 50 aa, Artificial Sequence
  Linker peptide
GGGGSGGGGSGGGGSGGGGSGGGGSGGGGSGGGGSGGGGSGGGGSGGGGS

SEQ ID NO: 468 - protein, 7 aa, Artificial Sequence
  Linker peptide
GGGGSGS

SEQ ID NO: 469 - protein, 12 aa, Artificial Sequence
  Linker peptide
GGGGSGGGGSGS

SEQ ID NO: 470 - protein, 17 aa, Artificial Sequence
  Linker peptide
GGGGSGGGGSGGGGSGS

SEQ ID NO: 471 - protein, 22 aa, Artificial Sequence
  Linker peptide
GGGGSGGGGSGGGGSGGGGSGS

SEQ ID NO: 472 - protein, 27 aa, Artificial Sequence
  Linker peptide
GGGGSGGGGSGGGGSGGGGSGGGGSGS

SEQ ID NO: 473 - protein, 32 aa, Artificial Sequence
  Linker peptide
GGGGSGGGGSGGGGSGGGGSGGGGSGGGGSGS

SEQ ID NO: 474 - protein, 37 aa, Artificial Sequence
  Linker peptide
GGGGSGGGGSGGGGSGGGGSGGGGSGGGGSGGGGSGS

SEQ ID NO: 475 - protein, 42 aa, Artificial Sequence
  Linker peptide
GGGGSGGGGSGGGGSGGGGSGGGGSGGGGSGGGGSGGGGSGS

SEQ ID NO: 476 - protein, 47 aa, Artificial Sequence
  Linker peptide
GGGGSGGGGSGGGGSGGGGSGGGGSGGGGSGGGGSGGGGSGGGGSGS

SEQ ID NO: 477 - protein, 52 aa, Artificial Sequence
  Linker peptide
GGGGSGGGGSGGGGSGGGGSGGGGSGGGGSGGGGSGGGGSGGGGSGGGGSGS

SEQ ID NO: 478 - protein, 9 aa, Artificial Sequence
  Linker peptide
GGGGSGSGS

SEQ ID NO: 479 - protein, 14 aa, Artificial Sequence
  Linker peptide
GGGGSGGGGSGSGS

SEQ ID NO: 480 - protein, 25 aa, Artificial Sequence
  Linker peptide
GGGGSGGGGSGGGGSGGGGSGGGGS

SEQ ID NO: 481 - protein, 24 aa, Artificial Sequence
  Linker peptide
GGGGSGGGGSGGGGSGGGGSGSGS

SEQ ID NO: 482 - protein, 29 aa, Artificial Sequence
  Linker peptide
GGGGSGGGGSGGGGSGGGGSGGGGSGSGS

SEQ ID NO: 483 - protein, 34 aa, Artificial Sequence
  Linker peptide
GGGGSGGGGSGGGGSGGGGSGGGGSGGGGSGSGS

SEQ ID NO: 484 - protein, 39 aa, Artificial Sequence
  Linker peptide
GGGGSGGGGSGGGGSGGGGSGGGGSGGGGSGGGGSGSGS

SEQ ID NO: 485 - protein, 44 aa, Artificial Sequence
  Linker peptide
GGGGSGGGGSGGGGSGGGGSGGGGSGGGGSGGGGSGGGGSGSGS

SEQ ID NO: 486 - protein, 49 aa, Artificial Sequence
  Linker peptide
GGGGSGGGGSGGGGSGGGGSGGGGSGGGGSGGGGSGGGGSGGGGSGSGS

SEQ ID NO: 487 - protein, 54 aa, Artificial Sequence
  Linker peptide
GGGGSGGGGSGGGGSGGGGSGGGGSGGGGSGGGGSGGGGSGGGGSGGGGSGSGS

SEQ ID NO: 488 - protein, 11 aa, Artificial Sequence
  Linker peptide
GGGGSGSGSGS

SEQ ID NO: 489 - protein, 16 aa, Artificial Sequence
  Linker peptide
GGGGSGGGGSGSGSGS

SEQ ID NO: 490 - protein, 21 aa, Artificial Sequence
  Linker peptide
GGGGSGGGGSGGGGSGSGSGS

SEQ ID NO: 491 - protein, 26 aa, Artificial Sequence
  Linker peptide
GGGGSGGGGSGGGGSGGGGSGSGSGS

SEQ ID NO: 492 - protein, 31 aa, Artificial Sequence
  Linker peptide
GGGGSGGGGSGGGGSGGGGSGGGGSGSGSGS

SEQ ID NO: 493 - protein, 36 aa, Artificial Sequence
  Linker peptide
GGGGSGGGGSGGGGSGGGGSGGGGSGGGGSGSGSGS

SEQ ID NO: 494 - protein, 41 aa, Artificial Sequence
  Linker peptide
GGGGSGGGGSGGGGSGGGGSGGGGSGGGGSGGGGSGSGSGS

SEQ ID NO: 495 - protein, 46 aa, Artificial Sequence
  Linker peptide
GGGGSGGGGSGGGGSGGGGSGGGGSGGGGSGGGGSGGGGSGSGSGS

SEQ ID NO: 496 - protein, 51 aa, Artificial Sequence
  Linker peptide
GGGGSGGGGSGGGGSGGGGSGGGGSGGGGSGGGGSGGGGSGGGGSGSGSGS

SEQ ID NO: 497 - protein, 56 aa, Artificial Sequence
  Linker peptide
GGGGSGGGGSGGGGSGGGGSGGGGSGGGGSGGGGSGGGGSGGGGSGGGGSGSGSGS

SEQ ID NO: 498 - protein, 13 aa, Artificial Sequence
  Linker peptide
GGGGSGSGSGSGS

SEQ ID NO: 499 - protein, 18 aa, Artificial Sequence
  Linker peptide
GGGGSGGGGSGSGSGSGS

SEQ ID NO: 500 - protein, 23 aa, Artificial Sequence
  Linker peptide
GGGGSGGGGSGGGGSGSGSGSGS

SEQ ID NO: 501 - protein, 28 aa, Artificial Sequence
  Linker peptide
GGGGSGGGGSGGGGSGGGGSGSGSGSGS

SEQ ID NO: 502 - protein, 33 aa, Artificial Sequence
  Linker peptide
GGGGSGGGGSGGGGSGGGGSGGGGSGSGSGSGS

SEQ ID NO: 503 - protein, 38 aa, Artificial Sequence
  Linker peptide
GGGGSGGGGSGGGGSGGGGSGGGGSGGGGSGSGSGSGS

SEQ ID NO: 504 - protein, 43 aa, Artificial Sequence
  Linker peptide
GGGGSGGGGSGGGGSGGGGSGGGGSGGGGSGGGGSGSGSGSGS

SEQ ID NO: 505 - protein, 48 aa, Artificial Sequence
  Linker peptide
GGGGSGGGGSGGGGSGGGGSGGGGSGGGGSGGGGSGGGGSGSGSGSGS

SEQ ID NO: 506 - protein, 53 aa, Artificial Sequence
  Linker peptide
GGGGSGGGGSGGGGSGGGGSGGGGSGGGGSGGGGSGGGGSGGGGSGSGSGSGS

SEQ ID NO: 507 - protein, 58 aa, Artificial Sequence
  Linker peptide
GGGGSGGGGSGGGGSGGGGSGGGGSGGGGSGGGGSGGGGSGGGGSGGGGSGSGSGSGS

SEQ ID NO: 508 - protein, 6 aa, Artificial Sequence
  Linker peptide
TVAAPS

SEQ ID NO: 509 - protein, 12 aa, Artificial Sequence
  Linker peptide
TVAAPSTVAAPS

SEQ ID NO: 510 - protein, 18 aa, Artificial Sequence
  Linker peptide
TVAAPSTVAAPSTVAAPS

SEQ ID NO: 511 - protein, 24 aa, Artificial Sequence
  Linker peptide
TVAAPSTVAAPSTVAAPSTVAAPS

SEQ ID NO: 512 - protein, 30 aa, Artificial Sequence
  Linker peptide
TVAAPSTVAAPSTVAAPSTVAAPSTVAAPS

SEQ ID NO: 513 - protein, 36 aa, Artificial Sequence
  Linker peptide
TVAAPSTVAAPSTVAAPSTVAAPSTVAAPSTVAAPS

SEQ ID NO: 514 - protein, 42 aa, Artificial Sequence
  Linker peptide
TVAAPSTVAAPSTVAAPSTVAAPSTVAAPSTVAAPSTVAAPS

SEQ ID NO: 515 - protein, 48 aa, Artificial Sequence
  Linker peptide
TVAAPSTVAAPSTVAAPSTVAAPSTVAAPSTVAAPSTVAAPSTVAAPS

SEQ ID NO: 516 - protein, 54 aa, Artificial Sequence
  Linker peptide
TVAAPSTVAAPSTVAAPSTVAAPSTVAAPSTVAAPSTVAAPSTVAAPSTVAAPS

SEQ ID NO: 517 - protein, 60 aa, Artificial Sequence
  Linker peptide
TVAAPSTVAAPSTVAAPSTVAAPSTVAAPSTVAAPSTVAAPSTVAAPSTVAAPSTVAAPS

SEQ ID NO: 518 - protein, 26 aa, Artificial Sequence
  Linker peptide
TVAAPSTVAAPSTVAAPSTVAAPSGS

SEQ ID NO: 519 - protein, 32 aa, Artificial Sequence
  Linker peptide
TVAAPSTVAAPSTVAAPSTVAAPSTVAAPSGS

SEQ ID NO: 520 - protein, 38 aa, Artificial Sequence
  Linker peptide
TVAAPSTVAAPSTVAAPSTVAAPSTVAAPSTVAAPSGS

SEQ ID NO: 521 - protein, 44 aa, Artificial Sequence
  Linker peptide
TVAAPSTVAAPSTVAAPSTVAAPSTVAAPSTVAAPSTVAAPSGS

SEQ ID NO: 522 - protein, 50 aa, Artificial Sequence
  Linker peptide
TVAAPSTVAAPSTVAAPSTVAAPSTVAAPSTVAAPSTVAAPSTVAAPSGS

SEQ ID NO: 523 - protein, 56 aa, Artificial Sequence
  Linker peptide
TVAAPSTVAAPSTVAAPSTVAAPSTVAAPSTVAAPSTVAAPSTVAAPSTVAAPSGS

SEQ ID NO: 524 - protein, 62 aa, Artificial Sequence
  Linker peptide
TVAAPSTVAAPSTVAAPSTVAAPSTVAAPSTVAAPSTVAAPSTVAAPSTVAAPSTVAAPSGS

SEQ ID NO: 525 - protein, 10 aa, Artificial Sequence
  Linker peptide
TVAAPSGSGS

SEQ ID NO: 526 - protein, 16 aa, Artificial Sequence
  Linker peptide
TVAAPSTVAAPSGSGS

SEQ ID NO: 527 - protein, 22 aa, Artificial Sequence
  Linker peptide
TVAAPSTVAAPSTVAAPSGSGS

SEQ ID NO: 528 - protein, 28 aa, Artificial Sequence
  Linker peptide
TVAAPSTVAAPSTVAAPSTVAAPSGSGS

SEQ ID NO: 529 - protein, 34 aa, Artificial Sequence
  Linker peptide
TVAAPSTVAAPSTVAAPSTVAAPSTVAAPSGSGS

SEQ ID NO: 530 - protein, 40 aa, Artificial Sequence
  Linker peptide
TVAAPSTVAAPSTVAAPSTVAAPSTVAAPSTVAAPSGSGS

SEQ ID NO: 531 - protein, 46 aa, Artificial Sequence
  Linker peptide
TVAAPSTVAAPSTVAAPSTVAAPSTVAAPSTVAAPSTVAAPSGSGS

SEQ ID NO: 532 - protein, 52 aa, Artificial Sequence
  Linker peptide
TVAAPSTVAAPSTVAAPSTVAAPSTVAAPSTVAAPSTVAAPSTVAAPSGSGS

SEQ ID NO: 533 - protein, 58 aa, Artificial Sequence
  Linker peptide
TVAAPSTVAAPSTVAAPSTVAAPSTVAAPSTVAAPSTVAAPSTVAAPSTVAAPSGSGS

SEQ ID NO: 534 - protein, 64 aa, Artificial Sequence
  Linker peptide
TVAAPSTVAAPSTVAAPSTVAAPSTVAAPSTVAAPSTVAAPSTVAAPSTVAAPSTVAAPSGSGS

SEQ ID NO: 535 - protein, 12 aa, Artificial Sequence
  Linker peptide
TVAAPSGSGSGS

SEQ ID NO: 536 - protein, 18 aa, Artificial Sequence
  Linker peptide
TVAAPSTVAAPSGSGSGS

SEQ ID NO: 537 - protein, 24 aa, Artificial Sequence
  Linker peptide
TVAAPSTVAAPSTVAAPSGSGSGS

SEQ ID NO: 538 - protein, 30 aa, Artificial Sequence
  Linker peptide
TVAAPSTVAAPSTVAAPSTVAAPSGSGSGS

SEQ ID NO: 539 - protein, 36 aa, Artificial Sequence
  Linker peptide
TVAAPSTVAAPSTVAAPSTVAAPSTVAAPSGSGSGS

SEQ ID NO: 540 - protein, 42 aa, Artificial Sequence
  Linker peptide
TVAAPSTVAAPSTVAAPSTVAAPSTVAAPSTVAAPSGSGSGS

SEQ ID NO: 541 - protein, 48 aa, Artificial Sequence
  Linker peptide
TVAAPSTVAAPSTVAAPSTVAAPSTVAAPSTVAAPSTVAAPSGSGSGS

SEQ ID NO: 542 - protein, 54 aa, Artificial Sequence
  Linker peptide
TVAAPSTVAAPSTVAAPSTVAAPSTVAAPSTVAAPSTVAAPSTVAAPSGSGSGS

SEQ ID NO: 543 - protein, 60 aa, Artificial Sequence
  Linker peptide
TVAAPSTVAAPSTVAAPSTVAAPSTVAAPSTVAAPSTVAAPSTVAAPSTVAAPSGSGSGS

SEQ ID NO: 544 - protein, 66 aa, Artificial Sequence
  Linker peptide
TVAAPSTVAAPSTVAAPSTVAAPSTVAAPSTVAAPSTVAAPSTVAAPSTVAAPSTVAAPSGSGSGS

SEQ ID NO: 545 - protein, 14 aa, Artificial Sequence
  Linker peptide
TVAAPSGSGSGSGS

SEQ ID NO: 546 - protein, 20 aa, Artificial Sequence
  Linker peptide
TVAAPSTVAAPSGSGSGSGS

SEQ ID NO: 547 - protein, 26 aa, Artificial Sequence
  Linker peptide
TVAAPSTVAAPSTVAAPSGSGSGSGS

SEQ ID NO: 548 - protein, 32 aa, Artificial Sequence
  Linker peptide
TVAAPSTVAAPSTVAAPSTVAAPSGSGSGSGS

SEQ ID NO: 549 - protein, 38 aa, Artificial Sequence
  Linker peptide
TVAAPSTVAAPSTVAAPSTVAAPSTVAAPSGSGSGSGS

SEQ ID NO: 550 - protein, 44 aa, Artificial Sequence
  Linker peptide
TVAAPSTVAAPSTVAAPSTVAAPSTVAAPSTVAAPSGSGSGSGS

SEQ ID NO: 551 - protein, 50 aa, Artificial Sequence
  Linker peptide
TVAAPSTVAAPSTVAAPSTVAAPSTVAAPSTVAAPSTVAAPSGSGSGSGS

SEQ ID NO: 552 - protein, 56 aa, Artificial Sequence
  Linker peptide
TVAAPSTVAAPSTVAAPSTVAAPSTVAAPSTVAAPSTVAAPSTVAAPSGSGSGSGS

SEQ ID NO: 553 - protein, 62 aa, Artificial Sequence
  Linker peptide
TVAAPSTVAAPSTVAAPSTVAAPSTVAAPSTVAAPSTVAAPSTVAAPSTVAAPSGSGSGSGS

SEQ ID NO: 554 - protein, 68 aa, Artificial Sequence
  Linker peptide
TVAAPSTVAAPSTVAAPSTVAAPSTVAAPSTVAAPSTVAAPSTVAAPSTVAAPSTVAAPSGSGSGSGS

SEQ ID NO: 555 - protein, 8 aa, Artificial Sequence
  Linker peptide
TVAAPSGS

SEQ ID NO: 556 - protein, 16 aa, Artificial Sequence
  Linker peptide
TVAAPSGSTVAAPSGS

SEQ ID NO: 557 - protein, 24 aa, Artificial Sequence
  Linker peptide
TVAAPSGSTVAAPSGSTVAAPSGS

SEQ ID NO: 558 - protein, 32 aa, Artificial Sequence
  Linker peptide
TVAAPSGSTVAAPSGSTVAAPSGSTVAAPSGS

SEQ ID NO: 559 - protein, 40 aa, Artificial Sequence
  Linker peptide
TVAAPSGSTVAAPSGSTVAAPSGSTVAAPSGSTVAAPSGS

SEQ ID NO: 560 - protein, 48 aa, Artificial Sequence
  Linker peptide
TVAAPSGSTVAAPSGSTVAAPSGSTVAAPSGSTVAAPSGSTVAAPSGS

SEQ ID NO: 561 - protein, 56 aa, Artificial Sequence
  Linker peptide
TVAAPSGSTVAAPSGSTVAAPSGSTVAAPSGSTVAAPSGSTVAAPSGSTVAAPSGS

SEQ ID NO: 562 - protein, 64 aa, Artificial Sequence
  Linker peptide
TVAAPSGSTVAAPSGSTVAAPSGSTVAAPSGSTVAAPSGSTVAAPSGSTVAAPSGSTVAAPSGS

SEQ ID NO: 563 - protein, 72 aa, Artificial Sequence
  Linker peptide
TVAAPSGSTVAAPSGSTVAAPSGSTVAAPSGSTVAAPSGSTVAAPSGSTVAAPSGSTVAAPSGSTVAAPSGS

SEQ ID NO: 564 - protein, 80 aa, Artificial Sequence
  Linker peptide
TVAAPSGSTVAAPSGSTVAAPSGSTVAAPSGSTVAAPSGSTVAAPSGSTVAAPSGSTVAAPSGSTVAAPSGSTVAAPSGS

SEQ ID NO: 565 - protein, 58 aa, Artificial Sequence
  Linker peptide
GSTVAAPSGSTVAAPSGSTVAAPSGSTVAAPSGSTVAAPSGSTVAAPSGSTVAAPSGS

SEQ ID NO: 566 - protein, 66 aa, Artificial Sequence
  Linker peptide
GSTVAAPSGSTVAAPSGSTVAAPSGSTVAAPSGSTVAAPSGSTVAAPSGSTVAAPSGSTVAAPSGS

SEQ ID NO: 567 - protein, 74 aa, Artificial Sequence
  Linker peptide
GSTVAAPSGSTVAAPSGSTVAAPSGSTVAAPSGSTVAAPSGSTVAAPSGSTVAAPSGSTVAAPSGSTVAAPSGS

SEQ ID NO: 568 - protein, 82 aa, Artificial Sequence
  Linker peptide
GSTVAAPSGSTVAAPSGSTVAAPSGSTVAAPSGSTVAAPSGSTVAAPSGSTVAAPSGSTVAAPSGSTVAAPSGSTVAAPSGS

SEQ ID NO: 569 - protein, 12 aa, Artificial Sequence
  Linker peptide
GSGSTVAAPSGS

SEQ ID NO: 570 - protein, 20 aa, Artificial Sequence
  Linker peptide
GSGSTVAAPSGSTVAAPSGS

SEQ ID NO: 571 - protein, 28 aa, Artificial Sequence
  Linker peptide
GSGSTVAAPSGSTVAAPSGSTVAAPSGS

SEQ ID NO: 572 - protein, 36 aa, Artificial Sequence
  Linker peptide
GSGSTVAAPSGSTVAAPSGSTVAAPSGSTVAAPSGS

SEQ ID NO: 573 - protein, 44 aa, Artificial Sequence
  Linker peptide
GSGSTVAAPSGSTVAAPSGSTVAAPSGSTVAAPSGSTVAAPSGS

SEQ ID NO: 574 - protein, 52 aa, Artificial Sequence
  Linker peptide
GSGSTVAAPSGSTVAAPSGSTVAAPSGSTVAAPSGSTVAAPSGSTVAAPSGS

SEQ ID NO: 575 - protein, 60 aa, Artificial Sequence
  Linker peptide
GSGSTVAAPSGSTVAAPSGSTVAAPSGSTVAAPSGSTVAAPSGSTVAAPSGSTVAAPSGS

SEQ ID NO: 576 - protein, 68 aa, Artificial Sequence
  Linker peptide
GSGSTVAAPSGSTVAAPSGSTVAAPSGSTVAAPSGSTVAAPSGSTVAAPSGSTVAAPSGSTVAAPSGS

SEQ ID NO: 577 - protein, 76 aa, Artificial Sequence
  Linker peptide
GSGSTVAAPSGSTVAAPSGSTVAAPSGSTVAAPSGSTVAAPSGSTVAAPSGSTVAAPSGSTVAAPSGSTVAAPSGS

SEQ ID NO: 578 - protein, 84 aa, Artificial Sequence
  Linker peptide
GSGSTVAAPSGSTVAAPSGSTVAAPSGSTVAAPSGSTVAAPSGSTVAAPSGSTVAAPSGSTVAAPSGSTVAAPSGSTVAAPSGS

SEQ ID NO: 579 - protein, 14 aa, Artificial Sequence
  Linker peptide
GSGSGSTVAAPSGS

SEQ ID NO: 580 - protein, 22 aa, Artificial Sequence
  Linker peptide
GSGSGSTVAAPSGSTVAAPSGS

SEQ ID NO: 581 - protein, 30 aa, Artificial Sequence
  Linker peptide
GSGSGSTVAAPSGSTVAAPSGSTVAAPSGS

SEQ ID NO: 582 - protein, 38 aa, Artificial Sequence
  Linker peptide
GSGSGSTVAAPSGSTVAAPSGSTVAAPSGSTVAAPSGS

SEQ ID NO: 583 - protein, 46 aa, Artificial Sequence
  Linker peptide
GSGSGSTVAAPSGSTVAAPSGSTVAAPSGSTVAAPSGSTVAAPSGS

SEQ ID NO: 584 - protein, 54 aa, Artificial Sequence
  Linker peptide
GSGSGSTVAAPSGSTVAAPSGSTVAAPSGSTVAAPSGSTVAAPSGSTVAAPSGS

SEQ ID NO: 585 - protein, 62 aa, Artificial Sequence
  Linker peptide
GSGSGSTVAAPSGSTVAAPSGSTVAAPSGSTVAAPSGSTVAAPSGSTVAAPSGSTVAAPSGS

SEQ ID NO: 586 - protein, 70 aa, Artificial Sequence
  Linker peptide
GSGSGSTVAAPSGSTVAAPSGSTVAAPSGSTVAAPSGSTVAAPSGSTVAAPSGSTVAAPSGSTVAAPSGS

SEQ ID NO: 587 - protein, 78 aa, Artificial Sequence
  Linker peptide
GSGSGSTVAAPSGSTVAAPSGSTVAAPSGSTVAAPSGSTVAAPSGSTVAAPSGSTVAAPSGSTVAAPSGSTVAAPSGS

SEQ ID NO: 588 - protein, 86 aa, Artificial Sequence
  Linker peptide
GSGSGSTVAAPSGSTVAAPSGSTVAAPSGSTVAAPSGSTVAAPSGSTVAAPSGSTVAAPSGSTVAAPSGSTVAAPSGSTVAAPSGS

SEQ ID NO: 589 - protein, 16 aa, Artificial Sequence
  Linker peptide
GSGSGSGSTVAAPSGS

SEQ ID NO: 590 - protein, 24 aa, Artificial Sequence
  Linker peptide
GSGSGSGSTVAAPSGSTVAAPSGS

SEQ ID NO: 591 - protein, 32 aa, Artificial Sequence
  Linker peptide
GSGSGSGSTVAAPSGSTVAAPSGSTVAAPSGS

SEQ ID NO: 592 - protein, 40 aa, Artificial Sequence
  Linker peptide
GSGSGSGSTVAAPSGSTVAAPSGSTVAAPSGSTVAAPSGS

SEQ ID NO: 593 - protein, 48 aa, Artificial Sequence
  Linker peptide
GSGSGSGSTVAAPSGSTVAAPSGSTVAAPSGSTVAAPSGSTVAAPSGS

SEQ ID NO: 594 - protein, 56 aa, Artificial Sequence
  Linker peptide
GSGSGSGSTVAAPSGSTVAAPSGSTVAAPSGSTVAAPSGSTVAAPSGSTVAAPSGS

SEQ ID NO: 595 - protein, 64 aa, Artificial Sequence
  Linker peptide
GSGSGSGSTVAAPSGSTVAAPSGSTVAAPSGSTVAAPSGSTVAAPSGSTVAAPSGSTVAAPSGS

SEQ ID NO: 596 - protein, 72 aa, Artificial Sequence
  Linker peptide
GSGSGSGSTVAAPSGSTVAAPSGSTVAAPSGSTVAAPSGSTVAAPSGSTVAAPSGSTVAAPSGSTVAAPSGS

SEQ ID NO: 597 - protein, 80 aa, Artificial Sequence
  Linker peptide
GSGSGSGSTVAAPSGSTVAAPSGSTVAAPSGSTVAAPSGSTVAAPSGSTVAAPSGSTVAAPSGSTVAAPSGSTVAAPSGS

SEQ ID NO: 598 - protein, 88 aa, Artificial Sequence
  Linker peptide
GSGSGSGSTVAAPSGSTVAAPSGSTVAAPSGSTVAAPSGSTVAAPSGSTVAAPSGSTVAAPSGSTVAAPSGSTVAAPSGSTVAAPSGS

SEQ ID NO: 599 - protein, 6 aa, Artificial Sequence
  Linker peptide
PAVPPP

SEQ ID NO: 600 - protein, 12 aa, Artificial Sequence
  Linker peptide
PAVPPPPAVPPP

SEQ ID NO: 601 - protein, 18 aa, Artificial Sequence
  Linker peptide
PAVPPPPAVPPPPAVPPP

SEQ ID NO: 602 - protein, 24 aa, Artificial Sequence
  Linker peptide
PAVPPPPAVPPPPAVPPPPAVPPP

SEQ ID NO: 603 - protein, 30 aa, Artificial Sequence
  Linker peptide
PAVPPPPAVPPPPAVPPPPAVPPPPAVPPP

SEQ ID NO: 604 - protein, 36 aa, Artificial Sequence
  Linker peptide
PAVPPPPAVPPPPAVPPPPAVPPPPAVPPPPAVPPP

SEQ ID NO: 605 - protein, 42 aa, Artificial Sequence
  Linker peptide
PAVPPPPAVPPPPAVPPPPAVPPPPAVPPPPAVPPPPAVPPP

SEQ ID NO: 606 - protein, 48 aa, Artificial Sequence
  Linker peptide
PAVPPPPAVPPPPAVPPPPAVPPPPAVPPPPAVPPPPAVPPPPAVPPP

SEQ ID NO: 607 - protein, 54 aa, Artificial Sequence
  Linker peptide
PAVPPPPAVPPPPAVPPPPAVPPPPAVPPPPAVPPPPAVPPPPAVPPPPAVPPP

SEQ ID NO: 608 - protein, 60 aa, Artificial Sequence
  Linker peptide
PAVPPPPAVPPPPAVPPPPAVPPPPAVPPPPAVPPPPAVPPPPAVPPPPAVPPPPAVPPP

SEQ ID NO: 609 - protein, 8 aa, Artificial Sequence
  Linker peptide
PAVPPPGS

SEQ ID NO: 610 - protein, 14 aa, Artificial Sequence
  Linker peptide
PAVPPPPAVPPPGS

SEQ ID NO: 611 - protein, 20 aa, Artificial Sequence
  Linker peptide
PAVPPPPAVPPPPAVPPPGS

SEQ ID NO: 612 - protein, 26 aa, Artificial Sequence
  Linker peptide
PAVPPPPAVPPPPAVPPPPAVPPPGS

SEQ ID NO: 613 - protein, 32 aa, Artificial Sequence
  Linker peptide
PAVPPPPAVPPPPAVPPPPAVPPPPAVPPPGS

SEQ ID NO: 614 - protein, 38 aa, Artificial Sequence
  Linker peptide
PAVPPPPAVPPPPAVPPPPAVPPPPAVPPPPAVPPPGS

SEQ ID NO: 615 - protein, 44 aa, Artificial Sequence
  Linker peptide
PAVPPPPAVPPPPAVPPPPAVPPPPAVPPPPAVPPPPAVPPPGS

SEQ ID NO: 616 - protein, 50 aa, Artificial Sequence
  Linker peptide
PAVPPPPAVPPPPAVPPPPAVPPPPAVPPPPAVPPPPAVPPPPAVPPPGS

SEQ ID NO: 617 - protein, 56 aa, Artificial Sequence
  Linker peptide
PAVPPPPAVPPPPAVPPPPAVPPPPAVPPPPAVPPPPAVPPPPAVPPPPAVPPPGS

SEQ ID NO: 618 - protein, 62 aa, Artificial Sequence
  Linker peptide
PAVPPPPAVPPPPAVPPPPAVPPPPAVPPPPAVPPPPAVPPPPAVPPPPAVPPPPAVPPPGS

SEQ ID NO: 619 - protein, 10 aa, Artificial Sequence
  Linker peptide
PAVPPPGSGS

SEQ ID NO: 620 - protein, 16 aa, Artificial Sequence
  Linker peptide
PAVPPPPAVPPPGSGS

SEQ ID NO: 621 - protein, 22 aa, Artificial Sequence
  Linker peptide
PAVPPPPAVPPPPAVPPPGSGS

SEQ ID NO: 622 - protein, 28 aa, Artificial Sequence
  Linker peptide
PAVPPPPAVPPPPAVPPPPAVPPPGSGS

SEQ ID NO: 623 - protein, 34 aa, Artificial Sequence
  Linker peptide
PAVPPPPAVPPPPAVPPPPAVPPPPAVPPPGSGS

SEQ ID NO: 624 - protein, 40 aa, Artificial Sequence
  Linker peptide
PAVPPPPAVPPPPAVPPPPAVPPPPAVPPPPAVPPPGSGS

SEQ ID NO: 625 - protein, 46 aa, Artificial Sequence
  Linker peptide
PAVPPPPAVPPPPAVPPPPAVPPPPAVPPPPAVPPPPAVPPPGSGS

SEQ ID NO: 626 - protein, 52 aa, Artificial Sequence
  Linker peptide
PAVPPPPAVPPPPAVPPPPAVPPPPAVPPPPAVPPPPAVPPPPAVPPPGSGS

SEQ ID NO: 627 - protein, 58 aa, Artificial Sequence
  Linker peptide
PAVPPPPAVPPPPAVPPPPAVPPPPAVPPPPAVPPPPAVPPPPAVPPPPAVPPPGSGS

SEQ ID NO: 628 - protein, 64 aa, Artificial Sequence
  Linker peptide
PAVPPPPAVPPPPAVPPPPAVPPPPAVPPPPAVPPPPAVPPPPAVPPPPAVPPPPAVPPPGSGS

SEQ ID NO: 629 - protein, 12 aa, Artificial Sequence
  Linker peptide
PAVPPPGSGSGS

SEQ ID NO: 630 - protein, 18 aa, Artificial Sequence
  Linker peptide
PAVPPPPAVPPPGSGSGS

SEQ ID NO: 631 - protein, 24 aa, Artificial Sequence
  Linker peptide
PAVPPPPAVPPPPAVPPPGSGSGS

SEQ ID NO: 632 - protein, 30 aa, Artificial Sequence
  Linker peptide
PAVPPPPAVPPPPAVPPPPAVPPPGSGSGS

SEQ ID NO: 633 - protein, 36 aa, Artificial Sequence
  Linker peptide
PAVPPPPAVPPPPAVPPPPAVPPPPAVPPPGSGSGS

SEQ ID NO: 634 - protein, 42 aa, Artificial Sequence
  Linker peptide
PAVPPPPAVPPPPAVPPPPAVPPPPAVPPPPAVPPPGSGSGS

SEQ ID NO: 635 - protein, 48 aa, Artificial Sequence
  Linker peptide
PAVPPPPAVPPPPAVPPPPAVPPPPAVPPPPAVPPPPAVPPPGSGSGS

SEQ ID NO: 636 - protein, 54 aa, Artificial Sequence
  Linker peptide
PAVPPPPAVPPPPAVPPPPAVPPPPAVPPPPAVPPPPAVPPPPAVPPPGSGSGS

SEQ ID NO: 637 - protein, 60 aa, Artificial Sequence
  Linker peptide
PAVPPPPAVPPPPAVPPPPAVPPPPAVPPPPAVPPPPAVPPPPAVPPPPAVPPPGSGSGS

SEQ ID NO: 638 - protein, 66 aa, Artificial Sequence
  Linker peptide
PAVPPPPAVPPPPAVPPPPAVPPPPAVPPPPAVPPPPAVPPPPAVPPPPAVPPPPAVPPPGSGSGS

SEQ ID NO: 639 - protein, 14 aa, Artificial Sequence
  Linker peptide
PAVPPPGSGSGSGS

SEQ ID NO: 640 - protein, 20 aa, Artificial Sequence
  Linker peptide
PAVPPPPAVPPPGSGSGSGS

SEQ ID NO: 641 - protein, 26 aa, Artificial Sequence
  Linker peptide
PAVPPPPAVPPPPAVPPPGSGSGSGS

SEQ ID NO: 642 - protein, 32 aa, Artificial Sequence
  Linker peptide
PAVPPPPAVPPPPAVPPPPAVPPPGSGSGSGS

SEQ ID NO: 643 - protein, 38 aa, Artificial Sequence
  Linker peptide
PAVPPPPAVPPPPAVPPPPAVPPPPAVPPPGSGSGSGS

SEQ ID NO: 644 - protein, 44 aa, Artificial Sequence
  Linker peptide
PAVPPPPAVPPPPAVPPPPAVPPPPAVPPPPAVPPPGSGSGSGS

SEQ ID NO: 645 - protein, 50 aa, Artificial Sequence
  Linker peptide
PAVPPPPAVPPPPAVPPPPAVPPPPAVPPPPAVPPPPAVPPPGSGSGSGS

SEQ ID NO: 646 - protein, 56 aa, Artificial Sequence
  Linker peptide
PAVPPPPAVPPPPAVPPPPAVPPPPAVPPPPAVPPPPAVPPPPAVPPPGSGSGSGS

SEQ ID NO: 647 - protein, 62 aa, Artificial Sequence
  Linker peptide
PAVPPPPAVPPPPAVPPPPAVPPPPAVPPPPAVPPPPAVPPPPAVPPPPAVPPPGSGSGSGS

SEQ ID NO: 648 - protein, 68 aa, Artificial Sequence
  Linker peptide
PAVPPPPAVPPPPAVPPPPAVPPPPAVPPPPAVPPPPAVPPPPAVPPPPAVPPPPAVPPPGSGSGSGS

SEQ ID NO: 649 - protein, 6 aa, Artificial Sequence
  Linker peptide
TVSDVP

SEQ ID NO: 650 - protein, 12 aa, Artificial Sequence
  Linker peptide
TVSDVPTVSDVP

SEQ ID NO: 651 - protein, 18 aa, Artificial Sequence
  Linker peptide
TVSDVPTVSDVPTVSDVP

SEQ ID NO: 652 - protein, 24 aa, Artificial Sequence
  Linker peptide
TVSDVPTVSDVPTVSDVPTVSDVP

SEQ ID NO: 653 - protein, 30 aa, Artificial Sequence
  Linker peptide
TVSDVPTVSDVPTVSDVPTVSDVPTVSDVP

SEQ ID NO: 654 - protein, 36 aa, Artificial Sequence
  Linker peptide
TVSDVPTVSDVPTVSDVPTVSDVPTVSDVPTVSDVP

SEQ ID NO: 655 - protein, 42 aa, Artificial Sequence
  Linker peptide
TVSDVPTVSDVPTVSDVPTVSDVPTVSDVPTVSDVPTVSDVP

SEQ ID NO: 656 - protein, 48 aa, Artificial Sequence
  Linker peptide
TVSDVPTVSDVPTVSDVPTVSDVPTVSDVPTVSDVPTVSDVPTVSDVP

SEQ ID NO: 657 - protein, 54 aa, Artificial Sequence
  Linker peptide
TVSDVPTVSDVPTVSDVPTVSDVPTVSDVPTVSDVPTVSDVPTVSDVPTVSDVP

SEQ ID NO: 658 - protein, 60 aa, Artificial Sequence
  Linker peptide
TVSDVPTVSDVPTVSDVPTVSDVPTVSDVPTVSDVPTVSDVPTVSDVPTVSDVPTVSDVP

SEQ ID NO: 659 - protein, 8 aa, Artificial Sequence
  Linker peptide
TVSDVPGS

SEQ ID NO: 660 - protein, 14 aa, Artificial Sequence
  Linker peptide
TVSDVPTVSDVPGS

SEQ ID NO: 661 - protein, 20 aa, Artificial Sequence
  Linker peptide
TVSDVPTVSDVPTVSDVPGS

SEQ ID NO: 662 - protein, 26 aa, Artificial Sequence
  Linker peptide
TVSDVPTVSDVPTVSDVPTVSDVPGS

SEQ ID NO: 663 - protein, 32 aa, Artificial Sequence
  Linker peptide
TVSDVPTVSDVPTVSDVPTVSDVPTVSDVPGS

SEQ ID NO: 664 - protein, 38 aa, Artificial Sequence
  Linker peptide
TVSDVPTVSDVPTVSDVPTVSDVPTVSDVPTVSDVPGS

SEQ ID NO: 665 - protein, 44 aa, Artificial Sequence
  Linker peptide
TVSDVPTVSDVPTVSDVPTVSDVPTVSDVPTVSDVPTVSDVPGS

SEQ ID NO: 666 - protein, 50 aa, Artificial Sequence
  Linker peptide
TVSDVPTVSDVPTVSDVPTVSDVPTVSDVPTVSDVPTVSDVPTVSDVPGS

SEQ ID NO: 667 - protein, 56 aa, Artificial Sequence
  Linker peptide
TVSDVPTVSDVPTVSDVPTVSDVPTVSDVPTVSDVPTVSDVPTVSDVPTVSDVPGS

SEQ ID NO: 668 - protein, 62 aa, Artificial Sequence
  Linker peptide
TVSDVPTVSDVPTVSDVPTVSDVPTVSDVPTVSDVPTVSDVPTVSDVPTVSDVPTVSDVPGS

SEQ ID NO: 669 - protein, 10 aa, Artificial Sequence
  Linker peptide
TVSDVPGSGS

SEQ ID NO: 670 - protein, 16 aa, Artificial Sequence
  Linker peptide
TVSDVPTVSDVPGSGS

SEQ ID NO: 671 - protein, 22 aa, Artificial Sequence
  Linker peptide
TVSDVPTVSDVPTVSDVPGSGS

SEQ ID NO: 672 - protein, 28 aa, Artificial Sequence
  Linker peptide
TVSDVPTVSDVPTVSDVPTVSDVPGSGS

SEQ ID NO: 673 - protein, 34 aa, Artificial Sequence
  Linker peptide
TVSDVPTVSDVPTVSDVPTVSDVPTVSDVPGSGS

SEQ ID NO: 674 - protein, 40 aa, Artificial Sequence
  Linker peptide
TVSDVPTVSDVPTVSDVPTVSDVPTVSDVPTVSDVPGSGS

SEQ ID NO: 675 - protein, 46 aa, Artificial Sequence
  Linker peptide
TVSDVPTVSDVPTVSDVPTVSDVPTVSDVPTVSDVPTVSDVPGSGS

SEQ ID NO: 676 - protein, 52 aa, Artificial Sequence
  Linker peptide
TVSDVPTVSDVPTVSDVPTVSDVPTVSDVPTVSDVPTVSDVPTVSDVPGSGS

SEQ ID NO: 677 - protein, 58 aa, Artificial Sequence
  Linker peptide
TVSDVPTVSDVPTVSDVPTVSDVPTVSDVPTVSDVPTVSDVPTVSDVPTVSDVPGSGS

SEQ ID NO: 678 - protein, 64 aa, Artificial Sequence
  Linker peptide
TVSDVPTVSDVPTVSDVPTVSDVPTVSDVPTVSDVPTVSDVPTVSDVPTVSDVPTVSDVPGSGS

SEQ ID NO: 679 - protein, 12 aa, Artificial Sequence
  Linker peptide
TVSDVPGSGSGS

SEQ ID NO: 680 - protein, 18 aa, Artificial Sequence
  Linker peptide
TVSDVPTVSDVPGSGSGS

SEQ ID NO: 681 - protein, 24 aa, Artificial Sequence
  Linker peptide
TVSDVPTVSDVPTVSDVPGSGSGS

SEQ ID NO: 682 - protein, 30 aa, Artificial Sequence
  Linker peptide
TVSDVPTVSDVPTVSDVPTVSDVPGSGSGS

SEQ ID NO: 683 - protein, 36 aa, Artificial Sequence
  Linker peptide
TVSDVPTVSDVPTVSDVPTVSDVPTVSDVPGSGSGS

SEQ ID NO: 684 - protein, 42 aa, Artificial Sequence
  Linker peptide
TVSDVPTVSDVPTVSDVPTVSDVPTVSDVPTVSDVPGSGSGS

SEQ ID NO: 685 - protein, 48 aa, Artificial Sequence
  Linker peptide
TVSDVPTVSDVPTVSDVPTVSDVPTVSDVPTVSDVPTVSDVPGSGSGS

SEQ ID NO: 686 - protein, 54 aa, Artificial Sequence
  Linker peptide
TVSDVPTVSDVPTVSDVPTVSDVPTVSDVPTVSDVPTVSDVPTVSDVPGSGSGS

SEQ ID NO: 687 - protein, 60 aa, Artificial Sequence
  Linker peptide
TVSDVPTVSDVPTVSDVPTVSDVPTVSDVPTVSDVPTVSDVPTVSDVPTVSDVPGSGSGS

SEQ ID NO: 688 - protein, 66 aa, Artificial Sequence
  Linker peptide
TVSDVPTVSDVPTVSDVPTVSDVPTVSDVPTVSDVPTVSDVPTVSDVPTVSDVPTVSDVPGSGSGS

SEQ ID NO: 689 - protein, 14 aa, Artificial Sequence
  Linker peptide
TVSDVPGSGSGSGS

SEQ ID NO: 690 - protein, 20 aa, Artificial Sequence
  Linker peptide
TVSDVPTVSDVPGSGSGSGS

SEQ ID NO: 691 - protein, 26 aa, Artificial Sequence
  Linker peptide
TVSDVPTVSDVPTVSDVPGSGSGSGS

SEQ ID NO: 692 - protein, 32 aa, Artificial Sequence
  Linker peptide
TVSDVPTVSDVPTVSDVPTVSDVPGSGSGSGS

SEQ ID NO: 693 - protein, 38 aa, Artificial Sequence
  Linker peptide
TVSDVPTVSDVPTVSDVPTVSDVPTVSDVPGSGSGSGS

SEQ ID NO: 694 - protein, 44 aa, Artificial Sequence
  Linker peptide
TVSDVPTVSDVPTVSDVPTVSDVPTVSDVPTVSDVPGSGSGSGS

SEQ ID NO: 695 - protein, 50 aa, Artificial Sequence
  Linker peptide
TVSDVPTVSDVPTVSDVPTVSDVPTVSDVPTVSDVPTVSDVPGSGSGSGS

SEQ ID NO: 696 - protein, 56 aa, Artificial Sequence
  Linker peptide
TVSDVPTVSDVPTVSDVPTVSDVPTVSDVPTVSDVPTVSDVPTVSDVPGSGSGSGS

SEQ ID NO: 697 - protein, 62 aa, Artificial Sequence
  Linker peptide
TVSDVPTVSDVPTVSDVPTVSDVPTVSDVPTVSDVPTVSDVPTVSDVPTVSDVPGSGSGSGS

SEQ ID NO: 698 - protein, 68 aa, Artificial Sequence
  Linker peptide
TVSDVPTVSDVPTVSDVPTVSDVPTVSDVPTVSDVPTVSDVPTVSDVPTVSDVPTVSDVPGSGSGSGS

SEQ ID NO: 699 - protein, 6 aa, Artificial Sequence
  Linker peptide
TGLDSP

SEQ ID NO: 700 - protein, 12 aa, Artificial Sequence
  Linker peptide
TGLDSPTGLDSP

SEQ ID NO: 701 - protein, 18 aa, Artificial Sequence
  Linker peptide
TGLDSPTGLDSPTGLDSP

SEQ ID NO: 702 - protein, 24 aa, Artificial Sequence
  Linker peptide
TGLDSPTGLDSPTGLDSPTGLDSP

SEQ ID NO: 703 - protein, 30 aa, Artificial Sequence
  Linker peptide
TGLDSPTGLDSPTGLDSPTGLDSPTGLDSP

SEQ ID NO: 704 - protein, 36 aa, Artificial Sequence
  Linker peptide
TGLDSPTGLDSPTGLDSPTGLDSPTGLDSPTGLDSP

SEQ ID NO: 705 - protein, 42 aa, Artificial Sequence
  Linker peptide
TGLDSPTGLDSPTGLDSPTGLDSPTGLDSPTGLDSPTGLDSP

SEQ ID NO: 706 - protein, 48 aa, Artificial Sequence
  Linker peptide
TGLDSPTGLDSPTGLDSPTGLDSPTGLDSPTGLDSPTGLDSPTGLDSP

SEQ ID NO: 707 - protein, 54 aa, Artificial Sequence
  Linker peptide
TGLDSPTGLDSPTGLDSPTGLDSPTGLDSPTGLDSPTGLDSPTGLDSPTGLDSP

SEQ ID NO: 708 - protein, 60 aa, Artificial Sequence
  Linker peptide
TGLDSPTGLDSPTGLDSPTGLDSPTGLDSPTGLDSPTGLDSPTGLDSPTGLDSPTGLDSP

SEQ ID NO: 709 - protein, 8 aa, Artificial Sequence
  Linker peptide
TGLDSPGS

SEQ ID NO: 710 - protein, 14 aa, Artificial Sequence
  Linker peptide
TGLDSPTGLDSPGS

SEQ ID NO: 711 - protein, 20 aa, Artificial Sequence
  Linker peptide
TGLDSPTGLDSPTGLDSPGS

SEQ ID NO: 712 - protein, 26 aa, Artificial Sequence
  Linker peptide
TGLDSPTGLDSPTGLDSPTGLDSPGS

SEQ ID NO: 713 - protein, 32 aa, Artificial Sequence
  Linker peptide
TGLDSPTGLDSPTGLDSPTGLDSPTGLDSPGS

SEQ ID NO: 714 - protein, 38 aa, Artificial Sequence
  Linker peptide
TGLDSPTGLDSPTGLDSPTGLDSPTGLDSPTGLDSPGS

SEQ ID NO: 715 - protein, 44 aa, Artificial Sequence
  Linker peptide
TGLDSPTGLDSPTGLDSPTGLDSPTGLDSPTGLDSPTGLDSPGS

SEQ ID NO: 716 - protein, 50 aa, Artificial Sequence
  Linker peptide
TGLDSPTGLDSPTGLDSPTGLDSPTGLDSPTGLDSPTGLDSPTGLDSPGS

SEQ ID NO: 717 - protein, 56 aa, Artificial Sequence
  Linker peptide
TGLDSPTGLDSPTGLDSPTGLDSPTGLDSPTGLDSPTGLDSPTGLDSPTGLDSPGS

SEQ ID NO: 718 - protein, 62 aa, Artificial Sequence
  Linker peptide
TGLDSPTGLDSPTGLDSPTGLDSPTGLDSPTGLDSPTGLDSPTGLDSPTGLDSPTGLDSPGS

SEQ ID NO: 719 - protein, 10 aa, Artificial Sequence
  Linker peptide
TGLDSPGSGS

SEQ ID NO: 720 - protein, 16 aa, Artificial Sequence
  Linker peptide
TGLDSPTGLDSPGSGS

SEQ ID NO: 721 - protein, 22 aa, Artificial Sequence
  Linker peptide
TGLDSPTGLDSPTGLDSPGSGS

SEQ ID NO: 722 - protein, 28 aa, Artificial Sequence
  Linker peptide
TGLDSPTGLDSPTGLDSPTGLDSPGSGS

SEQ ID NO: 723 - protein, 34 aa, Artificial Sequence
  Linker peptide
TGLDSPTGLDSPTGLDSPTGLDSPTGLDSPGSGS

SEQ ID NO: 724 - protein, 40 aa, Artificial Sequence
  Linker peptide
TGLDSPTGLDSPTGLDSPTGLDSPTGLDSPTGLDSPGSGS

SEQ ID NO: 725 - protein, 46 aa, Artificial Sequence
  Linker peptide
TGLDSPTGLDSPTGLDSPTGLDSPTGLDSPTGLDSPTGLDSPGSGS

SEQ ID NO: 726 - protein, 52 aa, Artificial Sequence
  Linker peptide
TGLDSPTGLDSPTGLDSPTGLDSPTGLDSPTGLDSPTGLDSPTGLDSPGSGS

SEQ ID NO: 727 - protein, 58 aa, Artificial Sequence
  Linker peptide
TGLDSPTGLDSPTGLDSPTGLDSPTGLDSPTGLDSPTGLDSPTGLDSPTGLDSPGSGS

SEQ ID NO: 728 - protein, 64 aa, Artificial Sequence
  Linker peptide
TGLDSPTGLDSPTGLDSPTGLDSPTGLDSPTGLDSPTGLDSPTGLDSPTGLDSPTGLDSPGSGS

SEQ ID NO: 729 - protein, 12 aa, Artificial Sequence
  Linker peptide
TGLDSPGSGSGS

SEQ ID NO: 730 - protein, 18 aa, Artificial Sequence
  Linker peptide
TGLDSPTGLDSPGSGSGS

SEQ ID NO: 731 - protein, 24 aa, Artificial Sequence
  Linker peptide
TGLDSPTGLDSPTGLDSPGSGSGS

SEQ ID NO: 732 - protein, 30 aa, Artificial Sequence
  Linker peptide
TGLDSPTGLDSPTGLDSPTGLDSPGSGSGS

SEQ ID NO: 733 - protein, 36 aa, Artificial Sequence
  Linker peptide
TGLDSPTGLDSPTGLDSPTGLDSPTGLDSPGSGSGS

SEQ ID NO: 734 - protein, 42 aa, Artificial Sequence
  Linker peptide
TGLDSPTGLDSPTGLDSPTGLDSPTGLDSPTGLDSPGSGSGS

SEQ ID NO: 735 - protein, 48 aa, Artificial Sequence
  Linker peptide
TGLDSPTGLDSPTGLDSPTGLDSPTGLDSPTGLDSPTGLDSPGSGSGS

SEQ ID NO: 736 - protein, 54 aa, Artificial Sequence
  Linker peptide
TGLDSPTGLDSPTGLDSPTGLDSPTGLDSPTGLDSPTGLDSPTGLDSPGSGSGS

SEQ ID NO: 737 - protein, 60 aa, Artificial Sequence
  Linker peptide
TGLDSPTGLDSPTGLDSPTGLDSPTGLDSPTGLDSPTGLDSPTGLDSPTGLDSPGSGSGS

SEQ ID NO: 738 - protein, 66 aa, Artificial Sequence
  Linker peptide
TGLDSPTGLDSPTGLDSPTGLDSPTGLDSPTGLDSPTGLDSPTGLDSPTGLDSPTGLDSPGSGSGS

SEQ ID NO: 739 - protein, 14 aa, Artificial Sequence
  Linker peptide
TGLDSPGSGSGSGS

SEQ ID NO: 740 - protein, 20 aa, Artificial Sequence
  Linker peptide
TGLDSPTGLDSPGSGSGSGS

SEQ ID NO: 741 - protein, 26 aa, Artificial Sequence
  Linker peptide
TGLDSPTGLDSPTGLDSPGSGSGSGS

SEQ ID NO: 742 - protein, 32 aa, Artificial Sequence
  Linker peptide
TGLDSPTGLDSPTGLDSPTGLDSPGSGSGSGS

SEQ ID NO: 743 - protein, 38 aa, Artificial Sequence
  Linker peptide
TGLDSPTGLDSPTGLDSPTGLDSPTGLDSPGSGSGSGS

SEQ ID NO: 744 - protein, 44 aa, Artificial Sequence
  Linker peptide
TGLDSPTGLDSPTGLDSPTGLDSPTGLDSPTGLDSPGSGSGSGS

SEQ ID NO: 745 - protein, 50 aa, Artificial Sequence
  Linker peptide
TGLDSPTGLDSPTGLDSPTGLDSPTGLDSPTGLDSPTGLDSPGSGSGSGS

SEQ ID NO: 746 - protein, 56 aa, Artificial Sequence
  Linker peptide
TGLDSPTGLDSPTGLDSPTGLDSPTGLDSPTGLDSPTGLDSPTGLDSPGSGSGSGS

SEQ ID NO: 747 - protein, 62 aa, Artificial Sequence
  Linker peptide
TGLDSPTGLDSPTGLDSPTGLDSPTGLDSPTGLDSPTGLDSPTGLDSPTGLDSPGSGSGSGS

SEQ ID NO: 748 - protein, 68 aa, Artificial Sequence
  Linker peptide
TGLDSPTGLDSPTGLDSPTGLDSPTGLDSPTGLDSPTGLDSPTGLDSPTGLDSPTGLDSPGSGSGSGS

SEQ ID NO: 749 - protein, 576 aa, Artificial Sequence; identical to SEQ ID NO: 10
  bnAb-Adnectin fusion polypeptide

SEQ ID NO: 750 - protein, 580 aa, Artificial Sequence; identical to SEQ ID NO: 9
  bnAb-Adnectin fusion polypeptide

SEQ ID NO: 751 - protein, 570 aa, Artificial Sequence
  bnAb-Adnectin fusion polypeptide
QVQLVQSGGQMKKPGESMRISCRASGYEFIDCTLNWIRLAPGKRPEWMGWLKPRGGAVNYARPLQGRVTMTRDVYSDTAFLELRSLTVDDTAVYFCTRGKNCDYNWDFEHWGRGTPVIVSSPSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGVSDVPRDLEVVAATPTSLLISWDAPAVTVHSYHIQYWPLGSYQRYQVFSVPGSKSTATISGLKPGVEYQIRVYAETGGADSDQSFGWIQIGYRTP

SEQ ID NO: 752 - protein, 331 aa, Artificial Sequence
  bnAb-Adnectin fusion polypeptide
EIVLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGECGGGGSGGGGSGGGGSGGGGSGGGGSGVSDVPRDLEVVAATPTSLLISWDAPAVTVHSYHIQYWPLGSYQRYQVFSVPGSKSTATISGLKPGVEYQIRVYAETGGADSDQSFGWIQIGYRTP

SEQ ID NO: 753 - protein, 619 aa, Artificial Sequence
  bnAb-Adnectin fusion polypeptide
EVRLVESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWSGYPPGEEYFQDWGQGTLVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKESPEPETPEDEGVSDVPRDLEVVAATPTSLLISWEYKVHPYRYYRITYGETGGNSPVQEFTVPSVLSTAEISGLKPGVDYTITVYAVTRGVDSAPISINYRTPGGGGSGGGGSGGGGSGGGGSTIAEYAARIEALIRAAQEQQEKNEAALRELYKWAS

SEQ ID NO: 754 - protein, 373 aa, Artificial Sequence
  bnAb-Adnectin fusion polypeptide
ASELTQDPAVSVALKQTVTITCRGDSLRSHYASWYQKKPGQAPVLLFYGKNNRPSGIPDRFSGSASGNRASLTITGAQAEDEADYYCSSRDKSGSRLSVFGGGTKLTVLSQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSESPEPETPEDEGVSDVPRDLEVVAATPTSLLISWEYKVHPYRYYRITYGETGGNSPVQEFTVPSVLSTAEISGLKPGVDYTITVYAVTRGVDSAPISINYRTPGGGGSGGGGSGGGGSGGGGSTIAEYAARIEALIRAAQEQQEKNEAALRELYKWAS

SEQ ID NO: 755 - protein, 576 aa, Artificial Sequence; identical to SEQ ID NO: 10
  bnAb-Adnectin fusion polypeptide

SEQ ID NO: 756 - protein, 580 aa, Artificial Sequence; identical to SEQ ID NO: 9
  bnAb-Adnectin fusion polypeptide

SEQ ID NO: 757 - protein, 595 aa, Artificial Sequence
  bnAb-Adnectin fusion polypeptide
EVRLVESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWSGYPPGEEYFQDWGQGTLVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPESPEPETPEDESPEPETPEDEMASTSGSASYLIPSDLEVVAATPTSLSIYWYPVASTIINFRITYVETGGNSPVQEFTVPGSKSTATISGLKPGVDYTITVYAVHYEQKYSEYWIGHPISINYRTEGSGSHHHHHH

SEQ ID NO: 758 - protein, 599 aa, Artificial Sequence
  bnAb-Adnectin fusion polypeptide
EVRLVESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWSGYPPGEEYFQDWGQGTLVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSMASTSGSASYLIPSDLEVVAATPTSLSIYWYPVASTIINFRITYVETGGNSPVQEFTVPGSKSTATISGLKPGVDYTITVYAVHYEQKYSEYWIGHPISINYRTEGSGSHHHHHH

SEQ ID NO: 759 - protein, 570 aa, Artificial Sequence; identical to SEQ ID NO: 751
  bnAb-Adnectin fusion polypeptide

SEQ ID NO: 760 - protein, 331 aa, Artificial Sequence; identical to SEQ ID NO: 752
  bnAb-Adnectin fusion polypeptide

SEQ ID NO: 761 - protein, 589 aa, Artificial Sequence
  bnAb-Adnectin fusion polypeptide
QVQLVQSGGQMKKPGESMRISCRASGYEFIDCTLNWIRLAPGKRPEWMGWLKPRGGAVNYARPLQGRVTMTRDVYSDTAFLELRSLTVDDTAVYFCTRGKNCDYNWDFEHWGRGTPVIVSSPSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSMASTSGSASYLIPSDLEVVAATPTSLSIYWYPVASTIINFRITYVETGGNSPVQEFTVPGSKSTATISGLKPGVDYTITVYAVHYEQKYSEYWIGHPISINYRTEGSGSHHHHHH

SEQ ID NO: 762 - protein, 350 aa, Artificial Sequence
  bnAb-Adnectin fusion polypeptide
EIVLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGECGGGGSGGGGSGGGGSGGGGSGGGGSMASTSGSASYLIPSDLEVVAATPTSLSIYWYPVASTIINFRITYVETGGNSPVQEFTVPGSKSTATISGLKPGVDYTITVYAVHYEQKYSEYWIGHPISINYRTEGSGSHHHHHH

SEQ ID NO: 763 - protein, 618 aa, Artificial Sequence
  bnAb-Adnectin fusion polypeptide
EVRLVESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWSGYPPGEEYFQDWGQGTLVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKESPEPETPEDEGVSDVPRDLEVVAATPTSLLISWEYKVHPYRYYRITYGETGGNSPVQEFTVPSVLSTAEISGLKPGVDYTITVYAVTRGVDSAPISINYRTPGGGGSGGGGSGGGGSGGGGTIAEYAARIEALIRAAQEQQEKNEAALRELYKWAS

SEQ ID NO: 764 - protein, 372 aa, Artificial Sequence
  bnAb-Adnectin fusion polypeptide
ASELTQDPAVSVALKQTVTITCRGDSLRSHYASWYQKKPGQAPVLLFYGKNNRPSGIPDRFSGSASGNRASLTITGAQAEDEADYYCSSRDKSGSRLSVFGGGTKLTVLSQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSESPEPETPEDEGVSDVPRDLEVVAATPTSLLISWEYKVHPYRYYRITYGETGGNSPVQEFTVPSVLSTAEISGLKPGVDYTITVYAVTRGVDSAPISINYRTPGGGGSGGGGSGGGGSGGGGTIAEYAARIEALIRAAQEQQEKNEAALRELYKWAS

SEQ ID NO: 765 - protein, 595 aa, Artificial Sequence; identical to SEQ ID NO: 757
  bnAb-Adnectin fusion polypeptide

SEQ ID NO: 766 - protein, 355 aa, Artificial Sequence
  bnAb-Adnectin fusion polypeptide
ASELTQDPAVSVALKQTVTITCRGDSLRSHYASWYQKKPGQAPVLLFYGKNNRPSGIPDRFSGSASGNRASLTITGAQAEDEADYYCSSRDKSGSRLSVFGGGTKLTVLSQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSGGGGSGGGGSGGGGSGGGGSMASTSGSASYLIPSDLEVVAATPTSLSIYWYPVASTIINFRITYVETGGNSPVQEFTVPGSKSTATISGLKPGVDYTITVYAVHYEQKYSEYWIGHPISINYRTEGSGSHHHHHH

SEQ ID NO: 767 - protein, 351 aa, Artificial Sequence
  bnAb-Adnectin fusion polypeptide
ASELTQDPAVSVALKQTVTITCRGDSLRSHYASWYQKKPGQAPVLLFYGKNNRPSGIPDRFSGSASGNRASLTITGAQAEDEADYYCSSRDKSGSRLSVFGGGTKLTVLSQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSESPEPETPEDESPEPETPEDEMASTSGSASYLIPSDLEVVAATPTSLSIYWYPVASTIINFRITYVETGGNSPVQEFTVPGSKSTATISGLKPGVDYTITVYAVHYEQKYSEYWIGHPISINYRTEGSGSHHHHHH

SEQ ID NO: 768 - protein, 461 aa, Artificial Sequence
  10E8v2 Heavy Chain
EVRLVESGGGLVKPGGSLRLSCSASGFNFDDAWMTWVRQPPGKGLEWVGRISGPGEGWSVDYAESVKGRFTISRLNSINFLYLEMNNVRTEDTGYYFCARTGKHYDFWSGYPPGEEYFQDWGQGTLVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 769 - protein, 461 aa, Artificial Sequence
  10E8v3 Heavy Chain
EVQLVESGGDLVKPGGSLRLSCSASGFSFKNTWMTWVRQAPGKGLEWVGRITGPGEGWTSDYAATVQGRFTISRNNMIDMLYLEMNRLRTDDTGLYYCVHTEKYYNFWGGYPPGEEYFQHWGRGTLVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 770 - protein, 461 aa, Artificial Sequence
  10E8v4-V5R-100cF Heavy chain
EVRLRESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWFGYPPGEEYFQDWGQGTLVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 771 - protein, 215 aa, Artificial Sequence
  10E8v2 Light chain
ASELTQDPAVSVALKQTVTITCRGDSLRSHYASWYQKKPGQAPILLFYGKNNRPSGVPDRFSGSASGNRASLTISGAQAEDDAEYYCSSRDKSGSRLSVFGGGTKLTVLSQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 772 - protein, 215 aa, Artificial Sequence
  10E8 v3 Light Chain
ASELTQETGVSVALGRTVTITCRGDSLRSHYASWYQKKPGQAPILLFYGKNNRPSGIHDRFSGSASGNRASLTISGAQAEDDAEYYCSSRDKSGSRLSVFGGGTKLTVLSQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

(19) United States
(12) Patent Application Publication
(10) Pub. No.: US 2024/0239877 A1
(43) Pub. Date: Jul. 18, 2024

(54) ANTIGEN BINDING POLYPEPTIDES

(71) Applicant: VIIV HEALTHCARE UK (No. 5) LIMITED, Brentford, Middlesex (GB)

(72) Inventors: Mark R. KRYSTAL, Branford, CT (US); David L. WENSEL, Branford, CT (US)

(21) Appl. No.: 16/954,988

(22) PCT Filed: Dec. 18, 2018
(86) PCT No.: PCT/IB2018/060271

§ 371 Date: Jun. 17, 2020

Related U.S. Application Data
(60) Provisional application No. 62/599,902, filed on Dec. 18, 2017.

(51) Int. Cl. C07K 16/10; C07K 16/28; C07K 16/46; A61K 39/00

(52) U.S. Cl. CPC C07K 16/1063; C07K 16/2812; C07K 16/468; A61K 2039/505; C07K 2317/14; C07K 2317/31; C07K 2317/565; C07K 2317/76; C07K 2318/20; C07K 2319/33

(57) ABSTRACT

The invention is directed to an antigen binding polypeptide comprising a CD4-binding polypeptide and an anti-HIV broadly neutralizing antibody (anti-HIV bnAb) polypeptide and combinations thereof. More specifically, the present invention relates to antigen binding polypeptides comprising a fibronectin-based scaffold domain protein that binds CD4 and an anti-HIV bnAb polypeptide or combinations thereof. Optionally, a fibronectin-based scaffold domain polypeptide that binds the N17 domain of gp41 (N17Adnectin™) and/or a HIV fusion peptide inhibitor moiety that binds gp41 may also be present in the antigen binding polypeptide. The invention also relates to the use of the innovative antigen binding polypeptides of the invention in therapeutic applications to treat HIV.

### CROSS REFERENCE TO RELATED APPLICATIONS

The instant application claims priority to U.S. Provisional Patent Application Ser. No. 62/599,902 filed Dec. 18, 2017 and PCT/US2018/060271 filed Dec. 18, 2018. The contents of these applications are incorporated by reference herein in its entirety.

### FIELD OF THE INVENTION

The invention is directed to antigen binding polypeptides comprising a CD4-binding polypeptide and an anti-HIV broadly neutralizing antibody (anti-HIV bnAb). More specifically, the present invention relates to polypeptides comprising a fibronectin-based scaffold domain polypeptide that binds CD4 (CD4 Adnectin™) and an anti-HIV bnAb polypeptide moiety, or combinations thereof. Additional polypeptide moieties such as a fibronectin-based scaffold domain polypeptide that binds the N17 domain of gp41 (N17 Adnectin™) and/or a HIV fusion peptide inhibitor moiety that binds gp41 may also be present. The invention also relates to the use of the innovative polypeptides with broadly neutralizing antibodies in therapeutic and prophylactic applications to treat and/or prevent HIV.

### SEQUENCE LISTING

The instant application contains a Sequence Listing which has been submitted electronically in ASCII format and is hereby incorporated by reference in its entirety. Said ASCII copy, created on Nov. 1, 2021, is named PR66475_US_SeqList_Nov2021.txt and is 586,985 bytes in size.

### BACKGROUND OF THE INVENTION

Acquired immunodeficiency syndrome (AIDS) is the result of infection by the retrovirus known as human immunodeficiency virus (HIV). It remains a major medical problem, with an estimated 36.7 million people infected worldwide at the end of 2015, of which 1.8 million where children less than 15 years of age. Moreover, there were 2.1 million new infections in 2015, and over 35 million people have died from AIDS-related conditions since the start of the epidemic, which includes 1.1 million in 2015 alone (www.hiv.gov).

Current therapy for HIV-infected individuals consists of a combination of approved anti-retroviral agents. Over two dozen drugs are currently approved for treating HIV infection (https://aidsinfo.nih.gov/understanding-hiv-aids/fact-sheets/21/58/fda-approved-hiv-medicines), either as single agents, fixed dose combinations or single tablet regimens, the latter two containing 2-4 approved agents. These agents belong to a number of different classes, targeting either a viral enzyme or the function of a viral protein during the virus life cycle. Thus, agents are classified as either nucleotide reverse transcriptase inhibitors (NRTIs), non-nucleotide reverse transcriptase inhibitors (NNRTIs), protease inhibitors (PIs), integrase inhibitors (INIs), or entry inhibitors (maraviroc, targets the host CCR5 protein; enfuvirtide is a peptide that targets the gp41 region of the viral gp160 protein; ibalizumab is a monoclonal antibody that targets the host CD4 protein). In addition, a pharmacokinetic enhancer with no antiviral activity (cobicistat) has been approved for use in combinations with antiretroviral agents (ARVs) that require boosting.

Despite the armamentarium of agents and drug combinations, there remains a medical need for new anti-retroviral agents, due in part to the need for chronic dosing to combat infection. Significant problems related to long-term toxicities are documented, creating a need to address and prevent these co-morbidities (e.g., CNS, CV/metabolic, renal disease). Also, increasing failure rates on current therapies continue to be a problem, due either to the presence or emergence of resistant strains or to non-compliance attributed to drug holidays or adverse side effects. For example, despite therapy, it has been estimated that 63% of subjects receiving combination therapy remained viremic, as they had viral loads >500 copies/ml (Oette, M. et al., “Primary HIV Drug Resistance and Efficacy of First-Line Antiretroviral Therapy Guided by Resistance Testing”, J. Acq. Imm. Def. Synd., 41(5):573-581 (2006)). Among these patients, 76% had viruses that were resistant to one or more classes of antiretroviral agents. As a result, new drugs are needed that are more convenient, have high genetic barriers to the development of resistance and have improved safety over current agents.

It is now well known that cells can be infected by HIV through a process by which fusion occurs between the cellular membrane and the viral membrane. The generally accepted model of this process is that the viral envelope glycoprotein complex (gp120/gp41) interacts with cell surface receptors on the membranes of the target cells. Following binding of gp120 to cellular receptors (e.g., CD4 in combination with a chemokine co-receptor such as CCR5 or CXCR4), a conformational change is induced in the gp120/gp41 complex that allows gp41 to insert into the membrane of the target cell and mediate membrane fusion. As these entry processes occur on the cell membrane, they are amenable for inhibition by macromolecules, which include biologic peptides (Haqqani et al., Antiviral Res., 98:158 (2013)). For instance, the approved antiviral peptide enfuvirtide (FUZEON®) targets a region of gp41 involved in membrane fusion. Larger polypeptides such as monoclonal antibodies can also inhibit different aspects of virus entry. A monoclonal antibody (ibalizumab; Bruno et al., J. Antimicrob. Chemother., 65:1839 (2010)) targeting an early step of virus entry, inhibition of gp160 conformational changes that occur after binding to the cellular receptor CD4 has recently been approved for use in highly treatment experienced (HTE) patients, while another monoclonal antibody targeting the co-receptor CCR5 (PRO-140; Tenorio, Curr. HIV/AIDS Rep., 8:1 (2011)) is currently in Phase 3 trials. These antibodies also have the property of being long acting antiretrovirals, with ibalizumab already approved as a biweekly medication delivered via intravenous infusion (https://www.accessdata.fda.gov/drugsatfda_docs/label/2018/7610651bl.pdf, Jacobson et al., J. Infect. Dis., 201:1481 (2010); Jacobson et al., Antimicrob. Agents Chemother., 53:450 (2009)).

Another property of peptide entry inhibitors is that enhanced or synergistic potency can be obtained if two peptide inhibitors are attached to each other, or if a single inhibitor is localized near the site of action through binding to membrane biomolecules. Thus, attaching a fusion peptide inhibitor to a monoclonal antibody targeting CCR5 (Kopetzki et al., Virol. J., 5:56 (2008)) or attaching a cholesterol moiety to the C-terminus of a peptide fusion inhibitor to place it at the surface of the target cell membrane (Ingallinela et al., Proc. Natl. Acad. Sci. USA, 106:5801 (2009); Augusto et al., J. Antimicrob. Chemother., 69:1286 (2014)) drastically increases the potency of the combined molecule compared to the separate molecules. Similarly, bispecific antibodies consisting of anti-HIV-1 neutralizing antibody fragments targeting gp120 fused to ibalizumab showed synergistic increases in potency compared to the individual inhibitors (Sun et al., J. Acquir. Immune Defic. Syndr., 66:473 (2014)).

The immune response to HIV is complex, and not fully understood. HIV envelope surface spikes comprising the gp120 protein and gp41 subunit interact with the CD4 and CCR5 cell surface receptors to facilitate viral entry into the cell. In an immune response to HIV, only antibodies which bind to the envelope surface spike can directly neutralize HIV virions. HIV has developed a means to evade the humoral immune response, particularly by evading the elicitation and recognition by neutralizing antibodies of the HIV envelope surface spike. Broadly neutralizing antibodies (bnAbs) are antibodies which can act immunologically against a wide range of viruses, but in the case of HIV, the elicitation and recognition of the HIV envelope surface spikes is hindered by difficult access to the bnAb epitopes on the spikes. Recently, isolation of potent bnAbs active against HIV-1 have been reported. Certain anti-HIV bnAbs, such as 10E8, have been shown to neutralize >97% of HIV strains, with a median IC₅₀ potency of 0.40 μg/mL, by recognizing the membrane proximal external region (MPER) of the HIV-1 envelope (Env), (see Huang et al., “Broad and potent neutralization of HIV-1 by a gp41-specific human antibody” Nature, 491, 4006-412 (2012)). Related 10E8 bnAb variants include 10E8v4, a solubility-improved variant (Kwon et al., “Optimization of the solubility of HIV-1-neutralizing antibody 10E8 though somatic variation and structure-based design” J. Virol. 90, 5899-5912 (2016)), incorporated by reference herein; those described in Huang et al. “Engineered bispecific antibodies with exquisite HIV-1-neutralizing activity” Cell 165, 1621-1631 (2016); and 10E8 variants 10E8v4-100cW, 10E8v4-100cF and 10E8v4-5R+100cF, described in Kwon et al. Cell Reports 22 1798-1809 (2018), incorporated by reference herein.

### SUMMARY OF THE INVENTION

Potent anti-HIV bnAbs, in combination with fusion proteins/polypeptides described herein, represent a novel and exciting way to augment HIV therapy by targeting the HIV envelope surface spike directly, while also targeting other components of the HIV life cycle. Anti-HIV bnAbs that could be used in fusion polypeptides as described herein include 4E10 (Cardoso et al. “Broadly neutralizing anti-HIV 4E10 recognizes a helical conformation of a highly conserved fusion-associated motif in gp41” Immunity, 22, 163-173 (2005), incorporated by reference herein; N6, a broadly neutralizing antibody that targets the CD4-binding site (Huang et al. “Identification of a CD4-binding site antibody to HIV that evolved near-pan neutralization breadth” Immunity, 45, 1109-1121 (2016), incorporated by reference herein, and combinations of 10E8, 4E10 and N6 or fragments thereof.

The antigen binding fusion polypeptide molecules as described herein, combined with bnAbs, makes use of this therapeutic strategy.

The invention is directed to antigen binding polypeptides which bind (a) CD4 receptors of the target cell and (b) the HIV envelope comprising: (i) a CD4 binding polypeptide and an anti-HIV broadly neutralizing antibody (anti-HIV bnAb) polypeptide, optionally with an additional gp41 binding polypeptide; (ii) a CD4 Adnectin™ and an anti-HIV bnAb, optionally with an N17 Adnectin™; (iii) a CD4 binding polypeptide which may be a CD4 Adnectin™, an anti-HIV bnAb polypeptide, and optionally a gp41 binding polypeptide which may be an N17 Adnectin™, and optionally further comprising a HIV fusion peptide inhibitor; or (iv) combinations of said CD4 binding polypeptide, and/or gp41 binding polypeptide, and/or HIV fusion peptide inhibitor with an anti-HIV bnAb polypeptide (e.g. an anti-HIV bnAb or anti-HIV bnAb fragment.

One embodiment of the invention is directed to antigen binding polypeptides comprising (i) a fibronectin-based scaffold domain polypeptide that binds CD4 which is linked to an anti-HIV bnAb (e.g., anti-HIV bnAb polypeptide); and/or (ii) a fibronectin-based scaffold domain polypeptide that binds the N17 domain of gp41, linked to the anti-HIV bnAb which is linked to the CD4 fibronectin-based polypeptide; and/or (iii) a HIV fusion peptide inhibitor attached to the anti-HIV bnAb which is linked to the anti-CD4 fibronectin-based polypeptide.

In one embodiment of the invention, the (i) a fibronectin-based scaffold domain polypeptide that binds CD4, (ii) the fibronectin-based scaffold domain polypeptide that binds the N17 domain of gp41, and/or the HIV fusion peptide inhibitor is connected to the anti-HIV bnAb moiety or anti-HIV bnAb fragment by a linker, e.g. a linker described herein. In another embodiment of the invention, the CD4 binding polypeptide and/or N17 binding polypeptide and/or HIV fusion peptide inhibitor may be connected to the anti-HIV bnAb moiety or anti-HIV bnAb fragment in any order.

The invention is also directed to combinations of a fibronectin-based scaffold domain polypeptide that binds CD4 (the anti-CD4 Adnectin™) and an anti-HIV bnAb polypeptide or anti-HIV bnAb fragment, and optionally a fibronectin-based scaffold domain polypeptide that binds gp41 (the anti-gp41 Adnectin™); and/or optionally a HIV fusion peptide inhibitor. In one embodiment of the invention, the anti-CD4 Adnectin™ and optionally the anti-gp41 Adnectin™ and/or the HIV fusion peptide inhibitor are connected to each other or to the anti-HIV bnAb moiety or anti-HIV bnAb fragment by a linker. In another embodiment of the invention, the anti-CD4 Adnectin™ and optionally the anti-gp41 Adnectin™; and/or optionally the HIV fusion peptide inhibitor, and the anti-HIV bnAb moiety or anti-HIV bnAb fragment, may be connected to each other in any order.

Another embodiment of the invention provides polypeptides comprising a CD4 Adnectin™ and an anti-HIV bnAb polypeptide moiety or fragment thereof, wherein the anti-CD4 Adnectin™ comprises a CD and/or FG loop region set forth in Table 3. Another embodiment of the invention provides polypeptides comprising an anti-CD4 Adnectin™ and an anti-HIV bnAb moiety or fragment thereof, wherein the anti-CD4 Adnectin™ comprises a CD/FG loop combination set forth in Table 3.

In some embodiments, the invention provides polypeptides comprising an CD4 Adnectin™ and an anti-HIV bnAb moiety or fragment thereof, wherein the CD4 Adnectin™ comprises an amino acid sequence at least 80%, 85%, 90%, 95%, 98%, 99% or 100% identical to any one of the CD or FG loop regions of SEQ ID NOs: 13-16.

In some embodiments, the invention provides polypeptides comprising an anti-CD4 Adnectin™ and an anti-HIV bnAb moiety or fragment thereof, wherein the anti-CD4 Adnectin™ comprises an amino acid sequence at least 80%, 85%, 90%, 95%, 98%, 99% or 100% identical to any one of the CD loop regions of SEQ ID NOs: 13 or 15.

In some embodiments, the invention provides polypeptides comprising an anti-CD4 Adnectin™ and an anti-HIV bnAb polypeptide or fragment thereof, wherein the anti-CD4 Adnectin™ comprises an amino acid sequence at least 80%, 85%, 90%, 95%, 98%, 99% or 100% identical to any one of the FG loop regions of SEQ ID NOs: 14 or 16.

In another embodiment of the invention, the CD4 Adnectin™ comprises an amino acid sequence at least 80%, 85%, 90%, 95%, 98%, 99% or 100% identical to the non-linker regions of SEQ ID NO: 72-91.

Another embodiment provides polypeptides comprising an anti-CD4 Adnectin™ and an anti-HIV bnAb polypeptide or fragment thereof, wherein the anti-HIV bnAb moiety or anti-HIV bnAb fragment thereof is an anti-HIV bnAb moiety or anti-HIV bnAb fragment thereof described in Table 8.

Particular embodiments provide polypeptides comprising a CD4 Adnectin™ and an anti-HIV-1 bnAb polypeptide or fragment thereof, wherein the HIV-1 bnAb moiety or fragment thereof is a PG9, PGT125-131, VRC01, 3BNC117, NIH45-46, 3BC176, N6, 4E10 or 10E8/10E8v4 HIV-1 bnAb or fragment thereof.

Particular embodiments provide polypeptides comprising a CD4 Adnectin™ and an HIV-1 bnAb polypeptide moiety or fragment thereof, wherein the anti-HIV-1 bnAb moiety or fragment thereof is a VRC01, N6 or 10E8/10E8v4 anti-HIV-1 bnAb or fragment thereof.

Particular embodiments provide polypeptides comprising a CD4 Adnectin™ and an anti-HIV bnAb moiety or fragment thereof, wherein the anti-HIV bnAb or fragment thereof is from a class of HIV-1 bnAb selected from an Apex (V1/V2) bnAb, a N332 glycan bnAb, a CD4 binding site (CD4bs) bnAb, a gp120-gp41 interface bnAb, and a MPER bnAb.

In another embodiment of the invention, a pharmacokinetic (PK) moiety is attached to the polypeptides of the invention. Examples of a PK moiety include but are not limited to polyethylene glycol, sialic acid, Fc, Fc fragment, transferrin, serum albumin (HSA), a serum albumin binding protein, and a serum immunoglobulin binding protein. In one embodiment of the invention, the PK moiety may be attached to the linker, or the N- or C-terminus of the polypeptide of the invention.

The invention is also directed to a pharmaceutical composition comprising one or more of the polypeptides of the invention and a carrier. The invention also provides a method of treating HIV in a subject comprising administering an effective amount of a polypeptide of the invention. The invention also provides a polypeptide of the invention for use in treating HIV. The invention also provides use of a polypeptide of the invention for the preparation of a medicament for treating HIV.

One embodiment provides an antigen binding polypeptide comprising a first and second polypeptide domain wherein the first polypeptide domain is (i) a CD4 binding polypeptide or (ii) an N17 Adnectin™ and the second polypeptide domain is an anti-HIV broadly neutralizing antibody (anti-HIV bnAb) polypeptide (e.g., anti-HIV bnAb) or a fragment thereof.

One embodiment provides an antigen binding polypeptide comprising a first and second polypeptide domain wherein the first polypeptide domain is (i) a CD4 Adnectin™ polypeptide or (ii) an N17 Adnectin™ polypeptide and the second polypeptide domain is an anti-HIV broadly neutralizing antibody (anti-HIV bnAb) polypeptide moiety or a fragment thereof.

One embodiment provides a bispecific antigen binding polypeptide comprising a first and second polypeptide domains wherein the first polypeptide domain is a CD4 Adnectin™ polypeptide and the second polypeptide domain is (i) an anti-HIV bnAb moiety or a fragment thereof; (ii) a CDR variant of (i) wherein the variant has 1, 2 or 3 amino acid modifications; (iii) a LC region of (i) comprising a sequence at least 80%, 85%, 90%, 95%, 98%, 99% or 100% identical to the sequence of SEQ ID NO: 6, SEQ ID NO: 76880 or SEQ ID NO: 769; and/or (iv) a HC of (i) region comprising a sequence at least 80%, 85%, 90%, 95%, 98%, 99% or 100% identical to the sequence of SEQ ID NO: 7, SEQ ID NO: 8, SEQ ID NO: 770, SEQ ID NO: 771 or SEQ ID NO: 772.

One embodiment provides a method of treating HIV in a subject comprising administering an

effective amount of the antigen binding polypeptide or pharmaceutical composition thereof as described herein. One embodiment provides an effective amount of the antigen binding polypeptide or pharmaceutical composition thereof as described herein for use in treating HIV. One embodiment provides use of an effective amount of the antigen binding polypeptide or pharmaceutical composition thereof as described herein in the preparation of a medicament for treating HIV.

One embodiment provides a nucleic acid sequence which encodes an antigen binding polypeptide as described herein.

One embodiment provides an expression vector comprising a nucleic acid sequence which encodes an antigen binding polypeptide as herein described.

One embodiment provides a recombinant host cell comprising a nucleic acid sequence which encodes an antigen binding polypeptide as described herein.

One embodiment provides a method for the production of an antigen binding polypeptide as described herein, which method comprises culturing a host cell that comprises a nucleic acid sequence which encodes an antigen binding polypeptide as described herein or comprises an expression vector comprising a nucleic acid sequence which encodes an antigen binding polypeptide as herein described under conditions suitable for expression of said nucleic acid sequence or vector, whereby a polypeptide of the disclosure is produced. In some embodiments, the method further comprises isolating the polypeptide from cell culture media in which the host cell was grown or from cell extracts.

### BRIEF DESCRIPTION OF THE FIGURES

FIG. 1A and FIG. 1B show a diagram of alternative polypeptides comprising a CD4 Adnectin™ and anti-HIV bnAb moiety. The different components of the polypeptide are connected to each other in any order by linkers and can be connected to either the bnAb light or heavy chain.

FIG. 2 shows the anti-HIV potency (IC₅₀) of a CD4 Adnectin™ alone, a 108Ev4 bnAb alone, a mixture of a CD4 Adnectin™ and a 108Ev4 bnAb, or two fusion polypeptides 1 and 2 comprising a CD4 Adnectin™ linked to a 108Ev4 bnAb.

FIGS. 3A, 3B, 3C and 3D are schematics of anti-HIV bnAb Heavy and Light Chain polypeptides fused to Adnectin™ polypeptides. FIG. 3A shows an anti-HIV bnAb Heavy chain fused to CD4 Adnectin™ FIG. 3B shows an anti-HIV bnAb Light chain fused to N17 Adnectin™ and HIV fusion peptide inhibitor. FIG. 3C shows an anti-HIV bnAb with heavy chains fused to anti-CD4 Adnectin,™ and Light Chains Fused to N17 Adnectins™ and HIV anti-gp41 fusion peptide inhibitors. FIG. 3D shows an anti-HIV bnAb with Light chains fused to anti-CD4 Adnectins™ and Heavy Chains Fused to N17 Adnectins™ and HIV anti-gp41 fusion peptide inhibitors.

FIG. 4 shows how the modularity of Adnectins with anti-HIV bnAbs can be used to produce fusion peptides having bispecific and trispecific activity against HIV.

FIG. 5A shows a schematic of the binding of an N17 Adnectin™ to the HIV envelope protein.

FIG. 5B shows a schematic of the binding of a CD4 A Adnectin™ or a CD4 B Adnectin™ to the domain 2 of CD4 or domain 4 of CD4, respectively.

FIG. 6A shows antiviral (HIV) activity of N17 Adnectin™/bnAb fusion polypeptides FIG. 6B shows antiviral (HIV) activity of CD4 A Adnectin™/or CD4 B Adnectin™/bnAb fusion polypeptides.

FIG. 7 shows that enhanced cell binding of B vs A CD4 Adnectins™ in 10E8v4 bnAb fusion polypeptides contributes to the improved potency observed with such fusion polypeptides.

FIG. 8 shows enhanced cell binding of B vs A CD4 Adnectins™ in DH511.12P fusion polypeptides and in 10E8v4 fusion polypeptides.

FIG. 9 shows the activity of the CD4 B/10E8v4 bnAb fusion polypeptide on cells infected with an HIV strain reported to be resistant to 10E8v4 bnAb using 10E8 resistance mutations (W680E/K683Q) engineered onto the replicating NL₄₋₃ backbone.

FIG. 10A shows the binding EC₅₀ of CD4 A/10E8v4 fusion or a CD4 B/10E8v4 fusion compared to 10E8v4, CD4 A or CD4 B alone, for 123 different clinical envelope proteins. As used herein, A refers to the anti-CD4 Domain 2 Adnectin™ and B refers to the anti-CD4 Domain 4 Adnectin™, but it is possible that CD4 A and CD4 B Adnectins™ bind to more than one domain within CD4 (.e.g. domain 3).

FIG. 10B shows the Maximum Percent Inhibition (MPI) by the CD4 B/10E8v4 fusion polypeptide against 123 different clinical envelope proteins. B refers to the anti-CD4 Domain 4 Adnectin™ polyprotein.

### DETAILED DESCRIPTION OF THE INVENTION

### Definitions

Unless defined otherwise, all technical and scientific terms used herein have the same meaning as commonly understood by the skilled artisan. Although any methods and compositions similar or equivalent to those described herein can be used in practice or testing of the present invention, the preferred methods and compositions are described herein.

The term “antigen binding protein” as used herein refers to antibodies and other protein constructs, such as domains, which are capable of binding to CD4 and/or gp120 and/or gp41 of the HIV envelope.

The term “Adnectin™” means a family of therapeutic polypeptides based on the 10^{th} fibronectin type III domain (10Fn3), which are designed and selected to bind with high affinity and selectivity to therapeutically-relevant targets. Adnectin™ may be selected using in vitro evolution methods, including mRNA display, phage display and yeast display.

An “antibody” is defined as a polypeptide including at least a light chain or heavy chain immunoglobulin variable region which specifically recognizes and binds an epitope of an antigen, or a fragment thereof, e.g., a fragment that specifically recognizes and binds an epitope of an antigen. Antibodies are composed of a heavy and a light chain, each of which has a variable region, termed the variable heavy (VH) region and the variable light (VL) region. Together, the VH region and the VL region are responsible for binding the antigen recognized by the antibody. The term antibody includes intact immunoglobulins, as well the variants and portions thereof, such as a single variable domain (e.g., VH, VHH, VL, domain antibody (DAB)), Fab′ fragments, F(ab)′2 fragments, single chain Fv proteins (“scFv”), disulfide stabilized Fv proteins (“dsFv”), diabodies, TANDABS etc. and modified versions of any of the foregoing. A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, IL); Kuby, J., Immunology, 3rd Ed., W. H. Freeman & Co., New York, 1997.

The term “broadly neutralizing antibody” or “anti-HIV broadly neutralizing antibody” (bNAb or anti-HIV bnAb) is defined as an antibody which inhibits viral attachment or cell entry via binding to the HIV envelope glycoprotein (Env) (e.g., gp160), as a non-limiting example, by a 50% inhibition of infection in vitro, in more than 50%, 60%, 70%, 80%, 90%, 95%, 99% or greater, of a large panel of (greater than 100) HIV-1 envelope pseudotyped viruses and viral isolates. See e.g., US Published Patent Application No. 20120121597. The term “domain” refers to a folded protein structure which retains its tertiary structure independent of the rest of the protein. Generally, domains are responsible for discrete functional properties of proteins and in many cases may be added, removed or transferred to other proteins without loss of function of the remainder of the protein and/or of the domain. The term “single variable domain” refers to a folded polypeptide domain comprising sequences characteristic of antibody variable domains. It therefore includes complete antibody variable domains such as VH, VHH and VL and modified antibody variable domains. Modified antibody variable domain means, for example, a variable domain in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain. A single variable domain is capable of binding an antigen or epitope independently of a different variable region or domain. A single variable domain may be a human single variable domain, but also includes single variable domains from other species such as rodent, nurse shark, cynomolgus monkey and Camelid VHH. Camelid VHH are immunoglobulin single variable domain polypeptides that are derived from species including camel, llama, alpaca, dromedary, and guanaco, which produce heavy chain antibodies naturally devoid of light chains. Such VHH domains may be humanised according to standard techniques available in the art, and such domains are considered to be “single variable domains”. As used herein VH includes camelid VHH domains.

Typically, a naturally occurring immunoglobulin has heavy (H) chains and light (L) chains interconnected by disulfide bonds. There are two types of light chain, lambda (A) and kappa (κ). There are five main heavy chain classes (or isotypes) which determine the functional activity of an antibody molecule: IgM, IgD, IgG, IgA and IgE.

Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarily-determining regions” or “CDRs”.

“CDRs” are defined as the complementarity determining region amino acid sequences of an antigen binding protein. These are the hypervariable regions of immunoglobulin heavy and light chains. There are three heavy chain and three light chain CDRs (or CDR regions) in the variable portion of an immunoglobulin. Thus, “CDRs” as used herein refers to all three heavy chain CDRs, all three light chain CDRs, all heavy and light chain CDRs, or at least two CDRs.

Throughout this specification, amino acid residues in variable domain sequences and full length antibody sequences are numbered according to the Kabat numbering convention. Similarly, the terms “CDR”, “CDRL1”, “CDRL2”, “CDRL3”, “CDRH1”, “CDRH2”, “CDRH3” used in the Examples follow the Kabat numbering convention. For further information, see Kabat et al., Sequences of Proteins of Immunological Interest, 5th Ed., U.S. Department of Health and Human Services, National Institutes of Health (1991).

It will be apparent to those skilled in the art that there are alternative numbering conventions for amino acid residues in variable domain sequences and full length antibody sequences. There are also alternative numbering conventions for CDR sequences, for example those set out in Chothia et al. (1989) Nature 342: 877-883. The structure and protein folding of the antibody may mean that other residues are considered part of the CDR sequence and would be understood to be so by a skilled person.

Other numbering conventions for CDR sequences available to a skilled person include “AbM” (University of Bath) and “contact” (University College London) methods. The minimum overlapping region using at least two of the Kabat, Chothia, AbM and contact methods can be determined to provide the “minimum binding unit”. The minimum binding unit may be a sub-portion of a CDR.

Table 1 below represents one definition using each numbering convention for each CDR or binding unit. The Kabat numbering scheme is used in Table 1 to number the variable domain amino acid sequence. It should be noted that some of the CDR definitions may vary depending on the individual publication used.

TABLE 1

| | | | | | Minimum |
|---|---|---|---|---|---|
| | Kabat | Chothia | AbM | Contact | binding |
| | CDR | CDR | CDR | CDR | unit |
| H1 | 31-35/35A/ | 26-32/33/ | 26-35/35A/ | 30-35/35A/ | 31-32 |
| | 35B | 34 | 35B | 35B | |
| H2 | 50-65 | 52-56 | 50-58 | 47-58 | 52-56 |
| H3 | 95-102 | 95-102 | 95-102 | 93-101 | 95-101 |
| L1 | 24-34 | 24-34 | 24-34 | 30-36 | 30-34 |
| L2 | 50-56 | 50-56 | 50-56 | 46-55 | 50-55 |
| L3 | 89-97 | 89-97 | 89-97 | 89-96 | 89-96 |

Accordingly, a bispecific antigen binding protein is provided, which comprises any one or a combination of modified CDRs from an anti-HIV bnAb.

### CDR Variant

CDRs or minimum binding units may be modified by at least one amino acid substitution, deletion or addition, wherein the variant antigen binding protein substantially retains the biological characteristics of the unmodified protein, such as 10e8/10E8v4 bnAb or any unmodified bnAb listed in Table 8.

It will be appreciated that each of CDR H1, H2, H3, L1, L2, L3 may be modified alone or in combination with any other CDR, in any permutation or combination. In one embodiment, a CDR is modified by the substitution, deletion or addition of up to 3 amino acids, for example 1 or 2 amino acids, for example 1 amino acid. Typically, the modification is a substitution, particularly a conservative substitution, for example as shown in Table 2 below.

TABLE 2

| Side chain | Members |
|---|---|
| Hydrophobic | Met, Ala, Val, Leu, Ile |
| Neutral hydrophilic | Cys, Ser, Thr |
| Acidic | Asp, Glu |
| Basic | Asn, Gln, His, Lys, Arg |
| Residues that influence chain | Gly, Pro |
| orientation | |
| Aromatic | Trp, Tyr, Phe |

For example, in a variant CDR, the amino acid residues of the minimum binding unit may remain the same, but the flanking residues that comprise the CDR as part of the Kabat or Chothia definition(s) may be substituted with a conservative amino acid residue.

Such antigen binding proteins comprising modified CDRs or minimum binding units as described above may be referred to herein as “functional CDR variants” or “functional binding unit variants”.

### Epitope

The term “epitope” as used herein refers to that portion of the antigen that makes contact with a particular binding domain of the antigen binding protein. An epitope may be linear or conformational/discontinuous. A conformational or discontinuous epitope comprises amino acid residues that are separated by other sequences, i.e. not in a continuous sequence in the antigen's primary sequence. Although the residues may be from different regions of the peptide chain, they are in close proximity in the three-dimensional structure of the antigen. In the case of multimeric antigens, a conformational or discontinuous epitope may include residues from different peptide chains. Particular residues comprised within an epitope can be determined through computer modelling programs or via three-dimensional structures obtained through methods known in the art, such as X-ray crystallography.

### Competition

Competition between the antigen binding protein (e.g., that is or contains an anti-HIV bnAb) and a reference antibody may be determined by competition ELISA, FMAT or BIAcore. There are several possible reasons for this competition: the two proteins may bind to the same or overlapping epitopes, there may be steric inhibition of binding, or binding of the first protein may induce a conformational change in the antigen that prevents or reduces binding of the second protein.

The reduction or inhibition in biological activity may be partial or total. A neutralising antigen binding protein (e.g., that is or contains an anti-HIV bnAb) may neutralise the activity of HIV by at least 20%, 30% 40%, 50%, 55%, 60%, 65%, 70%, 75%, 80%, 82%, 84%, 86%, 88%, 90%, 92%, 94%, 95%, 96%, 97%, 98%, 99% or 100% relative to HIV activity in the absence of the antigen binding protein.

Neutralisation may be determined or measured using one or more assays known to the skilled person or as described herein.

### Canonicals

The CDRs L1, L2, L3, H1 and H2 tend to structurally exhibit one of a finite number of main chain conformations. The particular canonical structure class of a CDR is defined by both the length of the CDR and by the loop packing, determined by residues located at key positions in both the CDRs and the framework regions (structurally determining residues or SDRs). Martin and Thornton (1996; J Mol Biol 263:800-815) have generated an automatic method to define the “key residue” canonical templates. Cluster analysis is used to define the canonical classes for sets of CDRs, and canonical templates are then identified by analysing buried hydrophobics, hydrogen-bonding residues, and conserved glycines and prolines. The CDRs of antibody sequences can be assigned to canonical classes by comparing the sequences to the key residue templates and scoring each template using identity or similarity matrices.

There may be multiple variant CDR canonical positions per CDR, per corresponding CDR, per binding unit, per heavy or light chain variable region, per heavy or light chain, and per antigen binding protein, and therefore any combination of substitution may be present in the antigen binding protein of the invention, provided that the canonical structure of the CDR is maintained such that the antigen binding protein is capable of specifically binding its receptor. For example, for the antigen binding polypeptides of the invention, the variant CDR sequences for an anti-HIV bnAb may be substituted in a multitude of ways, as described, provided that the canonical structure of the anti-HIV bnAb CDR is maintained such that the anti-HIV bnAb is capable of binding to its targets—i.e. HIV envelope spikes—and maintains its ability to act as a neutralizing antibody.

As discussed above, the particular canonical structure class of a CDR is defined by both the length of the CDR and by the loop packing, determined by residues located at key positions in both the CDRs and the framework regions.

### Protein Scaffold

An antigen binding fragment may be provided by means of arrangement of one or more CDRs on antibody or non-antibody protein scaffolds. “Protein Scaffold” as used herein includes but is not limited to an immunoglobulin (Ig) scaffold, for example an IgG scaffold, which may be a four chain or two chain antibody, or which may comprise only the Fc region of an antibody, or which may comprise one or more constant regions from an antibody, which constant regions may be of human or primate origin, or which may be an artificial chimera of human and primate constant regions. The protein scaffold may be an Ig scaffold, for example an IgG, or IgA scaffold. The IgG scaffold may comprise some or all the domains of an antibody (i.e. CH1, CH2, CH3, VH, VL). The antigen binding protein may comprise an IgG scaffold selected from IgG1, IgG2, IgG3, IgG4 or IgG4PE. For example, the scaffold may be IgG1. The scaffold may consist of, or comprise, the Fc region of an antibody, or is a part thereof.

The protein scaffold may be a derivative of a scaffold selected from the group consisting of CTLA-4, lipocalin, Protein A derived molecules such as Z-domain of Protein A (Affibody, SpA), A-domain (Avimer/Maxibody); heat shock proteins such as GroEI and GroES; transferrin (trans-body); ankyrin repeat protein (DARPin); peptide aptamer; C-type lectin domain (Tetranectin); human γ-crystallin and human ubiquitin (affilins); PDZ domains; scorpion toxin kunitz type domains of human protease inhibitors; and fibronectin/Adnectin™; which has been subjected to protein engineering in order to obtain binding to an antigen, such as CD4 or gp41, other than the natural ligand.

The term “gp120” is defined as an envelope protein from HIV. This envelope protein is initially synthesized as a longer precursor protein of 845-870 amino acids in size, designated gp160. gp160 is cleaved by a cellular protease into gp120 and gp41. gp120 contains most of the external, surface-exposed, domains of the HIV envelope glycoprotein complex, and it is gp120 which binds both to cellular CD4 receptors and to cellular chemokine receptors (such as CCR5). See e.g., U.S. Patent Publication No. 20160009789.

The term “gp41” is defined as an HIV protein that contains a transmembrane domain and remains in a trimeric configuration; it interacts with gp120 in a non-covalent manner. The envelope protein of HIV-1 is initially synthesized as a longer precursor protein of 845-870 amino acids in size, designated gp160. gp160 forms a homotrimer and undergoes glycosylation within the Golgi apparatus. In vivo, it is then cleaved by a cellular protease into gp120 and gp41. The amino acid sequence of an example of gp41 is set forth in GENBANK® Accession No. CAD20975 (as available on Oct. 16, 2009) which is incorporated by reference herein. It is understood that the sequence of gp41 can vary from that given in GENBANK® Accession No. CAD20975. gp41 contains a transmembrane domain and typically remains in a trimeric configuration; it interacts with gp120 in a non-covalent manner. See e.g., U.S. Patent Publication No. 20160009789, issued as U.S. Pat. No. 9,783,703 on Aug. 22, 2017.) The term “gp160” refers to an envelope protein having a molecular weight of 160 kDa and contains various glycosylation sites. Gp160 acts as a precursor for both gp41 and gp120. For the purposes of the invention, gp160 is a representative envelope glycoprotein, and HXB2D is a non-limiting example of an envelope sequence. See e.g., https://www.hiv.lanl.gov/content/sequence/HIV/REVIEWS/HXB2.html, regarding HXB2D, the contents of which are incorporated by reference.

The term “envelope glycoprotein” or “glycoprotein” or “EnV” refers to a protein that contains oligosaccharide chains (glycans) covalently attached to polypeptide side-chains and that is exposed on the surface of the HIV envelope. For the purposes of the present invention, after administration of the fusion polypeptide to a subject, an HIV gp160 envelope glycoprotein is bound by the fusion polypeptide. In some embodiments, the HIV gp160 envelope glycoprotein is bound to the antibody portion of the fusion polypeptide.

“Polypeptide” as used herein refers to any sequence of two or more amino acids, regardless of length, post-translation modification, or function. “Polypeptide”, “peptide”, and “protein” are used interchangeably herein. Polypeptides can be modified in any of a variety of standard chemical ways (e.g., an amino acid can be modified with a protecting group; the carboxy-terminal amino acid can be made into a terminal amide group; the amino-terminal residue can be modified with groups to, e.g., enhance lipophilicity; or the polypeptide can be chemically glycosylated or otherwise modified to increase stability or in vivo half-life). Polypeptide modifications can include the attachment of another structure such as a cyclic compound or other molecule to the polypeptide and can also include polypeptides that contain one or more amino acids in an altered configuration (i.e., R or S; or, L or D).

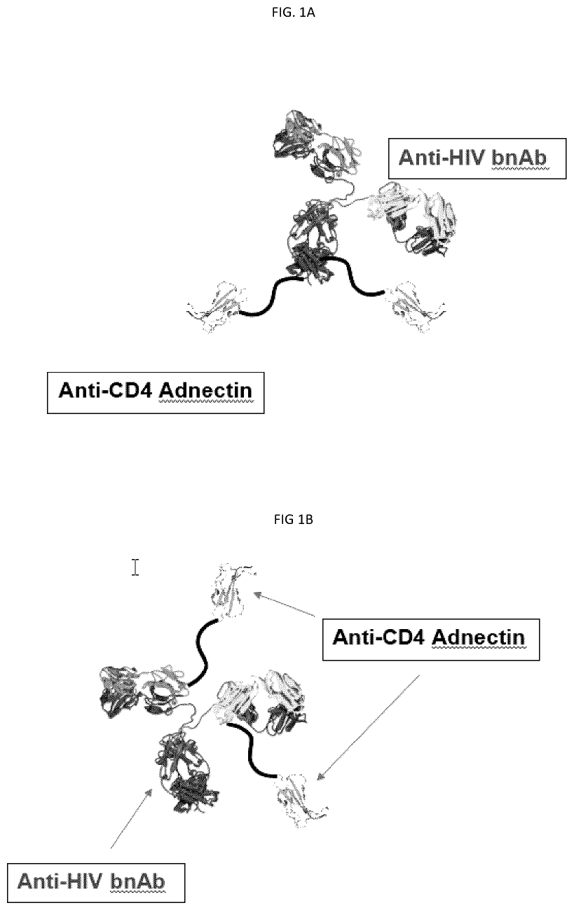

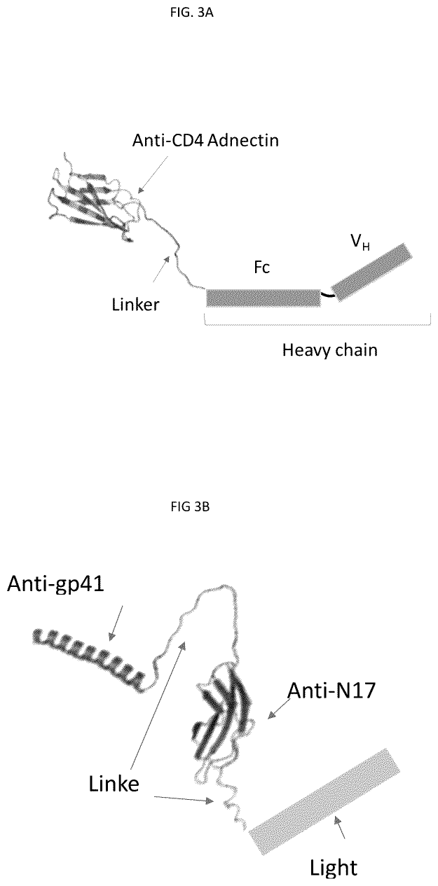

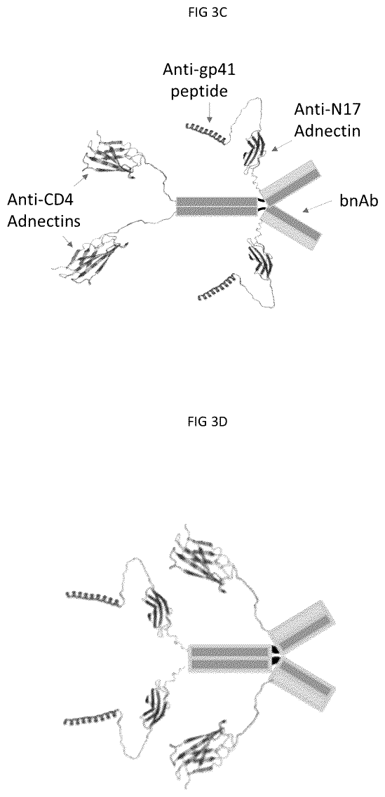

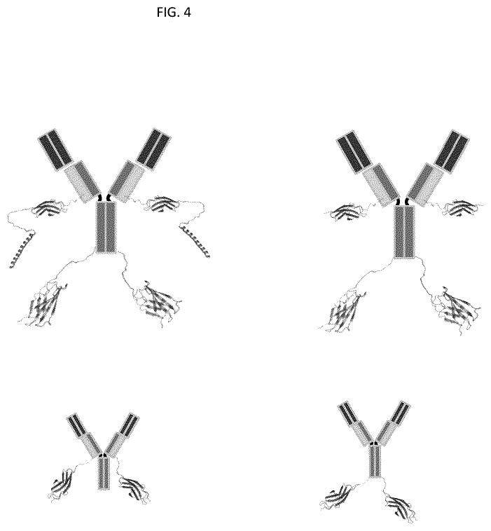

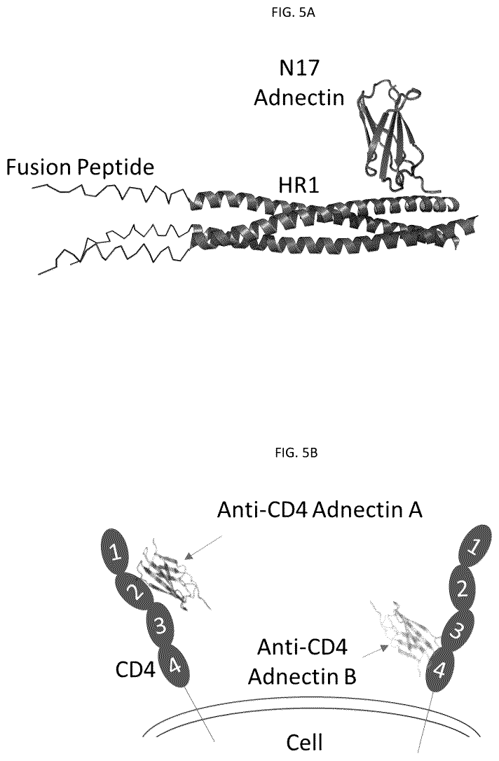

The polypeptides of the invention may include, for example, polypeptides derived from the tenth type III domain of fibronectin that have been modified to bind to CD4 domain and are referred to herein as, “anti-CD4 Adnectin™”, “CD4 Adnectin™.” The polypeptides of the invention may also include, for example, proteins derived from the tenth type III domain of fibronectin that have been modified to bind to the N17 domain of gp-41 and are referred to herein as, “anti-gp41 Adnectin™”, “N17 Adnectin™” or anti-N17 Adnectin™. The polypeptides of the invention also include polypeptides comprising a CD4 Adnectin™ linked to an anti-HIV bnAb (FIG. 1). The polypeptides of the invention also include polypeptides comprising an N17 Adnectin™ linked to an anti-HIV bnAb (FIG. 3). Alternatively, the polypeptides comprise a CD4 Adnectin™ and/or an N17 Adnectin™ linked to an anti-HIV bnAb described in Table 8 or anti-HIV bnAb fragment thereof. The polypeptides of the invention may also include an HIV fusion peptide inhibitor, which may be linked to the N17 Adnectin™.

An “isolated” polypeptide is one that has been identified and separated and/or recovered from a component of its natural environment. Contaminant components of its natural environment are materials that would interfere with diagnostic or therapeutic uses for the polypeptide, and may include recombinant host cell proteins and other proteinaceous or nonproteinaceous solutes. In one embodiment, the polypeptides will be purified (1) to greater than 95% by weight of polypeptide as determined by the Lowry method, and most preferably more than 99% by weight, or (2) to homogeneity by SDSPAGE under reducing or nonreducing condition using Coomassie blue or silver stain. Ordinarily, isolated polypeptide will be prepared by at least one purification step.

“Percent (%) amino acid sequence identity” herein is defined as the percentage of amino acid residues in a candidate sequence that are identical with the amino acid residues in a selected sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR®) software. Those skilled in the art can readily determine appropriate parameters for measuring alignment, including any algorithms needed to achieve maximal alignment over the full length of the sequences being compared. For example, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. As used herein, “conservative substitution” denotes the replacement of an amino acid residue by another, without altering the overall conformation and function of the peptide, including, but not limited to, replacement of an amino acid with one having similar properties (such as, for example, polarity, hydrogen bonding potential, acidic, basic, shape, hydrophobic, aromatic, and the like). Amino acids with similar properties are well known in the art.

For example, arginine, histidine and lysine are hydrophilic basic amino acids and may be interchangeable. Similarly, isoleucine, a hydrophobic amino acid, may be replaced with leucine, methionine or valine. Neutral hydrophilic amino acids, which can be substituted for one another, include asparagine, glutamine, serine and threonine. By “substituted” or “modified” the present invention includes those amino acids that have been altered or modified from naturally occurring amino acids. The % identity may be determined across the entire length of the query sequence, including the CDR(s) or the CD or FG loops of Adnectins™. Alternatively, the % identity may exclude the CDR(s) or CD or FG loop(s), for example the CDR(s) or CD/FG loop(s) is 100% identical to the subject sequence and the % identity variation is in the remaining portion of the query sequence, so that the CDR or CD/FG loop sequence(s) is fixed/intact.

The variant sequence substantially retains the biological characteristics of the unmodified protein, such as the anti-HIV bnAB, or the unmodified CD4 or N17Adnectin™ sequence.

### Sequence Variation

The VH or VL (or HC or LC) sequence may be a variant sequence with up to 10 amino acid substitutions, additions or deletions, or the CD or FG loop sequence may be a variant sequence with up to 5 amino acid substitutions. For example, the VH or VL variant sequence may have up to 9, 8, 7, 6, 5, 4, 3, 2 or 1 amino acid substitution(s), addition(s) or deletion(s); or the CD or FG loop variant sequence may have up to 5, 4, 3, 2 or 1 amino acid substitution(s), addition(s) or deletion(s).

The sequence variation may exclude the CDR(s) or CD/FG loop(s), for example the CDR(s) is the same as the VH or VL (or HC or LC) sequence and the variation is in the remaining portion of the VH or VL (or HC or LC) sequence, so that the CDR sequence is fixed/intact. Or the CD/FG loop in the variant CD4 or N17 Adnectin™ is the same as the CD or FG (or AB or EF) sequence and the variation is in the remaining portion of the variant Adnectin™ sequence, so that the CD/FG loop(s) sequence is fixed/intact.

The variant sequence substantially retains the biological characteristics of the unmodified protein, such as the anti-HIV bnAb or the CD4 or N17 Adnectin™. Examples of CD4 Adnectins™ with modified CD/FG loop sequences is shown in Table 3, respectively.

As such it should be understood that in the context of the present invention a conservative substitution is recognized in the art as a substitution of one amino acid for another amino acid that has similar properties.

As used herein, the term “binding site” or “antigen binding site” refers to the site or portion of a protein (e.g., CD4 or gp41) that interacts or binds to a particular protein of the invention (e.g., as an epitope is recognized by an antibody). Binding sites can be formed from contiguous amino acids or noncontiguous amino acids juxtaposed by tertiary folding of a protein. Binding sites formed by contiguous amino acids are typically retained on exposure to denaturing solvents, whereas binding sites formed by tertiary folding are typically lost on treatment of denaturing solvents.

The binding site for an anti-CD4 or CD4 binding moiety of the invention may be determined by application of standard techniques typically used for epitope mapping of antibodies including, but not limited to protease mapping and mutational analysis. Alternatively, a binding site can be determined by competition assay using a reference protein (e.g., another Adnectin™ or antibody) which binds to the same polypeptide, e.g., CD4. If the test protein and reference molecule (e.g., another Adnectin™ or antibody) compete, then they bind to the same binding site or to binding sites sufficiently proximal such that binding of one molecule interferes with the other.

The terms “specifically binds”, “specific binding”, “selective binding”, and “selectively binds”, as used interchangeably herein refers to a protein that exhibits affinity for a CD4 or gp41 or gp120 (as relevant to the particular Adnectin™ or fusion polypeptide being evaluated), but does not significantly bind (e.g., less than about 10% binding) to a different polypeptide as measured by a technique available in the art such as, but not limited to, Scatchard analysis and/or competitive binding assays (e.g., competition ELISA, BIACORE® SPR assay). The term is also applicable where e.g., a binding domain of a protein of the invention is specific for CD4, gp41 or gp120 (as relevant).

The term “preferentially binds” as used herein refers to the situation in which the Adnectins™ or polypeptides of the invention bind CD4, gp120 or gp41 at least about 20% greater than it binds a different polypeptide as measured by a technique available in the art such as, but not limited to, Scatchard analysis and/or competitive binding assays (e.g., competition ELISA, BIACORE® SPR assay).

As used herein, the term “cross-reactivity” refers to a protein which binds to more than one distinct protein having identical or very similar binding sites.

The term “Kd”, as used herein, is intended to refer to the dissociation equilibrium constant for the binding of a particular isolated Adnectin™, antibody, antibody fragment, or fusion polypeptide inhibitor comprising one or more Adnectins™, antibodies, or antibody fragments for a target protein (e.g., CD4), as measured using a surface plasmon resonance assay or a cell binding assay.

The term “IC₅₀”, as used herein, refers to the concentration of, for example, a polypeptide of the invention that inhibits a response, either in an in vitro or an in vivo assay, to a level that is 50% of the maximal inhibitory response, i.e., halfway between the maximal inhibitory response and the untreated response.

The terms “inhibit” or “neutralize” as used herein with respect to an activity of a polypeptide of the invention means the ability to substantially antagonize, prohibit, prevent, restrain, slow, disrupt, eliminate, stop, reduce or reverse e.g., progression or severity of that which is being inhibited including, but not limited to, a biological activity or property, a disease or a condition. The inhibition or neutralization is preferably at least about 10%, 20%, 30%, 40%, 50%, 60%, 70%, 80%, 90%, 95% or higher. The term “neutralizes” as used throughout the present specification means that the biological activity of the HIV envelope protein is reduced in the presence of an anti-HIV bnAb as described herein in comparison to the activity of HIV envelope protein in the absence of the anti-HIV bnAb, in vitro or in vivo. Neutralization may be due to one or more of blocking HIV envelope protein binding to its receptor, preventing HIV envelope protein from activating its receptor, down regulating HIV envelope protein or its receptor, or affecting effector functionality. For example, the potency methods described in Example 2 may be used to assess the neutralising capability of an anti-HIV bnAb.

The term “PK” is an acronym for “pharmacokinetic” and encompasses properties of a compound including, by way of example, absorption, distribution, metabolism, and elimination by a subject. A “PK modulation protein” or “PK moiety” as used herein refers to any protein, peptide, or moiety that affects the pharmacokinetic properties of a biologically active molecule when fused to or administered together with the biologically active molecule. Examples of a PK modulation protein or PK moiety include PEG, human serum albumin (HSA) binders (as disclosed in U.S. Publication No. 2005/0287153, U.S. Pat. No. 7,696,320, PCT Publication Nos. WO 2009/083804 and WO 2009/133208), human serum albumin, Fc or Fc fragments and variants thereof, and sugars (e.g., sialic acid).

The term “CD4 binding moiety” or “CD4 binding polypeptide” refers to any moiety or polypeptide that binds to CD4 and i) blocks HIV surface protein gp120 binding to the CD4 receptor on CD4+ T cells, or ii) allows gp120 to bind to CD4, but blocks subsequent interaction of gp120 with co-receptor (CCR5 or CCR4) or other event required for cell entry. The CD4 binding moiety may be: a CD4 Adnectin™, also referred to as an anti-CD4 Adnectin™; an antibody (such as ibalizumab); a domain antibody (dAb); an antibody fragments (such as a Fab); a bispecific antibody; or a fusion protein thereof.

A “HIV fusion peptide inhibitor moiety” or “HIV fusion peptide inhibitor” refers to any moiety that inhibits fusion by binding the heptad repeat 1 (HR1) region of gp41. Examples of fusion peptide inhibitor moiety include peptides derived from the NHR and CHR regions of gp41, designated NHR and CHR peptides, respectively. Enfuvirtide is an example of a CHR peptide.

The term “gp41 binding moiety” or “gp41 binding polypeptide” refers to any moiety or polypeptide that interferes with the interaction of the viral envelope glycoprotein complex (gp120/gp41) with T cells. The gp41 binding moiety may be anti-gp41 -Adnectin™, -antibody (Ab), -domain antibody (dAb), -antibody fragments (e.g. a Fab), -bispecific antibody and fusion protein thereof.

The polypeptides of the invention may include, for example, a CD4 Adnectin™ comprising a sequence as listed in Table 4, and an anti-HIV bnAb described in Table 8 or a fragment thereof. Alternatively, the polypeptides of the invention may include a CD4 Adnectin™ comprising a sequence selected from SEQ ID NO: 3, SEQ ID NO:4, SEQ ID NO:5 and SEQ ID NO: 78, and an anti-HIV bnAb described in Table 8 or a fragment thereof. Another embodiment of the invention provides a CD4 Adnectin™ comprising a sequence as listed in Table 4, and an N17 Adnectin™ comprising a sequence as listed in Table 5, and an anti-HIV bnAb described in Table 8 or a fragment thereof. Polypeptides of the invention may also include an HIV fusion peptide inhibitor.

The “half-life” of a polypeptide can generally be defined as the time taken for the serum concentration of the polypeptide to be reduced by 50%, in vivo, for example, due to degradation of the polypeptide and/or clearance or sequestration of the polypeptide by natural mechanisms. The half-life can be determined in any manner known per se, such as by pharmacokinetic analysis. Suitable techniques will be clear to the person skilled in the art, and may, for example, generally involve the steps of suitably administering to a subject a suitable dose of the polypeptide of the invention; collecting blood samples or other samples from the subject at regular intervals; determining the level or concentration of the polypeptide of the invention in said blood sample; and calculating, from (a plot of) the data thus obtained, the time until the level or concentration of the polypeptide of the invention has been reduced by 50% compared to the initial level upon dosing. Reference is, for example, made to the standard handbooks, such as Kenneth, A. et al., Chemical Stability of Pharmaceuticals: A Handbook for Pharmacists and in Peters et al., Pharmacokinetic Analysis: A Practical Approach (1996). Reference is also made to Gibaldi, M. et al., Pharmacokinetics, Second Rev. Edition, Marcel Dekker (1982).

Half-life can be expressed using parameters such as the t1/2-alpha, t1/2-beta, HL_Lambda_z, and the area under the curve (AUC). In the present specification, an “increase in half-life” refers to an increase in any one of these parameters, any two of these parameters, any three of these parameters or all four of these parameters.

Modifications that Affect the Half-Life of an Antigen Binding Protein

The long half-life of IgG antibodies is reported to be dependent on their binding to FcRn. Therefore, substitutions that increase the binding affinity of IgG to FcRn at pH 6.0 (the pH in the endosome) while maintaining the pH dependence of the interaction with target, by engineering the constant region, have been extensively studied (Ghetie et al., Nature Biotech. 15: 637-640, 1997; Hinton et al., JBC 279: 6213-6216, 2004; Dall'Acqua et al., 10 J Immunol 117: 1129-1138, 2006). The in-vivo half-life of antigen binding proteins of the present invention may be altered by modification of a heavy chain constant domain or an FcRn (Fc receptor neonate) binding domain therein.

In adult mammals, FcRn, also known as the neonatal Fc receptor, plays a key role in maintaining serum antibody levels by acting as a protective receptor that binds and salvages antibodies of the IgG isotype from degradation. IgG molecules are endocytosed by endothelial cells and, if they bind to FcRn, are recycled out of the cells back into circulation. In contrast, IgG molecules that enter the cells and do not bind to FcRn and are targeted to the lysosomal pathway where they are degraded.

FcRn is believed to be involved in both antibody clearance and the transcytosis across tissues (see Junghans R. P (1997) Immunol. Res 16. 29-57 and Ghetie et al (2000) Annu. Rev. Immunol. 18, 739-766). Human IgG1 residues determined to interact directly with human FcRn include Ile253, Ser254, Lys288, Thr307, Gln311, Asn434 and His435. Switches at any of these positions may enable increased serum half-life and/or altered effector properties of antigen binding proteins of the invention.

### Mutations to Increase Half-Life by Adding or Increasing FcRn Binding

Antigen binding proteins of the present invention may have amino acid modifications that increase the affinity of the constant domain or fragment thereof for FcRn. Increasing the half-life (i.e., serum half-life) of therapeutic and diagnostic IgG antibodies and other bioactive molecules has many benefits including reducing the amount and/or frequency of dosing of these molecules. In one embodiment, an antigen binding protein of the invention comprises all or a portion (an FcRn binding portion) of an IgG constant domain having one or more of the following amino acid modifications.

For example, with reference to IgG1, M252Y/S254T/T256E (commonly referred to as “YTE” mutations) and M428L/N434S (commonly referred to as “LS” mutations) increase FcRn binding at pH 6.0 (Wang et al., Protein Cell. 2018 January; 9(1): 63-73).

Half-life can also be enhanced by T250Q/M428L, V2591,V308F/M428L, N434A, and T307A/E388A′/N434A mutations (with reference to IgG1 and Kabat numbering) (Monnet et al., 2014 mAbs, 6:2, 422-436).

Half-life and FcRn binding can also be extended by introducing H433K and N434F mutations (commonly referred to as “HN” or “NHance” mutations) (with reference to IgG1) (WO2006/130834).

The notations “mpk”, “mg/kg”, or “mg per kg” refer to milligrams per kilogram. All notations are used interchangeably throughout the present disclosure.

The terms “individual”, “subject”, and “patient”, used interchangeably herein, refer to an animal, preferably a mammalian (including a nonprimate and a primate), including, but not limited to, murines, simians, humans, mammalian farm animals (e.g., bovine, porcine, ovine), mammalian sport animals (e.g., equine), and mammalian pets (e.g., canine and feline); preferably the term refers to humans. In a certain embodiment, the subject, is a mammal, is preferably a human and is infected with HIV.

The term “therapeutically effective amount” refers to at least the minimal dose, but less than a toxic dose, of an agent which is necessary to impart a therapeutic benefit to a subject. For example, a therapeutically effective amount of a polypeptide (e.g., antigen binding polypeptide or fusion polypeptide) of the invention is an amount which in mammals, preferably humans, results in a significant decline in circulating HIV within the infected individual.

### Overview

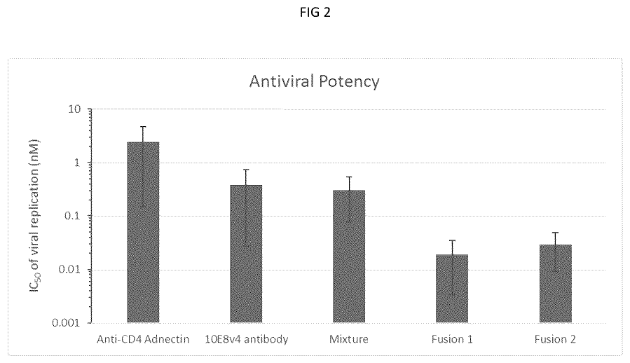

The Applicants have found that by linking a CD4 binding polypeptide (an anti-CD4 polypeptide such as a CD4 Adnectin™) to an anti-HIV broadly neutralizing antibody (anti-HIV bnAb), the effects of the resulting anti-CD4-anti-HIV bnAb fusion polypeptide in disrupting HIV envelope fusion are significantly enhanced compared to the effects of the anti-HIV bnAb alone, the CD4 binding polypeptide, or to a mixture of the CD4 binding polypeptide and anti-HIV bnAb. Applicants have surprisingly found that the effect the anti-CD4-anti-HIV bnAb fusion polypeptide has on HIV fusion appears to be synergistic. FIG. 2 shows the antiviral potency of the various components based on the IC₅₀ of HIV-1 replication (concentration at which HIV replication is inhibited by 50%) for a host cell after treatment of those HIV-1-infected cells with an anti-CD4 Adnectin™ alone, or with an anti-HIV bnAb alone (10E8v4 antibody), or with a mixture of anti-CD4 Adnectin™ and anti-HIV bnAb, or two fusion polypeptides 1 and 2 comprising anti-CD4-anti-HIV bnAb. As can be seen in FIG. 2, the IC₅₀ value obtained for the anti-CD4 Adnectin™-anti-HIV bnAb fusion polypeptide is much lower than the IC₅₀ value obtained for single components or the mixture (CD4 Adnectin™, anti-HIV bnAb, or mixture of CD4 Adnectin™ and anti-HIV bnAb). As can be seen, the effect on viral replication (and thus its antiviral potency) for the anti-CD4 Adnectin™-anti-HIV bnAb fusion polypeptides is greater than a mere addition of the effects of the individual anti-CD4 Adnectin™ and anti-HIV bnAb components on HIV replication, and greater than the effect of the mixture of the anti-CD4 Adnectin™ and anti-HIV bnAb on HIV replication.

Although not wishing to be bound by theory, Applicants believe that, in addition to exerting its inherent antiviral activity, the CD4 binding polypeptide acts as a targeting moiety for the target cell, resulting in coating of the target cell with the CD4 binding polypeptide and positioning the bnAb component more optimally for interaction with its target on the HIV gp160 envelope protein. By linking the anti-HIV bnAb to the CD4 binding polypeptide, Applicants have found a way to concentrate the anti-HIV bnAb at the site of action, thus increasing the effects of the anti-HIV bnAb on HIV fusion disruption in a synergistic way.

An embodiment of the present invention provides novel polypeptides (e.g. fusion polypeptides, e.g. antigen binding polypeptides) that bind to CD4. The polypeptides comprise a CD4 binding moiety and an anti-HIV broadly neutralizing antibody moiety, covalently linked, or as a combination thereof. More specifically, embodiments of the present invention relate to polypeptides comprising a fibronectin-based scaffold domain protein that binds CD4 and an anti-HIV broadly-neutralizing antibody (bnAb) or combinations thereof.

In order to identify CD4 Adnectins™, soluble CD4 (extracellular domain) was presented to large synthetic libraries of Adnectins™. Adnectins™ that survived several rounds of selection were screened for CD4 binding, for biophysical properties, and for HIV-1 inhibitory activity. The best anti-CD4 Adnectin™ sequences that emerged from the screenings were mutated and subjected to further rounds of selection with increased selective pressure, achieved by lowering the target concentration and/or selecting for anti-CD4 Adnectins™ with fast on-rates and/or slow off-rates. From this optimization process, multiple families of CD4 Adnectins™ were identified as HIV-1 specific inhibitors with favorable biochemical and biophysical activity. See Discovery and Characterization of a Novel CD4-Binding Adnectin™ with Potent Anti-HIV Activity. Antimicrob Agents Chemother. 2017 Jul. 25; 61(8), the entire contents of which is incorporated by reference herein.

To identify N17 Adnectins™, a similar process was utilized. See A Novel gp41-Binding Adnectin™ with Potent Anti-HIV Activity Is Highly Synergistic when Linked to a CD4-Binding Adnectin™. J Virol. 2018 May 9, the entire contents of which is incorporated by reference herein.

A particular embodiment provides an antigen binding polypeptide comprising a first and second polypeptide domain wherein the first polypeptide domain is (i) a CD4 binding polypeptide or (ii) a fibronectin-based scaffold polypeptide that binds the N17 domain of gp41 and the second polypeptide domain is an anti-HIV broadly neutralizing antibody (bnAb) polypeptide or a fragment thereof.

Another particular embodiment provides an antigen binding polypeptide comprising a first, second and third polypeptide domain wherein the first polypeptide domain is a fibronectin-based scaffold polypeptide that binds CD4, the second polypeptide domain is a fibronectin-based scaffold polypeptide that binds the N17 domain of gp41, and the third polypeptide domain is an anti-HIV bnAb or fragment thereof. Certain embodiments provide an antigen binding polypeptide comprising an anti-HIV bnAb as described herein, wherein the fibronectin-based scaffold polypeptide that binds CD4 and/or the fibronectin-based scaffold polypeptide that binds the N17 domain of gp41 are each connected to the anti-HIV bnAb or fragment thereof by a linker.

In certain embodiments the antigen binding polypeptide comprising an anti-HIV bnAb and a fibronectin-based scaffold polypeptide that binds CD4 and/or a fibronectin-based scaffold polypeptide that binds the N17 domain of gp41 as described herein, the fibronectin-based scaffold polypeptide that binds CD4 and/or the fibronectin-based scaffold polypeptide that binds the N17 domain of gp41 are each connected to the HC region or the LC region of the anti-HIV bnAb. In certain embodiments, the antigen binding polypeptide comprising an anti-HIV bnAb as described herein has a HC region of the anti-HIV bnAb that comprises a sequence at least 80%, 85%, 90%, 95%, 98%, 99% or 100% identical to the sequence of SEQ ID NO: 7, SEQ ID NO: 8, SEQ ID NO: 770, SEQ ID NO: 771 or SEQ ID NO: 772.

Certain embodiments provide an antigen binding polypeptide comprising an anti-HIV bnAb and a fibronectin-based scaffold polypeptide that binds CD4 and/or a fibronectin-based scaffold polypeptide that binds the N17 domain of gp41 as described herein, the antigen binding polypeptide according to claim 4, wherein the fibronectin-based scaffold polypeptide that binds CD4 and the fibronectin-based scaffold polypeptide that binds the N17 domain of gp41 are each connected to the LC region of the anti-HIV bnAb. In certain embodiments of antigen binding polypeptides comprising an anti-HIV bnAb and a fibronectin-based scaffold polypeptide that binds CD4 and/or a fibronectin-based scaffold polypeptide that binds the N17 domain of gp41 as described herein, the fibronectin-based scaffold polypeptide that binds CD4 and the fibronectin-based scaffold polypeptide that binds the N17 domain of gp41 are each connected to the HC region of the anti-HIV bnAb.

In certain embodiments of antigen binding polypeptides comprising an anti-HIV bnAb and a fibronectin-based scaffold polypeptide that binds CD4 and/or a fibronectin-based scaffold polypeptide that binds the N17 domain of gp41 as described herein, the fibronectin-based scaffold polypeptide that binds CD4 is connected to the LC of the anti-HIV bnAb and the fibronectin-based scaffold polypeptide that binds the N17 domain of gp41 is connected to the HC of the anti-HIV bnAb.

In certain embodiments of antigen binding polypeptides comprising an anti-HIV bnAb and a fibronectin-based scaffold polypeptide that binds CD4 and/or a fibronectin-based scaffold polypeptide that binds the N17 domain of gp41 as described herein, the fibronectin-based scaffold polypeptide that binds CD4 is connected to the HC of the anti-HIV bnAb and the fibronectin-based scaffold polypeptide that binds the N17 domain of gp41 is connected to the LC of the anti-HIV bnAb.

In certain embodiments of antigen binding polypeptides comprising an anti-HIV bnAb and a fibronectin-based scaffold polypeptide that binds CD4 and/or a fibronectin-based scaffold polypeptide that binds the N17 domain of gp41 as described herein, the LC region of the anti-HIV bnAb comprises a sequence selected from SEQ NO. 6, SEQ ID NO: 768, or SEQ ID NO: 769 and the HC region of the anti-HIV bnAb comprises a sequence selected from SEQ ID NO: 7, SEQ ID NO: 8, SEQ ID NO: 770, SEQ ID NO: 771, or SEQ ID NO: 772.

In certain embodiments of antigen binding polypeptides comprising an anti-HIV bnAb and a fibronectin-based scaffold polypeptide that binds CD4 and/or a fibronectin-based scaffold polypeptide that binds the N17 domain of gp41 as described herein, the LC region of the anti-HIV bnAb comprises a sequence at least 80%, 85%, 90%, 95%, 98%, 99% or 100% identical to the sequence of SEQ ID NO: 6. In certain embodiments of antigen binding polypeptides comprising an anti-HIV bnAb and a fibronectin-based scaffold polypeptide that binds CD4 and/or a fibronectin-based scaffold polypeptide that binds the N17 domain of gp41 as described herein, the LC region of the anti-HIV bnAb comprises a sequence at least 80%, 85%, 90%, 95%, 98%, 99% or 100% identical to the sequence of SEQ ID NO: 768. In certain embodiments of antigen binding polypeptides comprising an anti-HIV bnAb and a fibronectin-based scaffold polypeptide that binds CD4 and/or a fibronectin-based scaffold polypeptide that binds the N17 domain of gp41 as described herein, the LC region of the anti-HIV bnAb comprises a sequence at least 80%, 85%, 90%, 95%, 98%, 99% or 100% identical to the sequence of SEQ ID NO: 769.

In certain embodiments of antigen binding polypeptides comprising an anti-HIV bnAb and a fibronectin-based scaffold polypeptide that binds CD4 and/or a fibronectin-based scaffold polypeptide that binds the N17 domain of gp41 as described herein, the HC region of the anti-HIV bnAb comprises a sequence at least 80%, 85%, 90%, 95%, 98%, 99% or 100% identical to the sequence of SEQ ID NO: 7. In certain embodiments of antigen binding polypeptides comprising an anti-HIV bnAb and a fibronectin-based scaffold polypeptide that binds CD4 and/or a fibronectin-based scaffold polypeptide that binds the N17 domain of gp41 as described herein, the HC region of the anti-HIV bnAb comprises a sequence at least 80%, 85%, 90%, 95%, 98%, 99% or 100% identical to the sequence of SEQ ID NO: 8. In certain embodiments of antigen binding polypeptides comprising an anti-HIV bnAb and a fibronectin-based scaffold polypeptide that binds CD4 and/or a fibronectin-based scaffold polypeptide that binds the N17 domain of gp41 as described herein, the HC region of the anti-HIV bnAb comprises a sequence at least 80%, 85%, 90%, 95%, 98%, 99% or 100% identical to the sequence of SEQ ID NO: 770. In certain embodiments of antigen binding polypeptides comprising an anti-HIV bnAb and a fibronectin-based scaffold polypeptide that binds CD4 and/or a fibronectin-based scaffold polypeptide that binds the N17 domain of gp41 as described herein, the HC region of the anti-HIV bnAb comprises a sequence at least 80%, 85%, 90%, 95%, 98%, 99% or 100% identical to the sequence of SEQ ID NO: 771. In certain embodiments of antigen binding polypeptides comprising an anti-HIV bnAb and a fibronectin-based scaffold polypeptide that binds CD4 and/or a fibronectin-based scaffold polypeptide that binds the N17 domain of gp41 as described herein, the HC region of the anti-HIV bnAb comprises a sequence at least 80%, 85%, 90%, 95%, 98%, 99% or 100% identical to the sequence of SEQ ID NO: 772.

In certain embodiments of antigen binding polypeptides comprising an anti-HIV bnAb and a fibronectin-based scaffold polypeptide that binds CD4 and/or a fibronectin-based scaffold polypeptide that binds the N17 domain of gp41 as described herein, the antigen binding polypeptide further comprises an HIV fusion peptide inhibitor. In certain embodiments of antigen binding polypeptides comprising an anti-HIV bnAb and a fibronectin-based scaffold polypeptide that binds CD4 and/or a fibronectin-based scaffold polypeptide that binds the N17 domain of gp41 as described herein, further comprising a HIV fusion peptide inhibitor, the HIV fusion peptide inhibitor is connected to the fibronectin-based scaffold polypeptide that binds the N17 domain of gp41.

Certain embodiments provide an antigen binding polypeptides comprising a first and second polypeptide domain, wherein the first polypeptide domain is a fibronectin-based scaffold polypeptide that binds CD4 and the second polypeptide domain is an anti-HIV broadly neutralizing antibody (bnAb) polypeptide or a fragment thereof.

Certain embodiments provide an antigen binding polypeptide comprising a first and second polypeptide domain, wherein the first polypeptide domain is a fibronectin-based scaffold polypeptide that binds CD4 and the second polypeptide domain is (i) an anti-HIV bnAb or a fragment thereof; (ii) a CDR variant of (i) wherein the variant has 1, 2 or 3 amino acid modifications; (iii) a LC region comprising a sequence at least 80%, 85%, 90%, 95%, 98%, 99% or 100% identical to the sequence of SEQ ID NO: 6, SEQ ID NO: 768 or SEQ ID NO: 769; and/or (iv) a HC region comprising a sequence at least 80%, 85%, 90%, 95%, 98%, 99% or 100% identical to the sequence of SEQ ID NO: 7, SEQ ID NO: 8, SEQ ID NO: 770, SEQ ID NO: 771, or SEQ ID NO: 772; or (v) a fragment thereof.

In certain embodiments of antigen binding polypeptides comprising a first and second polypeptide domain, the first polypeptide domain comprises a FG loop sequence or CD loop sequence at least 80%, 85%, 90%, 95%, 98%, 99% or 100% identical to any one of SEQ ID NOs: 13-71, and the second polypeptide domain comprises an anti-HIV bnAb described in Table 8 or a fragment thereof. In certain embodiments the antigen binding polypeptide comprising an anti-HIV bnAb and a fibronectin-based scaffold polypeptide that binds CD4 and/or a fibronectin-based scaffold polypeptide that binds the N17 domain of gp41 as described herein, the fibronectin-based scaffold polypeptide that binds CD4 comprises an amino acid sequence at least 80%, 85%, 90%, 95%, 98%, 99% or 100% identical to any one amino acid sequence of SEQ ID NOs: 72-91. In certain embodiments the antigen binding polypeptide comprising an anti-HIV bnAb and a fibronectin-based scaffold polypeptide that binds CD4 and/or a fibronectin-based scaffold polypeptide that binds the N17 domain of gp41 as described herein, the anti-HIV bnAb polypeptide is selected from a class of anti-HIV bnAb selected from an Apex (V1/V2) bnAb, a N332 glycan bnAb, a CD4 binding site (CD4bs) bnAb, a gp120-gp41 interface bnAb, and a MPER bnAb or a fragment thereof.

Certain embodiments provide an antigen binding polypeptide comprising an anti-HIV bnAb and a fibronectin-based scaffold polypeptide that binds CD4 and/or a fibronectin-based scaffold polypeptide that binds the N17 domain of gp41 as described herein, wherein the fibronectin-based scaffold polypeptide that binds CD4 comprises a sequence that is at least 80%, 85%, 90%, 95%, 98%, 99% or 100% identical to SEQ ID No: 3, SEQ ID NO: 4, SEQ ID NO: 5, or SEQ ID NO: 78. In certain embodiments, the antigen binding polypeptide comprising an anti-HIV bnAb and a fibronectin-based scaffold polypeptide that binds CD4 as described herein, further comprises a fibronectin-based scaffold polypeptide that binds the N17 d domain of gp41.

In certain embodiments, the antigen binding polypeptide comprising an anti-HIV bnAb, a fibronectin-based scaffold polypeptide that binds CD4 as described herein and a fibronectin-based scaffold polypeptide that binds the N17 d domain of gp41 as described herein, the fibronectin-based scaffold polypeptide that binds the N17 domain of gp41 comprises a sequence that is at least 80%, 85%, 90%, 95%, 98%, 99% or 100% identical to any one amino acid sequence of SEQ ID NOs: 92-348.

In certain embodiments, the antigen binding polypeptide comprises an anti-HIV bnAb and a fibronectin-based scaffold polypeptide that binds CD4 and/or a fibronectin-based scaffold polypeptide that binds the N17 domain of gp41 as described herein, and the first and second and/or third polypeptide domains are connected to each other in any order by a linker.

In certain embodiments, the antigen binding polypeptide comprising an anti-HIV bnAb and a fibronectin-based scaffold polypeptide that binds CD4 and/or a fibronectin-based scaffold polypeptide that binds the N17 domain of gp41 as described herein, the anti-HIV bnAb or fragment thereof is selected from an N6 bnAb, a VRC01 anti-HIV bnAb and a 10E8v4 anti-HIV bnAb.

In certain embodiments, the antigen binding polypeptide comprising an anti-HIV bnAb and a fibronectin-based scaffold polypeptide that binds CD4 and/or a fibronectin-based scaffold polypeptide that binds the N17 domain of gp41 as described herein comprises any one of SEQ ID NO: 9, SEQ ID NO: 10, SEQ ID NO: 11, SEQ ID NO: 12, SEQ ID NO: 49, SEQ ID NO: 750, SEQ ID NO: 751, SEQ ID NO: 752, SEQ ID NO: 753, SEQ ID NO: 754, SEQ ID NO: 755, SEQ ID NO: 756, SEQ ID NO: 757, SEQ ID NO: 758, SEQ ID NO: 759, SEQ ID NO: 760, SEQ ID NO: 761, SEQ ID NO: 762, SEQ ID NO: 763, SEQ ID NO: 764, SEQ ID NO: 765, SEQ ID NO: 766 or SEQ ID NO: 767.

In certain embodiments of antigen binding polypeptides comprising a first and second polypeptide domain, the second polypeptide domain comprises an anti-HIV bnAb that is 100% identical to SEQ ID NO: 6, SEQ ID NO: 7, SEQ ID NO: 8, SEQ ID NO: 768, SEQ ID NO: 769, SEQ ID NO: 770, SEQ ID NO: 771, or SEQ ID NO: 772.

In certain embodiments, the antigen binding polypeptide comprising an anti-HIV bnAb and a fibronectin-based scaffold polypeptide that binds CD4 and/or a fibronectin-based scaffold polypeptide that binds the N17 domain of gp41 as described herein, the CD4 binding polypeptide does not cross-react with cynomolgus monkey CD4. In certain embodiments, the antigen binding polypeptide comprising an anti-HIV bnAb and a fibronectin-based scaffold polypeptide that binds CD4 and/or a fibronectin-based scaffold polypeptide that binds the N17 domain of gp41 as described herein, the antigen binding polypeptide binds a conformational epitope within the secondary or tertiary structure of HIV CD4.

Certain embodiments provide an antigen binding polypeptide comprising an anti-HIV bnAb and a fibronectin-based scaffold polypeptide that binds CD4 and/or a fibronectin-based scaffold polypeptide that binds the N17 domain of gp41 as described herein for use in therapy. Certain embodiments provide an antigen binding polypeptide comprising an anti-HIV bnAb and a fibronectin-based scaffold polypeptide that binds CD4 and/or a fibronectin-based scaffold polypeptide that binds the N17 domain of gp41 as described herein for use in therapy wherein the therapy is for an HIV infection.

Certain embodiments provide a pharmaceutical composition comprising an antigen binding polypeptide comprising an anti-HIV bnAb and a fibronectin-based scaffold polypeptide that binds CD4 and/or a fibronectin-based scaffold polypeptide that binds the N17 domain of gp41 as described herein, and a carrier.

Certain embodiments provide an antigen binding polypeptide comprising an antigen binding polypeptide comprising an anti-HIV bnAb and a fibronectin-based scaffold polypeptide that binds CD4 and/or a fibronectin-based scaffold polypeptide that binds the N17 domain of gp41, or a pharmaceutical composition thereof as described herein, for use in the treatment of an HIV infection.

Certain embodiments provide use of an antigen binding polypeptide comprising an antigen binding polypeptide comprising an anti-HIV bnAb and a fibronectin-based scaffold polypeptide that binds CD4 and/or a fibronectin-based scaffold polypeptide that binds the N17 domain of gp41 as described herein for the preparation of a medicament for HIV infection.

Certain embodiments provide a method of treating HIV in a subject comprising administering an effective amount of an antigen binding polypeptide comprising an antigen binding polypeptide comprising an anti-HIV bnAb and a fibronectin-based scaffold polypeptide that binds CD4 and/or a fibronectin-based scaffold polypeptide that binds the N17 domain of gp41 as described herein, or pharmaceutical composition thereof.

Certain embodiments provide a nucleic acid sequence which encodes an antigen binding polypeptide comprising an anti-HIV bnAb and a fibronectin-based scaffold polypeptide that binds CD4 and/or a fibronectin-based scaffold polypeptide that binds the N17 domain of gp41 as described herein.

Certain embodiments provide a nucleic acid sequence which encodes an antigen binding polypeptide comprising an anti-HIV bnAb and a fibronectin-based scaffold polypeptide that binds CD4 and/or a fibronectin-based scaffold polypeptide that binds the N17 domain of gp41 as described herein, wherein the nucleic acid sequence encoding the bnAb polypeptide comprises a nucleic acid sequence that encodes the bnAb polypeptide region of SEQ ID NOs: 9, SEQ ID NO: 10, SEQ ID NO: 11, SEQ ID NO: 12, SEQ ID NO: 749, SEQ ID NO: 750, SEQ ID NO: 751, SEQ ID NO: 752, SEQ ID NO: 753, SEQ ID NO: 754, SEQ ID NO: 755, SEQ ID NO: 756, SEQ ID NO: 757, SEQ ID NO: 758, SEQ ID NO: 759, SEQ ID NO: 760, SEQ ID NO: 761, SEQ ID NO: 762, SEQ ID NO: 763, SEQ ID NO: 764, SEQ ID NO: 765, SEQ ID NO: 766 or SEQ ID NO: 767.

Certain embodiments provide a nucleic acid sequence which encodes an antigen binding polypeptide comprising an anti-HIV bnAb and a fibronectin-based scaffold polypeptide that binds CD4 and/or a fibronectin-based scaffold polypeptide that binds the N17 domain of gp41 as described herein, wherein the sequence encoding the CD4 binding polypeptide comprises a nucleic acid sequence that encodes the CD4 binding polypeptide region of SEQ ID NO: 9, SEQ ID NO: 10, SEQ ID NO: 11, SEQ ID NO: 12, SEQ ID NO: 749, SEQ ID NO: 750, SEQ ID NO: 751, SEQ ID NO: 752, SEQ ID NO: 753, SEQ ID NO: 754, SEQ ID NO: 755, SEQ ID NO: 756, SEQ ID NO: 757, SEQ ID NO: 758, SEQ ID NO: 759, SEQ ID NO: 760, SEQ ID NO: 761, SEQ ID NO: 762, SEQ ID NO: 763, SEQ ID NO: 764, SEQ ID NO: 765, SEQ ID NO: 766 or SEQ ID NO: 767.

Certain embodiments provide an expression vector comprising a nucleic acid sequence that encodes an antigen binding polypeptide comprising an anti-HIV bnAb and a fibronectin-based scaffold polypeptide that binds CD4 and/or a fibronectin-based scaffold polypeptide that binds the N17 domain of gp41 as described herein.

Certain embodiments provide a recombinant host cell comprising a nucleic acid sequence that encodes an antigen binding polypeptide comprising an anti-HIV bnAb and a fibronectin-based scaffold polypeptide that binds CD4 and/or a fibronectin-based scaffold polypeptide that binds the N17 domain of gp41 as described herein, or comprising an expression vector thereof.

Certain embodiments provide a method for the production of an antigen binding polypeptide comprising an anti-HIV bnAb and a fibronectin-based scaffold polypeptide that binds CD4 and/or a fibronectin-based scaffold polypeptide that binds the N17 domain of gp41 as described herein, which method comprises culturing host cells comprising a nucleic acid sequence that encodes such antigen binding polypeptide or culturing host cells comprising an expression vector for such antigen binding polypeptide comprising an anti-HIV bnAb and a fibronectin-based scaffold polypeptide that binds CD4 and/or a fibronectin-based scaffold polypeptide that binds the N17 domain of gp41 as described herein, wherein the host cells are cultured under conditions suitable for expression of said nucleic acid sequence or vector, and whereby an antigen binding polypeptide comprising a CD4 binding polypeptide and an anti-HIV bnAb polypeptide is produced.

### I. Fibronectin Based Scaffolds—Adnectins™

One aspect of the application provides anti-CD4 and anti-gp41 Adnectins™ comprising a fibronectin type III (Fn3) domain in which part or all of one or more of the solvent accessible loops has been randomized or mutated. In some embodiments, one or more residues in one or more of the non-loop beta strands has also been randomized or mutated. In some embodiments, the Fn3 domain is an Fn3 domain derived from the tenth type 3 module of human fibronectin (¹⁰Fn3):

(SEQ ID NO: 1)
VSDVPRDLEVVAATPTSLLISWDAPAVTVRYYRITYGETGGNSPVQEFTV
PGSKSTATISGLKPGVDYTITVYAVTGRGDSPASSKPISINYRT
(BC,CD,DE, and FG loops are underlined)

In other embodiments, the non-ligand binding sequences of ¹⁰Fn3, i.e., the “¹⁰Fn3 scaffold”, may be altered, provided that the ¹⁰Fn3 retains ligand binding function and/or structural stability. A variety of mutant ¹⁰Fn3 scaffolds have been reported. In one aspect, one or more of Asp 7, Glu 9, and Asp 23 of SEQ ID NO: 1 is replaced by another amino acid, such as, for example, a non-negatively charged amino acid residue (e.g., Asn, Lys, etc.). These mutations have been reported to have the effect of promoting greater stability of the mutant ¹⁰Fn3 at neutral pH as compared to the wild-type form (see, e.g., PCT Publication No. WO 02/04523). A variety of additional alterations in the ¹⁰Fn3 scaffold that are either beneficial or neutral have been disclosed. See, for example, Batori et al., Protein Eng., 15(12):1015-1020 (December 2002); Koide et al., Biochemistry, 40(34):10326-10333 (Aug. 28, 2001).

Both variant and wild-type ¹⁰Fn3 proteins are characterized by the same structure, namely seven beta-strand domain sequences designated A through G and six loop regions (AB loop, BC loop, CD loop, DE loop, EF loop, and FG loop) which connect the seven beta-strand domain sequences. The beta strands positioned closest to the N- and C-termini may adopt a beta-like conformation in solution. In SEQ ID NO: 1, the AB loop corresponds to residues 14-17, the BC loop corresponds to residues 23-31, the CD loop corresponds to residues 37-47, the DE loop corresponds to residues 51-56, the EF loop corresponds to residues 63-67, and the FG loop corresponds to residues 75-87.

Accordingly, in some embodiments, the anti-CD4 or anti-gp41 Adnectin™ of the invention is a ¹⁰Fn3 polypeptide that is at least 40%, 50%, 60%, 65%, 70%, 75%, 80%, 85%, 90%, 95%, 98% or 99% identical to the human ¹⁰Fn3 domain, shown in SEQ ID NO: 1. Much of the variability will generally occur in one or more of the loops. Each of the beta or beta-like strands of a ¹⁰Fn3 polypeptide may consist essentially of an amino acid sequence that is at least 40%, 50%, 60%, 65%, 70%, 75%, 80%, 85%, 90%, 95%, 98, 99% or 100% identical to the sequence of a corresponding beta or beta-like strand of SEQ ID NO:1, provided that such variation does not disrupt the stability of the polypeptide in physiological conditions.

In some embodiments, the invention provides one or more Adnectins™ comprising a tenth fibronectin type III (¹⁰Fn3) domain, wherein the ¹⁰Fn3 domain comprises a loop, AB; a loop, BC; a loop, CD; a loop, DE; a loop EF; and a loop FG; and has at least one loop selected from loop BC, CD, DE, and FG with an altered amino acid sequence relative to the sequence of the corresponding loop of the human ¹⁰Fn3 domain. In some embodiments, the Adnectins™ of the present invention comprise an ¹⁰Fn3 domain comprising an amino acid sequence at least 40%, 50%, 60%, 65%, 70%, 75%, 80%, 85%, 90%, 95%, 98%, 99% or 100% identical to the non-loop regions of SEQ ID NO: 1, wherein at least one loop selected from BC, CD, DE, and FG is altered. In some embodiments, the BC and FG loops are altered, and in some embodiments, the BC, DE, and FG loops are altered, i.e., the ¹⁰Fn3 domains comprise non-naturally occurring loops.

In some embodiments, the AB, CD and/or the EF loops are altered. In some embodiments the CD and FG loops are altered. In some embodiments the solvent-accessible amino acids in the strands between loops are altered, with or without alteration of the adjoining loops. By “altered” is meant one or more amino acid sequence alterations relative to a template sequence (corresponding human fibronectin domain) and includes amino acid additions, deletions, substitutions or a combination thereof. Altering an amino acid sequence may be accomplished through intentional, blind, or spontaneous sequence variation, generally of a nucleic acid coding sequence, and may occur by any technique, for example, PCR, error-prone PCR, or chemical DNA synthesis.

In some embodiments, one or more loops selected from BC, CD, DE, and FG may be extended or shortened in length relative to the corresponding human fibronectin loop. In some embodiments, the length of the loop may be extended by 1-25 amino acids. In some embodiments, the length of the loop may be decreased by 1-11 amino acids. To optimize antigen binding, therefore, a loop of ¹⁰Fn3 may be altered in length as well as in sequence to obtain the greatest possible flexibility and affinity in antigen binding.

In some embodiments, the Adnectins™ comprise a Fn3 domain that comprises an amino acid sequence at least 40%, 50%, 60%, 65%, 70%, 75%, 80%, 85%, 90%, 95%, 98%, 99%, or 100% identical to the non-loop regions of SEQ ID NO: 1, wherein at least one loop selected from BC, CD, DE, and FG is altered. In some embodiments, the altered BC loop has up to 1, 2, 3, 4, 5, 6, 7, 8 or 9 amino acid substitutions, up to 1, 2, 3, or 4 amino acid deletions, up to 1, 2, 3, 4, 5, 6, 7, 8, 9, or 10 amino acid insertions, or a combination thereof. In some embodiments, the altered CD loop has up to 1, 2, 3, 4, 5, 6, 7, 8, 9, 10, or 11 amino acid substitutions, up to 1, 2, 3, 4, 5, or 6 amino acid deletions, up to 1, 2, 3, 4, 5, 6, 7, 8, 9, or 10 amino acid insertions, or a combination thereof. In some embodiments, the altered DE loop has up to 1, 2, 3, 4, 5, or 6 amino acid substitutions, up to 1, 2, 3, or 4 amino acid deletions, up to 1, 2, 3, 4, 5, 6, 7, 8, 9, 10, 11, 12, or 13 amino acid insertions, or a combination thereof. In some embodiments, the FG loop has up to 1, 2, 3, 4, 5, 6, 7, 8, 9, 10, 11, 12 or 13 amino acid substitutions, up to 1, 2, 3, 4, 5, 6, 7, 8, 9, 10, or 11 amino acid deletions, up to 1, 2, 3, 4, 5, 6, 7, 8, 9, 10, 11, 12, 13, 14, 15, 16, 17, 18, 19, 20, 21, 22, 23, 24, or 25 amino acid insertions, or a combination thereof.

### Extension Sequences

In certain embodiments, the Adnectin™ molecules of the present invention may be modified to comprise an N-terminal extension sequence and/or a C-terminal extension. For example, an MG sequence may be placed at the N-terminus of the ¹⁰Fn3 defined by SEQ ID NO: 1. The M will usually be cleaved off, leaving a G at the N-terminus. The Adnectins™ described herein may also comprise alternative C-terminal tail sequences, referred to herein as truncated C-terminal or C-terminal extension sequences. Further, truncated version may be used as therapeutic molecules in the truncated form, or alternative C-terminal extensions, such as His6 tag, may be added to the truncated version. In certain embodiments, the C-terminal extension sequences (also called “tails”), comprise E and D residues, and may be between 8 and 50, 10 and 30, 10 and 20, 5 and 10, and 2 and 4 amino acids in length. In certain embodiments, the first residue of a C-terminal extension is a proline. In certain other embodiments, the first residue of a C-terminal extension is a glutamic acid.

In some embodiments, the N-terminus may be extended by up to 1, 2, 3, 4, 5, 6, 7, 8, 9, 10, 11, 12, 13, 14, 15, or more amino acids, which may be altered in any way, before or after rounds of selection, in order to improve target binding, stability, or both. In other embodiments, the C-terminus may be extended by up to 1, 2, 3, 4, 5, 6, 7, 8, 9, 10, 11, 12, 13, 14, 15, or more amino acids, which may be altered in any way, before or after rounds of selection, in order to improve target binding, stability, or both. In still other embodiments, both the N- and C-termini may be extended in this manner.

### Anti-CD4 Adnectin™

The anti-CD4 Adnectin™ domains of the invention include the following sequences:

Anti-CD4 Adnectin™
(SEQ ID NO: 3)
GVSDVPRDLEVVAATPTSLLISWDAPAVTVHSYHIQYWPLGSYQRYQVFS
VPGSKSTATISGLKPGVEYQIRVYAETGRGESDQSLGWIQIGYRTE
Anti-CD4 Adnectin™
(SEQ ID NO: 4)
GVSDVPRDLEVVAATPTSLLISWDAPAVTVHSYHIQYWPLGSYQRYQVFS
VPGSKSTATISGLKPGVEYQIRVYAETGRGESDQSLGWIQIGYRTP
Anti-CD4 Adnectin™
(SEQ ID NO: 5)
GVSDVPRDLEVVAATPTSLLISWDAPAVTVHSYHIQYWPLGSYQRYQVFS
VPGSKSTATISGLKPGVEYQIRVYAETGGADSDQSFGWIQIGYRTP

In an embodiment, the polypeptide of the invention provides an anti-CD4 Adnectin™ as described in any of SEQ ID No: 3, SEQ ID NO: 4, SEQ ID NO: 5, or SEQ ID NO: 78 linked to the light chain (LC) or heavy chain (HC) of an anti-HIV bnAb as described herein, particularly the LC or HC of any of the anti-HIV bnAbs described in Table 8, more particularly the LC or HC of a 10E8 bnAb, VRC01 bnAb or N6.

In an embodiment, the polypeptide of the invention provides an anti-CD4 Adnectin™ that comprises an amino acid sequence at least 70%, 75%, 80%, 85%, 90%, 95%, 98%, 99% or 100% identical to any one of SEQ ID No: 3, 4, 5, or 78 linked to the light chain (LC) or heavy chain (HC) of an anti-HIV bnAb as described herein, particularly the LC or HC of any of the anti-HIV bnAbs described in Table 8, more particularly the LC or HC of a 10E8 bnAb or a VRC01 bnAb or N6.

In an embodiment, the polypeptide of the invention comprises an anti-CD4 Adnectin™, an anti-HIV bnAb, and optionally further comprises an anti-gp41 Adnectin™. In an embodiment, the anti-CD4 Adnectin™/anti-HIV bnAb polypeptide of the invention (and optional anti-gp41 Adnectin™) has any one of the sequences set forth in SEQ ID NO: 9, SEQ ID NO: 10, SEQ ID NO: 11, SEQ ID NO: 12, SEQ ID NO: 749, SEQ ID NO: 750, SEQ ID NO: 751, SEQ ID NO: 752, SEQ ID NO: 753, SEQ ID NO: 754, SEQ ID NO: 755, SEQ ID NO: 756, SEQ ID NO: 757, SEQ ID NO: 758, SEQ ID NO: 759, SEQ ID NO: 760, SEQ ID NO: 761, SEQ ID NO: 762, SEQ ID NO: 763, SEQ ID NO: 764, SEQ ID NO: 765, SEQ ID NO: 766 or SEQ ID NO: 767.

### Anti-CD4 Adnectin™ Loop Regions CD and FG

The amino acid sequence of anti-CD4 Adnectin™ loop region CD and FG of the invention include but are not limited to those sequence combinations listed in Table 3 below. The CD loops described in Table 3 replace R30 through T49 of ¹⁰Fn3 defined by SEQ ID NO: 1. The FG loops described in Table 3 replace D67 through N91 of ¹⁰Fn3 defined by SEQ ID NO:1.

Table 3 also lists the IC₅₀ values for each anti-CD4 Adnectin™ comprising the listed CD/FG loop sequence combinations.

TABLE 3
CD4 Adnectin™ - CD and FG loop combinations

| CD loop | FG loop | IC₅₀, uM | SEQ ID NO |
|---|---|---|---|
| HSYHIQYWPLGSYQRY | EYQIRVYAETGGADSDQSM | 0.0025 | 13, 14 |
| QVFS | GWIQIG | | |
| LSYHIQYWPLGLYQAY | EYQIRVYAETGRGESPASFG | 0.0060 | 15, 16 |
| QVFS | WIQIG | | |
| HAYHIQYWPLGFYQGY | EYQIRVYAETGLGDAHQSLG | 0.0072 | 17, 18 |
| QVFS | WIQIG | | |
| LAYHIQYWPLGWYQR | EYQIRVYAETGRGESPASFG | 0.0075 | 19, 16 |
| YQIFS | WIQIG | | |
| LAYHIQYWPLGWYQR | EYQIRVYAETGRGESPASFG | 0.0082 | 20, 16 |
| YQVFS | WIQIG | | |
| HFYHIQYWPLGLYHLY | EYQIRVYAETGRGESPASFG | 0.0087 | 21, 16 |
| QVFS | WIQIG | | |
| YSYHIQYWPLGWYHR | EYQIRVYAETGADDPVQAL | 0.0099 | 22, 23 |
| YQVFS | GWIQIG | | |
| RCYHIQYWPLGLYPLY | EYQIRVYAETGDESSVQPFG | 0.0115 | 24, 25 |
| QVFS | WIQIG | | |
| YSYHIQYWPLGWYQR | EYQIRVYAETDGGRSQQSFG | 0.0118 | 26, 27 |
| YQVFS | WIQIG | | |
| SSYHIQYWPLGAYQRY | EYQIRVYAETGRGESPASFG | 0.0158 | 28, 16 |
| QVFS | WIQIG | | |
| HAYHIQYWPLGLYQRY | EYQIRVYAETGRGESPASFG | 0.0165 | 29, 16 |
| QVFS | WIQIG | | |
| HAYYIQYWPLGSYQFY | EYQIRVYAETGRGESPASFG | 0.0213 | 30, 16 |
| QVFA | WIQIG | | |
| HSYHIQYWPLGSYLRY | EYQIRVYAETGRGESPASFG | 0.0214 | 31, 16 |
| QVFS | WIQIG | | |
| LSYHIQYWPLGFYQRY | EYQIRVYAETGRGESPASFG | 0.0230 | 32, 16 |
| QVFS | WIQIG | | |
| SAYHIQYWPLGWYHR | EYQIRVYAETGRGESPASFG | 0.0239 | 33, 16 |
| YQIFS | WIQIG | | |
| YSYHIQYWPLGAYSRH | EYQIRVYAETGGDGSEMYF | 0.0260 | 34, 35 |
| QLFS | GWIQIG | | |
| LAYHIQYWPLGWYHL | EYQIRVYAETGRGESPASFG | 0.0272 | 36, 16 |
| YQVFS | WIQIG | | |
| LAYHIQYWPLGWYQL | DYQIRVYAETSGESSEQYLG | 0.0287 | 437, 38 |
| YKVFS | WIQIG | | |
| HSYHIQYWPLGWYQL | EYQIRVYAETEVDSGQHSFG | 0.0290 | 39, 40 |
| YQVFS | WIQIG | | |
| LAYHIQYWPLGWYQR | EYQIRVYAETGESGAQQSFG | 0.0297 | 41, 42 |
| YQIFS | WIQIG | | |
| QSYHIQYWPLGAYQLY | EYQIRVYAETGRGESPASFG | 0.0323 | 43, 16 |
| QLFS | WIQIG | | |
| HAYHIQYWPLGFYQGY | EYQIRVYADTGRGYQLSYS | 0.0353 | 44, 45 |
| QVFS | WIQIGY | | |
| FRYHIQYWPLGGYERY | EYQIRVYAETGRGESPASFG | 0.0410 | 46, 16 |
| QVFT | WIQIG | | |
| HSYHIQYWPLGSYHLY | EYQIRVYAETGRGESPASFG | 0.0411 | 47, 16 |
| QLFS | WIQIG | | |
| HSYHIQYWPLGWYQL | EYQIRVYAETGGFGSPPNFG | 0.0436 | 48, 49 |
| YQVFT | WIQIG | | |
| QFYHIQYWPLGSYQRY | EYQIRVYAETGRGESPASFG | 0.0509 | 50, 16 |
| QVFS | WIQIG | | |
| NSYHIQYWPLGWYHR | EYQIRVYAETGRGESPASFG | 0.0517 | 51, 16 |
| YQVFS | WIQIG | | |
| HSYHIQYWPLGRYQLY | EYQIRVYAETGRGESPASFG | 0.0562 | 52, 16 |
| QVFS | WIQIG | | |
| LAYHIQYWPLGWYHL | EYQIRVYAETGGVGWHHSF | 0.0587 | 53, 54 |
| YQIFS | GWIQIG | | |
| HVYHIQYWPLGWYPR | EYQIRVYAETGRGESPASFG | 0.0604 | 55, 16 |
| YQVFS | WIQIG | | |
| HSYHIPYWELAWYQRY | EYQIRVYAETGRGESPASFG | 0.0637 | 56, 16 |
| QVFS | WIQIG | | |
| ESYHIQYWPLGLYHRY | EYQIRVYAETGRGESPASFG | 0.0688 | 57, 16 |
| QVFS | WIQIG | | |
| LAYHIQYWPLGWYQA | EYQIRVYAETGRGESPASFG | 0.0703 | 58, 16 |
| YQVFS | WIQIG | | |
| YLYHIQYWPLGWYHR | EYQIRVYAETGRGESPASFG | 0.0715 | 59, 16 |
| YQVFT | WIQIG | | |
| RFYHIQYWPLGWYHC | EYQIRVYAQTGDGSSQEYFG | 0.0839 | 60, 61 |
| YQVFV | WIQIG | | |
| HSYHIQYWPLGWYYR | EYQIRVYAETGGSGSQQYW | 0.0869 | 62, 63 |
| YQVFS | GWIQIG | | |
| HAYHIQYWPLGFYQGY | EYQIRVYAETGRGESPASFG | 0.0875 | 64, 16 |
| QVFS | WIQIG | | |
| HSYHIQYWPLGLYVLY | EYQIRVYAETGAGGSEHSFG | 0.1059 | 65, 66 |
| QVFS | WIQIG | | |
| LSYHIQYWPLGRYERY | EYQIRVYAETVGGESLDSFS | 0.1277 | 67, 68 |
| QVFS | WIQIG | | |
| LSYHIQYWPLGWYQLY | EYQIRVYAETRVGGSVASFG | 0.1600 | 69, 70 |
| QVFY | WIQIG | | |
| LAYHIQYWPLGRYQLY | EYQIRVYAETGRGESPASFG | 0.4281 | 71, 16 |
| QVFS | WIQIG | | |

In some embodiments, anti-CD4 Adnectin™ of the invention comprises an amino acid sequence at least 80%, 85%, 90%, 95%, 98%, 99% or 100% identical to the CD/FG loop region sequence combinations of SEQ ID NOs: 13-71.

In some embodiments, anti-CD4 Adnectin™ of the invention comprises an amino acid sequence at least 80%, 85%, 90%, 95%, 98%, 99% or 100% identical to any one of the CD loop regions of SEQ ID NOs: 13, 15, 17, 19, 20, 21, 22, 24, 26, 28, 29, 30, 31, 32, 33, 34, 36, 37, 39, 41, 43, 44, 46, 47, 48, 50, 51, 52, 53, 55, 56, 57, 58, 59, 60, 62, 64, 65, 67, 69 and 71.

In some embodiments, anti-CD4 Adnectin™ of the invention comprises an amino acid sequence at least 80%, 85%, 90%, 95%, 98%, 99% or 100% identical to any one of the FG loop regions of SEQ ID NOs: 14, 16, 18, 23, 25, 27, 35, 38, 40, 42, 45, 49, 54, 63, 66, 68 and 70.

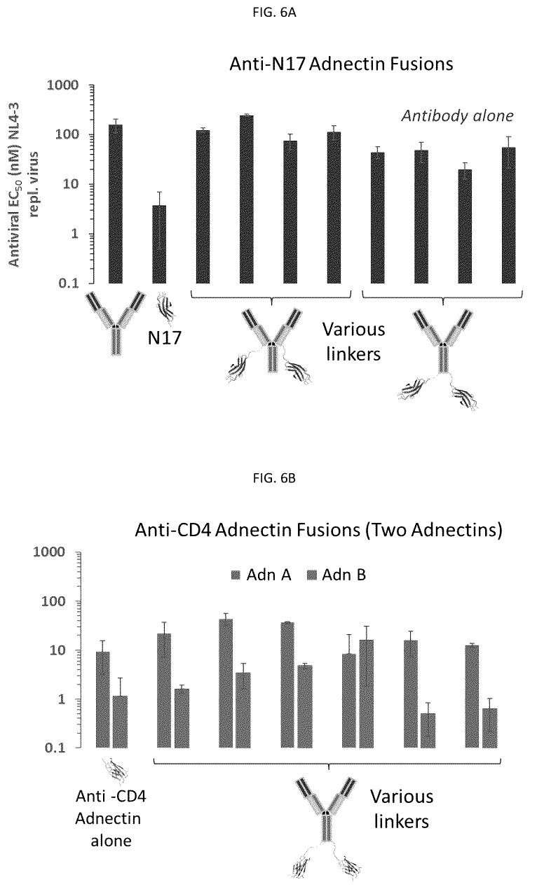

WebLogo (weblogo.berkeley.edu) was used to identify consensus sequences for anti-CD4 Adnectin™. Y32, I34, Y36, Q46 and F48 of the anti-CD4 Adnectin™ CD loop of SEQ ID NO: 1 are conserved amino acids (see FIG. 6 of WO2016/171980). In some embodiments, the anti-CD4 Adnectin™ comprises one or more of the conserved amino acids Y32, 134, Y36, Q46 and F48.

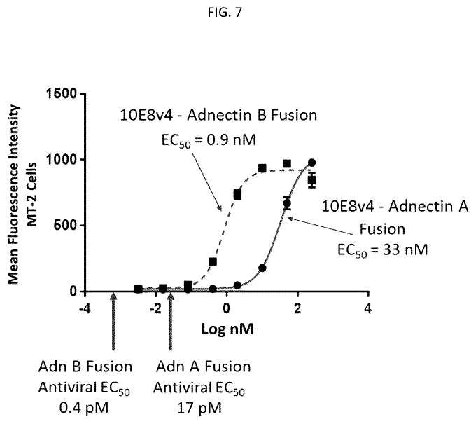

WebLogo identified Y68, 170, V72, A74, T76, 188 and 190 of SEQ ID NO: 1 of the anti-CD4 Adnectin™ FG loop as conserved amino acids (see FIG. 7 of WO2016/171980). In some embodiments, the anti-CD4 Adnectin™ comprises one or more of the conserved amino acids Y68, 170, V72, A74, T76, 188 and 190 of SEQ ID NO: 1

In some embodiments, the anti-CD4 Adnectin™ comprises one or more of the conserved amino acids Y32, I34, Y36, Q46, F48, Y68, 170, V72, A74, T76, 188 and 190 of SEQ ID NO: 1.

The full length amino acid sequences of anti-CD4 Adnectins™ of the invention include but are not limited to those listed in Table 4 below. Table 4 also lists the antiviral EC₅₀ values for each anti-CD4 Adnectin™.

TABLE 4
Anti-CD4 Adnectin™

| | Antiviral | |
|---|---|---|
| SEQ ID NO | IC₅₀ (nM) | Anti-CD4 Adnectin™ Protein Sequence |
| 72 | 48 | MASTSGVSDVPRDLEVVAATPTSLLISWDAPAVTVHS |
| | | YHIQYWPLGWYQRYQVFSVPGSKSTATISGLKPEVEY |
| | | QIRVYAETGGGGSQQSFGWIQIGYRTEGSGSHHHHHH |
| 73 | 7.8 | MGVSDVPRDLEVVAATPTSLLISWDAPAVTVQSYHIQ |
| | | YWPLGSYQRYQVFSVPGSKSTATISGLKPGVEYQIRV |
| | | YAETGGADSDQSMGWIQIGYRTEGDKPSQHHHHHH |
| 74 | 11 | MGVSDVPRDLEVVAATPTSLLISWDAPAVTVHSYHIQ |
| | | YWPLGWYQRYQVFSVPGSKSTATISGLKPGVEYQIRV |
| | | YAETRSGLADESFGWIQIGYRTEGDKPSQHHHHHH |
| 75 | 4.9 | MGVSDVPRDLEVVAATPTSLLISWDAPAVTVHSYHIQ |
| | | YWPLGSYQRYQVFSVPGSKSTATISGLKPGVEYQIRV |
| | | YAETGGADSDQSMGWIQIGYRTEGDKPSQHHHHHH |
| 76 | 8.5 | MGHHHHHHGGVSDVPRDLEVVAATPTSLLISWDAPA |
| | | VTVHSYHIQYWPLGSYQRYQVFSVPGSKSTATISGLK |
| | | PGVEYQIRVYAETGGADSDQSFGWIQIGYRTPES |
| 77 | 6 | MASTSGSSSYLMPSDLEVVAATPTSLYIHWYPIASTIIN |
| | | FRITYGETGGNSPVQEFTVPGSQVHATISGLKPGVDYT |
| | | ITVYAVHYEHKYSELWMGHPISINYRTEGSGSHHHHH |
| | | H |
| 78 | >400 | MASTSGSASYLIPSDLEVVAATPTSLSIYWYPVASTIIN |
| | | FRITYVETGGNSPVQEFTVPGSKSTATISGLKPGVDYTI |
| | | TVYAVHYEQKYSEYWIGHPISINYRTEGSGSHHHHHH |
| 79 | >5500 | MASTSGSSPYLMPYDLEVVAATPTSLFIRWYGSASSIV |
| | | KFRITYGETGGNSPVQEFTVGGTQLHATISGLKPGVD |
| | | YTITVYAVHFEHKYSELWIGHPISINYRTEGSGSHHHH |
| | | HH |
| 80 | 255 | MASTSGYTSYPIPYDLEVVAATPTSLYIHWYWIAATII |
| | | SFRITYGETGGNSPVQEFTVPAGQDHATISGLKPGVDY |
| | | TITVYAVHYEEEYSEFWTGHPISINYRTEGSGSHHHHH |
| | | H |
| 81 | 500 | MASTSGTHWFYSIPHDLEVVAATPTSLTIAWEPPHHT |
| | | AMGYRITYGETGGNSPVQEFTVPGGYTTAYISGLKPG |
| | | VDYTITVYAAYYEREYSEHWISHPISINYRTEGSGSHH |
| | | HHHH |
| 82 | 300 | MASTSGEFYHTKYPYDLEVVAATPTSLEISWRSPTRD |
| | | WQWFRITYGETGGNSPVQEFTVAGPYRNAIISGLKPG |
| | | VDYTITVYADVYMPSEGGLVVDTYHPISINYRTEGSG |
| | | SHHHHHH |
| 83 | 2400 | MASTSGQAYPEYYFVDLEVVAATPTSLLISWSKPYYN |
| | | AYSYRITYGETGGNSPVQEFTVLGHDTRAVISGLKPG |
| | | VDYTITVYAMFIEYIDQEIWHAHPISINYRTEGSGSHH |
| | | HHHH |
| 84 | 500 | MGVSDVPRDLEVVAATPTSLLISWDEHTDIYRYYRIT |
| | | YGETGGNSPVQEFTVPAMEHTATISGLKPGVDYTITV |
| | | YAVTHVYPIMIHQYPISINYRTEIDKPSQHHHHHH |
| 85 | 170 | MASTSGVSDVPRDLEVVAATPTSLLISWDAPAVTVLE |
| | | YQIDYHPAAVWHALQRFTVPGSKSTATISGLKPGVHY |
| | | KISVTATTHADNESIMWHPISIYYRTEGSGSHHHHHH |
| 86 | 320 | MGVSDVPRDLEVVAATPTSLLISWDYPTVTPRYYRIT |
| | | YGETGGNSPVQEFTVPEYIGTATISGLKPGVDYTITVY |
| | | AVTNDTTIYSISRPISINYRTEIDKPSQHHHHHH |
| 87 | 28.6 | MGHHHHHHGGVSDVPRDLEVVAATPTSLLISWDAPA |
| | | VTVHSYHIQYWPLGSYQRYQVFSVPGSKSTATISGLK |
| | | PGVEYQIRVYAETGRGESDQSLGWIQIGYRTEES |
| 88 | 20.0 | MGHHHHHHGGVSDVPRDLEVVAATPTSLLISWDAPA |
| | | VTVHSYHIQYWPLGSYQRYQVFSVPGSKSTATISGLK |
| | | PGVEYQIRVYAETGRGESDQSFGWIQIGYRTEES |
| 89 | 18.4 | MGHHHHHHGGVSDVPRDLEVVAATPTSLLISWDAPA |
| | | VTVHSYHIQYWPLGSYQRYQVFSVPGSKSTATISGLK |
| | | PGVEYQIRVYAETGRGESDQSLGWIQIGYRTPES |
| 90 | 15.4 | MGHHHHHHGGVSDVPRDLEVVAATPTSLLISWDAPA |
| | | VTVHSYHIQYWPLGSYQRYQVFSVPGSKSTATISGLK |
| | | PGVEYQIRVYAETGRGESDQSFGWIQIGYRTPES |
| 91 | 12.6 | MGHHHHHHGGVSDVPRDLEVVAATPTSLLISWDAPA |
| | | VTVHAYHIQYWPLGFYQGYQVFSVPGSKSTATISGLK |
| | | PGVEYQIRVYAETGLGDAHQSLGWIQIGYRTPES |

In some embodiments, the anti-CD4 Adnectin™ of the invention comprises an amino acid sequence at least 70%, 75%, 80%, 85%, 90%, 95%, 98%, 99% or 100% identical to any one of SEQ ID NOs: 72-91.

In some embodiments, the anti-CD4 Adnectin™ of the invention comprises an amino acid sequence at least 70%, 75%, 80%, 85%, 90%, 95%, 98%, 99% or 100% identical to any one of SEQ ID NOs: 72-91, excluding any N-terminus extended region.

In some embodiments, the anti-CD4 Adnectin™ of the invention comprises an amino acid sequence at least 70%, 75%, 80%, 85%, 90%, 95%, 98%, 99% or 100% identical to any one of SEQ ID NOs: 72-91, excluding any C-terminus extended region.

In some embodiments, the anti-CD4 Adnectin™ of the invention comprises an amino acid sequence at least 70%, 75%, 80%, 85%, 90%, 95%, 98%, 99% or 100% identical to any one of SEQ ID NOs: 72-91, excluding both the N-terminus and C-terminus extended regions.

In other embodiments, anti-CD4 Adnectin™ comprises an amino acid sequence at least 80%, 85%, 90%, 95%, 98%, 99% or 100% identical to the N-terminal region (highlighted in SEQ ID NO: 78), the BC loop, CD loop and FG loop regions of SEQ ID NOs: 72-91.

In particular embodiments, the DE loop of the anti-N17 Adnectin™ comprises one or more of the conserved DE loop amino acids S52, V53, L54 and S55 of SEQ ID NO: 1. In particular embodiments, position 26 of the anti-N17 Adnectin™ BC loop is valine or leucine.

In particular embodiments, anti-gp41 (N17) Adnectin™ comprises an amino acid sequence at least 80%, 85%, 90%, 95%, 98%, 99% or 100% identical to the BC loop, DE loop and/or FG loop regions of SEQ ID NOs: 92-348. In particular embodiments, the anti-N17 Adnectin™ comprises one or more of the conserved amino acids G78 and S85 of SEQ ID NO: 1. In particular embodiments, position 79 of the anti-N17 Adnectin™ FG loop of SEQ ID NO: 1 is valine or isoleucine.

In particular embodiments, the anti-N17 Adnectin™ comprises one or more of the conserved amino acids corresponding to Y24, V26, L26, S52, V53, L54, S55 G78, V79, 179 and S85 of SEQ ID NO: 1.

Point mutation analysis of the anti-N17 Adnectin™ showed advantages of mutating several ¹⁰Fn3 non-loop scaffold positions. Specifically, mutating positions T56 and T58 of SEQ ID NO: 1 boosted potency. In some embodiments, the anti-N17 Adnectin comprises ¹⁰Fn3 non-loop mutations of T58N, T58E, or T58Q of SEQ ID NO: 1.

### Anti-Gp41 (N17) Adnectin™-bnAb Fusion Polypeptides

In an embodiment, the polypeptide of the invention comprises an anti-gp41 Adnectin™ (N17 Adnectin™) sequence as described in Table 5 linked to an anti-HIV bnAb, or fragment thereof.

The full length amino acid sequence of N17 Adnectin™ polypeptides for use with anti-HIV bnAbs polypeptides of the invention include but are not limited to those listed in Table 5 below. Table 5 also lists the antiviral EC₅₀ values for each N17 Adnectin™. Anti-HIV bnAb-N17 Adnectin™ peptide fusions can be paired with the corresponding heavy or light chain of an anti-HIV bnAb described in Table 8, linked to a CD4 Adnectin™ from Table 3 or 4, to make a bnAb fusion comprising CD4 Adnectin™, N17 Adnectin™, and an anti-HIV bnAb. FIGS. 3A-3D show some exemplary polypeptides of the invention comprising a CD4 Adnectin™, an N17 Adnectin™, and an anti-HIV bnAb.

TABLE 5
Anti-gp41 (N17) Adnectin™

| SEQ ID | Antiviral | |
|---|---|---|
| NO | EC₅₀ (nM) | Anti-gp41 (N17) Adnectin™ Polypeptide Sequence |
| 92 | 130 | MGVSDVPRDLEVVAATPTSLLISWEYKVNNYRYYRITY |
| | | GETGGNSPVQEFTVPSVLSTATISGLKPGVDYTITVYAV |
| | | TYGVHSSPISINYRTEIDKPSQHHHHHH |
| 93 | 50 | MASTSGVSDVPRDLEVVAATPTSLLISWDAPAVTVEQY |
| | | YIAYYVEGEPSSYQYFRVPGSKSTATISGLKPGVLYHIY |
| | | VNAVTGSGLRPEFSLPIRIKYRTEGSGSHHHHHH |
| 94 | 42 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGDDSAPISINYRTEID |
| 95 | 28 | MGVSDVPRDLEVVAATPTSLLISWKYKVHPYRYYRITY |
| | | GETGGNSPVQEFTVPSVLSTATISGLKPGVDYTITVYAV |
| | | TYGVNSLPISINYRTEIDKPSQHHHHHH |
| 96 | >1,000 | MGVSDVPRDLEVVAATPTSLLISWDAPAVTVGWYHIGY |
| | | NVEGEPASYQYFRVPGSKSTATISGLKPGVEYMIFVNAV |
| | | TGSGAREEFSLPISINYRTEGSGSHHHHHH |
| 97 | 39 | MGHHHHHHGGVSDVPRDLEVVAATPTSLLISWEYNVN |
| | | PYRYYRITYGETGGNSPVQEFTVPSVLSSAQISGLKPGV |
| | | DYTITVYAVTRGVDSAPISINYRTPGG |
| 98 | 57 | MGHHHHHHGGVSDVPRDLEVVAATPTSLLISWEYNVN |
| | | PYRYYRITYGETGGNSPVQEFTVPSVLSTAEISGLKPGV |
| | | DYTITVYAVTYGVDSDPISINYRTPGG |
| 99 | 6 | MGHHHHHHGGVSDVPRDLEVVAATPTSLLISWQYKVH |
| | | PYRYYRITYGETGGNSPVQEFTVPSVLSTAEISGLKPGV |
| | | DYTITVYAVTRGVDSAPISINYRTPGG |
| 100 | 28 | MGVSDVPRDLEVVAATPTSLLISWEYKVHPYRYYRITY |
| | | GETGGNSPVQEFTVPSVLSTATISGLKPGVDYTITVYAV |
| | | TRGVDSAPISINYRTEIDKPSQHHHHHH |
| 101 | 6 | MGVSDVPRDLEVVAATPTSLLISWEYKVHPYRYYRITY |
| | | GETGGNSPVQEFTVPSVLSTAEISGLKPGVDYTITVYAV |
| | | TRGVDSAPISINYRTPIDKPSQHHHHHH |
| 102 | 4342 | MGHHHHHHGGVSDVPRDLEVVAATPTSLLISWEYKVH |
| | | PYRYYRITYGETGGNSPVQEFTVPSVLSTATISGLKPGV |
| | | DYTITVYAVTYGVQSDPISINYRTPGG |
| 103 | 8 | MGVSDVPRDLEVVAATPTSLLISWEYKVHPYRYYRITY |
| | | GETGGNSPVQEFTVPSVLSTATISGLKPGVDYTITVYAV |
| | | TYGIQSPPISINYRTEIDKPSQHHHHHH |
| 104 | 9 | MGHHHHHHGGVSDVPRDLEVVAATPTSLLISWQYKVH |
| | | PYRYYRITYGETGGNSPVQEFTVPSVLSSAQISGLKPGV |
| | | DYTITVYAVTYGIESSPISINYRTPGG |
| 105 | 18 | MGHHHHHHGGVSDVPRDLEVVAATPTSLLISWEYKVH |
| | | PYRYYRITYGETGGNSPVQEFTVPSVLSSAEISGLKPGVD |
| | | YTITVYAVTYGIDSSPISINYRTPGG |
| 106 | 11 | MGVSDVPRDLEVVAATPTSLLISWEYKVHPYRYYRITY |
| | | GETGGNSPVQEFTVPSVLSSAEISGLKPGVDYTITVYAV |
| | | TYGIDSPPISINYRTEIDKPSQHHHHHH |
| 107 | 42 | MGHHHHHHGGSVSDVPRDLEVVAATPTSLLISWEYNV |
| | | HYDRYYRITYGETGGNSPVQEFTVPSVLSTATISGLKPG |
| | | VDYTITVYAVTDGVHSSPISINYRTEID |
| 108 | 5 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGDLSAPISINYRTEID |
| 109 | 5 | MGHHHHHHGGSVSDVPRDLEVVAATPTSLLISWDAPA |
| | | VTVEEYYIGYYVEFEPSSYQWFTVPGSKSTATISGLKPG |
| | | VEYSIYVNAVTGMGMQPEMSLPISINYRTEGS |
| 110 | 42 | MGHHHHHHGGSVSDVPRDLEVVAATPTSLLISWEYKV |
| | | HYDRYYRITYGETGGNSPVQEFTVPSVLSTATISGLKPG |
| | | VDYTITVYAVTYGVDSDPISINYRTEID |
| 111 | 5 | MGHHHHHHGGSVSDVPRDLEVVAATPTSLLISWDAPA |
| | | VTVEQYYIAYYDEKEPSSYQYFRVPGSKSTATISGLKPG |
| | | VEYAIFVNAVTRSGVLPEFSLPISINYRTEGS |
| 112 | >1,000 | MGHHHHHHGGSVSDVPRDLEVVAATPTSLLISWKYNV |
| | | NAYRYYRITYGETGGNSPVQEFTVPSVLSTATISGLKPG |
| | | VDYTITVYAVTYGVHSSPISINYRTEID |
| 113 | 5 | MGHHHHHHGGSVSDVPRDLEVVAATPTSLLISWDAPA |
| | | VTVEQYYIGYYVEAEPSSYQYFFVPGSKSTATISGLKPG |
| | | VDYAIFVNAVTASGRGPEYSLPISINYRTEGS |
| 114 | 80 | MGVSDVPRDLEVVAATPTSLLISWHDGIGEERYYRITYG |
| | | ETGGNSPVQEFTVPMDDITATISGLKPGVDYTITVYAVT |
| | | VGDVISVLHEPISINYRTEIDKPSQHHHHHH |
| 115 | 3300 | MGVSDVPRDLEVVAATPTSLLISWHYPFEGYVTYYRIT |
| | | YGETGGNSHVQEFTVPVGYTTATISGLKPGVDYTITVYA |
| | | VTSSKGYVYFPISINYRTEIDKPSQHHHHHH |
| 116 | 130 | MGVSDVPRDLEVVAATPTSLLISWEDPEAAVRYYRITY |
| | | GETGGNSPVQEFTVPINDLHSYLSGLKPGVDYTITVYAV |
| | | TEATVMYVLDEPISINYRTEIDKPSQHHHHHH |
| 117 | 950 | MGVSDVPRDLEVVAATPTSLLISWDLLEDMSRYYRITY |
| | | GETGGNSPVQEFTVPTDAYTATISGLKPGVDYTITVYAV |
| | | TQDSHVIELSYPISINYRTEIDKPSQHHHHHH |
| 118 | 3 | MGVSDVPRDLEVVAATPTSLLISWQYKVHPYRYYRITY |
| | | GETGGNSPVQEFTVPSVLSTATISGLKPGVDYTITVYAV |
| | | TYGIQSPPISINYRTEIDKPSQHHHHHH |
| 119 | 55 | MGVSDVPRDLEVVAATPTSLLISWRYRVHPYRYYRITY |
| | | GETGGNSPVQEFTVPSVLSTATISGLKPGVDYTITVYAV |
| | | TDGVQSSPISINYRTEIDKPSQHHHHHH |
| 120 | 20 | MGVSDVPRDLEVVAATPTSLLISWEYNVNPYRYYRITY |
| | | GETGGNSPVQEFTVPSVLSTATISGLKPGVDYTITVYAV |
| | | TYGIESSPISINYRTEIDKPSQHHHHHH |
| 121 | 20 | MGVSDVPRDLEVVAATPTSLLISWQYKVHPYRYYRITY |
| | | GETGGNSPVQEFTVPSVLSTATISGLKPGVDYTITVYAV |
| | | TYGVESSPISINYRTEIDKPSQHHHHHH |
| 122 | 11 | MGVSDVPRDLEVVAATPTSLLISWEYKVHPYRYYRITY |
| | | GETGGNSPVQEFTVPSVLSTATISGLKPGVDYTITVYAV |
| | | TYGVNSLPISINYRTEIDKPSQHHHHHH |
| 123 | 11 | MGVSDVPRDLEVVAATPTSLLISWEYKVHPYRYYRITY |
| | | GETGGNSPVQEFTVPSVLSTATISGLKPGVDYTITVYAV |
| | | TYGIDSPPISINYRTEIDKPSQHHHHHH |
| 124 | 28 | MGVSDVPRDLEVVAATPTSLLISWEYKVHPYRYYRITY |
| | | GETGGNSPVQEFTVPSVLSTATISGLKPGVDYTITVYAV |
| | | TYGVDSDPISINYRTEIDKPSQHHHHHH |
| 125 | 14 | MGVSDVPRDLEVVAATPTSLLISWEYNVNPYRYYRITY |
| | | GETGGNSPVQEFTVPSVLSTATISGLKPGVDYTITVYAV |
| | | TYGIDSPPISINYRTEIDKPSQHHHHHH |
| 126 | 3 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGKDSAPISINYRTEID |
| 127 | 18 | MGVSDVPRDLEVVAATPTSLLISWEYNVNPYRYYRITY |
| | | GETGGNSPVQEFTVPSVLSTATISGLKPGVDYTITVYAV |
| | | TYGVESSPISINYRTEIDKPSQHHHHHH |
| 128 | 11 | MGVSDVPRDLEVVAATPTSLLISWEYKVHPYRYYRITY |
| | | GETGGNSPVQEFTVPSVLSTATISGLKPGVDYTITVYAV |
| | | TYGIDSSPISINYRTEIDKPSQHHHHHH |
| 129 | 20 | MGVSDVPRDLEVVAATPTSLLISWEYNVNPYRYYRITY |
| | | GETGGNSPVQEFTVPSVLSTATISGLKPGVDYTITVYAV |
| | | TYGVQSDPISINYRTEIDKPSQHHHHHH |
| 130 | 8 | MGHHHHHHGGSVSDVPRDLEVVAATPTSLLISWEYKV |
| | | HPYRYYRITYGETGGNSPVQEFTVPSVLSTAQISGLKPG |
| | | VDYTITVYAVTYGDDSAPISINYRTEID |
| 131 | 8 | MGVSDVPRDLEVVAATPTSLLISWEYNVNPYRYYRITY |
| | | GETGGNSPVQEFTVPSVLSTATISGLKPGVDYTITVYAV |
| | | TYGVNSLPISINYRTEIDKPSQHHHHHH |
| 132 | 9 | MGVSDVPRDLEVVAATPTSLLISWQYKVHPYRYYRITY |
| | | GETGGNSPVQEFTVPSVLSTATISGLKPGVDYTITVYAV |
| | | THGVHSAPISINYRTEIDKPSQHHHHHH |
| 133 | 43 | MGVSDVPRDLEVVAATPTSLLISWEYKVNPWRYYRITY |
| | | GETGGNSPVQEFTVPSVLSTATISGLKPGVDYTITVYAV |
| | | TYGIESSPISINYRTEIDKPSQHHHHHH |
| 134 | 43 | MGVSDVPRDLEVVAATPTSLLISWQYKVHPYRYYRITY |
| | | GETGGNSPVQEFTVPSVLSTATISGLKPGVDYTITVYAV |
| | | TYGVNSIPISINYRTEIDKPSQHHHHHH |
| 135 | 85 | MGVSDVPRDLEVVAATPTSLLISWEYKVHYDRYYRITY |
| | | GETGGNSPVQEFTVPSVLSTATISGLKPGVDYTITVYAV |
| | | TYGIQSPPISINYRTEIDKPSQHHHHHH |
| 136 | 23 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGDDSAPISIPYRTPGG |
| 137 | 58 | MGVSDVPRDLEVVAATPTSLLISWEYKVDPYRYYRITY |
| | | GETGGNSPVQEFTVPSVLSTATISGLKPGVDYTITVYAV |
| | | TYGVESSPISINYRTEIDKPSQHHHHHH |
| 138 | 13 | MGHHHHHHGGSVSDVPRDLEVVAATPTSLLISWEYKV |
| | | HPYRYYRITYGETGGNSPVQEFTVPSVLSSAEISGLKPGV |
| | | DYTITVYAVTYGDDSAPISINYRTEID |
| 139 | 35 | MGHHHHHHGGSVSDVPRDLEVVAATPTSLLISWEYKV |
| | | HPYRYYRITYGETGGNSPVQEFTVPSVLSSAQISGLKPG |
| | | VDYTITVYAVTYGDDSAPISINYRTEID |
| 140 | 35 | MGHHHHHHGGSVSDVPRYLEVVAATPTSLLISWQYKV |
| | | HPYRWYRITYGETGGNSPVQEFTVPSVLSTATISGLKPG |
| | | VDYTITVYAVTYGVNSIPISINYRTEID |
| 141 | 35 | MGHHHHHHGGSVSDVPRDLEVVAATPTSLLISWKYQV |
| | | HAYRYYRITYGETGGNSPVQEFTVPSVLSTATISGLKPG |
| | | VDYTITVYAVTYGVYSAPISINYRTEID |
| 142 | 42 | MGHHHHHHGGSVSDVPRDLEVVAATPTSLLISWEYKV |
| | | NPYRYYRITYGETGGNSPVQEFTVPSVLSTATISGLKPG |
| | | VDYTITVYAVTYGVHSSPISINYRTEID |
| 143 | 50 | MGHHHHHHGGSVSDVPRDLEVVAATPTSLLISWKYNL |
| | | HPYRYYRITYGETGGNSPVQEFTVPSVLSTATISGLKPG |
| | | VDYTITVYAVTYGIISEPISINYRTEID |
| 144 | 42 | MGHHHHHHGGSVSDVPRDLEVVAATPTSLLISWEYRV |
| | | NPYRYYRITYGETGGNSPVQEFTVPSVLSTATISGLKPG |
| | | VDYTITVYAVTYGVQSPPISINYRTEID |
| 145 | 42 | MGHHHHHHGGSVSDVPRDLEVVAATPTSLLISWEYKV |
| | | NAYRYYRITYGETGGNSPVQEFTVPSVLSTATISGLKPG |
| | | VDYTITVYAVTYGVLSPPISINYRTEID |
| 146 | 5 | MGHHHHHHGGSVSDVPRDLEVVAATPTSLLISWEYNV |
| | | NPYRYYRITYGETGGNSPVQEFTVPSVLSTATISGLKPG |
| | | VDYTITVYAVTRGVDSAPISINYRTEID |
| 147 | 5 | MGHHHHHHGGSVSDVPRDLEVVAATPTSLLISWEYKVS |
| | | PYRYYRITYGETGGNSPVQEFTVPSVLSTATISGLKPGV |
| | | DYTITVYAVTFGIRSSPISINYRTEID |
| 148 | 35 | MGHHHHHHGGSVSDVPRDLEVVAATPTSLLISWKYQV |
| | | HAYRYYRITYGETGGNSPVQEFTVPSVLSTATISGLKPG |
| | | VDYTITVYAVTYGIISEPISINYRTEID |
| 149 | 5 | MGHHHHHHGGSVSDVPRDLEVVAATPTSLLISWEYKV |
| | | DPYRYYRITYGETGGNSPVQEFTVPSVLSTATISGLKPG |
| | | VDYTITVYAVTYGIDSSPISINYRTEID |
| 150 | 5 | MGHHHHHHGGAVSDVPRDLEVVAATPTSLLISWEYKV |
| | | NPWRYYRITYGETGGNSPVQEFTVPSVLSTATISGLKPG |
| | | VDYTITVYAVTYGVHSSPISINYRTEID |
| 151 | 5 | MGHHHHHHGGSVSDVPRDLEVVAATPTSLLISWEYNV |
| | | NAYRYYRITYGETGGNSPVQEFTVPSVLSTATISGLKPG |
| | | VDYTITVYAVTYGIISEPISINYRTEID |
| 152 | 5 | MGHHHHHHGGSVSDVPRDLEVVAATPTSLLISWEYKV |
| | | HYDRYYRITYGETGGNSPVQEFTVPSVLSTATISGLKPG |
| | | VDYTITVYAVTYGVQSTPISINYRTEID |
| 153 | >1,000 | MGHHHHHHGGSVSDVPRDLEVVAATPTSLLISWEYKV |
| | | HHDRYYRITYGETGGNSPVQEFTVPSVLSTATISGLKPG |
| | | VDYTITVYAVTYGIESSPISINYRTEID |
| 154 | 252 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGDDSAPISIPYRTPGG |
| 155 | 631 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGXDSXPISINYRTEID |
| 156 | >1,000 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGDDSRPISINYRTEID |
| 157 | >1,000 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGKDSAPISINYRTEID |
| 158 | 631 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGDHSAPISINYRTEID |
| 159 | >1,000 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGDQSAPISINYRTEID |
| 160 | 20 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGIDSAPISINYRTEID |
| 161 | 20 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGDVSAPISINYRTEID |
| 162 | 252 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGXDSAPISINYRTEID |
| 163 | 2830 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGEDSAPISINYRTEID |
| 164 | 86 | MGHHHHHHGGSVSDVPRDLEVVAATPTSLLISWEYKV |
| | | HPYRYYRITYGETGGNSPVQEFKVPSVLSTATISGLKPG |
| | | VDYTITVYAVTYGDDSAPISINYRTEID |
| 165 | 86 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGDLSAPISINYRTEID |
| 166 | >1,000 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGKDSAPISINYRTEID |
| 167 | >1,000 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGADSAPISINYRTEID |
| 168 | >1,000 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGTDSAPISINYRTEID |
| 169 | 2830 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGTDSAPISINYRTEID |
| 170 | 252 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGKDSAPISINYRTEID |
| 171 | >1,000 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGDPSAPISINYRTEID |
| 172 | >1,000 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGDDSQPISINYRTEID |
| 173 | 86 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGDVSAPISINYRTEID |
| 174 | 81 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGDDSGPISINYRTEID |
| 175 | 2830 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGDDSAPISIQYRTEID |
| 176 | >1,000 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGDDSQPISINYRTEID |
| 177 | >1,000 | MGHHHHHHGGSVSDVPRDLEVVAATPTSLLISWEYKV |
| | | HPYRYYRITYTETGGNSPVQEFTVPSVLSTATISGLKPGV |
| | | DYTITVYAVTYGDDSAPISINYRTEID |
| 178 | 20 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGDQSAPISINYRTEID |
| 179 | >1,000 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGDDSKPISINYRTEID |
| 180 | 86 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGDDSGPISINYRTEID |
| 181 | 0 | MGHHHHHHGGSVSDVPRDLEVVAATPTSLLISWEYKV |
| | | HPYRYYRITYRETGGNSPVQEFTVPSVLSTATISGLKPGV |
| | | DYTITVYAVTYGDDSAPISINYRTEID |
| 182 | 86 | MGHHHHHHGGSVSDVPRDLEVVAATPTSLLISWEYDV |
| | | HPYRYYRITYGETGGNSPVQEFTVPSVLSTATISGLKPG |
| | | VDYTITVYAVTYGDDSAPISINYRTEID |
| 183 | 237 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGDDSKPISINYRTEID |
| 184 | >1,000 | MGHHHHHHGGSVSDVPRDLEVVAATPTSLLISWEYKV |
| | | HPYRYYRITYWETGGNSPVQEFTVPSVLSTATISGLKPG |
| | | VDYTITVYAVTYGDDSAPISINYRTEID |
| 185 | >1,000 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGDDSTPISINYRTEID |
| 186 | 20 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGDDSTPISINYRTEID |
| 187 | 143 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGDDSSPISINYRTEID |
| 188 | 631 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTRGDDSAPISINYRTEID |
| 189 | 86 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTHGDDSAPISINYRTEID |
| 190 | >1,000 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGDDSWPISINYRTEID |
| 191 | >1,000 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTHGDDSAPISINYRTEID |
| 192 | >1,000 | MGHHHHHHGGSVSDVPRDLEVVAATPTSLLISWKYDV |
| | | HPYRYYRITYGETGGNSPVQEFTVPSVLSTATISGLKPG |
| | | VDYTITVYAVTYGDDSAPISINYRTEID |
| 193 | >1,000 | MGHHHHHHGGSVSDVPRDLEVVAATPTSLLISWEYKV |
| | | HPYRYYRITYGETGGNSPVQEFTVPSVLSTATISDLKPG |
| | | VDYTITVYAVTYGDDSHPISINYRTEID |
| 194 | >1,000 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTRGDDSAPISINYRTEID |
| 195 | 62 | MGHHHHHHGGSVSDVPRDLEVVAATPTSLLISWEYKV |
| | | HPYRYYRITYGETGGNSPVQEFTVPSVLSTATIRGLKPG |
| | | VDYTITVYAVTYGDDSAPISINYRTEID |
| 196 | >1,000 | MGHHHHHHGGSVSDVPRDLEVVAATPTSLLISWKYKV |
| | | HPYRYYRITYGETGGNSPVQEFTVPSVLSTATISGLKPG |
| | | VDYTITVYAVTYGDDSAPISINYRTEID |
| 197 | 86 | MGHHHHHHCGSVSDVPRDLEVVAATPTSLLISWEYKV |
| | | AHNRYYRITYGETGGNSPVQEFTVPSVLSTATISGLKPG |
| | | VDYTITVYAVTYGDDSAPISINYRTEID |
| 198 | >1,000 | MGHHHHHHGGSVSDVPRDLEVVAATPTSLLISWEYKV |
| | | YGYRYYRITYGETGGNSPVQEFTVPSVLSTATISGLKPG |
| | | VDYTITVYAVTYGDDSAPISINYRTEID |
| 199 | >1,000 | MGHHHHHHGGSVSDVPRDLEVVAATPTSLLISWEYKV |
| | | DHQRYYRITYGETGGNSPVQEFTVPSVLSTATISGLKPG |
| | | VDYTITVYAVTYGDDSAPISINYRTEID |
| 200 | >1,000 | MGHHHHHHGGSVSDVPRDLEVVAATPTSLLISWEYKV |
| | | DYRRYYRITYGETGGNSPVQEFTVPSVLSTATISGLKPG |
| | | VDYTITVYAVTYGDDSAPISINYRTEID |
| 201 | >1,000 | MGHHHHHHGGSVSDVPRDLEVVAATPTSLLISWEYKV |
| | | AYDRYYRITYGETGGNSPVQEFTVPSVLSTATISGLKPG |
| | | VDYTITVYAVTYGDDSAPISINYRTEID |
| 202 | >1,000 | MGHHHHHHGGSVSDVPRDLEVVAATPTSLLISWEYKV |
| | | TSYRYYRITYGETGGNSPVQEFTVPSVLSTATISGLKPGV |
| | | DYTITVYAVTYGDDSAPISINYRTEID |
| 203 | 631 | MGHHHHHHGGSVSDVPRDLEVVAATPTSLLISWEYKV |
| | | HPYRYYRIIHETGGNSPVQEFTVPSVLSTATISGLKPGVD |
| | | YTITVYAVTYGDDSAPISINYRTEID |
| 204 | >1,000 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGDDSAPISINYRKEAEL |
| 205 | >1,000 | MGHHHHHHGGSVSDVPRDLEVVAATPTSLLISWEYKV |
| | | NHQRYYRITYGETGGNSPVQEFTVPSVLSTATISGLKPG |
| | | VDYTITVYAVTYGDDSAPISINYRTEID |
| 206 | >1,000 | MGHHHHHHGGSVSDVPRDLEVVAATPTSLLISWEYKV |
| | | DHRRYYRITYGETGGNSPVQEFTVPSVLSTATISGLKPG |
| | | VDYTITVYAVTYGDDSAPISINYRTEID |
| 207 | 17 | MGVSDVPRDLEVVAATPTSLLISWEYKVHPYRYYRITY |
| | | GETGGNSPVQEFTVPSVLSTATISGLKPGVDYTITVYAV |
| | | TYGVDSDPISINYRTDDKPSQHHHHHH |
| 208 | 10 | MGVSDVPRDLEVVAATPTSLLISWEYKVHPYRYYRITY |
| | | GETGGNSPVQEFTVPSVLSTATISGLKPGVDYTITVYAV |
| | | TYGVNSIPISINYRTEIDKPSQHHHHHH |
| 209 | 47 | MGVSDVPRDLEVVAATPTSLLISWEYKVNAYRYYRITY |
| | | GETGGNSPVQEFTVPSVLSTATISGLKPGVDYTITVYAV |
| | | TYGIESSPISINYRTEIDKPSQHHHHHH |
| 210 | 55 | MGVSDVPRDLEVVAATPTSLLISWEYKVHPYRYYRITY |
| | | GETGGNSPVQEFTVPSVLSTATISGLKPGVDYTITVYAV |
| | | TDGVQSSPISINYRTEIDKPSQHHHHHH |
| 211 | 20 | MGVSDVPRDLEVVAATPTSLLISWEYKVNAYRYYRITY |
| | | GETGGNSPVQEFTVPSVLSTATISGLKPGVDYTITVYAV |
| | | TYGIDSSPISINYRTEIDKPSQHHHHHH |
| 212 | 29 | MGVSDVPRDLEVVAATPTSLLISWEYKVHYDRYYRITY |
| | | GETGGNSPVQEFTVPSVLSTATISGLKPGVDYTITVYAV |
| | | TNGVLSSPISINYRTEIDKPSQHHHHHH |
| 213 | 85 | MGVSDVPRDLEVVAATPTSLLISWEYKVNPWRYYRITY |
| | | GETGGNSPVQEFTVPSVLSTATISGLKPGVDYTITVYAV |
| | | TRGVDSAPISINYRTEIDKPSQHHHHHH |
| 214 | 31 | MGVSDVPRDLEVVAATPTSLLISWEYKVHPXRYYRITY |
| | | GETGGNSPVQEFTVPSVLSTATISGLKPGVDYTITVYAV |
| | | TYGVBSXPISINYRTEIDKPSQHHHHHH |
| 215 | 11 | MGVSDVPRDLEVVAATPTSLLISWEYKVNPWRYYRITY |
| | | GETGGNSPVQEFTVPSVLSTATISGLKPGVDYTITVYAV |
| | | TYGIQSPPISINYRTEIDKPSQHHHHHH |
| 216 | 4 | MGVSDVPRDLEVVAATPTSLLISWQYKVHPYRWYRITY |
| | | GETGGNSPVQEFTVPSVLSTATISGLKPGVDYTITVYAV |
| | | TYGVNSIPISINYRTEIDKPSQHHHHHH |
| 217 | 5 | MGVSDVPRDLEVVAATPTSLLISWEYKVSPYRYYRITY |
| | | GETGGNSPVQEFTVPSVLSTATISGLKPGVDYTITVYAV |
| | | TYGVNSIPISINYRTEIDKPSQHHHHHH |
| 218 | 8 | MGVSDVPRDLEVVAATPTSLLISWEYKVNPWRYYRITY |
| | | GETGGNSPVQEFTVPSVLSTATISGLKPGVDYTITVYAV |
| | | TYGVNSLPISINYRTEIDKPSQHHHHHH |
| 219 | 0 | MGVSDVPRDLEVVAATPTSLLISWEYNVHYDRYYRITY |
| | | GETGGNSPVQEFTVPSVLSTATISGLKPGVDYTITVYAV |
| | | TYGVDSDPISINYRTEIDKPSQHHHHHH |
| 220 | 48 | MGVSDVPRDLEVVAATPTSLLISWEYKVNPWRYYRITY |
| | | GETGGNSPVQEFTVPSVLSTATISGLKPGVDYTITVYAV |
| | | TYGVDSDPISINYRTEIDKPSQHHHHHH |
| 221 | 58 | MGVSDVPRDLEVVAATPTSLLISWEYKVDPYRYYRITY |
| | | GETGGNSPVQEFTVPSVLSTATISGLKPGVDYTITVYAV |
| | | TYGVDSDPISINYRTEIDKPSQHHHHHH |
| 222 | 50 | MGHHHHHHGGSVSDVPRDLEVVAATPTSLLISWKYQV |
| | | HAYRYYRITYGETGGNSPVQEFTVPSVLSTATINGLKPG |
| | | VDYTITVYAVTYGIISEPISINYRTEID |
| 223 | >1,000 | MGHHHHHHGGSVSDVPRDLEVVAATPTSLLISWEYKV |
| | | NYNRYYRITYGETGGNSPVQEFTVPSVLSTATISGLKPG |
| | | VDYTITVYAVTEGVQSAPISINYRTEID |
| 224 | 2 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGDDSAPISINYRTPGG |
| 225 | >1,000 | MGHHHHHHGGSVSDVPRDLEVVAATPTSLLISWEYKV |
| | | HPYRYYRITYGETGGNTPVQEFRVPSVLSTATISGLKPG |
| | | VDYTITVYAVTYADASAPISINYRTEID |
| 226 | 83 | MGHHHHHHGGSVSDVPRDLEVVAATPTSLLISWEYKV |
| | | HPYRYYRITYGETGGNSPVQEFTVPSVLSSATLNGLKPG |
| | | VDYTITVYAVTYGDDSAPISINYRTEID |
| 227 | 218 | MGHHHHHHGGSVSDVPRDLEVVAATPTSLLISWEYKV |
| | | HPYRYYRITYGETGGNSPVQEFTVPSVLSSAKISGLKPG |
| | | VDYTITVYAVTYGDDSAPISINYRTEID |
| 228 | 186 | MGHHHHHHGGSVSDVPRDLEVVAATPTSLLISWEYKV |
| | | HPYRYYRITYGETGGNSPVQEFNVPSVLSTATISGLKPG |
| | | VDYTITVYAVTYGDDSAPISINYRTEID |
| 229 | >1,000 | MGHHHHHHGGSVSDVPRDLEVVAATPTSLLISWEYKV |
| | | AHRRYYRITYGETGGNSPVQEFTVPSVLSTATISGLKPG |
| | | VDYTITVYAVTYGDDSAPISINYRTEID |
| 230 | >1,000 | MGHHHHHHGGSVSDVPRDLEVVAATPTSLLISWEYKV |
| | | DAYRYYRITYGETGGNSPVQEFTVPSVLSTATISGLKPG |
| | | VDYTITVYAVTYGDDSAPISINYRTEID |
| 231 | >1,000 | MGHHHHHHGGSVSDVPRDLEVVAATPTSLLISWEYKV |
| | | DSYRYYRITYGETGGNSPVQEFTVPSVLSTATISGLKPG |
| | | VDYTITVYAVTYGDDSAPISINYRTEID |
| 232 | 136 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGDDSAPISINYRTEID |
| 233 | 170 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGDASAPISINYRTEID |
| 234 | >1,000 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTNGDDSAPISINYRTEID |
| 235 | 195 | MGHHHHHHGGSVSDVPRDLEVVAATPTSLLISWEYKV |
| | | HPYRYYRITYGETGGNSPVQEFTVPSVLSSATIRGLKPG |
| | | VDYTITVYAVTYGDDSAPISINYRTEID |
| 236 | 135 | MGHHHHHHGGSVSDVPRDLEVVAATPTSLLISWEYKV |
| | | HPYRYYRITYGETGGNSPVQEFTVPSVLSTAKISGLKPG |
| | | VDYTITVYAVTYGDDSAPISINYRTEID |
| 237 | 188 | MGHHHHHHGGSVSDVPRDLEVVAATPTSLLISWEYKV |
| | | HPYRYYRITYGETGGNSPVQEFRVPSVLSTATISGLKPG |
| | | VDYTITVYAVTYGDDSAPISINYRTEID |
| 238 | >1,000 | MGHHHHHHGGSVSDVPRDLEVVAATPTSLLISWEYKV |
| | | APWRYYRITYGETGGNSPVQEFTVPSVLSTATISGLKPG |
| | | VDYTITVYAVTYGDDSAPISINYRTEID |
| 239 | >1,000 | MGHHHHHHGGSVSDVPRDLEVVAATPTSLLISWEYKV |
| | | DGWRYYRITYGETGGNSPVQEFTVPSVLSTATISGLKPG |
| | | VDYTITVYAVTYGDDSAPISINYRTEID |
| 240 | >1,000 | MGHHHHHHGGSVSDVPRDLEVVAATPTSLLISWEYKV |
| | | DTWRYYRITYGETGGNSPVQEFTVPSVLSTATISGLKPG |
| | | VDYTITVYAVTYGDDSAPISINYRTEID |
| 241 | 209 | MGHHHHHHGGSVSDVPRDLEVVAATPTXLLISWEYKV |
| | | HPYRYYRITYGETGGNSPVQEFTVPSVLSTATISGLKPG |
| | | VDYTITVYAVTYGDDSAPISINYRTEID |
| 242 | 63 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGADSAPISINYRTEID |
| 243 | >1,000 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTRGDDSAPISINYRTEID |
| 244 | >1,000 | MGHHHHHHGGSVSDVPRDLEVVAATPTSLLISWEYKV |
| | | HPYRYYRITYGETGGNSPVQDFTVPSVLSTATIRGLKPG |
| | | VDYTITVYAVTYGDDSAPISINYRTEID |
| 245 | 2 | MGHHHHHHGGSVSDVPRDLEVVAATPTSLLISWEYKV |
| | | HPYRYYRITYGETGGNSPVQEFTVPSVLSTANISGLKPG |
| | | VDYTITVYAVTYGDDSAPISINYRTEID |
| 246 | 184 | MGHHHHHHGGSVSDVPRDLEVVAATPTSLLISWEYKV |
| | | HPYRYYRITYGETGGNSPVQAFTVPSVLSTATISGLKPG |
| | | VDYTITVYAVTYGDDSAPISINYRTEID |
| 247 | >1,000 | MGHHHHHHGGSVSDVPRDLEVVAATPTSLLISWEYKV |
| | | APYRYYRITYGETGGNSPVQEFTVPSVLSTATISGLKPG |
| | | VDYTITVYAVTYGDDSAPISINYRTEID |
| 248 | >1,000 | MGHHHHHHGGSVSDVPRDLEVVAATPTSLLISWEYKV |
| | | DGYRYYRITYGETGGNSPVQEFTVPSVLSTATISGLKPG |
| | | VDYTITVYAVTYGDDSAPISINYRTEID |
| 249 | >1,000 | MGHHHHHHGGSVSDVPRDLEVVAATPTSLLISWEYKV |
| | | DTYRYYRITYGETGGNSPVQEFTVPSVLSTATISGLKPG |
| | | VDYTITVYAVTYGDDSAPISINYRTEID |
| 250 | >1,000 | MGHHHHHHGGSVSDVPRDLEVVAATPTSLLISWEYEV |
| | | HPYRYYRITYGETGGNSPVQEFTVPSVLSTATISGLKPG |
| | | VDYTITVYAVTYGDDSAPISINYRTEID |
| 251 | 115 | MGHHHHHHGGSVSDVPRDLEVVAATPTSLLISWEYRV |
| | | HPYRYYRITYGETGGNSPVQEFTVPSVLSTATISGLKPG |
| | | VDYTITVYAVTYGDDSAPISINYRTEID |
| 252 | 180 | MGHHHHHHGGSVSDVPRDLEVVAATPTSLLISWEYKV |
| | | HPYRYYRITYGETGGNSPVQSFTVPSVLSTATISGLKPGV |
| | | DYTITVYAVTYGADSAPISINYRTEID |
| 253 | 16 | MGHHHHHHGGSVSDVPRDLEVVAATPTSLLISWEYKV |
| | | HPYRYYRITYGETGGNSPVQEFTVPSVLSTATIRGLKPG |
| | | VDYTITVYAVTYGDDSAPISINYRTEID |
| 254 | >1,000 | MGHHHHHHGGSVSDVPRDLEVVAATPTSLLISWEYKV |
| | | HPYRYYRITYGETGGNSPVQDFTVPSVLSTATISGLKPG |
| | | VDYTITVYAVTYGDDSAPISINYRTEID |
| 255 | >1,000 | MGHHHHHHGGSVSDVPRDLEVVAATPTSLLISWEYKV |
| | | ARWRYYRITYGETGGNSPVQEFTVPSVLSTATISGLKPG |
| | | VDYTITVYAVTYGDDSAPISINYRTEID |
| 256 | >1,000 | MGHHHHHHGGSVSDVPRDLEVVAATPTSLLISWEYKV |
| | | DHQRYYRITYGETGGNSPVQEFTVPSVLSTATISGLKPG |
| | | VDYTITVYAVTYGDDSAPISINYRTEID |
| 257 | >1,000 | MGHHHHHHGGSVSDVPRDLEVVAATPTSLLISWEYKV |
| | | DYGRYYRITYGETGGNSPVQEFTVPSVLSTATISGLKPG |
| | | VDYTITVYAVTYGDDSAPISINYRTEID |
| 258 | >1,000 | MGHHHHHHGGSVSDVPRDLEVVAATPTSLLISWKYGV |
| | | HPYRYYRITYGETGGNSPVQEFTVPSVLSTATISGLKPG |
| | | VDYTITVYAVTYGDDSAPISINYRTEID |
| 259 | 56 | MGHHHHHHGGSVSDVPRDLEVVAATPTSLLISWEYSVH |
| | | PYRYYRITYGETGGNSPVQEFTVPSVLSTATISGLKPGV |
| | | DYTITVYAVTYGDDSAPISINYRTEID |
| 260 | >1,000 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTDGDDSAPISINYRTEID |
| 261 | >1,000 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTSGDDSAPISINYRTEID |
| 262 | 397 | MGHHHHHHGGSVSDVPRDLEVVAATPTSLLISWEYKV |
| | | HPYRYYRITYGETGGNSPVQEFTVPSVLSSATLRGLKPG |
| | | VDYTITVYAVTYGDDSAPISINYRTEID |
| 263 | >1,000 | MGHHHHHHGGSVSDVPRDLEVVAATPTSLLISWEYKV |
| | | AAWRYYRITYGETGGNSPVQEFTVPSVLSTATISGLKPG |
| | | VDYTITVYAVTYGDDSAPISINYRTEID |
| 264 | >1,000 | MGHHHHHHGGSVSDVPRDLEVVAATPTSLLISWEYKV |
| | | ATWRYYRITYGETGGNSPVQEFTVPSVLSTATISGLKPG |
| | | VDYTITVYAVTYGDDSAPISINYRTEID |
| 265 | >1,000 | MGHHHHHHGGSVSDVPRDLEVVAATTTSLLISWEYKV |
| | | DYHRYYRITYGETGGNSPVQEFTVPSVLSTATISGLKPG |
| | | VDYTITVYAVTYGDDSAPISINYRTEID |
| 266 | >1,000 | MGHHHHHHGGSVSDVPRDLEVVAATPTSLLISWEYGV |
| | | HPYRYYRITYGETGGNSPVQEFTVPSVLSTATISGLKPG |
| | | VDYTITVYAVTYGDDSAPISINYRTEID |
| 267 | 138 | MGHHHHHHGGSVSDVPRDLEVVAATPTSLLISWKYXV |
| | | HPYRYYRITYGETGGNSPVQEFTVPSVLSTATISGLKPG |
| | | VDYTITVYAVTYGDDSAPISINYRTEID |
| 268 | >1,000 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTGGDDSAPISINYRTEID |
| 269 | 52 | MGHHHHHHGGSVSDVPRDLEVVAATPTSLLISWEYKV |
| | | HPYRYYRITYGETGGNSPVQEFTVPSVLSTATIHGLKPG |
| | | VDYTITVYAVTYGDDSAPISINYRTEID |
| 270 | >1,000 | MGHHHHHHGGSVSDVPRDLEVVAATPTSLLISWEYKV |
| | | HPYRYYRITYGETGGNSPVQEFTVPSVLSTATLRGLKPG |
| | | VDYTITVYAVTYGDDSAPISINYRTEID |
| 271 | 101 | MGHHHHHHGGSVSDVPRDLEVVAATPTSLLISWEYKV |
| | | HPYRYYRITYGETGGNSPVQEFTVPSVLSSATISGLKPGV |
| | | DYTITVYAVTYGDDSAPISINYRTEID |
| 272 | >1,000 | MGHHHHHHGGSVSDVPRDLEVVAATPTSLLISWEYKV |
| | | AAYRYYRITYGETGGNSPVQEFTVPSVLSTATISGLKPG |
| | | VDYTITVYAVTYGDDSAPISINYRTEID |
| 273 | >1,000 | MGHHHHHHGGSVSDVPRDLEVVAATPTSLLISWEYKV |
| | | ATYRYYRITYGETGGNSPVQEFTVPSVLSTATISGLKPG |
| | | VDYTITVYAVTYGDDSAPISINYRTEID |
| 274 | 30 | MGHHHHHHGGSVSDVPRDLEVVAATPTSLLISWEYKV |
| | | DPWRYYRITYGETGGNSPVQEFTVPSVLSTATISGLKPG |
| | | VDYTITVYAVTYGDDSAPISINYRTEID |
| 275 | >1,000 | MGHHHHHHGGSVSDVPRDLEVVAATPTSLLISWEYKV |
| | | DYQRYYRITYGETGGNSPVQEFTVPSVLSTATISGLKPG |
| | | VDYTITVYAVTYGDDSAPISINYRTEID |
| 276 | >1,000 | MGHHHHHHGGSVSDVPRDLEVVAATPTSLLISWKYKV |
| | | HPYRYYRITYGETGGNSPVQEFTVPSVLSTATISGLKPG |
| | | VDYTITVYAVTYGDDSAPISINYRTEID |
| 277 | 132 | MGHHHHHHGGSVSDVPRDLEVVAATPTSLLISWEYXV |
| | | HPYRYYRITYGETGGNSPVQEFTVPSVLSTATISGLKPG |
| | | VDYTITVYAVTYGDDSAPISINYRTEID |
| 278 | >1,000 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTHGDDFAPISINYRTEMLI |
| 279 | 4 | MGHHHHHHGGSVSDVPRDLEVVAATPTSLLISWEYKV |
| | | HPYRYYRITYGETGGNSPVQEFTVPSVLSSATINGLKPG |
| | | VDYTITVYAVTYGDDSAPISINYRTEID |
| 280 | 109 | MGHHHHHHGGSVSDVPRDLEVVAATPTSLLISWEYKV |
| | | HPYRYYRITYGETGGNSPVQEFKVPSVLSTATISGLKPG |
| | | VDYTITVYAVTYGDDSAPISINYRTEID |
| 281 | >1,000 | MGHHHHHHGGSVSDVPRDLEVVAATPTSLLISWEYKV |
| | | AHDRYYRITYGETGGNSPVQEFTVPSVLSTATISGLKPG |
| | | VDYTITVYAVTYGDDSAPISINYRTEID |
| 282 | 83 | MGHHHHHHGGSVSDVPRDLEVVAATPTSLLISWEYKV |
| | | DPYRYYRITYGETGGNSPVQEFTVPSVLSTATISGLKPG |
| | | VDYTITVYAVTYGDDSAPISINYRTEID |
| 283 | >1,000 | MGHHHHHHGGSVSDVPRDLEVVAATPTSLLISWEYKV |
| | | DYRRYYRITYGETGGNSPVQEFTVPSVLSTATISGLKPG |
| | | VDYTITVYAVTYGDDSAPISINYRTEID |
| 284 | 205 | MGHHHHHHGGSVSDVPRDLEVVAATPTGLLISWEYKV |
| | | HPYRYYRITYGETGGNSPVQEFTVPSVLSTATISGLKPG |
| | | VDYTITVYAVTYGDDSAPISINYRTEID |
| 285 | >1,000 | MGHHHHHHGGSVSDVPRDLEVVAATPTSLLISWEYKV |
| | | NAWRYYRITYGETGGNSPVQEFTVPSVLSTATISGLKPG |
| | | VDYTITVYAVTYGDDSAPISINYRTEID |
| 286 | >1,000 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTHGDDSAPISINYRTEID |
| 287 | 62 | MGHHHHHHGGSVSDVPRDLEVVAATPTSLLISWEYKV |
| | | HPYRYYRITYGETGGNSPVQEFTVPSVLSTATINGLKPG |
| | | VDYTITVYAVTYGDDSAPISINYRTEID |
| 288 | 42 | MGHHHHHHGGSVSDVPRDLEVVAATPTSLLISWEYKV |
| | | HPYRYYRITYGETGGNSPVQEFTVPSVLSTAEISGLKPG |
| | | VDYTITVYAVTYGDDSAPISINYRTEID |
| 289 | 37 | MGHHHHHHGGSVSDVPRDLEVVAATPTSLLISWEYKV |
| | | HPYRYYRITYGETGGNSPVQEFLVPSVLSTATISGLKPG |
| | | VDYTITVYAVTYGDDSAPISINYRTEID |
| 290 | >1,000 | MGHHHHHHGGSVSDVPRDLEVVAATPTSLLISWEYKV |
| | | AHGRYYRITYGETGGNSPVQEFTVPSVLSTATISGLKPG |
| | | VDYTITVYAVTYGDDSAPISINYRTEID |
| 291 | >1,000 | MGHHHHHHGGSVSDVPRDLEVVAATPTSLLISWEYKV |
| | | AYKRYYRITYGETGGNSPVQEFTVPSVLSTATISGLKPG |
| | | VDYTITVYAVTYGDDSAPISINYRTEID |
| 292 | >1,000 | MGHHHHHHGGSVSDVPRDLEVVAATPTSLLISWEYKV |
| | | DRWRYYRITYGETGGNSPVQEFTVPSVLSTATISGLKPG |
| | | VDYTITVYAVTYGDDSAPISINYRTEID |
| 293 | 331 | MGHHHHHHGGSVSDVPRDLEVVAATPTSLLISWEYAV |
| | | HPYRYYRITYGETGGNSPVQEFTVPSVLSTATISGLKPG |
| | | VDYTITVYAVTYGDDSAPISINYRTEID |
| 294 | >1,000 | MGHHHHHHGGSVSDVPRDLEVVAATPTSLLISWEYKV |
| | | NAYRYYRITYGETGGNSPVQEFTVPSVLSTATISGLKPG |
| | | VDYTITVYAVTYGDDSAPISINYRTEID |
| 295 | >1,000 | MGHHHHHHGGSVSDVPRDLEVVAATPTSLLISWEYKV |
| | | NGYRYYRITYGETGGNSPVQEFTVPSVLSTATISGLKPG |
| | | VDYTITVYAVTYGDDSAPISINYRTEID |
| 296 | >1,000 | MGHHHHHHGGSVSDVPRDLEVVAATPTSLLISWEYKVS |
| | | HGRYYRITYGETGGNSPVQEFTVPSVLSTATISGLKPGV |
| | | DYTITVYAVTYGDDSAPISINYRTEID |
| 297 | >1,000 | MGHHHHHHGGSVSDVPRDLEVVAATPTSLLISWEYKV |
| | | TTWRYYRITYGETGGNSPVQEFTVPSVLSTATISGLKPG |
| | | VDYTITVYAVTYGDDSAPISINYRTEID |
| 298 | 55 | MGHHHHHHGGSVSDVPRDLEVVAATPTSLLISWEYKV |
| | | HPYRYYRITYGETGGNSPVQEFTVPSVLSSAZLSGLKPG |
| | | VDYTITVYAVTYGDDSAPISINYRTEID |
| 299 | 96 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGTDSAPISINYRTEID |
| 300 | 126 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGDQSAPISINYRTEID |
| 301 | 487 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGDDSNPISINYRTEID |
| 302 | >1,000 | MGHHHHHHGGSVSDVPRDLEVVAATPTSLLISWEYKV |
| | | NHDRYYRITYGETGGNSPVQEFTVPSVLSTATISGLKPG |
| | | VDYTITVYAVTYGDDSAPISINYRTEID |
| 303 | >1,000 | MGHHHHHHGGSVSDVPRDLEVVAATPTSLLISWEYKVS |
| | | HRRYYRITYGETGGNSPVQEFTVPSVLSTATISGLKPGV |
| | | DYTITVYAVTYGDDSAPISINYRTEID |
| 304 | >1,000 | MGHHHHHHGGSVSDVPRDLEVVAATPTSLLISWEYKV |
| | | TYERYYRITYGETGGNSPVQEFTVPSVLSTATISGLKPGV |
| | | DYTITVYAVTYGDDSAPISINYRTEID |
| 305 | 63 | MGHHHHHHGGSVSDVPRDLEVVAATPTSLLISWEYKV |
| | | HPYRYYRITYGETGGNSPVQEFTVPSVLSSATXXGLKPG |
| | | VDYTITVYAVTYGDDSAPISINYRTEID |
| 306 | 102 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGDVSAPISINYRTEID |
| 307 | 99 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGDDSPPISINYRTEID |
| 308 | 100 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGDDSXPISINYRTEID |
| 309 | >1,000 | MGHHHHHHGGSVSDVPRDLEVVAATPTSLLISWEYKV |
| | | NRWRYYRITYGETGGNSPVQEFTVPSVLSTATISGLKPG |
| | | VDYTITVYAVTYGDDSAPISINYRTEID |
| 310 | >1,000 | MGHHHHHHGGSVSDVPRDLEVVAATPTSLLISWEYKVS |
| | | PWRYYRITYGETGGNSPVQEFTVPSVLSTATISGLKPGV |
| | | DYTITVYAVTYGDDSAPISINYRTEID |
| 311 | >1,000 | MGHHHHHHGGSVSDVPRDLEVVAATPTSLLISWEYKV |
| | | TYRRYYRITYGETGGNSPVQEFTVPSVLSTATISGLKPG |
| | | VDYTITVYAVTYGDDSAPISINYRTEID |
| 312 | 112 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGDESAPISINYRTEID |
| 313 | 171 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGDDSDPISINYRTEID |
| 314 | >1,000 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGDDSYPISINYRTEID |
| 315 | >1,000 | MGHHHHHHGGSVSDVPRDLEVVAATPTSLLISWEYKV |
| | | NRYRYYRITYGETGGNSPVQEFTVPSVLSTATISGLKPG |
| | | VDYTITVYAVTYGDDSAPISINYRTEID |
| 316 | >1,000 | MGHHHHHHGGSVSDVPRDLEVVAATPTSLLISWEYKVS |
| | | RWRYYRITYGETGGNSPVQEFTVPSVLSTATISGLKPGV |
| | | DYTITVYAVTYGDDSAPISINYRTEID |
| 317 | >1,000 | MGHHHHHHGGSVSDVPRDLEVVAATPTSLLISWEYKV |
| | | XXYRYYRITYGETGGNSPVQEFTVPSVLSTATISGLKPG |
| | | VDYTITVYAVTYGDDSAPISINYRTEID |
| 318 | >1,000 | MGHHHHHHGGSVSDVPRDLEVVAATPTSLLISWEYKV |
| | | HPYRYYRITYGETGGNSPVQSFTVPSVLSTATISGLKPGV |
| | | DYTITVYAVTYGDDSAPISINYRTEID |
| 319 | >1,000 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGIDSAPISINYRTEID |
| 320 | 122 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGDHSAPISINYRTEID |
| 321 | >1,000 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGDDSEPISINYRTEID |
| 322 | >1,000 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGDDSRPISINYRTEID |
| 323 | 83 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGDDSAPISIQYRTEID |
| 324 | >1,000 | MGHHHHHHGGSVSDVPRDLEVVAATPTSLLISWEYKV |
| | | NYRRYYRITYGETGGNSPVQEFTVPSVLSTATISGLKPG |
| | | VDYTITVYAVTYGDDSAPISINYRTEID |
| 325 | >1,000 | MGHHHHHHGGSVSDVPRDLEVVAATPTSLLISWEYKVS |
| | | TWRYYRITYGETGGNSPVQEFTVPSVLSTATISGLKPGV |
| | | DYTITVYAVTYGDDSAPISINYRTEID |
| 326 | >1,000 | MGHHHHHHGGSVSDVPRDLEVVAATPTSLLISWEYKV |
| | | YAYRYYRITYGETGGNSPVQEFTVPSVLSTATISGLKPG |
| | | VDYTITVYAVTYGDDSAPISINYRTEID |
| 327 | 191 | MGHHHHHHGGSVSDVPRDLEVVAATPTSLLISWEYKV |
| | | HPYRYYRITYGETGGNSPVQTFTVPSVLSTATISGLKPG |
| | | VDYTITVYAVTYGDDSAPISINYRTEID |
| 328 | 215 | MGHHHHHHGGSVSDVPRDLEVVAATPTSLLISWEYKV |
| | | HPYRYYRITYGETGGNSPVQAFTVPSVLSTATISGLKPG |
| | | VDYTITVYAVTYGKDSAPISINYRTEID |
| 329 | 77 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGDLSAPISINYRTEID |
| 330 | 19 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGDDSGPISINYRTEID |
| 331 | >1,000 | MGHHHHHHGGSVSDVPRDLEVVAATPTSLLISWEYKVS |
| | | AWRYYRITYGETGGNSPVQEFTVPSVLSTATISGLKPGV |
| | | DYTITVYAVTYGDDSAPISINYRTEID |
| 332 | >1,000 | MGHHHHHHGGSVSDVPRDLEVVAATPTSLLISWEYKVS |
| | | TYRYYRITYGETGGNSPVQEFTVPSVLSTATISGLKPGV |
| | | DYTITVYAVTYGDDSAPISINYRTEID |
| 333 | 31 | MGHHHHHHGGSVSDVPRDLEVVAATPTSLLISWEYKV |
| | | HPYRYYRITYGETGGNSPVQEFTVPSVLSTAELSGLKPG |
| | | VDYTITVYAVTYGDDSAPISINYRTEID |
| 334 | 178 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGDLSAPISINYRTEID |
| 335 | >1,000 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGDDSTPISINYRTEID |
| 336 | >1,000 | MGHHHHHHGGSVSDVPRDLEVVAATPTSLLISWEYKVS |
| | | AYRYYRITYGETGGNSPVQEFTVPSVLSTATISGLKPGV |
| | | DYTITVYAVTYGDDSAPISINYRTEID |
| 337 | >1,000 | MGHHHHHHGGSVSDVPRDLEVVAATPTSLLISWEYKV |
| | | TGYRYYRITYGETGGNSPVQEFTVPSVLSTATISGLKPG |
| | | VDYTITVYAVTYGDDSAPISINYRTEID |
| 338 | >1,000 | MGHHHHHHGGSVSDVPRDLEVVAATPTSLLISWEYKV |
| | | YHGRYYRITYGETGGNSPVQEFTVPSVLSTATISGLKPG |
| | | VDYTITVYAVTYGDDSAPISINYRTEID |
| 339 | 121 | MGHHHHHHGGSVSDVPRDLEVVAATPTSLLISWEYKV |
| | | HPYRYYRITYGETGGNSPVQEFTVPSVLSTAKLSGLKPG |
| | | VDYTITVYAVTYGDDSAPISINYRTEID |
| 340 | 130 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGNDSAPISINYRTEID |
| 341 | 89 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGDPSAPISINYRTEID |
| 342 | >1,000 | MGHHHHHHGGSVSDVPRDLEVVAATPTSLLISWEYKVS |
| | | GYRYYRITYGETGGNSPVQEFTVPSVLSTATISGLKPGV |
| | | DYTITVYAVTYGDDSAPISINYRTEID |
| 343 | >1,000 | MGHHHHHHGGSVSDVPRDLEVVAATPTSLLISWEYKV |
| | | YHNRYYRITYGETGGNSPVQEFTVPSVLSTATISGLKPG |
| | | VDYTITVYAVTYGDDSAPISINYRTEID |
| 344 | 300 | MGHHHHHHGGSVSDVPRDLEVVAATPTSLLISWEYKV |
| | | HPYRYYRITYGETGGNSPVQEFTVPSVLSSAQLSGLKPG |
| | | VDYTITVYAVTYGDDSAPISINYRTEID |
| 345 | 62 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGTDSAPISINYRTEID |
| 346 | 155 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGDPSAPISINYRTEID |
| 347 | 335 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGDDSKPISINYRTEID |
| 348 | 114 | MGHHHHHHGGSVSDVPRDLEVVAATPTSLLISWEYKV |
| | | HPYRYYRITYGETGGNSPVQEFTVPSVLSTATISGLKPG |
| | | VDYTITVYAVTYGIDSTPISINYRTEID |

In some embodiments, the N17 Adnectin™ of the invention comprises an amino acid sequence at least 70%, 75%, 80%, 85%, 90%, 95%, 98%, 99% or 100% identical to any one of SEQ ID NOs: 92-348.

In some embodiments, the anti-N 17 Adnectin™ of the invention comprises an amino acid sequence at least 70%, 75%, 80%, 85%, 90%, 95%, 98%, 99% or 100% identical to any one of SEQ ID NOs: 92-348, excluding any N-terminus extended region.

In some embodiments, the N17 Adnectin™ of the invention comprises an amino acid sequence at least 70%, 75%, 80%, 85%, 90%, 95%, 98%, 99% or 100% identical to any one of SEQ ID NOs: 92-348, excluding any C-terminus extended region.

In some embodiments, the N17 Adnectin™ of the invention comprises an amino acid sequence at least 70%, 75%, 80%, 85%, 90%, 95%, 98%, 99% or 100% identical to any one of SEQ ID NOs: 92-348, excluding both the N-terminus and C-terminus extended regions.

In other embodiments, N17 Adnectin™ comprises an amino acid sequence at least 80%, 85%, 90%, 95%, 98%, 99% or 100% identical to the BC loop, DE loop and FG loop regions of SEQ ID NOs: 92-348.

Analysis of the sequences above indicate that S52, V53, L54 and S55 of the N17 Adnectin™ DE loop of SEQ ID NO: 1 are conserved amino acids. In some embodiments, the N17 Adnectin™ comprises one or more of the conserved amino acids S52, V53, L54 and S55 of SEQ ID NO: 1. Additionally, analysis of the sequences above indicates that Y24 of the anti-CD4 Adnectin™ BC loop of SEQ ID NO: 1 is a conserved amino acid. In some embodiments, position 26 of the N17 Adnectin™ BC loop of SEQ ID NO: 1 is valine or leucine.

Analysis of the sequences above indicates that G78 and S85 of the anti-N17 Adnectin™ FG loop of SEQ ID NO: 1 are conserved amino acids. In some embodiments, the N17 Adnectin™ comprises one or more of the conserved amino acids G78 and S85. In some embodiments, position 79 of the N17 Adnectin™ FG loop of SEQ ID NO: 1 is valine or isoleucine. In some embodiments, the N17 Adnectin™ comprises one or more of the conserved amino acids Y24, V26, L26, S52, V53, L54, S55 G78, V79, 179 and S85 of SEQ ID NO: 1.

Point mutation analysis of the N17 Adnectin™ showed advantages of mutating several ¹⁰Fn3 non-loop scaffold positions. Specifically, mutating positions T56 and T58 of SEQ ID NO: 1 boosted potency. In some embodiments, the N17 Adnectin™ comprises mutating T58 of SEQ ID NO: 1 to Asn, Glu, or Gln.

### II. HIV Fusion Peptide Inhibitors

The amino acid sequence of gp41, and its variation among different strains of HIV-1, is well known. The fusogenic domain (often called the fusion peptide, or FP) is believed to be involved in insertion into and disruption of the target cell membrane. The transmembrane domain, containing the transmembrane anchor sequence, is located toward the C-terminal end of the protein. Between the fusogenic domain and transmembrane anchor are two distinct regions, known as heptad repeat (HR) regions, each region having a plurality of heptads. The amino acid sequence comprising the HR1 region and the amino acid sequence comprising the HR2 region are each relatively conserved regions in the HIV-1 envelope protein. A representative sequence of the external domain of gp41 (clade B consensus) is as follows:

| (SEQ ID NO: 2) | |
|---|---|
| 512 | AVGIGAMFL GFLGAAGSTM GAASVTLTVQ ARQLLSGIVQ QQNNLLRAIE |
| 561 | AQQHLLQLTV WGIKQLQARV LAVERYLKDQ QLLGIWGCSG KLICTTAVPW |
| 611 | NASWSNKSLD EIWNNMTWME WEREIDNYTG LIYTLIEESQ NQQEKNEQEL |
| 661 | LELDKWASLW NWFDITNWLW YIK |

The fusion peptide comprises approximately the first 23 amino acids, Ala512-Ser534. The HR1 region has a plurality of contiguous 7 amino acid residue stretches or “heptads” (the 7 amino acids in each heptad designated “a” through “g”), with a predominance of hydrophobic residues at the first (“a”) and fourth (“d”) positions which interact homotypically to form the core of the 3-helix bundle structure. Neutral polar amino acids such as Glutamine may also occupy these positions. One representative heptad begins with Leu545. A highly conserved portion of HR1 consists of the 17 residues from Leu565 to Leu581, and is called “N17”.

The C-terminal portion of gp41 comprises the HR2 region, which is believed to form an alpha helical structure during the fusion process, and bind into the grooves of the HR1 triple helical structure. HR2 also comprises heptads, though they do not interact homotypically but rather interact with amino acids from HR1. One representative heptad begins at Trp628.

### HIV Fusion Peptide Inhibitor

The HIV fusion peptide inhibitors of the invention include but are not limited to the following sequences in Table 6:

TABLE 6

| | SEQ |
|---|---|
| | ID |
| HIV Fusion Peptide Inhibitor Sequence | NO |
| SEYEARIEALIRAAQEQQEKNEAALRELYKWAL | 349 |
| SEYEARIEALIRAAQEQQEKNEAALRELWKWAS | 350 |
| TIAEYAARIEALIRAAQEQQEKNEAALRELYKWAS | 351 |
| ARIEEYAARIEALIRAAQEQQEKNEAALRELYKWAS | 352 |
| SEYEARIEALIRAAQEQQEKNEAALRELYKWAS | 353 |
| TEYEARIEALIRAAQEQQEKNEAALRELKEWASIWN | 354 |
| SEYEARIEALIRAAQEQQEKNEAALRELDKWTGVWGNYEKV | 355 |
| SRIEALIRAAQEQQEKNEAALRELFKWAS | 356 |
| SRIEALIRAAQEQQQKNEAALRELDKWAS | 357 |
| SRIEALIRAAQEQQEKNEAALRELYKWAS | 358 |
| SRIEALIRAAQEQQEKNEAALRELLKWAS | 359 |
| SRIEALIRAAQEQQEKNEAALRELQKWAS | 360 |
| SRIEALIRAAQEQQEKNEAALRELDKWAS | 361 |
| AIAEYAARIEALIRAAQEQQEKNEAALRELDKWAS | 362 |
| TEYEARIEALIRAAQEQQEKNEAALRELDK | 363 |
| SRIEALIRAAQEQQEKNEAALRELYKWTS | 364 |
| SRIEALIRAAQEQQEKNEAALRELYKWASLWI | 365 |
| SRIEALIRAAQEQQEKNEAALRELYKWASRWN | 366 |
| SRIEALIRAAQEQQEKNEAALRELYKWASSWN | 367 |
| SRIEALIRAAQEQQEKNEAALRELYKWGS | 368 |
| SRIEALIRAAQEQQEKNEAALRELDK | 369 |

In some embodiments, the HIV fusion peptide inhibitor comprises an amino acid sequence at least 70%, 75%, 80%, 85%, 90%, 95%, 98%, 99% or 100% identical to any one of SEQ ID NOs: 349-369.

Point mutations in the C-terminal region of the HIV fusion peptide inhibitor were seen to boost potency. In some embodiments, a hydrophobic replacement of the aspartic acid (D) near the C-terminus of the HIV fusion peptide inhibitor provides at least a 10-fold increase in potency. In some embodiments, the HIV fusion peptide inhibitor comprises replacing “DK” with “YK”, “LK”, “FK” or “WK”.

Other point mutation studies in the C-terminal region showed how the C-terminal amino acids “WAS” can be mutated with good effects on potency. In some embodiments, the HIV fusion peptide inhibitor comprises replacing C-terminal amino acids “WAS” to “WFS” or “WAL”.

### III. Linkers

The various components of the polypeptide of the invention may be covalently or non-covalently linked. In some embodiments, the PK moiety may be directly or indirectly linked to a polypeptide of the invention via a polypeptide linker. Suitable linkers are those which allow the separate domains to fold independently of each other forming a three-dimensional structure that permits high affinity binding to a target molecule.

In some embodiments, the components (e.g., an Adnectin™ and an anti-HIV bnAb) may be directly or indirectly linked via a polypeptide linker. Suitable linkers are those which allow the separate domains (e.g., of an Adnectin™ and/or an anti-HIV bnAb) to fold independently of each other forming a three-dimensional structure that permits high affinity binding to a target molecule.

The disclosure provides a number of suitable linkers that meet these requirements, including glycine-serine based linkers, glycine-proline based linkers. The Examples described herein are designed such that the polypeptide of the invention domains joined via polypeptide linkers retain their target binding function. In some embodiments, the linker is a glycine-serine based linker. These linkers comprise glycine and serine residues and may be between 8 and 50, 10 and 30, and 10 and 20 amino acids in length. Examples include linkers having an amino acid sequence (GS)7 (SEQ ID NO: 370), G(GS)6 (SEQ ID NO: 371), and G(GS)7G (SEQ ID NO: 372). Other linkers contain glutamic acid, and include, for example, (GSE)5 (SEQ ID NO: 373) and GGSEGGSE (SEQ ID NO: 374). Other exemplary glycine-serine linkers include (GS)4 (SEQ ID NO: 375), (GGGGS)7 (SEQ ID NO: 376), (GGGGS)5 (SEQ ID NO: 377), (GGGGS)4 (SEQ ID NO: 378 (GGGGS)3G (SEQ ID NO: 379). In some embodiments, the linker is a glycine-proline based linker.

These linkers comprise glycine and proline residues and may be between 3 and 30, 10 and 30, and 3 and 20 amino acids in length. Examples include linkers having an amino acid sequence (GP)3G (SEQ ID NO: 380) and (GP)5G (SEQ ID NO: 381). In other embodiments, the linker may be a proline-alanine based linker having between 3 and 30, 10 and 30, and 3 and 20 amino acids in length. Examples of proline alanine based linkers include, for example, (PA)3 (SEQ ID NO: 382), (PA)6 (SEQ ID NO: 383) and (PA)9 (SEQ ID NO: 384). In some embodiments, the linker is a glutamic acid-proline based linker. These linkers comprise glutamic acid and proline residues and may be between 3 and 30, 10 and 30, and 3 and 20 amino acids in length. Examples include linkers having an amino acid sequence ESPEPETPEDE (SEQ ID NO: 385) and (ESPEPETPED)2E(SEQ ID NO: 386).

Anti-HIV bnAbs of the present invention may be linked to the CD4 Adnectin™ and/or an N17 Adnectin™—i.e. an epitope binding domain—by linkers comprised of amino acid sequences which may be from 1 amino acid to 150 amino acids in length, or from 1 amino acid to 140 amino acids, for example, from 1 amino acid to 130 amino acids, or from 1 to 120 amino acids, or from 1 to 80 amino acids, or from 1 to 50 amino acids, or from 1 to 20 amino acids, or from 1 to 10 amino acids, or from 5 to 18 amino acids. Such sequences may have their own tertiary structure, for example, a linker of the present invention may comprise a single variable domain. The size of a linker in one embodiment is equivalent to a single variable domain. Suitable linkers may be of a size from 1 to 20 angstroms, for example less than 15 angstroms, or less than 10 angstroms, or less than 5 angstroms.

In one embodiment of the present invention at least one of the epitope binding domains is directly attached to the anti-HIV bnAb with a linker comprising from 1 to 150 amino acids, for example 1 to 20 amino acids, for example 1 to 10 amino acids. Such linkers may be selected from any one of those set out in SEQ ID NOs: 370-415, shown in Table 7, or multiples of such linkers.

TABLE 7

| | SEQ |
|---|---|
| | ID |
| Linker Sequence | NO: |
| (GS)₇ | 370 |
| G(GS)₆ | 371 |
| G(GS)₆G | 372 |
| (GSE)₅ | 373 |
| GGSEGGSE | 374 |
| (GS)₄ | 375 |
| (GGGGS)₇ | 376 |
| (GGGGS)₅ | 377 |
| (GGGGS)₄ | 378 |
| (GGGGS)₃ | 379 |
| (GP)₃G | 380 |
| (GP)₅G | 381 |
| (PA)₃ | 382 |
| (PA)₆ | 383 |
| (PA)₉ | 384 |
| ESPEPETPEDE | 385 |
| (ESPEPETPED)₂E | 386 |
| GGGGS | 387 |
| TVAAPS | 388 |
| ASTKGPT | 389 |
| ASTKGPS | 390 |
| GS | 391 |
| TVAAPSGS | 392 |
| GSTVAAPS | 393 |
| GSTVAAPSGS | 394 |
| GSTVAAPSGSTVAAPSGS | 395 |
| GSTVAAPSGSTVAAPSGSTVAAPSGS | 396 |
| GSTVAAPSGSTVAAPSGSTVAAPSGSTVAAPSGS | 397 |
| GSTVAAPSGSTVAAPSGSTVAAPSGSTVAAPSGSTVAAPSG | 398 |
| S | |
| GSTVAAPSGSTVAAPSGSTVAAPSGSTVAAPSGSTVAAPSG | 399 |
| STVAAPSGS | |
| TVAAPSTVAAPSGS | 400 |
| TVAAPSTVAAPSTVAAPSGS | 401 |
| ELQLEESAAEAQEGELE | 402 |
| (EAAAK)₃ | 403 |
| EEEKRAAEERQRIK | 404 |
| GSGS | 405 |
| (ESPEPETPED)₃E | 406 |
| (G4S)₅ | 407 |
| (PAS)ₙ(GS)ₘ | 408- |
| | 457 |
| (GGGGS)ₙ(GS)ₘ | 458- |
| | 507 |
| (TVAAPS)ₙ(GS)ₘ | 508- |
| | 554 |
| (GS)ₘ(TVAAPSGS)ₙ | 555- |
| | 598 |
| (PAVPPP)ₙ(GS)ₘ | 599- |
| | 648 |
| (TVSDVP)ₙ(GS)ₘ | 649- |
| | 698 |
| (TGLDSP)ₙ(GS)ₘ | 699- |
| | 748 |

Linkers of use in the antigen-binding proteins of the present invention may comprise alone or in addition to other linkers, one or more sets of GS residues, for example ‘GSTVAAPS’ (SEQ ID NO: 393) or ‘TVAAPSGS’ (SEQ ID NO: 392) or ‘GSTVAAPSGS’ (SEQ ID NO: 394). In one embodiment the linker comprises SEQ ID NO: 388.

In one embodiment the epitope binding domain anti-CD4 Adnectin™ is linked to the anti-HIV bnAb by the linker ‘(PAS)ₙ(GS)ₘ’ (SEQ ID NOs: 408-457). In another embodiment the anti-CD4 Adnectin™ is linked to an anti-HIV bnAb by the linker ‘(GGGGS)ₙ(GS)ₘ’ (SEQ ID NOs: 458-507). In another embodiment the anti-CD4 Adnectin™ is linked to an anti-HIV bnAb by the linker ‘(TVAAPS)ₙ(GS)ₘ’ (SEQ IN NOs: 508-554). In another embodiment the epitope binding domain is linked to an anti-HIV bnAb by the linker ‘(GS)ₘ(TVAAPSGS)ₙ’ (SEQ ID NOs: 555-598). In another embodiment the epitope binding domain is linked to the anti-HIV bnAb by the linker ‘(PAVPPP)ₙ(GS)ₘ’ (SEQ ID NOs: 599-648). In another embodiment the epitope binding domain is linked to the anti-HIV bnAb by the linker ‘(TVSDVP)ₙ (GS)ₘ’ (SEQ ID NOs: 649-698). In another embodiment the epitope binding domain is linked to the anti-HIV bnAb by the linker ‘(TGLDSP)ₙ(GS)ₘ’ (SEQ ID NOs: 699-748). In all such embodiments described by SEQ ID NOs: 408-757, n=1-10, and m=0-4.

Examples of such linkers include (PAS)ₙ(GS)ₘ wherein n=1 and m=1 (SEQ ID NO: 418), (PAS)ₙ(GS)ₘ wherein n=2 and m=1 (SEQ ID NO: 419), (PAS)ₙ(GS)ₘ wherein n=3 and m=1 (SEQ ID NO: 420), (PAS)ₙ(GS)ₘ wherein n=4 and m=1 (SEQ ID NO: 421, (PAS)ₙ(GS)ₘ wherein n=2 and m=0 (SEQ ID NO: 409), (PAS)ₙ(GS)ₘ wherein n=3 and m=0 (SEQ ID NO: 410), (PAS)ₙ(GS)ₘ wherein n=4 and m=0 (SEQ ID NO: 411).

Examples of such linkers include (GGGGS)ₙ(GS)ₘ wherein n=1 and m=1 (SEQ ID NO: 468), (GGGGS)ₙ(GS)ₘ wherein n=2 and m=1 (SEQ ID NO: 469), (GGGGS)ₙ(GS)ₘ wherein n=3 and m=1 (SEQ ID NO: 470), (GGGGS)ₙ(GS)ₘ wherein n=4 and m=1 (SEQ ID NO: 471), (GGGGS)ₙ(GS)ₘ wherein n=2 and m=0 (SEQ ID NO: 459), (GGGGS)ₙ(GS)ₘ wherein n=3 and m=0 (SEQ ID NO: 460), (GGGGS)ₙ(GS)ₘ wherein n=4 and m=0 (SEQ ID NO: 461).

Examples of such linkers include (TVAAPS)ₙ(GS)ₘ wherein n=1 and m=1 (SEQ ID NO:392), (TVAAPS)ₙ(GS)ₘ wherein n=2 and m=1 (SEQ ID NO:400), (TVAAPS)ₙ(GS)ₘ wherein n=3 and m=1 (SEQ ID NO:401), (TVAAPS)ₙ(GS)ₘ wherein n=4 and m=1 (SEQ ID NO: 518), (TVAAPS)ₙ(GS)ₘ wherein n=2 and m=0 (SEQ ID NO: 509), (TVAAPS)ₙ(GS)ₘ wherein n=3 and m=0 (SEQ ID NO: 510), (TVAAPS)ₙ(GS)ₘ wherein n=4 and m=0 (SEQ ID NO: 511).

Examples of such linkers include (GS)ₘ(TVAAPSGS)ₙ wherein n=1 and m=1 (SEQ ID NO: 394), (GS)ₘ(TVAAPSGS)ₙ wherein n=2 and m=1 (SEQ ID NO: 395), (GS)ₘ(TVAAPSGS)ₙ wherein n=3 and m=1 (SEQ ID NO: 396), or (GS)ₘ(TVAAPSGS)ₙ wherein n=4 and m=1 (SEQ ID NO:397), (GS)ₘ(TVAAPSGS)ₙ wherein n=5 and m=1 (SEQ ID NO: 398), (GS)ₘ(TVAAPSGS)ₙ wherein n=6 and m=1 (SEQ ID NO: 399), (GS)ₘ(TVAAPSGS)ₙ wherein n=1 and m=0 (SEQ ID NO:555), (GS)ₘ(TVAAPSGS)ₙ wherein n=2 and m=4 (SEQ ID NO: 592), (GS)ₘ(TVAAPSGS)ₙ wherein n=3 and m=0 (SEQ ID NO: 557), or (GS)ₘ(TVAAPSGS)ₙ wherein n=2 and m=5 (SEQ ID NO: 593).

Examples of such linkers include (TGLDSP)ₙ(GS)ₘ wherein n=1 and m=1 (SEQ ID NO: 709), (TGLDSP)ₙ(GS)ₘ wherein n=2 and m=1 (SEQ ID NO: 710), (TGLDSP)ₙ(GS)ₘ wherein n=3 and m=1 (SEQ ID NO: 711), (TGLDSP)ₙ(GS)ₘ wherein n=4 and m=1 (SEQ ID NO: 712), (TGLDSP)ₙ(GS)ₘ wherein n=2 and m=0 (SEQ ID NO: 700), (TGLDSP)ₙ(GS)ₘ wherein n=3 and m=0 (SEQ ID NO: 701, (TGLDSP)ₙ(GS)ₘ wherein n=4 and m=0 (SEQ ID NO: 702).

In another embodiment there is no linker between the epitope binding domain and the anti-HIV bnAb. In another embodiment the epitope binding domain is linked to the anti-HIV bnAb by the linker ‘TVAAPS’ (SEQ ID NO: 388). In another embodiment the epitope binding domain, is linked to the anti-HIV bnAb by the linker ‘TVAAPSGS’ (SEQ ID NO: 392). In another embodiment the epitope binding domain is linked to the anti-HIV bnAb by the linker ‘GS’. In another embodiment the epitope binding domain is linked to the anti-HIV bnAb by the linker ‘ASTKGPT’ (SEQ ID NO: 389). It is contemplated, that the optimal linker length and its amino acid composition may be determined by routine experimentation by methods well known in the art.

Linkers or spacers, may be introduced at the N-terminus of the anti-CD4 moiety or at the C-terminus of the anti-CD4 moiety. The anti-HIV bnAb may be linked to either the N-terminus or the C-terminus of the anti-CD4 moiety. In some embodiments the preferred linkers between the anti-CD4 Adnectin™ and N17 Adnectin™ are short glutamine-proline rich linkers. In some embodiments the preferred linker between the N17 Adnectin™ and HIV fusion peptide inhibitor are flexible glycine-serine rich linkers.

10E8v4-HC-(G4S)5-CD4-A(anti-CD4 A linked to the heavy chain of the 10E8v4 bnAb)-
SEQ ID NO: 9
EVRLVESGGGLVKPGGSLRLSCSASGFDFDNAWMTWVRQPPGKGLEWVGRITGPGEGWSVDYAESVKGRFTI
SRDNTKNTLYLEMNNVRTEDTGYYFCARTGKYYDFWSGYPPGEEYFQDWGQGTLVIVSSASTKGPSVFPLAPSS
KSTSGGTAALGCLVKDYFPEPVTVSWNSGALTSGVHTFPAVLQSSGLYSLSSVVTVPSSSLGTQTYICNVNHKPS
NTKVDKKVEPKSCDKTHTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFFLYSKLTVDKSRWQQGNVFSCSVM
HEALHNHYTQKSLSLSPGGGGSGGGGSGGGGSGGGGSGGGGSGVSDVPRDLEVVAATPTSLLISWDAPAVTVH
SYHIQYWPLGSYQRYQVFSVPGSKSTATISGLKPGVEYQIRVYAETGGADSDQSFGWIQIGYRTP
10E8v4-HC-ESPEPETED2-CD4 A (anti-CD4 A linked to the heavy chain of the 10E8v4 bnAb)-
SEQ ID NO: 10
EVRLVESGGGLVKPGGSLRLSCSASGFDFDNAWMTWVRQPPGKGLEWVGRITGPGEGWSVDYAESVKGRFTI
SRDNTKNTLYLEMNNVRTEDTGYYFCARTGKYYDFWSGYPPGEEYFQDWGQGTLVIVSSASTKGPSVFPLAPSS
KSTSGGTAALGCLVKDYFPEPVTVSWNSGALTSGVHTFPAVLQSSGLYSLSSVVTVPSSSLGTQTYICNVNHKPS
NTKVDKKVEPKSCDKTHTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFFLYSKLTVDKSRWQQGNVFSCSVM
HEALHNHYTQKSLSLSPESPEPETPEDESPEPETPEDEGVSDVPRDLEVVAATPTSLLISWDAPAVTVHSYHIQYWP
LGSYQRYQVFSVPGSKSTATISGLKPGVEYQIRVYAETGGADSDQSFGWIQIGYRTP
10E8v4-LC-(G4S)5-CD4 A (anti-CD4 A linked to the light chain of the 10E8v4 bnAb)-
SEQ ID NO: 11
ASELTQDPAVSVALKQTVTITCRGDSLRSHYASWYQKKPGQAPVLLFYGKNNRPSGIPDRES
GSASGNRASLTITGAQAEDEADYYCSSRDKSGSRLSVFGGGTKLTVLSQPKAAPSVTLFPPS
SEELQANKATLVCLISDFYPGAVTVAWKADSSPVKAGVETTTPSKOSNNKYAASSYLSLTPE
QWKSHRSYSCOVTHEGSTVEKTVAPTECSGGGGSGGGGSGGGGSGGGGSGGGGSGVSDVPRDLEVVA
ATPTSLLISWDAPAVTVHSYHIQYWPLGSYQRYQVFSVPGSKSTATISGLKPGVEYQIRVYAETGGADSDQSFGWI
QIGYRTP
10E8v4-LC-ESPEPETED2-CD4 A(anti-CD4 A linked to the light chain of the 10E8v4 bnAb)-
SEQ ID NO: 12
ASELTQDPAVSVALKQTVTITCRGDSLRSHYASWYQKKPGQAPVLLFYGKNNRPSGIPDRES
GSASGNRASLTITGAQAEDEADYYCSSRDKSGSRLSVFGGGTKLTVLSQPKAAPSVTLFPPS
SEELQANKATLVCLISDFYPGAVTVAWKADSSPVKAGVETTTPSKQSNNKYAASSYLSLTPE
QWKSHRSYSCQVTHEGSTVEKTVAPTECSESPEPETPEDESPEPETPEDEGVSDVPRDLEVVAATPTSLLIS
WDAPAVTVHSYHIQYWPLGSYQRYQVFSVPGSKSTATISGLKPGVEYQIRVYAETGGADSDQSFGWIQIGYRTP
10E8v4-HC-(G4S)5-CD4 B (anti-CD4 B linked to the heavy chain of the 10E8v4 bnAb)-
SEQ ID NO: 764
EVRLVESGGGLVKPGGSLRLSCSASGFDFDNAWMTWVRQPPGKGLEWVGRITGPGEGWSVDYAESVKGRFTI
SRDNTKNTLYLEMNNVRTEDTGYYFCARTGKYYDFWSGYPPGEEYFQDWGQGTLVIVSSASTKGPSVFPLAPSS
KSTSGGTAALGCLVKDYFPEPVTVSWNSGALTSGVHTFPAVLQSSGLYSLSSVVTVPSSSLGTQTYICNVNHKPS
NTKVDKKVEPKSCDKTHTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFFLYSKLTVDKSRWQQGNVFSCSVM
HEALHNHYTQKSLSLSPGGGGSGGGGSGGGGSGGGGSGGGGSMASTSGSASYLIPSDLEVVAATPTSLSIYWYPV
ASTIINFRITYVETGGNSPVQEFTVPGSKSTATISGLKPGVDYTITVYAVHYEQKYSEYWIGHPISINYRTEGSGSHHH
HHH
10E8v4-HC-ESPEPETED2-CD4 B(anti-CD4 B linked to the heavy chain of the 10E8v4 bnAb)-
SEQ ID NO: 765
EVRLVESGGGLVKPGGSLRLSCSASGFDFDNAWMTWVRQPPGKGLEWVGRITGPGEGWSVDYAESVKGRFTI
SRDNTKNTLYLEMNNVRTEDTGYYFCARTGKYYDFWSGYPPGEEYFQDWGQGTLVIVSSASTKGPSVFPLAPSS
KSTSGGTAALGCLVKDYFPEPVTVSWNSGALTSGVHTFPAVLQSSGLYSLSSVVTVPSSSLGTQTYICNVNHKPS
NTKVDKKVEPKSCDKTHTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFFLYSKLTVDKSRWQQGNVFSCSVM
HEALHNHYTQKSLSLSPESPEPETPEDESPEPETPEDEMASTSGSASYLIPSDLEVVAATPTSLSIYWYPVASTIINFRI
TYVETGGNSPVQEFTVPGSKSTATISGLKPGVDYTITVYAVHYEQKYSEYWIGHPISINYRTEGSGSHHHHHH
10E8v4-LC-(G4S)5-CD4 B(anti-CD4 B linked to the light chain of the 10E8v4 bnAb)-
SEQ ID NO: 766
ASELTQDPAVSVALKQTVTITCRGDSLRSHYASWYQKKPGQAPVLLFYGKNNRPSGIPDRFSGSASGNRASLTIT
GAQAEDEADYYCSSRDKSGSRLSVFGGGTKLTVLSQPKAAPSVTLFPPSSEELQANKATLVCLISDFYPGAVTVA
WKADSSPVKAGVETTTPSKQSNNKYAASSYLSLTPEQWKSHRSYSCQVTHEGSTVEKTVAPTECSGGGGSGGG
GSGGGGSGGGGSGGGGSMASTSGSASYLIPSDLEVVAATPTSLSIYWYPVASTIINFRITYVETGGNSPVQEFTVPG
SKSTATISGLKPGVDYTITVYAVHYEQKYSEYWIGHPISINYRTEGSGSHHHHHH
10E8v4-LC-ESPEPETED2-CD4 B(anti-CD4 B linked to the light chain of the 10E8v4 bnAb)-
SEQ ID NO: 767
ASELTQDPAVSVALKQTVTITCRGDSLRSHYASWYQKKPGQAPVLLFYGKNNRPSGIPDRFSGSASGNRASLTIT
GAQAEDEADYYCSSRDKSGSRLSVFGGGTKLTVLSQPKAAPSVTLFPPSSEELQANKATLVCLISDFYPGAVTVA
WKADSSPVKAGVETTTPSKQSNNKYAASSYLSLTPEQWKSHRSYSCQVTHEGSTVEKTVAPTECSESPEPETPED
ESPEPETPEDEMASTSGSASYLIPSDLEVVAATPTSLSIYWYPVASTIINFRITYVETGGNSPVQEFTVPGSKSTATISGL
KPGVDYTITVYAVHYEQKYSEYWIGHPISINYRTEGSGSHHHHHH

In SEQ ID NOs: 9-12 and SEQ ID NOs: 764-767 above, the anti-CD4 Adnectin™ sequences are underlined. The anti-HIV bnAb portion is indicated in bold. The linker sequences are italicized.

### IV. Pharmacokinetic Moieties

In one aspect, the application provides for a polypeptide comprising an anti-CD4 moiety and an anti-HIV bnAb, and combinations thereof, wherein the polypeptide is modified to provide improved pharmacokinetics relative to unmodified polypeptide. Improved pharmacokinetics may be assessed according to the perceived therapeutic need. Often it is desirable to increase bioavailability and/or increase the time between doses, possibly by increasing the time that a protein remains available in the serum after dosing. In some instances, it is desirable to improve the continuity of the serum concentration of the protein over time (e.g., decrease the difference in serum concentration of the protein shortly after administration and shortly before the next administration).

### Immunoglobulin Fc Domain (and Fragments)

In some embodiments, the anti-HIV bnAb polypeptide part of the invention has a modified functional Fc domain, or a fragment or variant thereof. As used herein, a “functional Fc region” is an Fc domain or fragment thereof which retains the ability to bind FcRn. In some embodiments, a functional Fc region binds to FcRn, but does not possess effector functions. The ability of the Fc region or fragment thereof to bind to FcRn can be determined by standard binding assays known in the art. In other embodiments, the modified Fc region or fragment thereof binds to FcRn and improves at least one “effector function” of a native Fc region. Exemplary “effector functions” include C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc.

A “native sequence Fc region” comprises an amino acid sequence identical to the amino acid sequence of an Fc region found in nature. A “variant Fc region” comprises an amino acid sequence which differs from that of a native sequence Fc region by virtue of at least one amino acid modification. Preferably, the variant Fc region has at least one amino acid substitution compared to a native sequence Fc region or to the Fc region of a parent polypeptide, e.g., from about one to about ten amino acid substitutions, and preferably from about one to about five amino acid substitutions in a native sequence Fc region or in the Fc region of the parent polypeptide. The variant Fc region herein will preferably possess at least about 80% sequence identity with a native sequence Fc region and/or with an Fc region of a parent polypeptide; more preferably at least 90% sequence identity and most preferably at least about 95%, 96%, or 97% sequence identity therewith, and still more preferably at least about 98% sequence identity therewith.

### V. Nucleic Acid-Protein Fusion Technology

In one aspect, the invention provides an Adnectin™ comprising fibronectin type III domains that binds CD4 or the N17 domain of gp41. One way to rapidly make and test Fn3 domains with specific binding properties is the nucleic acid-protein fusion technology. This disclosure utilizes the in vitro expression and tagging technology, termed ‘PROfusion’ which exploits nucleic acid-protein fusions (RNA- and DNA-protein fusions) to identify novel polypeptides and amino acid motifs that are important for binding to proteins. Nucleic acid-protein fusion technology is a technology that covalently couples a protein to its encoding genetic information. For a detailed description of the RNA-protein fusion technology and fibronectin-based scaffold protein library screening methods see Szostak et al., U.S. Pat. Nos. 6,258,558, 6,261,804, 6,214,553, 6,281,344, 6,207,446, 6,518,018 and 6,818,418; Roberts et al., Proc. Natl. Acad. Sci., 94:12297-12302 (1997); and Kurz et al., Molecules, 5:1259-1264 (2000), all of which are herein incorporated by reference.

VI. Anti-CD4 Adnectin™ Linked to an Anti-HIV Broadly Neutralizing Antibody (bnAb)

The anti-CD4 Adnectin™ sequences described in Table 4 may be linked to an anti-HIV broadly neutralizing antibody (anti-HIV bnAb) or fragment thereof. Such anti-HIV bnAbs include but are not limited to the anti-HIV bnAbs described in Table 8:

TABLE 8
Anti-HIV broadly neutralizing antibodies^{†}

| HIV1 | | | | |
|---|---|---|---|---|
| bnAb | | Epitope binding | | |
| class | Clones | feature | Isolation year | References^{†} |
| Apex | PG9, PG16 | N156/N160 glycans, | 2009 | 39, 41, 42 |
| (V1/V2) | | V1V2 strand C | | |
| | CH01-04 | N156/N160 glycans, | 2011 | 41, 43, 44** |
| | | V1V2 strand C | | |
| | PGT141-145/ | N160 glycan, hole at | 2011 | 40, 41, 45, 46** |
| | PDGM1400-1412 | trimer apex | | |
| | CAP256-VRC26.01-33 | N156/N160 glycans, | 2014 | 17, 47 |
| | | V1V2 strand C | | |
| | N90-VRC38.01-11 | N156/N160 glycans, | 2016 | 48 |
| | | V1V2 strands A, B, C | | |
| N332 | 2G12 | Glycan only | 1994 | 49 |
| glycan | PGT121-123, PGT124 | GDIR motif, V1V2 | 2011 | 40, 50-52 |
| | | and/or V3 glycans | | |
| | 10-1074 | GDIR motif, V1V2 | 2011 | 40, 53 |
| | | and/or V3 glycans | | |
| | PGT125-131 | GDIR motif, V3 glycans | 2011 | 40, 54 |
| | PGT135-137 | V3 and V4 glycans | 2011 | |
| | PCDN-27A, PCDN-27B, | V3 glycans | 2016 | 18* |
| | PCDN-33A, PCDN-38A, | | | |
| | PCDN-38B | | | |
| | PGDM11-14, PGDM21 | GDIR motif, N156 and | 2016 | 55* |
| | | V3 glycans | | |
| | DH270.1-6, DH542 | GDIR motif, V3 glycans | 2017 | 56* |
| CD4 | b12 | CD4bs | 1991 | 30, 57 |
| binding | VRC13, VRC16 | CD4bs | 2010 | 58, 59 |
| site | CH103 | CD4bs | 2010 | 58, 59 |
| (CD4bs) | HJ16 | N276 requirement for | 2010 | 58, 59 |
| | | HJ16 | | |
| | VRC01, N6, VRC23, | CD4bs | 2010 | 38, 59-61, 62** |
| | VRC-PG04, | | | |
| | 3BNC117 | CD4bs | 2010 | 38, 59-61, 62** |
| | 8ANC131, 8ANC134, | CD4bs | 2011 | |
| | 1B2530 | | | |
| | IOMA | CD4bs | 2016 | 14** |
| | CH235 | CD4bs | 2014 | Cell 2016 |
| | | | | 165(2): 449-463 |
| | NIH45-46 | CD4bs | 2011 | Science 2011 333 |
| | | | | (6049): 1633-1637 |
| gp120- | PGT151-158 | Fusion peptide and | 2014 | 11**, 63, 64 |
| gp41 | | complex glycans at | | |
| interface | | N611 and N637 on gp41 | | |
| | 35022 | Glycan on N88 | 2014 | 65, 66 |
| | | requirement | | |
| | 8ANC195 | Glycans at N234, N276, | 2014 | 12**, 67, 68 |
| | | N637 | | |
| | 3BC315/3BC176 | Gp41-gp41 interface | 2015 | 69 |
| | VRC34.01-07 | N-term of gp41 fusion | 2016 | 70** |
| | | peptide and glycan at | | |
| | | N88 | | |
| | ACS201-205 | gp41 fusion peptide and | 2016 | 71* |
| | | glycan at N88 | | |
| | CAP248-2B | Includes gp120 C- | 2017 | 72* |
| | | terminus | | |
| MPER | 2F5 | N-terminal ELDKWAS | 1994 | 32, 73, 74 |
| | | core sequence | | |
| | 4E10 | C-terminal helical | 1994 | 33, 75, 76** |
| | | region of MPER and | | |
| | | membrane lipid | | |
| | z13e1 | MPER elbow region | 2001 | 33, 77, 78 |
| | 10E8/10E8v4 | C-terminal helical | 2012 | 11**, 79 |
| | | region | | |
| | DH511.1-6 | C-terminal helical | 2017 | 80** |
| | | region | | |

^{†}See Curr Opin HIV AIDS 2017, 12: 229-240
DOI: 10.1097/COH.0000000000000360 and references therein.

Particular anti-HIV bnAbs from Table 8 to combine with anti-CD4 Adnectin™ sequences from Table4 include PG9, PGT125-131, VRC01, 3BNC117, NIH45-46, 3BC176, 4E10, N6, 10E8,10E8v4, and other 10E8 variants. In an embodiment of the invention, the anti-HIV bnAb is a 10E8v4 bnAb, a PGT-128 bnAb, a DH511.2 or DH511.12P bnAb, a PG9 bnAb, a N6 bnAb or a VRC01 bnAb.

Other anti-HIV bnAbs may be combined with polypeptides described herein, including 4E10 and N6, or fragments thereof, and variants of 10E8. Examples of variants of 10E8 include the improved-solubility 10E8 bnAb known as 10E8v4, wherein the amino acid sequence of 10E8 has been modified to improve its pan-reactive HIV-neutralizing activity (Kwon et al., “Optimization of the solubility of HIV-1-neutralizing antibody 10E8 though somatic variation and structure-based design” J. Virol. 90, 5899-5912 (2016)), incorporated by reference herein. Other 10E8v4 variants including 10E8v4-100cW, 10E8v4-100cF and 10E8v4-5R+100cF described by Y. D. Kwon et al. in Cell Reports “Surface-Matrix Screening Identifies Semi-specific Interactions that Improve Potency of a Near Pan-reactive HIV-1-Neutralizing Antibody” Cell Reports 22, 1798-1809 (2018), (https://doi.org/10.1016/j.celrep.2018.01.023), incorporated by reference herein and those described by Huang et al. “Engineered bispecific antibodies with exquisite HIV-1-neutralizing activity” Cell 165, 1621-1631 (2016) are other such variants.

Other anti-HIV bnAbs that could be used in fusion polypeptides as described herein include 4E10 (Cardoso et al. “Broadly neutralizing anti-HIV 4E10 recognizes a helical conformation of a highly conserved fusion-associated motif in gp41” Immunity, 22, 163-173 (2005), incorporated by reference herein; N6, a broadly neutralizing antibody that targets the CD4-binding site (Huang et al. “Identification of a CD4-binding site antibody to HIV that evolved near-pan neutralization breadth” Immunity, 45, 1109-1121 (2016), incorporated by reference herein, and combinations of 10E8, 4E10 and N6 or fragments thereof.

In an embodiment of the invention, the anti-bnAb fusion polypeptide comprises 90%, 95%, 98%, 99% or 100% identity to the amino acid sequence of any one of SEQ ID NO: 9, SEQ ID NO: 10, SEQ ID NO: 11, SEQ ID NO: 12, SEQ ID NO: 749, SEQ ID NO: 750, SEQ ID NO: 751, SEQ ID NO: 752, SEQ ID NO: 753, SEQ ID NO: 754, SEQ ID NO: 755, SEQ ID NO: 756, SEQ ID NO: 757, SEQ ID NO: 758, SEQ ID NO: 759, SEQ ID NO: 760, SEQ ID NO: 761, SEQ ID NO: 762, SEQ ID NO: 763, SEQ ID NO: 764, SEQ ID NO: 765, SEQ ID NO: 766 or SEQ ID NO: 767.

In an embodiment of the invention, the 10E8 bnAb comprises 90%, 95%, 98%, 99% or 100% identity to the amino acid sequence of any of the 10E8 variants shown in Table 8.

In an embodiment, the 10E8 bnAb comprises 90%, 95%, 98%, 99% or 100% identity to any of the HC or LC sequences set forth in SEQ ID NO: 6, SEQ ID NO: 7, SEQ ID NO: 8, SEQ ID NO: 768, SEQ ID NO: SEQ ID NO: 769, SEQ ID NO: 770, SEQ ID NO: 771 and SEQ ID NO: 772 below:

10E8v4 LC
(SEQ ID NO: 6)
ASELTQDPAVSVALKQTVTITCRGDSLRSHYASWYQKKPGQAPVLLFYGKNNRPSGIPDRFSGSASGNRASLTITGA
QAEDEADYYCSSRDKSGSRLSVFGGGTKLTVLSQPKAAPSVTLFPPSSEELQANKATLVCLISDFYPGAVTVAWKAD
SSPVKAGVETTTPSKQSNNKYAASSYLSLTPEQWKSHRSYSCQVTHEGSTVEKTVAPTECS
10E8v4 HC-SLSLSP variant
(SEQ ID NO: 7)
EVRLVESGGGLVKPGGSLRLSCSASGFDFDNAWMTWVRQPPGKGLEWVGRITGPGEGWSVDYAESVKGRFTISR
DNTKNTLYLEMNNVRTEDTGYYFCARTGKYYDFWSGYPPGEEYFQDWGQGTLVIVSSASTKGPSVFPLAPSSKSTS
GGTAALGCLVKDYFPEPVTVSWNSGALTSGVHTFPAVLQSSGLYSLSSVVTVPSSSLGTQTYICNVNHKPSNTKVDK
KVEPKSCDKTHTCPPCPAPELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSRWQQGNVFSCSVMHEALHNHYTQKSL
SLSP
10E8v4 HC-SLSLSPGK variant
(SEQ ID NO: 8)
EVRLVESGGGLVKPGGSLRLSCSASGFDFDNAWMTWVRQPPGKGLEWVGRITGPGEGWSVDYAESVKGRFTISR
DNTKNTLYLEMNNVRTEDTGYYFCARTGKYYDFWSGYPPGEEYFQDWGQGTLVIVSSASTKGPSVFPLAPSSKSTS
GGTAALGCLVKDYFPEPVTVSWNSGALTSGVHTFPAVLQSSGLYSLSSVVTVPSSSLGTQTYICNVNHKPSNTKVDK
KVEPKSCDKTHTCPPCPAPELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSRWQQGNVFSCSVMHEALHNHYTQKSL
SLSPGK
10E8v2-HC (Heavy Chain)
SEQ ID NO: 768
EVRLVESGGGLVKPGGSLRLSCSASGFNFDDAWMTWVRQPPGKGLEWVGRISGPGEGWSVDYAESVKGRFTISR
LNSINFLYLEMNNVRTEDTGYYFCARTGKHYDFWSGYPPGEEYFQDWGQGTLVIVSSASTKGPSVFPLAPSSKSTS
GGTAALGCLVKDYFPEPVTVSWNSGALTSGVHTFPAVLQSSGLYSLSSVVTVPSSSLGTQTYICNVNHKPSNTKVDK
KVEPKSCDKTHTCPPCPAPELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSRWQQGNVFSCSVMHEALHNHYTQKSL
SLSPGK
10E8v3-HC (Heavy Chain)
SEQ ID NO: 769
EVOLVESGGDLVKPGGSLRLSCSASGFSFKNTWMTWVRQAPGKGLEWVGRITGPGEGWTSDYAATVQGRFTISR
NNMIDMLYLEMNRLRTDDTGLYYCVHTEKYYNFWGGYPPGEEYFQHWGRGTLVIVSSASTKGPSVFPLAPSSKST
SGGTAALGCLVKDYFPEPVTVSWNSGALTSGVHTFPAVLQSSGLYSLSSVVTVPSSSLGTQTYICNVNHKPSNTKVD
KKVEPKSCDKTHTCPPCPAPELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSCSVMHEALHNHYTQKS
LSLSPGK
10E8v4-V5R-100cF-HC (Heavy Chain)
SEQ ID NO: 770
EVRLRESGGGLVKPGGSLRLSCSASGFDFDNAWMTWVRQPPGKGLEWVGRITGPGEGWSVDYAESVKGRFTISR
DNTKNTLYLEMNNVRTEDTGYYFCARTGKYYDFWFGYPPGEEYFQDWGQGTLVIVSSASTKGPSVFPLAPSSKSTS
GGTAALGCLVKDYFPEPVTVSWNSGALTSGVHTFPAVLQSSGLYSLSSVVTVPSSSLGTQTYICNVNHKPSNTKVDK
KVEPKSCDKTHTCPPCPAPELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSRWQQGNVFSCSVMHEALHNHYTQKSL
SLSPGK
10E8v2-LC (light chain)
SEQ ID NO: 771
ASELTQDPAVSVALKQTVTITCRGDSLRSHYASWYQKKPGQAPILLFYGKNNRPSGVPDRFSGSASGNRASLTISGA
QAEDDAEYYCSSRDKSGSRLSVFGGGTKLTVLSQPKAAPSVTLFPPSSEELQANKATLVCLISDFYPGAVTVAWKAD
SSPVKAGVETTTPSKQSNNKYAASSYLSLTPEQWKSHRSYSCQVTHEGSTVEKTVAPTECS
10E8v3-LC (light chain)
SEQ ID NO: 772
ASELTQETGVSVALGRTVTITCRGDSLRSHYASWYQKKPGQAPILLFYGKNNRPSGIHDRFSGSASGNRASLTISGA
QAEDDAEYYCSSRDKSGSRLSVFGGGTKLTVLSQPKAAPSVTLFPPSSEELQANKATLVCLISDFYPGAVTVAWKAD
SSPVKAGVETTTPSKQSNNKYAASSYLSLTPEQWKSHRSYSCQVTHEGSTVEKTVAPTECS

### VII. Vectors and Polynucleotides

Nucleic acids encoding any of the various proteins or polypeptides (e.g., antigen binding polypeptides or fusion polypeptides) disclosed herein may be synthesized chemically. Codon usage may be selected so as to improve expression in a cell. Such codon usage will depend on the cell type selected. Specialized codon usage patterns have been developed for E. coli and other bacteria, as well as mammalian cells, plant cells, yeast cells and insect cells. See, for example, Mayfield et al., Proc. Natl. Acad. Sci. USA, 100(2):438-442 (Jan. 21, 2003); Sinclair et al., Protein Expr. Purif., 26(I):96-105 (October 2002); Connell, N. D., Curr. Opin. Biotechnol., 12(5):446-449 (October 2001); Makrides et al., Microbiol. Rev., 60(3):512-538 (September 1996); and Sharp et al., Yeast, 7(7):657-678 (October 1991).

General techniques for nucleic acid manipulation are described in, for example, Sambrook et al., Molecular Cloning: A Laboratory Manual, Second Edition, Vols. 1-3, Cold Spring Harbor Laboratory Press (1989), or Ausubel, F. et al., Current Protocols in Molecular Biology, Green Publishing and Wiley-Interscience, New York (1987) and periodic updates, herein incorporated by reference. Generally, the DNA encoding the polypeptide is operably linked to suitable transcriptional or translational regulatory elements derived from mammalian, viral, or insect genes. Such regulatory elements include a transcriptional promoter, an optional operator sequence to control transcription, a sequence encoding suitable mRNA ribosomal binding site, and sequences that control the termination of transcription and translation. The ability to replicate in a host, usually conferred by an origin of replication, and a selection gene to facilitate recognition of transformants is additionally incorporated.

The proteins described herein may be produced recombinantly not only directly, but also as a fusion polypeptide with a heterologous polypeptide, which is preferably a signal sequence or other polypeptide having a specific cleavage site at the N-terminus of the mature protein or polypeptide. The heterologous signal sequence selected preferably is one that is recognized and processed (i.e., cleaved by a signal peptidase) by the host cell.

For prokaryotic host cells that do not recognize and process a native signal sequence, the signal sequence is substituted by a prokaryotic signal sequence selected, for example, from the group of the alkaline phosphatase, penicillinase, 1 pp, or heat-stable enterotoxin II leaders.

For yeast secretion, the native signal sequence may be substituted by, e.g., a yeast invertase leader, a factor leader (including Saccharomyces and Kluyveromyces alphafactor leaders), or acid phosphatase leader, the C. albicans glucoamylase leader, or the signal sequence described in U.S. Pat. No. 5,631,144. In mammalian cell expression, mammalian signal sequences as well as viral secretory leaders, for example, the herpes simplex gD signal, are available. The DNA for such precursor regions may be ligated in reading frame to DNA encoding the protein.

Both expression and cloning vectors contain a nucleic acid sequence that enables the vector to replicate in one or more selected host cells. Generally, in cloning vectors this sequence is one that enables the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gram-negative bacteria, the 2 micron plasmid origin is suitable for yeast, and various viral origins (SV40, polyoma, adenovirus, VSV or BPV) are useful for cloning vectors in mammalian cells. Generally, the origin of replication component is not needed for mammalian expression vectors (the SV40 origin may typically be used only because it contains the early promoter).

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

Expression and cloning vectors usually contain a promoter that is recognized by the host organism and is operably linked to the nucleic acid encoding the protein of the invention, e.g., a fibronectin-based scaffold protein. Promoters suitable for use with prokaryotic hosts include the phoA promoter, beta-lactamase and lactose promoter systems, alkaline phosphatase, a tryptophan (trp) promoter system, and hybrid promoters such as the tan promoter. However, other known bacterial promoters are suitable.

Promoters for use in bacterial systems also will contain a Shine-Dalgarno (S.D.) sequence operably linked to the DNA encoding the protein of the invention. Promoter sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Examples of suitable promoting sequences for use with yeast hosts include the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Transcription from vectors in mammalian host cells can be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and most preferably Simian Virus 40 (SV40), from heterologous mammalian promoters, e.g., the actin promoter or an immunoglobulin promoter, from heat-shock promoters, provided such promoters are compatible with the host cell systems.

Transcription of a DNA encoding protein of the invention by higher eukaryotes is often increased by inserting an enhancer sequence into the vector. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature, 297:17-18 (1982) on enhancing elements for activation of eukaryotic promoters. The enhancer may be spliced into the vector at a position 5′ or 3′ to the peptide-encoding sequence, but is preferably located at a site 5′ from the promoter.

Expression vectors used in eukaryotic host cells (e.g., yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs.

These regions contain nucleotide segments transcribed as polyadenylated fragments in the untranslated portion of mRNA encoding the protein of the invention. One useful transcription termination component is the bovine growth hormone polyadenylation region. See WO 94/11026 and the expression vector disclosed therein.

The recombinant DNA can also include any type of protein tag sequence that may be useful for purifying the protein. Examples of protein tags include, but are not limited to, a histidine tag, a FLAG tag, a myc tag, an HA tag, or a GST tag. Appropriate cloning and expression vectors for use with bacterial, fungal, yeast, and mammalian cellular hosts can be found in Cloning Vectors: A Laboratory Manual, Elsevier, New York (1985), the relevant disclosure of which is hereby incorporated by reference.

The expression construct is introduced into the host cell using a method appropriate to the host cell, as will be apparent to one of skill in the art. A variety of methods for introducing nucleic acids into host cells are known in the art, including, but not limited to, electroporation; transfection employing calcium chloride, rubidium chloride, calcium phosphate, DEAE-dextran, or other substances; microprojectile bombardment; lipofection; and infection (where the vector is an infectious agent).

Suitable host cells include prokaryotes, yeast, mammalian cells, or bacterial cells. Suitable bacteria include gram negative or gram positive organisms, for example, E. coli or Bacillus spp.

Various mammalian or insect cell culture systems can also be employed to express recombinant proteins. Baculovirus systems for production of heterologous proteins in insect cells are reviewed by Luckow et al. (Bio/Technology, 6:47 (1988)). Examples of suitable mammalian host cell lines include endothelial cells, COS-7 monkey kidney cells, CV-1, L cells, C127, 3T3, Chinese hamster ovary (CHO), human embryonic kidney cells, HeLa, 293, 293T, and BHK cell lines. Purified polypeptides are prepared by culturing suitable host/vector systems to express the recombinant proteins. For many applications, the small size of many of the polypeptides disclosed herein would make expression in E. coli as the preferred method for expression. The protein is then purified from culture media or cell extracts.

### VIII. Protein Production

Creation and isolation of cell lines producing components of fusion protein (e.g., antigen binding polypeptide) such as an Adnectin™ can be accomplished using standard techniques known in the art, such as those described herein and in WO20160414, the entire contents of which is incorporated by reference herein.

Host cells are transformed with the herein-described expression or cloning vectors for protein production and cultured in conventional nutrient media modified as appropriate for inducing promoters, selecting transformants, or amplifying the genes encoding the desired sequences. In the examples shown here, the host cells used for high-throughput protein production (HTPP) and mid-scale production were those from the HMS174-bacterial strain.

The host cells used to produce the proteins as described herein may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma)) are suitable for culturing the host cells. In addition, many of the media described in Ham et al., Meth. Enzymol., 58:44 (1979), Barites et al., Anal. Biochem., 102:255 (1980), U.S. Pat. Nos. 4,767,704, 4,657,866, 4,927,762, 4,560,655, 5,122,469, 6,048,728, 5,672,502, or U.S. Pat. No. RE 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as Gentamycin drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

Proteins disclosed herein can also be produced using cell-free translation systems. For such purposes the nucleic acids encoding the polypeptide must be modified to allow in vitro transcription to produce mRNA and to allow cell-free translation of the mRNA in the particular cell-free system being utilized (eukaryotic such as a mammalian or yeast cell-free translation system or prokaryotic such as a bacterial cell-free translation system).

Proteins described herein can also be produced by chemical synthesis (e.g., by the methods described in Solid Phase Peptide Synthesis, Second Edition, The Pierce Chemical Co., Rockford, Ill. (1984)). Modifications to the protein can also be produced by chemical synthesis.

The proteins of the present invention can be purified by isolation/purification methods for proteins generally known in the field of protein chemistry. Non-limiting examples include extraction, recrystallization, salting out (e.g., with ammonium sulfate or sodium sulfate), centrifugation, dialysis, ultrafiltration, adsorption chromatography, ion exchange chromatography, hydrophobic chromatography, normal phase chromatography, reversed-phase chromatography, get filtration, gel permeation chromatography, affinity chromatography, electrophoresis, countercurrent distribution or any combinations of these. After purification, polypeptides may be exchanged into different buffers and/or concentrated by any of a variety of methods known to the art, including, but not limited to, filtration and dialysis.

The purified polypeptide is preferably at least 85% pure, or preferably at least 95% pure, and most preferably at least 98% pure. Regardless of the exact numerical value of the purity, the polypeptide is sufficiently pure for use as a pharmaceutical product.

### IX. Biophysical and Biochemical Characterization

Binding of the protein of the invention to a target molecule (e.g., CD4 or gp41) may be assessed in terms of equilibrium constants (e.g., dissociation, Kd) and in terms of kinetic constants (e.g., on-rate constant, kon and off-rate constant, koff). The protein of the invention will generally bind to a target molecule with a Kd of less than 500 nM, 100 nM, 10 nM, 1 nM, 500 pM, 200 pM, or 100 pM, although higher Kd values may be tolerated where the koff is sufficiently low or the kon, is sufficiently high.

### In Vitro Assays for Binding Affinity

Polypeptides (such as Adnectins™) that bind CD4 or gp41 can be identified using various in vitro assays. Preferably, the assays are high-throughput assays that allow for screening multiple candidates simultaneously.

In some embodiments, biomolecular interactions can be monitored in real time with the BIACORE® system, which uses SPR to detect changes in the resonance angle of light at the surface of a thin gold film on a glass support due to changes in the refractive index of the surface up to 300 nm away. BIACORE® analysis generates association rate constants, dissociation rate constants, equilibrium dissociation constants, and affinity constants. Binding affinity is obtained by assessing the association and dissociation rate constants using a BIACORE® surface plasmon resonance system (Biacore, Inc.). A biosensor chip is activated for covalent coupling of the target. The target is then diluted and injected over the chip to obtain a signal in response units of immobilized material. Since the signal in resonance units (RU) is proportional to the mass of immobilized material, this represents a range of immobilized target densities on the matrix. Association and dissociation data are fit simultaneously in a global analysis to solve the net rate expression for a 1:1 bimolecular interaction, yielding best fit values for kon, koff and Rmax (maximal response at saturation). Equilibrium dissociation constants for binding, Kd's are calculated from SPR measurements as koff/kon.

In some embodiments, the polypeptides of the invention exhibit a Kd of 100 nM or less. Preferably, the Kd is 10 nM or less. More preferably, the Kd is 1 nM or less.

In some embodiments, the polypeptides of the invention exhibit an IC₅₀ of 5 nM or less, 4 nM or less, 3 nM or less, 2.5 nM or less, 2 nM or less, 1.5 nM or less, 1 nM or less, 0.5 nM or less, 0.2 nM or less, or 0.1 nM or less. Preferably, the IC₅₀ is 1.5 nM or less. More preferably, the IC₅₀ is 0.5 nM or less.

It should be understood that the assays described herein above are exemplary, and that any method known in the art for determining the binding affinity between proteins (e.g., fluorescence based-transfer (FRET), enzyme-linked immunosorbent assay, and competitive binding assays (e.g., radioimmunoassays)) can be used to assess the binding affinities of the polypeptides of the invention.

In the present invention, ELISA assays were utilized for identifying Adnectins™ that bind to CD4, with affinities determined by BIACORE® SPR. FACS assays are also used to determine the EC₅₀ of binding of the CD4 Adnectin™ (alone and as part of the CD4 binding moiey-bnAb polypeptide) to CD4 as presented naturally on T-cell surfaces. Peptide affinities are measured by BIACORE® SPR.

### In Vitro Assays for Inhibition Activity

Various art-recognized in vitro systems exist that allow for examination of the potency of the bnAb fusion polypeptides of the invention (or of individual inhibitors or combinations thereof) against HIV infection, particularly HIV-1 infection. These include systems that allow for complete replication of laboratory-derived virus or clinical isolates of various strains in cultured cells or peripheral blood monocyte cultures. In addition, systems that recapitulate the early cell entry stages of infection, without using viable virus, could be used to analyze the effectiveness of the polypeptide of the invention, individual inhibitors or combinations thereof. These include, but are not limited to, “pseudotyped” viruses that contain deletions that make them unable to produce infectious virions or cells that express only the HIV gp160 gene that can be used to monitor the HIV specific fusion reaction to target cells.

### In Vivo Models

One skilled in the art would know of various art-recognized animal models that allow for replication and in some cases recapitulate the symptoms of HIV infection. These models can be used to test the efficacy of the polypeptide of the invention, individual inhibitors or combinations thereof of the invention.

### X. Therapeutic Applications

In one aspect, the present invention provides fusion polypeptides (e.g. antigen binding polypeptides) of the invention useful for the treatment or prophylaxis of HIV infection. Accordingly, in certain embodiments the invention provides methods for attenuating or inhibiting HIV fusion in a subject comprising administering an effective amount of the polypeptide of the invention to a subject. In some embodiments, the subject is a human. In some embodiments, the polypeptide of the invention of the invention is pharmaceutically acceptable to a mammal, in particular a human. A “pharmaceutically acceptable” polypeptide refers to a polypeptide that is administered to an animal without significant adverse medical consequences.

In some embodiments, the polypeptide of the present invention will be administered to a subject in combination (concurrently or separately) with an agent known in the art to be useful for the particular disorder or disease being treated.

In some embodiments, the target patient population for polypeptide of the invention therapy is one that is not amenable to standard therapy for the disease being treated due to, e.g., age, pre-existing conditions, genetic makeup, and/or co-morbidities. The polypeptide of the invention can serve as an alternative to existing therapies that are associated with substantial side effects or safety concerns.

In some embodiments, the target patient population for polypeptide of the invention therapy is comprised of uninfected individuals at high risk of infection, due to lifestyle or other aggravating factors. The polypeptide of the invention is used to protect these individuals from getting infected by HIV (pre-exposure prophylaxis).

### XI. Pharmaceutical Compositions

The present invention further provides pharmaceutical compositions comprising a polypeptide of the invention or fusion proteins thereof described herein, wherein the composition is essentially endotoxin free, or at least contain no more than acceptable levels of endotoxins as determined by the appropriate regulatory agency (e.g., FDA).

Compositions of the present invention can be in the form of a liquid for intravenous, subcutaneous, intramuscular or parenteral administration; or a gel, lotion, ointment, cream, or a polymer or other sustained release vehicle for local administration, or an atomizable suspension suitable for inhaled or intranasal administration.

Methods well known in the art for making compositions are found, for example, in Gennaro, A. R., ed., Remington: The Science and Practice of Pharmacy, 20th Edition, Lippincott Williams & Wilkins, Philadelphia, PA (2000). Compositions for parenteral administration may, for example, contain excipients, sterile water, saline, polyalkylene glycols such as polyethylene glycol, oils of vegetable origin, or hydrogenated naphthalenes. Biocompatible, biodegradable lactide polymer, lactide/glycolide copolymer, or polyoxyethylene-polyoxypropylene copolymers may be used to control the release of the compounds. Nanoparticulate compositions (e.g., biodegradable nanoparticles, solid lipid nanoparticles, liposomes) may be used to control the biodistribution of the compounds. Other potentially useful parenteral delivery systems include ethylene-vinyl acetate copolymer particles, osmotic pumps, implantable infusion systems, and liposomes. The concentration of the compound in the composition varies depending upon a number of factors, including the dosage of the drug to be administered, and the route of administration.

Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as Tween, PLURONIC® or polyethylene glycol (PEG).

The active ingredients may also be entrapped in a microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Osol, A., ed., Remington's Pharmaceutical Sciences, 16th Edition (1980).

Sustained-release preparations may be prepared. Suitable examples of sustained-release preparations include semipermeable matrices of solid hydrophobic polymers containing the proteins of the invention, which matrices are in the form of shaped articles, e.g., films, or microcapsule.

Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-Lglutamate, non-degradable ethylene-vinyl acetate, degradable lactic acid-glycolic acid copolymers such as the LUPRON DEPOT® (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated proteins of the invention may remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

The pharmaceutical composition to be used for in vivo administration typically must be sterile. This may be accomplished by filtration through sterile filtration membranes. Where the composition is lyophilized, sterilization using this method may be conducted either prior to or following lyophilization and reconstitution. The composition for parenteral administration may be stored in lyophilized form or in solution. In addition, parenteral compositions generally are placed into a container having a sterile access port, for example, an intravenous solution bag or vial having a stopper pierceable by a hypodermic injection needle.

Once the pharmaceutical composition has been formulated, it may be stored in sterile vials as a solution, suspension, gel, emulsion, solid, or a dehydrated or lyophilized powder. Such formulations may be stored either in a ready-to-use form or in a form (e.g., lyophilized) requiring reconstitution prior to administration.

The compositions herein may also contain more than one active compound as necessary for the particular indication being treated, preferably those with complementary activities that do not adversely affect each other. Such molecules are suitably present in combination in amounts that are effective for the purpose intended.

### XII. Administration

A pharmaceutical composition comprising a polypeptide of the invention or fusion protein thereof of the present invention can be administered to a subject with HIV using standard administration techniques including parenteral, pulmonary, transdermal, intramuscular, intranasal, buccal, sublingual, or suppository administration. Preferably, administration of the polypeptide of the invention is parenteral. The term parenteral as used herein includes intravenous, intramuscular, subcutaneous, rectal, vaginal, or intraperitoneal administration. Peripheral systemic delivery by intravenous or intraperitoneal or subcutaneous injection is preferred.

A therapeutically effective dose refers to a dose that produces the therapeutic effects for which it is administered. An effective amount of a pharmaceutical composition to be employed therapeutically will depend, for example, upon the therapeutic context and objectives. One skilled in the art will appreciate that the appropriate dosage levels for treatment will thus vary depending, in part, upon the molecule delivered, the indication for which the binding agent molecule is being used, the route of administration, and the size (body weight, body surface or organ size) and condition (the age and general health) of the patient.

For example, the therapeutically effective dose can be estimated initially either in cell culture assays or in animal models such as mice, rats, rabbits, dogs, pigs, or monkeys. An animal model may also be used to determine the appropriate concentration range and route of administration. Such information can then be used to determine useful doses and routes for administration in humans.

The exact dosage will be determined in light of factors related to the subject requiring treatment, and may be ascertained using standard techniques. Dosage and administration are adjusted to provide sufficient levels of the active compound or to maintain the desired effect.

Factors that may be taken into account include the severity of the disease state, the general health of the subject, the age, weight, and gender of the subject, time and frequency of administration, drug combination(s), reaction sensitivities, and response to therapy. In general, the polypeptides of the present invention are administered at about 0.01 mg/kg to about 50 mg/kg per day, preferably about 0.01 mg/kg to about 30 mg/kg per day, most preferably about 0.01 mg/kg to about 20 mg/kg per day.

In some embodiments, the polypeptides of the present invention are administered at weekly dosages of about 0.01 mg/kg to about 10 mg/kg, more preferably about 0.01 to about 5 mg/kg, most preferably about 0.01 to about 1 mg/kg. Alternatively, the polypeptides of the invention are administered at about 15 to about 100 mg/week, from about 20 to about 80 mg/week, from about 20 to about 60 mg/week, or about 20 to about 25 mg/week.

The frequency of dosing will depend upon the pharmacokinetic parameters of the binding agent molecule in the formulation used. Typically, a composition is administered until a dosage is reached that achieves the desired effect. The composition may therefore be administered as a single dose or as multiple doses (at the same or different concentrations/dosages) over time, or as a continuous infusion. Further refinement of the appropriate dosage is routinely made. Appropriate dosages may be ascertained through use of appropriate dose-response data. For example, the polypeptide of the invention may be given daily (e.g., once, twice, three times, or four times daily) or less frequently (e.g., once every other day, once or twice weekly, or monthly). In addition, as is known in the art, adjustments for age as well as the body weight, general health, sex, diet, time of administration, drug interaction, and the severity of the disease may be necessary, and will be ascertainable with routine experimentation by those skilled in the art. The polypeptide of the invention is suitably administered to the patient at one time or over a series of treatments.

Administration of a polypeptide of the invention or a fusion thereof, and one or more additional therapeutic agents, whether co-administered or administered sequentially, may occur as described above for therapeutic applications. Suitable pharmaceutically acceptable carriers, diluents, and excipients for co-administration will be understood by the skilled artisan to depend on the identity of the particular therapeutic agent being administered.

### XIII. Kits and Articles of Manufacture

The polypeptides of the invention can be provided in a kit, a packaged combination of reagents in predetermined amounts with instructions for use in the therapeutic or diagnostic methods of the invention.

For example, in one embodiment of the invention, an article of manufacture containing materials useful for the treatment or prevention of the disorders or conditions described above is provided. The article of manufacture comprises a container and a label. Suitable containers include, for example, bottles, vials, syringes, and test tubes. The containers may be formed from a variety of materials such as glass or plastic. The container holds a composition of the invention which is effective for treating HIV and may have a sterile access port (for example, the container may be an intravenous solution bag or a vial having a stopper pierceable by a hypodermic injection needle). The active agent in the composition is a polypeptide of the invention. The label on, or associated with, the container indicates that the composition is used for treating HIV. The article of manufacture may further comprise a second container comprising a pharmaceutically-acceptable buffer, such as phosphate-buffered saline, Ringer's solution and dextrose solution. It may further include other materials desirable from a commercial and user standpoint, including other buffers, diluents, filters, needles, syringes, and package inserts with instructions for use.

### EXAMPLES

The invention is now described by reference to the following examples, which are illustrative only, and are not intended to limit the present invention. While the invention has been described in detail and with reference to specific embodiments thereof, it will be apparent to one of skill in the art that various changes and modifications can be made thereto without departing from the spirit and scope thereof.

### Example 1-HIV Polypeptide-Fc Construct; Mammalian Cell Culture

DNA is transfected into appropriate mammalian cells.

Cells grown in cell culture.

Harvested by centrifugation and/or filtration.

Purification using affinity chromatography and ion exchange chromatography.

Formulated and concentrated using tangential flow filtration.

### Example 2—Polypeptide Potency Assay

MT-2 cells, HEK 293T cells and the proviral DNA clone of NL₄₋₃ were obtained from the NIH AIDS Research and Reference Reagent Program. MT-2 cells were propagated in RPMI 1640 medium supplemented with 10% heat-inactivated fetal bovine serum (FBS), 10 mM HEPES buffer pH 7.55, and 2 mM L-glutamine. HEK 293T cells were propagated in DMEM media supplemented with 10% heat-inactivated FBS, 10 Mm HEPES buffer pH 7.55 and 2 mM L-glutamine. Recombinant NL-Rluc virus, in which a section of the nef gene from the proviral clone of NL4-3 was replaced with the Renilla luciferase gene, was constructed at Bristol-Myers Squibb. The replication-competent virus was harvested 3 days after transfection of HEK 293T cells with the modified pNLRluc proviral clone. Transfections were performed using Lipofectamine Plus (Invitrogen, Carlsbad, CA), according to manufacturer's instruction. Virus was titered in MT-2 cells using luciferase enzyme activity as a biomarker. The NL-Rluc virus was used to infect MT-2 cells a multiplicity of 0.01 for 1 hour before adding to the peptides in 96-well plates. Peptides were serially diluted four-fold and 11 concentrations were plated in triplicate. After 4 days of incubation, cells were processed and quantitated for virus growth by the amount of expressed luciferase. Luciferase was quantitated using the Dual Luciferase kit from Promega (Madison, WI), with modifications to the manufacturer's protocol. The diluted Passive Lysis solution was pre-mixed with the re-suspended Luciferase Assay Reagent and then was re-suspended in STOP & GLO® Substrate (2:1:1 ratio). A total of 50 μL of the mixture was added to each aspirated well on assay plates and luciferase activity was measured immediately on a Wallac TriLux (Perkin-Elmer, Waltham, MA). The 50% effective concentration (EC₅₀) were calculated by comparing the amount of luciferase produced in the presence of inhibitory peptide compared to wells where no peptide is added. A 5-parameter sigmoidal equation was used to fit the resulting signal vs. concentration curves, and the concentration of each inhibitor that produced 50% maximal inhibition (EC₅₀) was determined. The results of three independent experiments were averaged and plotted, with error bars corresponding to 1 standard deviation.

### Example 3—Method for Producing the 10E8v4 bnAb and the 10E8v4 bnAb-CD4 Adnectin™ Fusion (FIG. 2)

The heavy and light chain genes encoding the 10E8v4 antibody and the heavy chain-CD4 A or B Adnectin™ fusion were cloned into the pTT5 expression vector. The resulting plasmids were co-transfected into 293HEK cells. Conditioned cell medium was harvested and subjected to purification by a Protein A-conjugated resin, by standard procedures used commonly for antibodies.

Mature Amino Acid Sequences (final sequence after removal of secretion signal peptide) correspond to SEQ ID NO: 6 (10E8v4 Light Chain) and SEQ ID NO: 7 or SEQ ID NO: 8 (10E8v4 Heavy Chain ending in SLSLSP or 10E8v4 Heavy Chain ending in SLSLSPGK, respectively). Additional amino acid sequences (after removal of secretion signal peptide) include SEQ ID NO: 770 (10E8v2 HC), SEQ ID NO: 771 (10E8v3 HC), SEQ ID NO: 772 (10E8v4-V5R-100cF HC), SEQ ID NO: 768 (10E8 v2 LC), and SEQ ID NO: 769 (20E8v3 LC).

10E8v4 (LC and HC) or VROC1 bnAbs and/or CD4 a Adnectins™ and/or HIV Fusion Peptide Inhibitors with Various Linkers

Particular embodiments of the invention provide fusion polypeptides comprising either the heavy chain or light chain of the 10E8v4 anti-HIV bnAb, with a CD4 A Adnectin™, optionally with a HIV fusion peptide inhibitor. Particular embodiments of the invention provide fusion polypeptides comprising a VRC01 anti-HIV bnAb, with a CD4 A Adnectin™, optionally with a HIV fusion peptide inhibitor. Other particular embodiments of the invention provide fusion polypeptides comprising either the heavy chain or light chain of a 10E8v4 anti-HIV bnAb, with an N17 Adnectin™, optionally with a HIV fusion peptide inhibitor. Linkers between the bnAb moiety and the Adnectin™ moiety and the optional HIV fusion peptide inhibitor may be varied as needed. Particular examples of such Adnectin™-anti-HIV bnAb fusion polypeptides are shown in SEQ ID NO: 749 through SEQ ID NO: 767

10E8v4-HC-ESPEPETED2-CD4 A (CD4 A Adnectin ™ linked to the heavy chain of bnAb
10E8v4)
(SEQ ID NO: 749)
EVRLVESGGGLVKPGGSLRLSCSASGFDFDNAWMTWVRQPPGKGLEWVGRITGPGEGWSVDYAESVKGRFTI
SRDNTKNTLYLEMNNVRTEDTGYYFCARTGKYYDFWSGYPPGEEYFQDWGQGTLVIVSSASTKGPSVFPLAPSS
KSTSGGTAALGCLVKDYFPEPVTVSWNSGALTSGVHTFPAVLQSSGLYSLSSVVTVPSSSLGTQTYICNVNHKPS
NTKVDKKVEPKSCDKTHTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFFLYSKLTVDKSRWQQGNVFSCSVM
HEALHNHYTQKSLSLSPESPEPETPEDESPEPETPEDEGVSDVPRDLEVVAATPTSLLISWDAPAVTVHSYHIQYWP
LGSYQRYQVFSVPGSKSTATISGLKPGVEYQIRVYAETGGADSDQSFGWIQIGYRTP
10E8v4-HC-(G4S)5-CD4 - A (CD4 A Adnectin ™ linked to the heavy chain of bnAb
10E8v4)
(SEQ ID NO: 750)
EVRLVESGGGLVKPGGSLRLSCSASGFDFDNAWMTWVRQPPGKGLEWVGRITGPGEGWSVDYAESVKGRFTI
SRDNTKNTLYLEMNNVRTEDTGYYFCARTGKYYDFWSGYPPGEEYFQDWGQGTLVIVSSASTKGPSVFPLAPSS
KSTSGGTAALGCLVKDYFPEPVTVSWNSGALTSGVHTFPAVLQSSGLYSLSSVVTVPSSSLGTQTYICNVNHKPS
NTKVDKKVEPKSCDKTHTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFFLYSKLTVDKSRWQQGNVFSCSVM
HEALHNHYTQKSLSLSPGGGGSGGGGSGGGGSGGGGSGGGGSGVSDVPRDLEVVAATPTSLLISWDAPAVTVH
SYHIQYWPLGSYQRYQVFSVPGSKSTATISGLKPGVEYQIRVYAETGGADSDQSFGWIQIGYRTP
VRC01-HC-(G4S)5-CD4 A (CD4 A Adnectin ™ linked to the light chain of a VRC01
bnAb
(SEQ ID NO: 751)
QVQLVQSGGQMKKPGESMRISCRASGYEFIDCTLNWIRLAPGKRPEWMGWLKPRGGAVNYARPLQGRVTM
TRDVYSDTAFLELRSLTVDDTAVYFCTRGKNCDYNWDFEHWGRGTPVIVSSPSTKGPSVFPLAPSSKSTSGGTA
ALGCLVKDYFPEPVTVSWNSGALTSGVHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSCSVMHEALHNHYT
QKSLSLSPGGGGSGGGGSGGGGSGGGGSGGGGSGVSDVPRDLEVVAATPTSLLISWDAPAVTVHSYHIQYWPLG
SYQRYQVFSVPGSKSTATISGLKPGVEYQIRVYAETGGADSDQSFGWIQIGYRTP
VRC01-LC-(G4S)5-CD4 A (CD4 A Adnectin ™ linked to the light chain of a VRC01
bnAb)
(SEQ ID NO: 752)
EIVLTQSPGTLSLSPGETAIISCRTSQYGSLAWYQQRPGQAPRLVIYSGSTRAAGIPDRFSGSRWGPDYNLTISNLE
SGDFGVYYCQQYEFFGQGTKVQVDIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLRSPVTKSFNRGECGGGGSGGGGSGGGG
SGGGGSGGGGSGVSDVPRDLEVVAATPTSLLISWDAPAVTVHSYHIQYWPLGSYQRYQVFSVPGSKSTATISGLKP
GVEYQIRVYAETGGADSDQSFGWIQIGYRTP
10E8v4-HC-ESPEPETPEDE-N17-(G4S)4-HIV fusion inhibitor (N17 Adnectin ™ linked to
the heavy chain of the 10E8v4 bnAb linked to a HIV fusion peptide inhibitor)
(SEQ ID NO: 753)
EVRLVESGGGLVKPGGSLRLSCSASGFDFDNAWMTWVRQPPGKGLEWVGRITGPGEGWSVDYAESVKGRFTI
SRDNTKNTLYLEMNNVRTEDTGYYFCARTGKYYDFWSGYPPGEEYFQDWGQGTLVIVSSASTKGPSVFPLAPSS
KSTSGGTAALGCLVKDYFPEPVTVSWNSGALTSGVHTFPAVLQSSGLYSLSSVVTVPSSSLGTQTYICNVNHKPS
NTKVDKKVEPKSCDKTHTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFFLYSKLTVDKSRWQQGNVFSCSVM
HEALHNHYTQKSLSLSPGKESPEPETPEDEGVSDVPRDLEVVAATPTSLLISWEYKVHPYRYYRITYGETGGNSPVQ

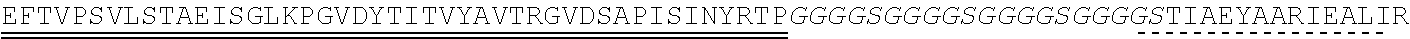

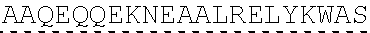

10E8v4-LC-ESPEPETPEDE -N17- GGGGS4-HIV fusion peptide (N17 Adnectin ™ linked to
the light chain of 10E8v4 bnAb linked to a HIV fusion peptide inhibitor)
(SEQ ID NO: 754)
ASELTQDPAVSVALKQTVTITCRGDSLRSHYASWYQKKPGQAPVLLFYGKNNRPSGIPDRFSGSASGNRASLTIT
GAQAEDEADYYCSSRDKSGSRLSVFGGGTKLTVLSQPKAAPSVTLFPPSSEELQANKATLVCLISDFYPGAVTVA
WKADSSPVKAGVETTTPSKQSNNKYAASSYLSLTPEQWKSHRSYSCQVTHEGSTVEKTVAPTECSESPEPETPED
EGVSDVPRDLEVVAATPTSLLISWEYKVHPYRYYRITYGETGGNSPVQEFTVPSVLSTAEISGLKPGVDYTITVYAVTR

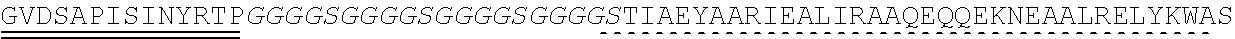

For above SEQ ID Nos: 749-754, the anti-HIV bnAb portion is in bold, the N17 Adnectin™ portion is double-underlined, the HIV fusion portion is dotted underlined, and the linker portions are italicized
10E8v4 bnAbs with CD4 A or CD4 B Adnectins™

Particular antigen binding polypeptides comprising the HC sequence from anti-HIV bnAb 10E8v4 and a CD4 Adnectin™, either a domain 2 binding (A) or domain 4 binding (B) CD4 Adnectin™, with different linkers are shown below as SEQ ID NO: 755 and SEQ ID NO: 756 (CD4 A) or SEQ ID NO: 757 and SEQ ID NO: 758 (CD4 B); and SEQ ID NO: 759 and SEQ ID NO: 760 (CD4 A) or SEQ ID NO: 761 and SEQ ID NO: 762 (CD4 B). For the various fusion polypeptides below, the CD4 Adnectin™ sequence is shown in underline; the linkers are italicized; the bnAb 10E8v4 or VRC01 heavy/light chain sequence is bolded; and if present, the N17 Adnectin™ is shown in double-underline, and if present, the HIV Fusion peptide inhibitor is shown in dotted-underline bold.

10E8v4-HC-ESPEPETED₂-CD4 A (CD4 A Adnectin ™ linked to the heavy chain of the 10E8v4 bnAb)
(SEQ ID NO: 755):
EVRLVESGGGLVKPGGSLRLSCSASGFDFDNAWMTWVRQPPGKGLEWVGRITGPGEGWSVDYAESVKGRFTI
SRDNTKNTLYLEMNNVRTEDTGYYFCARTGKYYDFWSGYPPGEEYFQDWGQGTLVIVSSASTKGPSVFPLAPSS
KSTSGGTAALGCLVKDYFPEPVTVSWNSGALTSGVHTFPAVLQSSGLYSLSSVVTVPSSSLGTQTYICNVNHKPS
NTKVDKKVEPKSCDKTHTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFFLYSKLTVDKSRWQQGNVFSCSVM
HEALHNHYTQKSLSLSPESPEPETPEDESPEPETPEDEGVSDVPRDLEVVAATPTSLLISWDAPAVTVHSYHIQYWP
LGSYQRYQVFSVPGSKSTATISGLKPGVEYQIRVYAETGGADSDQSFGWIQIGYRTP
10E8v4-HC-(G4S)₅-CD4 A (CD4 A Adnectin ™ linked to the heavy chain of the 10E8v4 bnAb)
(SEQ ID NO: 756):
EVRLVESGGGLVKPGGSLRLSCSASGFDFDNAWMTWVRQPPGKGLEWVGRITGPGEGWSVDYAESVKGRFTI
SRDNTKNTLYLEMNNVRTEDTGYYFCARTGKYYDFWSGYPPGEEYFQDWGQGTLVIVSSASTKGPSVFPLAPSS
KSTSGGTAALGCLVKDYFPEPVTVSWNSGALTSGVHTFPAVLQSSGLYSLSSVVTVPSSSLGTQTYICNVNHKPS
NTKVDKKVEPKSCDKTHTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFFLYSKLTVDKSRWQQGNVFSCSVM
HEALHNHYTQKSLSLSPGGGGSGGGGSGGGGSGGGGSGGGGSGVSDVPRDLEVVAATPTSLLISWDAPAVTVH
SYHIQYWPLGSYQRYQVFSVPGSKSTATISGLKPGVEYQIRVYAETGGADSDQSFGWIQIGYRTP
10E8v4-HC-ESPEPETED₂-CD4 B (CD4 B Adnectin ™ linked to the heavy chain of the 10E8v4 bnAb)
(SEQ ID NO: 757):
EVRLVESGGGLVKPGGSLRLSCSASGFDFDNAWMTWVRQPPGKGLEWVGRITGPGEGWSVDYAESVKGRFTI
SRDNTKNTLYLEMNNVRTEDTGYYFCARTGKYYDFWSGYPPGEEYFQDWGQGTLVIVSSASTKGPSVFPLAPSS
KSTSGGTAALGCLVKDYFPEPVTVSWNSGALTSGVHTFPAVLQSSGLYSLSSVVTVPSSSLGTQTYICNVNHKPS
NTKVDKKVEPKSCDKTHTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFFLYSKLTVDKSRWQQGNVFSCSVM
HEALHNHYTQKSLSLSPESPEPETPEDESPEPETPEDEMASTSGSASYLIPSDLEVVAATPTSLSIYWYPVASTIINFRI
TYVETGGNSPVQEFTVPGSKSTATISGLKPGVDYTITVYAVHYEQKYSEYWIGHPISINYRTEGSGSHHHHHH
10E8v4-HC-(G4S)₅-CD4 B (CD4 B Adnectin ™ linked to the heavy chain of the 10E8v4 bnAb)
(SEQ ID NO: 758):
EVRLVESGGGLVKPGGSLRLSCSASGFDFDNAWMTWVRQPPGKGLEWVGRITGPGEGWSVDYAESVKGRFTI
SRDNTKNTLYLEMNNVRTEDTGYYFCARTGKYYDFWSGYPPGEEYFQDWGQGTLVIVSSASTKGPSVFPLAPSS
KSTSGGTAALGCLVKDYFPEPVTVSWNSGALTSGVHTFPAVLQSSGLYSLSSVVTVPSSSLGTQTYICNVNHKPS
NTKVDKKVEPKSCDKTHTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFFLYSKLTVDKSRWQQGNVFSCSVM
HEALHNHYTQKSLSLSPGGGGSGGGGSGGGGSGGGGSGGGGSMASTSGSASYLIPSDLEVVAATPTSLSIYWYPV
ASTIINFRITYVETGGNSPVQEFTVPGSKSTATISGLKPGVDYTITVYAVHYEQKYSEYWIGHPISINYRTEGSGSHHH
HHH

Other embodiments of the invention provide bnAb-CD4 Adnectin™ fusions comprising anti-HIV bnAbs other than 10E8, shown below as SEQ ID NO: 768 and SEQ ID NO: 769, (VRC01 bnAb with CD4 A); and as SEQ ID NO: 770 and SEQ ID NO: 771 (VRC01 bnAb with CD4 B).

VRC01-HC-CD4 A Adnectin ™ (SEQ ID NO: 759):
QVQLVQSGGQMKKPGESMRISCRASGYEFIDCTLNWIRLAPGKRPEWMGWLKPRGGAVNYARPLQGRVTM
TRDVYSDTAFLELRSLTVDDTAVYFCTRGKNCDYNWDFEHWGRGTPVIVSSPSTKGPSVFPLAPSSKSTSGGTA
ALGCLVKDYFPEPVTVSWNSGALTSGVHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSCSVMHEALHNHYT
QKSLSLSPGGGGSGGGGSGGGGSGGGGSGGGGSGVSDVPRDLEVVAATPTSLLISWDAPAVTVHSYHIQYWPLG
SYQRYQVFSVPGSKSTATISGLKPGVEYQIRVYAETGGADSDQSFGWIQIGYRTP
VRC01-LC-CD4 A Adnectin ™ (SEQ ID NO: 760):
EIVLTQSPGTLSLSPGETAIISCRTSQYGSLAWYQQRPGQAPRLVIYSGSTRAAGIPDRFSGSRWGPDYNLTISNLE
SGDFGVYYCQQYEFFGQGTKVQVDIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLRSPVTKSFNRGECGGGGSGGGGSGGGG
SGGGGSGGGGSGVSDVPRDLEVVAATPTSLLISWDAPAVTVHSYHIQYWPLGSYQRYQVFSVPGSKSTATISGLKP
GVEYQIRVYAETGGADSDQSFGWIQIGYRTP
VRC01-HC-CD4 B Adnectin ™ (SEQ ID NO: 761):
QVQLVQSGGQMKKPGESMRISCRASGYEFIDCTLNWIRLAPGKRPEWMGWLKPRGGAVNYARPLQGRVTM
TRDVYSDTAFLELRSLTVDDTAVYFCTRGKNCDYNWDFEHWGRGTPVIVSSPSTKGPSVFPLAPSSKSTSGGTA
ALGCLVKDYFPEPVTVSWNSGALTSGVHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSCSVMHEALHNHYT
QKSLSLSPGGGGSGGGGSGGGGSGGGGSGGGGSMASTSGSASYLIPSDLEVVAATPTSLSIYWYPVASTIINFRITY
VETGGNSPVQEFTVPGSKSTATISGLKPGVDYTITVYAVHYEQKYSEYWIGHPISINYRTEGSGSHHHHHH
VRC01-LC-CD4 B Adnectin ™ (SEQ ID NO: 762):
EIVLTQSPGTLSLSPGETAIISCRTSQYGSLAWYQQRPGQAPRLVIYSGSTRAAGIPDRFSGSRWGPDYNLTISNLE
SGDFGVYYCQQYEFFGQGTKVQVDIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLRSPVTKSFNRGECGGGGSGGGGSGGGG
SGGGGSGGGGSMASTSGSASYLIPSDLEVVAATPTSLSIYWYPVASTIINFRITYVETGGNSPVQEFTVPGSKSTATIS
GLKPGVDYTITVYAVHYEQKYSEYWIGHPISINYRTEGSGSHHHHHH

bnAb-N17 Adnectin™-Peptide Fusions

Other embodiments of the invention provide bnAb-Adnectin™ fusions comprising anti-HIV bnAbs with N17 Adnectin™ instead of CD4 Adnectin™, shown below as SEQ ID NO: 763 and SEQ ID NO: 764 (10E8 bnAb with N17 Adnectin™). One particular antigen binding polypeptide comprising the HC sequence from anti-HIV bnAb 10E8v4, a fibronectin-based scaffold polypeptide that binds the N17 domain of gp41 (N17 Adnectin™) and an HIV fusion peptide inhibitor is shown below as SEQ ID NO: 763; and another particular antigen binding polypeptide comprising the LC sequences from anti-HIV bnAb 10E8v4, a fibronectin-based scaffold polypeptide that binds the N17 domain of gp41 (N17 Adnectin™), and an HIV fusion peptide inhibitor is shown below as SEQ ID NO: 764. For SEQ ID NOs: 763 and 764, the anti-HIV bnAb portion is in bold, the N17 Adnectin™ portion is double-underlined, the HIV fusion portion is dotted underlined, and the linker portions are italicized.

(10E8v4-HC-N17-Adn)
SEQ ID NO: 763
EVRLVESGGGLVKPGGSLRLSCSASGFDFDNAWMTWVRQPPGKGLEWVGRITGPGEGWSVDYAESVKGRFTI
SRDNTKNTLYLEMNNVRTEDTGYYFCARTGKYYDFWSGYPPGEEYFQDWGQGTLVIVSSASTKGPSVFPLAPSS
KSTSGGTAALGCLVKDYFPEPVTVSWNSGALTSGVHTFPAVLQSSGLYSLSSVVTVPSSSLGTQTYICNVNHKPS
NTKVDKKVEPKSCDKTHTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFFLYSKLTVDKSRWQQGNVFSCSVM
HEALHNHYTQKSLSLSPGKESPEPETPEDEGVSDVPRDLEVVAATPTSLLISWEYKVHPYRYYRITYGETGGNSPVQ

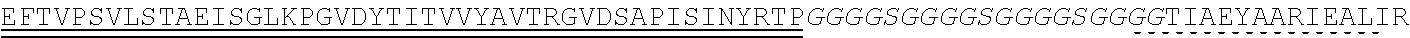

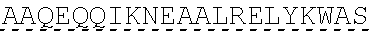

(10E8v4-LC-N17 Adn)
SEQ ID NO: 764
ASELTQDPAVSVALKQTVTITCRGDSLRSHYASWYQKKPGQAPVLLFYGKNNRPSGIPDRFSGSASGNRASLTIT
GAQAEDEADYYCSSRDKSGSRLSVFGGGTKLTVLSQPKAAPSVTLFPPSSEELQANKATLVCLISDFYPGAVTVA
WKADSSPVKAGVETTTPSKQSNNKYAASSYLSLTPEQWKSHRSYSCQVTHEGSTVEKTVAPTECSESPEPETPED
EGVSDVPRDLEVVAATPTSLLISWEYKVHPYRYYRITYGETGGNSPVQEFTVPSVLSTAEISGLKPGVDYTITVYAVTR

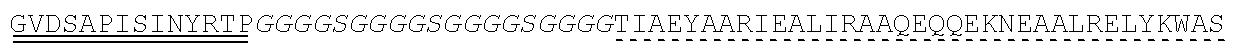

For SEQ ID NOs: 763 and 764, the anti-HIV bnAb portion is in bold, the N17 Adnectin™ portion is double-underlined, the HIV fusion portion is dotted underlined, and the linker portions are italicized.

In certain embodiments, Adnectin™ polypeptide fusions may comprise a bnAb heavy or light chain linked to one or more Adnectins™ and further linked to a HIV fusion peptide inhibitor to make an anti-HIV bnAb fusion with other components, (e.g. anti-HIV bnAb and/or CD4 Adnectin™ and/or N17 Adnectin™ and/or HIV Fusion peptide inhibitor).

### Example 4—General Method for Producing Anti-HIV bnAb and Anti-HIV bnAb-CD4 Adnectin™ Fusion (FIG. 4A-D)

A panel of anti-HIV bnAbs was chosen known to have different epitopes or mechanisms of action. Then, fusions to N17 Adnectin™ polypeptide and/or CD4 Adnectin™ polypeptide were made as described in Example 3. The potency of each bnAb-Adnectin™ polypeptide was compared to the parental bnAbs alone, or to non-fused combinations of the bnAb with the Adnectin™ polypeptide to identify which anti-HIV bnAb epitope(s) and/or mechanism(s) of action showed synergy and had the best potency, and to identify the best representative bnAb of that bnAb class from the full panel of bnAbs screened.

Table 9 below shows a bnAB panel that was used to screen for anti-HIV bnAbs to use in Adnectin™-bnAb fusion polypeptides. The bnAbs screened included representatives from the CD4b (VRC01 and CH235.12), V3 loop (PGT-128), MPER (10E8v4, DH511.2 and DH511.12P) and V1/V2 (PG9) classes of anti-HIV bnAbs. The Adnectins™ selected for preparation of the bnAb peptide fusion polypeptides included an N17 Adnectin™ that binds to a conserved site within gp41 and blocks 6-helilx bundle formation and subsequent membrane fusion by the HIV envelope protein, and a CD4 Adnectin™ that binds to either domain 2 or domain 4 of CD4, after CD4 binding by the envelope protein, to block conformational changes in gp120 of the envelope protein.

TABLE 9
bnAb Panel

| CD4bs | V3 loop | MPER | V1/V2 |
|---|---|---|---|
| VRC01 | PGT-128 | 10E8v4 | PG9 (TBD) |
| CH235.12 | | DH511.2/ | |
| | | DH511.12P | |

Other anti-HIV bnAbs may be found using a similar protocol and a different panel of prospective bnAbs. The resulting anti-HIV bnAbs identified can then be combined with a CD4 Adnectin™ polypeptide or N17 Adnectin™ polypeptide as described herein, using any of the amino acid linkers described herein, to create an antigen binding polypeptide that is a fusion polypeptide comprising a newly identified anti-HIV bnAb, and a CD4 binding polypeptide (e.g. CD4 A or CD4 B Adnectin™) and/or a gp41 binding polypeptide (e.g. an N17 Adnectin™). The resulting anti-HIV bnAb fusion polypeptide may also be optionally combined with a HIV fusion polypeptide inhibitor, as described herein, using an amino acid linker sequence described herein.

### Example 5 Activity of Adnectin™/Anti-HIV bnAb Fusion Polypeptides on Antiviral Activity Against HIV

Inhibition activity and binding affinity was measured as described above in Section IX, and in Example 2 using CD4/N17/anti-HIV bnAb fusion polypeptides as described herein.

In FIG. 6A and FIG. 6B it can be seen that the 108v4 (MPER) bnAb Adnectin™ fusions surprisingly showed enhancement of potency. The 10E8v4 bnAb-N17 Adnectin™ fusion showed up to a 5-fold increase in potency, depending on the linker, as seen in FIG. 6A. The 10E8v4-CD4 Adnectin™ fusions, with either the A or B CD4 Adnectin™, showed significant enhancement of potency, up to >2000-fold enhancement, with sub-picomolar activity (0.4 pM), compared to 10E8 bnAb alone or Adnectin™ alone (see FIG. 6B). Again, the linker impacted the results seen.

Without being bound by theory, it is possible that the N17 and CD4 Adnectins™ synergize well with the 10E8.v4 bnAbs because both Adnectins™ operate post-CD4 binding. For the CD4 Adnectin™s, synergy afforded by the anchoring effect of the anti-CD4 Adnectin™ may require viral CD4 binding, as it is known that the HIV virus must be in close proximity to the target cell for surface-bound inhibitors to be effective. For the N17 Adnectina™, these Adnectins™ function post-CD4 binding, because CD4 interaction is needed to reveal the N17 Adnectin™ binding site on gp41.

In contrast, CD4bs bnAbs block the interaction with CD4, so the virus does not contact the cell surface. And likewise, PGT-128 may act by cross-linking gp120 trimers and destabilizing them, which effect may be irrelevant after contact with CD4, thus affording no enhancement of activity with such Adnectin™-bnAb fusion polypeptides. Because 10E8 bnAbs operate post-CD4 binding to block membrane fusion, this may explain the enhancement activity and apparent synergy seen with 10E8 bnAb/Adnectin™ fusion polypeptides.

FIG. 7 shows that the enhanced cell binding of B vs A Adnectins™ in 10E8v4 bnAb fusion polypeptides contributes to the improved potency observed with such fusion polypeptides. The A fusion was with the CD4 A Adnectin™ (SEQ ID NO: 5) using linker (ESPEPETED)₂E and the heavy chain of 10E8v4 (SEQ ID NO: 7); and the B fusion was with CD4 B Adnectin™ (SEQ ID NO: 78) with linker (ESPEPETED)₂E and the heavy chain of 10E8v4 (SEQ ID NO: 7). Each fusion shows approximately a 2000-fold difference in binding EC₅₀ vs Antiviral EC₅₀ (See FIG. 7, wherein the B fusion shows a binding EC5₀ of 0.9 nM and an antiviral EC₅₀ of 0.4 pM; and the A fusion shows a binding EC₅₀ of 33 nM and an antiviral EC₅₀ of 17 pM). The two dose response curves measure the receptor occupancy of the 10E8v4-anti-CD4 Domain 2 Adnectin fusion protein (bold line) and the 10E8v4-anti-CD4 Domain 4 Adnectin fusion protein (broken line) on CD4 protein present on MT-2 cells. The arrows illustrate the antiviral EC₅₀ of each protein, which is much more potent than the receptor binding EC₅₀. The improved antiviral EC₅₀ of the B Fusion protein compared to the A Fusion protein may be related to the improved binding to CD4, as evidenced by the more potent receptor binding EC₅₀.

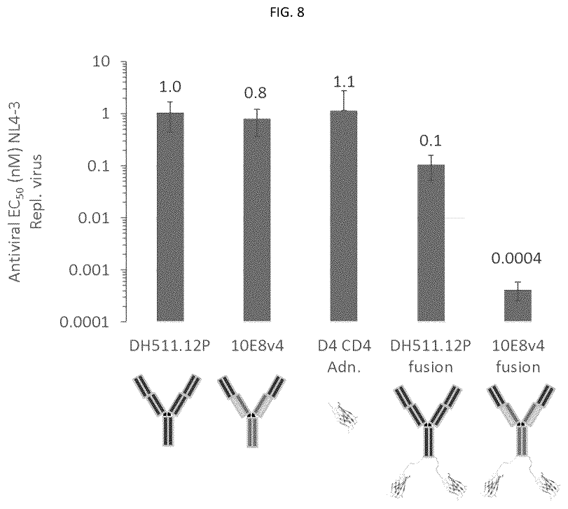

FIG. 8 shows results with another MPER bnAb fusion comprising DH511.12P (CD4 B Adnectin™ and linker sequence the same as described above for FIG. 7), although the enhanced activity with the DH511.12P bnAb fusion is not as great as that seen with fusions comprising 10E8v4 bnAbs.

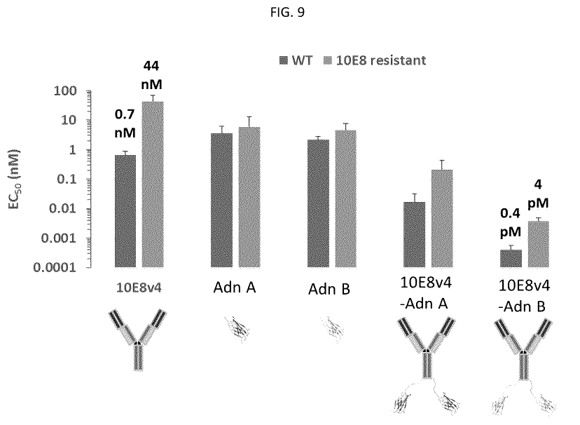

FIG. 9 shows the activity of the CD4 A/and CD4 B/10E8v4 bnAb fusion peptides on cells infected with an HIV strain reported to be resistant to 10E8v4 bnAb. The activity was determined as described in Example 2, using 10E8 resistance mutations (W680E/K683Q) engineered onto the replicating NL₄₋₃ backbone (see Huang et al. Nature (2012), 491, pp. 406-412). As can be seen, both the CD4 A/10E8v4 bnAb fusion and the CD4 B/10E8v4 bnAb fusion show some level of enhanced activity compared to 10E8v4, CD4 A Adnectin™ or CD4 B Adnectin™ alone, but surprisingly, the CD4 B/bnAb fusion polypeptide retains 4 pM activity against the cells infected with HIV shown to have resistance to 10E8v4 bnAb.

### Example 6—Cell-Cell Fusion Assay with 123 HIV Clinical Envelopes

The breadth of the activity of the CD4 A/or CD4 B/10E8v4 fusion polypeptides against 123 clinical envelopes was done.

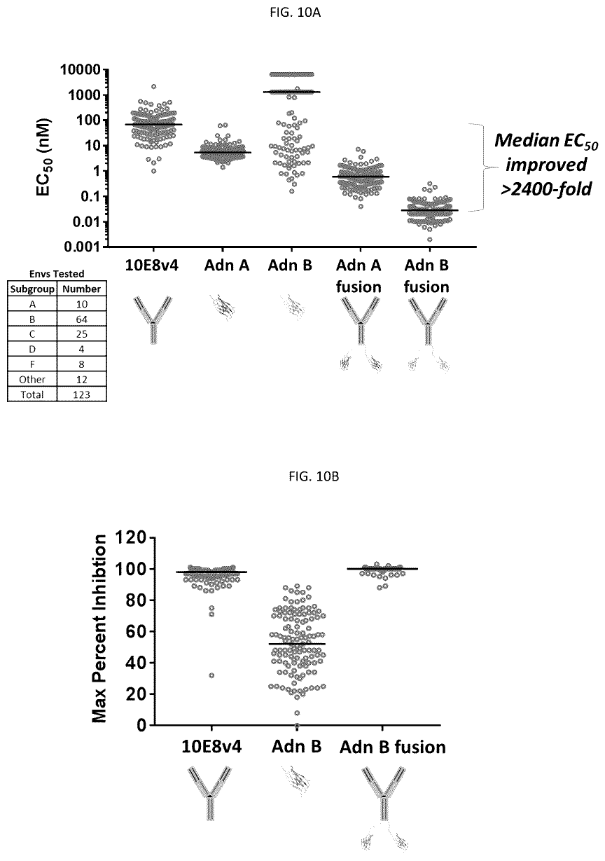

FIG. 10A shows the binding EC₅₀ of CD4 A/10E8v4 fusion or a CD4 B/10E8v4 fusion compared to 10E8v4, CD4 A or CD4 B alone, for 123 different clinical envelopes. The breakdown, by subgroup of envelopes tested was as follows: Subgroup A: 10; Subgroup B: 64; Subgroup C: 25; Subgroup D: 4; Subgroup F: 8; Subgroup Other: 12; for a total of 123 clinical envelopes.

The median EC₅₀ (nM) value for the A fusion was 0.59, and for the B fusion was 0.0028, compared to 68 (10E8v4 bnAb along), 5.3 (A Adnectin™ alone) and 1300 (B Adnectin™ alone). Similar values were determined for the Geometric mean EC₅₀ (nM) for the A and B fusion polypeptides, although the control values differed somewhat, especially for the B Adnectin™ alone. When expressed as g/mL, the Geometric mean EC₅₀ values for the A and B fusion polypeptides were 0.093 and 0.0048, respectively, compared to 8.7 (10E8v4 bnAb alone), 0.06 (A Adnectin™ along) and 1.8 (B Adnectin™ alone). All envelopes tested showed susceptibility to the CD4/10E8v4 fusion polypeptides, with EC₅₀ s all below 0.3. nM. For the D$ fusion, the median EC₅₀ value improved >2400 compared to the controls. Table 10 below shows the mean values determined for FIG. 10A.

TABLE 10

| | | | | 10E8v4- | 10E8v4- |
|---|---|---|---|---|---|
| | | CD4-A | CD4-B | CD4-A | CD4-B |
| | 10E8v4 | ADN | ADN | fusion | fusion |
| Median EC₅₀ (nM) | 68 | 5.3 | 1300 | 0.59 | 0.0028 |
| Geometric mean | 59 | 5.5 | 163 | 0.54 | 0.028 |
| EC₅₀ (nM) | | | | | |
| Geometric mean | 8.7 | 0.006 | 1.8 | 0.093 | 0.0048 |
| EC₅₀ (μg/mL) | | | | | |

FIG. 10B shows the Maximum Percent Inhibition by the CD4 B/10E8v4 fusion polypeptide against the clinical envelopes. As seen, the B fusion polypeptide showed 100% maximum inhibition compared to the CD4 B Adnectin™ alone. Although most of the viruses also exhibited 100% maximum percent inhibition to 10E8v4 alone, the few that were lower also showed improved MPI in the CD4 B/10E8v4 fusion polypeptide treated.

### Example 7—Evaluation of Polypeptide Pharmacokinetics

### Human CD4 Transgenic Mouse Model

Male and female homozygous human CD4 mice are obtained from GenOway, Lyon, France.

### WT Mouse PK Studies

8-21 day single IV bolus dose studies will be run in female C57Bl/6 WT mice to assess the PK properties of the various polypeptides of the invention. Adnectin™-bnAb fusion polypeptides of the invention can be dosed at 5 mg/kg up to 10 mg/kg. Plasma samples can be collected in citrate phosphate dextrose tubes (CPD) and stored at −80° C. until analysis.

hCD4 Mouse PK Studies

7-10 day single IV bolus dose studies can be run in heterozygous hCD4 mice to assess the PK properties of various polypeptides of the invention in the presence of target. Polypeptides of the invention doses and sample collection methods will be the same as described above for the WT mice.

### Cynomolgus Monkey Studies

A 1-week single dose study will be conducted in female cynos to determine the PK of polypeptide of the invention. Following a 1 mg/kg dose, serum samples will be collected at indicated times, aliquoted and quick frozen for MSD or LC/MS analysis.

### Pharmacokinetic Measurements

Drug levels can be measured in mouse or cyno plasma using the Mesoscale technology platform or colorimetric ELISA formats. Fc-polypeptide fusions can be captured via the protein PRD828 (BMS), a protein that specifically binds the peptide component of polypeptide of the invention and is detected using a Goat anti-Human IgG Fc-HRP conjugated pAb (Pierce #31413). HSA-polypeptide fusions can be captured via PRD828 and detected using a goat pAb against HSA (Bethyl, TX #A80-229A) that is ruthenium labeled. Sample concentrations can be calculated from a standard curve using a 5-parameter logarithmic fit. Non-compartmental analyses can be performed using Phoenix WINNONLIN® 6.3 (Pharsight Corporation, Mountain View, CA) using a plasma model and linear up/log down calculation method.

SEQUENCES
SEQ ID NO: 1
VSDVPRDLEVVAATPTSLLISWDAPAVTVRYYRITYGETGGNSPVQEFTVPGSKSTATISGLKPGVDY
TITVYAVTGRGDSPASSKPISINYRT
SEQ ID NO: 2
AVGIGAMFLGFLGAAGSTMGAASVTLTVQARQLLSGIVQQQNNLLRAIEAQQHLLQLTVWGIKQLQAR
VLAVERYLKDQQLLGIWGCSGKLICTTAVPWNASWSNKSLDEIWNNMTWMEWEREIDNYTGLIYTLIE
ESQNQQEKNEQELLELDKWASLWNWFDITNWLWYIK
SEQ ID NO: 3
GVSDVPRDLEVVAATPTSLLISWDAPAVTVHSYHIQYWPLGSYQRYQVFSVPGSKSTATISGLKPGVE
YQIRVYAETGRGESDQSLGWIQIGYRTE
SEQ ID NO: 4
GVSDVPRDLEVVAATPTSLLISWDAPAVTVHSYHIQYWPLGSYQRYQVFSVPGSKSTATISGLKPGVE
YQIRVYAETGRGESDQSLGWIQIGYRTP
SEQ ID NO: 5
GVSDVPRDLEVVAATPTSLLISWDAPAVTVHSYHIQYWPLGSYQRYQVFSVPGSKSTATISGLKPGVE
YQIRVYAETGGADSDQSFGWIQIGYRTP
(10E8v4 Light Chain)
SEQ ID NO: 6
ASELTQDPAVSVALKQTVTITCRGDSLRSHYASWYQKKPGQAPVLLFYGKNNRPSGIPDRESGSASGN
RASLTITGAQAEDEADYYCSSRDKSGSRLSVFGGGTKLTVLSQPKAAPSVTLFPPSSEELQANKATLV
CLISDFYPGAVTVAWKADSSPVKAGVETTTPSKQSNNKYAASSYLSLTPEQWKSHRSYSCQVTHEGST
VEKTVAPTECS
(10E8v4 Heavy Chain - SLSLSP variant)
SEQ ID NO: 7
EVRLVESGGGLVKPGGSLRLSCSASGFDFDNAWMTWVRQPPGKGLEWVGRITGPGEGWSVDYAESVKG
RFTISRDNTKNTLYLEMNNVRTEDTGYYFCARTGKYYDFWSGYPPGEEYFQDWGQGTLVIVSSASTKG
PSVFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWLNGKEYKCKVSNK
ALPAPIEKTISKAKGQPREPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLSP
(10E8v4 Heavy Chain - SLSLSPGK variant)
SEQ ID NO: 8
EVRLVESGGGLVKPGGSLRLSCSASGFDEDNAWMTWVRQPPGKGLEWVGRITGPGEGWSVDYAESVKG
RFTISRDNTKNTLYLEMNNVRTEDTGYYFCARTGKYYDFWSGYPPGEEYFQDWGQGTLVIVSSASTKG
PSVFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWLNGKEYKCKVSNK
ALPAPIEKTI SKAKGQPREPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLSPGK
(bnAb-Adnectin fusion polypeptide ™)
SEQ ID NO: 9
EVRLVESGGGLVKPGGSLRLSCSASGFDFDNAWMTWVRQPPGKGLEWVGRITGPGEGWSVDYAESVKG
RFTISRDNTKNTLYLEMNNVRTEDTGYYFCARTGKYYDFWSGYPPGEEYFQDWGQGTLVIVSSASTKG
PSVFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWLNGKEYKCKVSNK
ALPAPIEKTISKAKGQPREPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLSPGGGGSGGGGGGGGSGG
GGSGGGGSGVSDVPRDLEVVAATPTSLLISWDAPAVTVHSYHIQYWPLGSYQRYQVFSVPGSKSTATI
SGLKPGVEYQIRVYAETGGADSDQSFGWIQIGYRTP
(bnAb-Adnectin fusion polypeptide ™)
SEQ ID NO: 10
EVRLVESGGGLVKPGGSLRLSCSASGFDFDNAWMTWVRQPPGKGLEWVGRITGPGEGWSVDYAESVKG
RFTISRDNTKNTLYLEMNNVRTEDTGYYFCARTGKYYDFWSGYPPGEEYFQDWGQGTLVIVSSASTKG
PSVFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWLNGKEYKCKVSNK
ALPAPIEKTISKAKGQPREPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLSPESPEPETPEDESPEPET
PEDEGVSDVPRDLEVVAATPTSLLISWDAPAVTVHSYHIQYWPLGSYQRYQVFSVPGSKSTATISGLK
PGVEYQIRVYAETGGADSDQSFGWIQIGYRTP
(bnAb-Adnectin fusion polypeptide ™)
SEQ ID NO: 11
ASELTQDPAVSVALKQTVTITCRGDSLRSHYASWYQKKPGQAPVLLFYGKNNRPSGIPDRFSGSASGN
RASLTITGAQAEDEADYYCSSRDKSGSRLSVFGGGTKLTVLSQPKAAPSVTLFPPSSEELQANKATLV
CLISDFYPGAVTVAWKADSSPVKAGVETTTPSKQSNNKYAASSYLSLTPEQWKSHRSYSCQVTHEGST
VEKTVAPTECSGGGGSGGGGSGGGGSGGGGSGGGGSGVSDVPRDLEVVAATPTSLLISWDAPAVTVHS
YHIQYWPLGSYQRYQVFSVPGSKSTATISGLKPGVEYQIRVYAETGGADSDQSFGWIQIGYRTP
(bnAb-Adnectin fusion polypeptide ™)
SEQ ID NO: 12
ASELTQDPAVSVALKQTVTITCRGDSLRSHYASWYQKKPGQAPVLLFYGKNNRPSGIPDRFSGSASGN
RASLTITGAQAEDEADYYCSSRDKSGSRLSVFGGGTKLTVLSQPKAAPSVTLFPPSSEELQANKATLV
CLISDFYPGAVTVAWKADSSPVKAGVETTTPSKQSNNKYAASSYLSLTPEQWKSHRSYSCQVTHEGST
VEKTVAPTECSESPEPETPEDESPEPETPEDEGVSDVPRDLEVVAATPTSLLISWDAPAVTVHSYHIQ
YWPLGSYQRYQVFSVPGSKST
SEQ ID NO: 13
HSYHIQYWPLGSYQRYQVFS
SEQ ID NO: 14
EYQIRVYAETGGADSDQSMGWIQIG
SEQ ID NO: 15
LSYHIQYWPLGLYQAYQVFS
SEQ ID NO: 16
EYQIRVYAETGRGESPASFGWIQIG
SEQ ID NO: 17
HAYHIQYWPLGFYQGYQVFS
SEQ ID NO: 18
EYQIRVYAETGLGDAHQSLGWIQIG
SEQ ID NO: 19
LAYHIQYWPLGWYQRYQIFS
SEQ ID NO: 20
LAYHIQYWPLGWYQRYQVES
SEQ ID NO: 21
HFYHIQYWPLGLYHLYQVES
SEQ ID NO: 22
YSYHIQYWPLGWYHRYQVFS
SEQ ID NO: 23
EYQIRVYAETGADDPVQALGWIQIG
SEQ ID NO: 24
RCYHIQYWPLGLYPLYQVFS
SEQ ID NO: 25
EYQIRVYAETGDESSVQPFGWIQIG
SEQ ID NO: 26
YSYHIQYWPLGWYQRYQVES
SEQ ID NO: 27
EYQIRVYAETDGGRSQQSFGWIQIG
SEQ ID NO: 28
SSYHIQYWPLGAYQRYQVES
SEQ ID NO: 29
HAYHIQYWPLGLYQRYQVFS
SEQ ID NO: 30
HAYYIQYWPLGSYQFYQVFA
SEQ ID NO: 31
HSYHIQYWPLGSYLRYQVES
SEQ ID NO: 32
LSYHIQYWPLGFYQRYQVES
SEQ ID NO: 33
SAYHIQYWPLGWYHRYQIFS
SEQ ID NO: 34
YSYHIQYWPLGAYSRHQLFS
SEQ ID NO: 35
EYQIRVYAETGGDGSEMYFGWIQIG
SEQ ID NO: 36
LAYHIQYWPLGWYHLYQVES
SEQ ID NO: 37
LAYHIQYWPLGWYQLYKVFS
SEQ ID NO: 38
DYQIRVYAETSGESSEQYLGWIQIG
SEQ ID NO: 39
HSYHIQYWPLGWYQLYQVFS
SEQ ID NO: 40
EYQIRVYAETEVDSGQHSFGWIQIG
SEQ ID NO: 41
LAYHIQYWPLGWYQRYQIFS
SEQ ID NO: 42
EYQIRVYAETGESGAQQSFGWIQIG
SEQ ID NO: 43
QSYHIQYWPLGAYQLYQLES
SEQ ID NO: 44
HAYHIQYWPLGFYQGYQVES
SEQ ID NO: 45
EYQIRVYADTGRGYQLSYSWIQIGY
SEQ ID NO: 46
FRYHIQYWPLGGYERYQVFT
SEQ ID NO: 47
HSYHIQYWPLGSYHLYQLES
SEQ ID NO: 48
HSYHIQYWPLGWYQLYQVFT
SEQ ID NO: 49
EYQIRVYAETGGFGSPPNFGWIQIG
SEQ ID NO: 50
QFYHIQYWPLGSYQRYQVES
SEQ ID NO: 51
NSYHIQYWPLGWYHRYQVES
SEQ ID NO: 52
HSYHIQYWPLGRYQLYQVES
SEQ ID NO: 53
LAYHIQYWPLGWYHLYQIFS
SEQ ID NO: 54
EYQIRVYAETGGVGWHHSFGWIQIG
SEQ ID NO: 55
HVYHIQYWPLGWYPRYQVFS
SEQ ID NO: 56
HSYHIPYWELAWYQRYQVES
SEQ ID NO: 57
ESYHIQYWPLGLYHRYQVES
SEQ ID NO: 58
LAYHIQYWPLGWYQAYQVES
SEQ ID NO: 59
YLYHIQYWPLGWYHRYQVFT
SEQ ID NO: 60
RFYHIQYWPLGWYHCYQVFV
SEQ ID NO: 61
EYQIRVYAQTGDGSSQEYFGWIQIG
SEQ ID NO: 62
HSYHIQYWPLGWYYRYQVFS
SEQ ID NO: 63
EYQIRVYAETGGSGSQQYWGWIQIG
SEQ ID NO: 64
HAYHIQYWPLGFYQGYQVFS
SEQ ID NO: 65
HSYHIQYWPLGLYVLYQVES
SEQ ID NO: 66
EYQIRVYAETGAGGSEHSFGWIQIG
SEQ ID NO: 67
LSYHIQYWPLGRYERYQVES
SEQ ID NO: 68
EYQIRVYAETVGGESLDSFSWIQIG
SEQ ID NO: 69
LSYHIQYWPLGWYQLYQVFY
SEQ ID NO: 70
EYQIRVYAETRVGGSVASFGWIQIG
SEQ ID NO: 71
LAYHIQYWPLGRYQLYQVES
SEQ ID NOs: 72-91
(CD4 Adnectins ™) listed in Table 4
SEQ ID NOs: 92-348
(N17 Adnectins ™) listed in Table 5
SEQ ID NOS: 349-369
(HIV fusion peptide inhibitors) listed in Table 6
SEQ ID NOS: 370-748
(linkers) listed in Table 7
(10E8v4-HC-ESPEPETED2-CD4 - A)
SEQ ID NO: 749
EVRLVESGGGLVKPGGSLRLSCSASGFDFDNAWMTWVRQPPGKGLEWVGRITGPGEGWSVDYAESVKG
RFTISRDNTKNTLYLEMNNVRTEDTGYYFCARTGKYYDFWSGYPPGEEYFQDWGQGTLVIVSSASTKG
PSVFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWLNGKEYKCKVSNK
ALPAPIEKTISKAKGQPREPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLSPESPEPETPEDESPEPET
PEDEGVSDVPRDLEVVAATPTSLLISWDAPAVTVHSYHIQYWPLGSYQRYQVFSVPGSKSTATISGLK
PGVEYQIRVYAETGGADSDQSFGWIQIGYRTP
(10E8v4-HC-(G4S)5-CD4 - A)
SEQ ID NO: 750
EVRLVESGGGLVKPGGSLRLSCSASGFDFDNAWMTWVRQPPGKGLEWVGRITGPGEGWSVDYAESVKG
RFTISRDNTKNTLYLEMNNVRTEDTGYYFCARTGKYYDFWSGYPPGEEYFQDWGQGTLVIVSSASTKG
PSVFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWLNGKEYKCKVSNK
ALPAPIEKTISKAKGQPREPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLSPGGGGSGGGGSGGGGSGG
GGSGGGGSGVSDVPRDLEVVAATPTSLLISWDAPAVTVHSYHIQYWPLGSYQRYQVFSVPGSKSTATI
SGLKPGVEYQIRVYAETGGADSDQSFGWIQIGYRTP
(VRC01-HC-CD4- A):
SEQ ID NO: 751
QVQLVQSGGQMKKPGESMRISCRASGYEFIDCTLNWIRLAPGKRPEWMGWLKPRGGAVNYARPLQGRV
TMTRDVYSDTAFLELRSLTVDDTAVYFCTRGKNCDYNWDFEHWGRGTPVIVSSPSTKGPSVFPLAPSS
KSTSGGTAALGCLVKDYFPEPVTVSWNSGALTSGVHTFPAVLQSSGLYSLSSVVTVPSSSLGTQTYIC
NVNHKPSNTKVDKKVEPKSCDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNGQPENNYKTTPPVLDSDGSF
FLYSKLTVDKSRWQQGNVFSCSVMHEALHNHYTQKSLSLSPGGGGSGGGGSGGGGSGGGGSGGGGSGV
SDVPRDLEVVAATPTSLLISWDAPAVTVHSYHIQYWPLGSYQRYQVFSVPGSKSTATISGLKPGVEYQ
IRVYAETGGADSDQSFGWIQIGYRTP
(VRC01-LC-CD4-A):
SEQ ID NO: 752
EIVLTQSPGTLSLSPGETAIISCRTSQYGSLAWYQQRPGQAPRLVIYSGSTRAAGIPDRFSGSRWGPD
YNLTISNLESGDFGVYYCQQYEFFGQGTKVQVDIKRTVAAPSVFIFPPSDEQLKSGTASVVCLLNNFY
PREAKVQWKVDNALQSGNSQESVTEQDSKDSTYSLSSTLTLSKADYEKHKVYACEVTHQGLRSPVTKS
FNRGECGGGGSGGGGSGGGGSGGGGSGGGGSGVSDVPRDLEVVAATPTSLLISWDAPAVTVHSYHIQY
WPLGSYQRYQVFSVPGSKSTATISGLKPGVEYQIRVYAETGGADSDQSFGWIQIGYRTP
(10E8v4-HC-N17)
SEQ ID NO: 753
EVRLVESGGGLVKPGGSLRLSCSASGFDFDNAWMTWVRQPPGKGLEWVGRITGPGEGWSVDYAESVKG
RFTISRDNTKNTLYLEMNNVRTEDTGYYFCARTGKYYDFWSGYPPGEEYFQDWGQGTLVIVSSASTKG
PSVFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWLNGKEYKCKVSNK
ALPAPIEKTISKAKGQPREPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLSPGKESPEPETPEDEGVSD
VPRDLEVVAATPTSLLISWEYKVHPYRYYRITYGETGGNSPVQEFTVPSVLSTAEISGLKPGVDYTIT

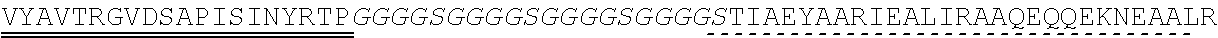

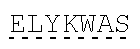

(10E8v4-LC-N17)
SEQ ID NO: 754
ASELTQDPAVSVALKQTVTITCRGDSLRSHYASWYQKKPGQAPVLLFYGKNNRPSGIPDRFSGSASGN
RASLTITGAQAEDEADYYCSSRDKSGSRLSVFGGGTKLTVLSQPKAAPSVTLFPPSSEELQANKATLV
CLISDFYPGAVTVAWKADSSPVKAGVETTTPSKQSNNKYAASSYLSLTPEQWKSHRSYSCQVTHEGST
VEKTVAPTECSESPEPETPEDEGVSDVPRDLEVVAATPTSLLISWEYKVHPYRYYRITYGETGGNSPV

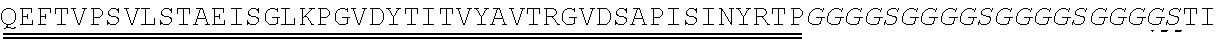

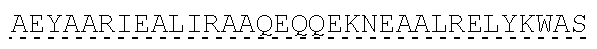

For above SEQ ID Nos: 749-754, the anti-HIV bnAb portion is in bold,
the N17 Adnectin ™ portion is double-underlined, the HIV fusion
portion is dotted underlined, and the linker portions are italicized
(10E8v4-HC-ESPEPETED₂-CD4 A)
SEQ ID NO: 755
EVRLVESGGGLVKPGGSLRLSCSASGFDFDNAWMTWVRQPPGKGLEWVGRITGPGEGWSVDYAESVKG
RFTISRDNTKNTLYLEMNNVRTEDTGYYFCARTGKYYDFWSGYPPGEEYFQDWGQGTLVIVSSASTKG
PSVFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWLNGKEYKCKVSNK
ALPAPIEKTISKAKGQPREPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLSPESPEPETPEDESPEPET
PEDEGVSDVPRDLEVVAATPTSLLISWDAPAVTVHSYHIQYWPLGSYQRYQVFSVPGSKSTATISGLK
PGVEYQIRVYAETGGADSDQSFGWIQIGYRTP
(10E8v4-HC-(G4S)₅-CD4 A)
SEQ ID NO: 756
EVRLVESGGGLVKPGGSLRLSCSASGFDFDNAWMTWVRQPPGKGLEWVGRITGPGEGWSVDYAESVKG
RFTISRDNTKNTLYLEMNNVRTEDTGYYFCARTGKYYDFWSGYPPGEEYFQDWGQGTLVIVSSASTKG
PSVFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWLNGKEYKCKVSNK
ALPAPIEKTISKAKGQPREPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLSPGGGGSGGGGSGGGGSGG
GGSGGGGSGVSDVPRDLEVVAATPTSLLISWDAPAVTVHSYHIQYWPLGSYQRYQVFSVPGSKSTATI
SGLKPGVEYQIRVYAETGGADSDQSFGWIQIGYRTP
(10E8v4-HC-ESPEPETED₂-CD4 B)
SEQ ID NO: 757
EVRLVESGGGLVKPGGSLRLSCSASGFDFDNAWMTWVRQPPGKGLEWVGRITGPGEGWSVDYAESVKG
RFTISRDNTKNTLYLEMNNVRTEDTGYYFCARTGKYYDFWSGYPPGEEYFQDWGQGTLVIVSSASTKG
PSVFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWLNGKEYKCKVSNK
ALPAPIEKTISKAKGQPREPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLSPESPEPETPEDESPEPET
PEDEMASTSGSASYLIPSDLEVVAATPTSLSIYWYPVASTIINFRITYVETGGNSPVQEFTVPGSKST
ATISGLKPGVDYTITVYAVHYEQKYSEYWIGHPISINYRTEGSGSHHHHHH
(10E8v4-HC-(G4S)₅-CD4 B)
SEQ ID NO: 758
EVRLVESGGGLVKPGGSLRLSCSASGFDFDNAWMTWVRQPPGKGLEWVGRITGPGEGWSVDYAESVKG
RFTISRDNTKNTLYLEMNNVRTEDTGYYFCARTGKYYDFWSGYPPGEEYFQDWGQGTLVIVSSASTKG
PSVFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWLNGKEYKCKVSNK
ALPAPIEKTISKAKGQPREPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLSPGGGGSGGGGSGGGGSGG
GGSGGGGSMASTSGSASYLIPSDLEVVAATPTSLSIYWYPVASTIINFRITYVETGGNSPVQEFTVPG
SKSTATISGLKPGVDYTITVYAVHYEQKYSEYWIGHPISINYRTEGSGSHHHHHH
(VRC01-HC-CD4 A)
SEQ ID NO: 759
QVQLVQSGGQMKKPGESMRISCRASGYEFIDCTLNWIRLAPGKRPEWMGWLKPRGGAVNYARPLQGRV
TMTRDVYSDTAFLELRSLTVDDTAVYFCTRGKNCDYNWDFEHWGRGTPVIVSSPSTKGPSVFPLAPSS
KSTSGGTAALGCLVKDYFPEPVTVSWNSGALTSGVHTFPAVLQSSGLYSLSSVVTVPSSSLGTQTYIC
NVNHKPSNTKVDKKVEPKSCDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNGQPENNYKTTPPVLDSDGSF
FLYSKLTVDKSRWQQGNVFSCSVMHEALHNHYTQKSLSLSPGGGGSGGGGSGGGGSGGGGSGGGGSGV
SDVPRDLEVVAATPTSLLISWDAPAVTVHSYHIQYWPLGSYQRYQVFSVPGSKSTATISGLKPGVEYQ
IRVYAETGGADSDQSFGWIQIGYRTP
(VRC01-LC-CD4 A)
SEQ ID NO: 760
EIVLTQSPGTLSLSPGETAIISCRTSQYGSLAWYQQRPGQAPRLVIYSGSTRAAGIPDRFSGSRWGPD
YNLTISNLESGDFGVYYCQQYEFFGQGTKVQVDIKRTVAAPSVFIFPPSDEQLKSGTASVVCLLNNFY
PREAKVQWKVDNALQSGNSQESVTEQDSKDSTYSLSSTLTLSKADYEKHKVYACEVTHQGLRSPVTKS
FNRGECGGGGSGGGGSGGGGSGGGGSGGGGSGVSDVPRDLEVVAATPTSLLISWDAPAVTVHSYHIQY
WPLGSYQRYQVFSVPGSKSTATISGLKPGVEYQIRVYAETGGADSDQSFGWIQIGYRTP
(VRC01-HC-CD4 B)
SEQ ID NO: 761
QVQLVQSGGQMKKPGESMRISCRASGYEFIDCTLNWIRLAPGKRPEWMGWLKPRGGAVNYARPLQGRV
TMTRDVYSDTAFLELRSLTVDDTAVYFCTRGKNCDYNWDFEHWGRGTPVIVSSPSTKGPSVFPLAPSS
KSTSGGTAALGCLVKDYFPEPVTVSWNSGALTSGVHTFPAVLQSSGLYSLSSVVTVPSSSLGTQTYIC
NVNHKPSNTKVDKKVEPKSCDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNGQPENNYKTTPPVLDSDGSF
FLYSKLTVDKSRWQQGNVFSCSVMHEALHNHYTQKSLSLSPGGGGSGGGGSGGGGSGGGGSGGGGSMA
STSGSASYLIPSDLEVVAATPTSLSIYWYPVASTIINFRITYVETGGNSPVQEFTVPGSKSTATISGL
KPGVDYTITVYAVHYEQKYSEYWIGHPISINYRTEGSGSHHHHHH
(VRC01-LC-CD4 B)
SEQ ID NO: 762
EIVLTQSPGTLSLSPGETAIISCRTSQYGSLAWYQQRPGQAPRLVIYSGSTRAAGIPDRFSGSRWGPD
YNLTISNLESGDFGVYYCQQYEFFGQGTKVQVDIKRTVAAPSVFIFPPSDEQLKSGTASVVCLLNNFY
PREAKVQWKVDNALQSGNSQESVTEQDSKDSTYSLSSTLTLSKADYEKHKVYACEVTHQGLRSPVTKS
FNRGECGGGGSGGGGSGGGGSGGGGSGGGGSMASTSGSASYLIPSDLEVVAATPTSLSIYWYPVASTI
INFRITYVETGGNSPVQEFTVPGSKSTATISGLKPGVDYTITVYAVHYEQKYSEYWIGHPISINYRTE
GSGSHHHHHH
In SEQ ID NOS: 755-762 above, the anti-CD4 Adnectin ™ sequences are
underlined. The anti-HIV bnAb portion is indicated in bold. The
linker sequences are italicized.
(10E8v4-HC-N17430)
SEQ ID NO: 763
EVRLVESGGGLVKPGGSLRLSCSASGFDFDNAWMTWVRQPPGKGLEWVGRITGPGEGWSVDYAESVKG
RFTISRDNTKNTLYLEMNNVRTEDTGYYFCARTGKYYDFWSGYPPGEEYFQDWGQGTLVIVSSASTKG
PSVFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWLNGKEYKCKVSNK
ALPAPIEKTISKAKGQPREPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLSPGKESPEPETPEDEGVSD
VPRDLEVVAATPTSLLISWEYKVHPYRYYRITYGETGGNSPVOEFTVPSVLSTAEISGLKPGVDYTIT

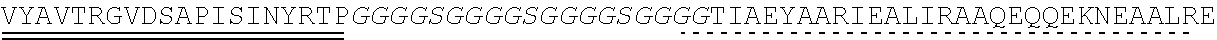

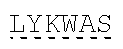

(10E8v4-LC-N17)
SEQ ID NO: 764
ASELTQDPAVSVALKQTVTITCRGDSLRSHYASWYQKKPGQAPVLLFYGKNNRPSGIPDRFSGSASGN
RASLTITGAQAEDEADYYCSSRDKSGSRLSVFGGGTKLTVLSQPKAAPSVTLFPPSSEELQANKATLV
CLISDFYPGAVTVAWKADSSPVKAGVETTTPSKQSNNKYAASSYLSLTPEQWKSHRSYSCQVTHEGST
VEKTVAPTECSESPEPETPEDEGVSDVPRDLEVVAATPTSLLISWEYKVHPYRYYRITYGETGGNSPV

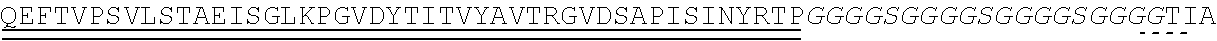

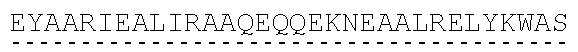

For SEQ ID NOs: 763 and 764, the anti-HIV bnAb portion is in bold,
the N17 Adnectin ™ portion is double-underlined, the HIV fusion
portion is dotted underlined, and the linker portions are
italicized.
(10E8v4-HC-CD4 B)
SEQ ID NO: 765
EVRLVESGGGLVKPGGSLRLSCSASGFDFDNAWMTWVRQPPGKGLEWVGRITGPGEGWSVDYAESVKG
RFTISRDNTKNTLYLEMNNVRTEDTGYYFCARTGKYYDFWSGYPPGEEYFQDWGQGTLVIVSSASTKG
PSVFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWLNGKEYKCKVSNK
ALPAPIEKTISKAKGQPREPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLSPESPEPETPEDESPEPET
PEDEMASTSGSASYLIPSDLEVVAATPTSLSIYWYPVASTIINFRITYVETGGNSPVQEFTVPGSKST
ATISGLKPGVDYTITVYAVHYEQKYSEYWIGHPISINYRTEGSGSHHHHHH
(10E8v4-LC-CD4 B)
SEQ ID NO: 766
ASELTQDPAVSVALKQTVTITCRGDSLRSHYASWYQKKPGQAPVLLFYGKNNRPSGIPDRFSGSASGN
RASLTITGAQAEDEADYYCSSRDKSGSRLSVFGGGTKLTVLSQPKAAPSVTLFPPSSEELQANKATLV
CLISDFYPGAVTVAWKADSSPVKAGVETTTPSKQSNNKYAASSYLSLTPEQWKSHRSYSCQVTHEGST
VEKTVAPTECSGGGGSGGGGSGGGGSGGGGSGGGGSMASTSGSASYLIPSDLEVVAATPTSLSIYWYP
VASTIINFRITYVETGGNSPVQEFTVPGSKSTATISGLKPGVDYTITVYAVHYEQKYSEYWIGHPISI
NYRTEGSGSHHHHHH
(10E8v4-LC-CD4 B)
SEQ ID NO: 767
ASELTQDPAVSVALKQTVTITCRGDSLRSHYASWYQKKPGQAPVLLFYGKNNRPSGIPDRFSGSASGN
RASLTITGAQAEDEADYYCSSRDKSGSRLSVFGGGTKLTVLSQPKAAPSVTLFPPSSEELQANKATLV
CLISDFYPGAVTVAWKADSSPVKAGVETTTPSKQSNNKYAASSYLSLTPEQWKSHRSYSCQVTHEGST
VEKTVAPTECSESPEPETPEDESPEPETPEDEMASTSGSASYLIPSDLEVVAATPTSLSIYWYPVAST
IINFRITYVETGGNSPVQEFTVPGSKSTATISGLKPGVDYTITVYAVHYEQKYSEYWIGHPISINYRT
EGSGSHHHHHH
In SEQ ID NOs: 765-767 above, the anti-CD4 Adnectin ™ sequences
are underlined. The anti-HIV bnAb portion is indicated in bold. The
linker sequences are italicized.
10E8v2-HC(Heavy Chain)
SEQ ID NO: 768
EVRLVESGGGLVKPGGSLRLSCSASGFNFDDAWMTWVRQPPGKGLEWVGRISGPGEGWSVDYAESVKG
RFTISRLNSINFLYLEMNNVRTEDTGYYFCARTGKHYDFWSGYPPGEEYFQDWGQGTLVIVSSASTKG
PSVFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSVFLFPPKPKDTLMISRTPE
VTCVVVDVSHEDPEVKENWYVDGVEVHNAKTKPREEQYNSTYRVVSVLTVLHQDWLNGKEYKCKVSNK
ALPAPIEKTISKAKGQPREPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLSPGK
10E8v3-HC (Heavy Chain)
SEQ ID NO: 769
EVQLVESGGDLVKPGGSLRLSCSASGFSFKNTWMTWVRQAPGKGLEWVGRITGPGEGWTSDYAATVQG
RFTISRNNMIDMLYLEMNRLRTDDTGLYYCVHTEKYYNFWGGYPPGEEYFQHWGRGTLVIVSSASTKG
PSVFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWLNGKEYKCKVSNK
ALPAPIEKTI SKAKGQPREPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLSPGK
10E8v4-V5R-100cF-HC (Heavy Chain)
SEQ ID NO: 770
EVRLRESGGGLVKPGGSLRLSCSASGFDEDNAWMTWVRQPPGKGLEWVGRITGPGEGWSVDYAESVKG
RFTISRDNTKNTLYLEMNNVRTEDTGYYFCARTGKYYDFWFGYPPGEEYFQDWGQGTLVIVSSASTKG
PSVFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWLNGKEYKCKVSNK
ALPAPIEKTISKAKGQPREPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLSPGK
10E8v2-LC (light chain)
SEQ ID NO: 771
ASELTQDPAVSVALKQTVTITCRGDSLRSHYASWYQKKPGQAPILLFYGKNNRPSGVPDRFSGSASGN
RASLTISGAQAEDDAEYYCSSRDKSGSRLSVFGGGTKLTVLSQPKAAPSVTLFPPSSEELQANKATLV
CLISDFYPGAVTVAWKADSSPVKAGVETTTPSKQSNNKYAASSYLSLTPEQWKSHRSYSCQVTHEGST
VEKTVAPTECS
10E8v3-LC (light chain)
SEQ ID NO: 772
ASELTQETGVSVALGRTVTITCRGDSLRSHYASWYQKKPGQAPILLFYGKNNRPSGIHDRFSGSASGN
RASLTISGAQAEDDAEYYCSSRDKSGSRLSVFGGGTKLTVLSQPKAAPSVTLFPPSSEELQANKATLV
CLISDFYPGAVTVAWKADSSPVKAGVETTTPSKQSNNKYAASSYLSLTPEQWKSHRSYSCQVTHEGST
VEKTVAPTECS

1.-46. (canceled)

47. An antigen binding polypeptide comprising a first and second polypeptide domain wherein the first polypeptide domain is (i) a CD4 binding polypeptide or (ii) a fibronectin-based scaffold polypeptide that binds the N17 domain of gp41 and the second polypeptide domain is an anti-HIV broadly neutralizing antibody (bnAb) polypeptide or a fragment thereof.

48. An antigen binding polypeptide according to claim 47, wherein the first polypeptide domain is a fibronectin-based scaffold polypeptide that binds CD4.

49. An antigen binding polypeptide comprising a first, second and third polypeptide domain wherein the first polypeptide domain is a fibronectin-based scaffold polypeptide that binds CD4, the second polypeptide domain is a fibronectin-based scaffold polypeptide that binds the N17 domain of gp41, and the third polypeptide domain is an anti-HIV bnAb or fragment thereof.

50. The antigen binding polypeptide according to claim 48, wherein the fibronectin-based scaffold polypeptides are each connected to the anti-HIV bnAb or fragment thereof by a linker.

51. The antigen binding polypeptide according to claim 50, wherein the fibronectin-based scaffold polypeptide is connected to the HC region or the LC region of the anti-HIV bnAb.

52. The antigen binding polypeptide according to claim 47, wherein the HC region of the anti-HIV bnAb comprises a sequence at least 80%, 85%, 90%, 95%, 98%, 99% or 100% identical to the sequence of SEQ ID NO: 7, SEQ ID NO: 8, SEQ ID NO: 768, SEQ ID NO: 769 or SEQ ID NO: 770.

53. The antigen binding polypeptide according to claim 50, wherein the fibronectin-based scaffold polypeptide that binds CD4 is connected to the LC of the anti-HIV bnAb and the fibronectin-based scaffold polypeptide that binds the N17 domain of gp41 is connected to the HC of the anti-HIV bnAb.

54. The antigen binding polypeptide according to claim 50, wherein the fibronectin-based scaffold polypeptide that binds CD4 is connected to the HC of the anti-HIV bnAb and the fibronectin-based scaffold polypeptide that binds the N17 domain of gp41 is connected to the LC of the anti-HIV bnAb.

55. The antigen binding polypeptide according to claim 47, wherein the LC region of the anti-HIV bnAb comprises a sequence at least 80%, 85%, 90%, 95%, 98%, 99% or 100% identical to the sequence of SEQ NO: 6, SEQ ID NO: 771 or SEQ ID NO: 772 and the HC region of the anti-HIV bnAb comprises a sequence at least 80%, 85%, 90%, 95%, 98%, 99% or 100% identical to the sequence of SEQ ID NO: 7, SEQ ID NO: 8, SEQ ID NO: 768, SEQ ID NO: 769 or SEQ ID NO: 770.

56. An antigen binding polypeptide according to any one of claim 47 further comprising an HIV Fusion peptide inhibitor.

57. An antigen binding polypeptide according to claim 56 wherein the HIV Fusion peptide inhibitor is connected to the fibronectin-based scaffold polypeptide that binds the N17 domain of gp41.

58. An antigen binding polypeptide comprising a first and second polypeptide domain wherein the first polypeptide domain is a fibronectin-based scaffold polypeptide that binds CD4 and the second polypeptide domain is (i) an anti-HIV bnAb or a fragment thereof, (ii) a CDR variant of (i) wherein the variant has 1, 2 or 3 amino acid modifications; (iii) a LC region comprising a sequence at least 80%, 85%, 90%, 95%, 98%, 99% or 100% identical to the sequence of SEQ NO: 6, SEQ ID NO: 771 or SEQ ID NO: 772; and/or (iv) a HC region comprising a sequence at least 80%, 85%, 90%, 95%, 98%, 99% or 100% identical to the sequence of SEQ ID NO: 7, SEQ ID NO: 8, SEQ ID NO: 768, SEQ ID NO: 769 or SEQ ID NO: 770.

59. The antigen binding polypeptide of claim 58 wherein the first polypeptide domain comprises a FG loop sequence or CD loop sequence at least 80%, 85%, 90%, 95%, 98%, 99% or 100% identical to any one of SEQ ID NOs: 13-71, and the second polypeptide domain comprises an anti-HIV bnAb described in Table 8 or a fragment thereof.

60. The antigen binding polypeptide of claim 58 wherein the fibronectin-based scaffold polypeptide that binds CD4 comprises an amino acid sequence at least 80%, 85%, 90%, 95%, 98%, 99% or 100% identical to any one amino acid sequence of SEQ ID NOs: 72-91.

61. The antigen binding polypeptide of any one of claim 58 wherein the anti-HIV bnAb polypeptide is selected from a class of anti-HIV bnAb selected from an Apex (V1/V2) bnAb, a N332 glycan bnAb, a CD4 binding site (CD4bs) bnAb, a gp120-gp41 interface bnAb, and a MPER bnAb or a fragment thereof.

62. The antigen binding polypeptide according to claim 61, wherein the fibronectin-based scaffold polypeptide that binds the N17 domain of gp41 comprises a sequence that is at least 80%, 85%, 90%, 95%, 98%, 99% or 100% identical to any one amino acid sequence of SEQ ID NOs: 92-348.

63. The antigen binding polypeptide of claim 61, wherein the anti-HIV bnAb or fragment thereof is selected from an N6 bnAb, a VRC01 anti-HIV bnAb and a 10E8v4 anti-HIV bnAb.

64. A pharmaceutical composition comprising an antigen binding polypeptide of claim 1, and a carrier.

65. A nucleic acid sequence which encodes the antigen binding polypeptide as defined in claim 47.

66. An expression vector comprising the nucleic acid sequence as defined in claim 65.

67. A recombinant host cell comprising the nucleic acid sequence as defined in claim 66.

68. A method for the production of the antigen binding polypeptide which method comprises culturing host cells as defined in claim 21 under conditions suitable for expression of said nucleic acid sequence or vector, whereby an antigen binding polypeptide comprising a CD4 binding polypeptide and an anti-HIV bnAb polypeptide is produced.

69. A method of treating HIV in a subject comprising administering an effective amount of the antigen binding polypeptide or pharmaceutical composition thereof according to claim 47.

70. The antigen binding polypeptide of claim 48 wherein the first polypeptide domain comprises a FG loop sequence or CD loop sequence at least 80%, 85%, 90%, 95%, 98%, 99% or 100% identical to any one of SEQ ID NOs: 13-71, and the second polypeptide domain comprises an anti-HIV bnAb described in Table 8 or a fragment thereof.

71. The antigen binding polypeptide of claim 48 wherein the fibronectin-based scaffold polypeptide that binds CD4 comprises an amino acid sequence at least 80%, 85%, 90%, 95%, 98%, 99% or 100% identical to any one amino acid sequence of SEQ ID NOs: 72-91.

72. The antigen binding polypeptide of any one of claim 1 wherein the anti-HIV bnAb polypeptide is selected from a class of anti-HIV bnAb selected from an Apex (V1/V2) bnAb, a N332 glycan bnAb, a CD4 binding site (CD4bs) bnAb, a gp120-gp41 interface bnAb, and a MPER bnAb or a fragment thereof.
